SEQUENCE LISTING (1104 sequences)

SEQ ID NO: 1-500 - skipped (500 entries)

SEQ ID NO: 501 - protein, 5 aa, synthetic construct
TYWMH

SEQ ID NO: 502 - protein, 17 aa, synthetic construct
NIYPGTGGSNFDEKFKN

SEQ ID NO: 503 - protein, 8 aa, synthetic construct
WTTGTGAY

SEQ ID NO: 504 - protein, 7 aa, synthetic construct
GYTFTTY

SEQ ID NO: 505 - protein, 6 aa, synthetic construct
YPGTGG

SEQ ID NO: 506 - protein, 117 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 507 - DNA, 351 bp, synthetic construct
  source 1..351: mol_type = other DNA
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 508 - protein, 443 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 509 - DNA, 1329 bp, synthetic construct
  source 1..1329: mol_type = other DNA
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 510 - protein, 17 aa, synthetic construct
KSSQSLLDSGNQKNFLT

SEQ ID NO: 511 - protein, 7 aa, synthetic construct
WASTRES

SEQ ID NO: 512 - protein, 9 aa, synthetic construct
QNDYSYPYT

SEQ ID NO: 513 - protein, 13 aa, synthetic construct
SQSLLDSGNQKNF

SEQ ID NO: 514 - skipped

SEQ ID NO: 515 - protein, 6 aa, synthetic construct
DYSYPY

SEQ ID NO: 516 - protein, 113 aa, synthetic construct
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 517 - DNA, 339 bp, synthetic construct
  source 1..339: mol_type = other DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 518 - protein, 220 aa, synthetic construct
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - DNA, 660 bp, synthetic construct
  source 1..660: mol_type = other DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 520 - protein, 113 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 521 - DNA, 339 bp, synthetic construct
  source 1..339: mol_type = other DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 522 - protein, 220 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 523 - DNA, 660 bp, synthetic construct
  source 1..660: mol_type = other DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 524 - DNA, 15 bp, synthetic construct
  source 1..15: mol_type = other DNA
acctactggatgcac

SEQ ID NO: 525 - DNA, 51 bp, synthetic construct
  source 1..51: mol_type = other DNA
aatatctaccccggcaccggcggctctaacttcgacgagaagtttaagaat

SEQ ID NO: 526 - DNA, 24 bp, synthetic construct
  source 1..24: mol_type = other DNA
tggactaccggcacaggcgcctac

SEQ ID NO: 527 - DNA, 21 bp, synthetic construct
  source 1..21: mol_type = other DNA
ggctacaccttcactacctac

SEQ ID NO: 528 - DNA, 18 bp, synthetic construct
  source 1..18: mol_type = other DNA
taccccggcaccggcggc

SEQ ID NO: 529 - DNA, 51 bp, synthetic construct
  source 1..51: mol_type = other DNA
aaatctagtcagtcactgctggatagcggtaatcagaagaacttcctgacc

SEQ ID NO: 530 - DNA, 21 bp, synthetic construct
  source 1..21: mol_type = other DNA
tgggcctctactagagaatca

SEQ ID NO: 531 - DNA, 27 bp, synthetic construct
  source 1..27: mol_type = other DNA
cagaacgactatagctacccctacacc

SEQ ID NO: 532 - DNA, 39 bp, synthetic construct
  source 1..39: mol_type = other DNA
agtcagtcactgctggatagcggtaatcagaagaacttc

SEQ ID NO: 533 - skipped

SEQ ID NO: 534 - DNA, 18 bp, synthetic construct
  source 1..18: mol_type = other DNA
gactatagctacccctac

SEQ ID NO: 535 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 536 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 537 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 538 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 539 - protein, 447 aa, synthetic construct
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 213 aa, synthetic construct
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 541 - protein, 10 aa, synthetic construct
GYTFTTYWMH

SEQ ID NO: 542-600 - skipped (59 entries)

SEQ ID NO: 601 - protein, 5 aa, synthetic construct
SYWMY

SEQ ID NO: 602 - protein, 17 aa, synthetic construct
RIDPNSGSTKYNEKFKN

SEQ ID NO: 603 - protein, 11 aa, synthetic construct
DYRKGLYAMDY

SEQ ID NO: 604 - protein, 7 aa, synthetic construct
GYTFTSY

SEQ ID NO: 605 - protein, 6 aa, synthetic construct
DPNSGS

SEQ ID NO: 606 - protein, 120 aa, synthetic construct
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 607 - DNA, 360 bp, synthetic construct
  source 1..360: mol_type = other DNA
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttca

SEQ ID NO: 608 - protein, 446 aa, synthetic construct
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 609 - protein, 11 aa, synthetic construct
KASQDVGTAVA

SEQ ID NO: 610 - protein, 7 aa, synthetic construct
WASTRHT

SEQ ID NO: 611 - protein, 9 aa, synthetic construct
QQYNSYPLT

SEQ ID NO: 612 - protein, 7 aa, synthetic construct
SQDVGTA

SEQ ID NO: 613 - skipped

SEQ ID NO: 614 - protein, 6 aa, synthetic construct
YNSYPL

SEQ ID NO: 615 - DNA, 1338 bp, synthetic construct
  source 1..1338: mol_type = other DNA
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 616 - protein, 107 aa, synthetic construct
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 617 - DNA, 321 bp, synthetic construct
  source 1..321: mol_type = other DNA
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaag

SEQ ID NO: 618 - protein, 214 aa, synthetic construct
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 619 - DNA, 642 bp, synthetic construct
  source 1..642: mol_type = other DNA
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 620 - protein, 120 aa, synthetic construct
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 621 - DNA, 360 bp, synthetic construct
  source 1..360: mol_type = other DNA
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttca

SEQ ID NO: 622 - protein, 446 aa, synthetic construct
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 623 - DNA, 1338 bp, synthetic construct
  source 1..1338: mol_type = other DNA
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 624 - protein, 107 aa, synthetic construct
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 625 - DNA, 321 bp, synthetic construct
  source 1..321: mol_type = other DNA
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaag

SEQ ID NO: 626 - protein, 214 aa, synthetic construct
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 627 - DNA, 642 bp, synthetic construct
  source 1..642: mol_type = other DNA
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 628 - DNA, 15 bp, synthetic construct
  source 1..15: mol_type = other DNA
agctactggatgtac

SEQ ID NO: 629 - DNA, 51 bp, synthetic construct
  source 1..51: mol_type = other DNA
agaatcgaccctaatagcggctctactaagtataacgagaagtttaagaat

SEQ ID NO: 630 - DNA, 33 bp, synthetic construct
  source 1..33: mol_type = other DNA
gactatagaaagggcctgtacgctatggactac

SEQ ID NO: 631 - DNA, 21 bp, synthetic construct
  source 1..21: mol_type = other DNA
ggctacaccttcactagctac

SEQ ID NO: 632 - DNA, 18 bp, synthetic construct
  source 1..18: mol_type = other DNA
gaccctaatagcggctct

SEQ ID NO: 633 - DNA, 33 bp, synthetic construct
  source 1..33: mol_type = other DNA
aaagcctctcaggacgtgggcaccgccgtggcc

SEQ ID NO: 634 - DNA, 21 bp, synthetic construct
  source 1..21: mol_type = other DNA
tgggcctctactagacacacc

SEQ ID NO: 635 - DNA, 27 bp, synthetic construct
  source 1..27: mol_type = other DNA
cagcagtataatagctaccccctgacc

SEQ ID NO: 636 - DNA, 21 bp, synthetic construct
  source 1..21: mol_type = other DNA
tctcaggacgtgggcaccgcc

SEQ ID NO: 637 - skipped

SEQ ID NO: 638 - DNA, 18 bp, synthetic construct
  source 1..18: mol_type = other DNA
tataatagctaccccctg

SEQ ID NO: 639 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 640 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 641 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 642 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 643 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 644 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 645 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 646 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 647 - protein, 10 aa, synthetic construct
GYTFTSYWMY

SEQ ID NO: 648-700 - skipped (53 entries)

SEQ ID NO: 701 - protein, 5 aa, synthetic construct
NYGMN

SEQ ID NO: 702 - protein, 17 aa, synthetic construct
WINTDTGEPTYADDFKG

SEQ ID NO: 703 - protein, 16 aa, synthetic construct
NPPYYYGTNNAEAMDY

SEQ ID NO: 704 - protein, 7 aa, synthetic construct
GFTLTNY

SEQ ID NO: 705 - protein, 6 aa, synthetic construct
NTDTGE

SEQ ID NO: 706 - protein, 125 aa, synthetic construct
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 707 - DNA, 375 bp, synthetic construct
  source 1..375: mol_type = other DNA
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagc

SEQ ID NO: 708 - DNA, 375 bp, synthetic construct
  source 1..375: mol_type = other DNA
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 709 - protein, 451 aa, synthetic construct
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 710 - protein, 11 aa, synthetic construct
SSSQDISNYLN

SEQ ID NO: 711 - protein, 7 aa, synthetic construct
YTSTLHL

SEQ ID NO: 712 - protein, 9 aa, synthetic construct
QQYYNLPWT

SEQ ID NO: 713 - protein, 7 aa, synthetic construct
SQDISNY

SEQ ID NO: 714 - skipped

SEQ ID NO: 715 - protein, 6 aa, synthetic construct
YYNLPW

SEQ ID NO: 716 - DNA, 1353 bp, synthetic construct
  source 1..1353: mol_type = other DNA
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagcgcgtccactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 717 - DNA, 1353 bp, synthetic construct
  source 1..1353: mol_type = other DNA
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 718 - protein, 107 aa, synthetic construct
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 719 - DNA, 321 bp, synthetic construct
  source 1..321: mol_type = other DNA
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaag

SEQ ID NO: 720 - DNA, 321 bp, synthetic construct
  source 1..321: mol_type = other DNA
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaag

SEQ ID NO: 721 - protein, 214 aa, synthetic construct
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 722 - DNA, 642 bp, synthetic construct
  source 1..642: mol_type = other DNA
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 723 - DNA, 642 bp, synthetic construct
  source 1..642: mol_type = other DNA
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 724 - protein, 125 aa, synthetic construct
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 725 - DNA, 375 bp, synthetic construct
  source 1..375: mol_type = other DNA
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagc

SEQ ID NO: 726 - DNA, 375 bp, synthetic construct
  source 1..375: mol_type = other DNA
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 727 - protein, 451 aa, synthetic construct
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 728 - DNA, 1353 bp, synthetic construct
  source 1..1353: mol_type = other DNA
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 729 - DNA, 1353 bp, synthetic construct
  source 1..1353: mol_type = other DNA
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 730 - protein, 107 aa, synthetic construct
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 731 - DNA, 321 bp, synthetic construct
  source 1..321: mol_type = other DNA
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaag

SEQ ID NO: 732 - DNA, 321 bp, synthetic construct
  source 1..321: mol_type = other DNA
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaag

SEQ ID NO: 733 - protein, 214 aa, synthetic construct
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 734 - DNA, 642 bp, synthetic construct
  source 1..642: mol_type = other DNA
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 735 - DNA, 642 bp, synthetic construct
  source 1..642: mol_type = other DNA
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 736 - DNA, 15 bp, synthetic construct
  source 1..15: mol_type = other DNA
aattacgggatgaac

SEQ ID NO: 737 - DNA, 15 bp, synthetic construct
  source 1..15: mol_type = other DNA
aactacggcatgaac

SEQ ID NO: 738 - DNA, 51 bp, synthetic construct
  source 1..51: mol_type = other DNA
tggattaacaccgacaccggggagcctacctacgcggacgatttcaaggga

SEQ ID NO: 739 - DNA, 51 bp, synthetic construct
  source 1..51: mol_type = other DNA
tggatcaacaccgacaccggcgagcctacctacgccgacgacttcaagggc

SEQ ID NO: 740 - DNA, 48 bp, synthetic construct
  source 1..48: mol_type = other DNA
aacccgccctactactacggaaccaacaacgccgaagccatggactac

SEQ ID NO: 741 - DNA, 48 bp, synthetic construct
  source 1..48: mol_type = other DNA
aaccccccttactactacggcaccaacaacgccgaggccatggactat

SEQ ID NO: 742 - DNA, 21 bp, synthetic construct
  source 1..21: mol_type = other DNA
ggattcaccctcaccaattac

SEQ ID NO: 743 - DNA, 21 bp, synthetic construct
  source 1..21: mol_type = other DNA
ggcttcaccctgaccaactac

SEQ ID NO: 744 - DNA, 18 bp, synthetic construct
  source 1..18: mol_type = other DNA
aacaccgacaccggggag

SEQ ID NO: 745 - DNA, 18 bp, synthetic construct
  source 1..18: mol_type = other DNA
aacaccgacaccggcgag

SEQ ID NO: 746 - DNA, 33 bp, synthetic construct
  source 1..33: mol_type = other DNA
agctctagtcaggatatctctaactacctgaac

SEQ ID NO: 747 - DNA, 33 bp, synthetic construct
  source 1..33: mol_type = other DNA
tcctccagccaggacatctccaactacctgaac

SEQ ID NO: 748 - DNA, 21 bp, synthetic construct
  source 1..21: mol_type = other DNA
tacactagcaccctgcacctg

SEQ ID NO: 749 - DNA, 21 bp, synthetic construct
  source 1..21: mol_type = other DNA
tacacctccaccctgcacctg

SEQ ID NO: 750 - DNA, 27 bp, synthetic construct
  source 1..27: mol_type = other DNA
cagcagtactataacctgccctggacc

SEQ ID NO: 751 - DNA, 27 bp, synthetic construct
  source 1..27: mol_type = other DNA
cagcagtactacaacctgccctggacc

SEQ ID NO: 752 - DNA, 21 bp, synthetic construct
  source 1..21: mol_type = other DNA
agtcaggatatctctaactac

SEQ ID NO: 753 - DNA, 21 bp, synthetic construct
  source 1..21: mol_type = other DNA
agccaggacatctccaactac

SEQ ID NO: 754-755 - skipped (2 entries)

SEQ ID NO: 756 - DNA, 18 bp, synthetic construct
  source 1..18: mol_type = other DNA
tactataacctgccctgg

SEQ ID NO: 757 - DNA, 18 bp, synthetic construct
  source 1..18: mol_type = other DNA
tactacaacctgccctgg

SEQ ID NO: 758 - DNA, 15 bp, synthetic construct
  source 1..15: mol_type = other DNA
aactacgggatgaac

SEQ ID NO: 759 - DNA, 51 bp, synthetic construct
  source 1..51: mol_type = other DNA
tggattaacaccgacaccggcgagcctacctacgccgacgactttaagggc

SEQ ID NO: 760 - DNA, 48 bp, synthetic construct
  source 1..48: mol_type = other DNA
aaccccccctactactacggcactaacaacgccgaggctatggactac

SEQ ID NO: 761 - DNA, 21 bp, synthetic construct
  source 1..21: mol_type = other DNA
ggcttcaccctgactaactac

SEQ ID NO: 762 - protein, 447 aa, synthetic construct
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 763 - protein, 214 aa, synthetic construct
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 764 - protein, 446 aa, synthetic construct
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 765 - protein, 220 aa, synthetic construct
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 766 - protein, 10 aa, synthetic construct
GFTLTNYGMN

SEQ ID NO: 767-800 - skipped (34 entries)

SEQ ID NO: 801 - protein, 5 aa, synthetic construct
SYNMH

SEQ ID NO: 802 - protein, 17 aa, synthetic construct
DIYPGNGDTSYNQKFKG

SEQ ID NO: 803 - protein, 9 aa, synthetic construct
VGGAFPMDY

SEQ ID NO: 804 - protein, 7 aa, synthetic construct
GYTFTSY

SEQ ID NO: 805 - protein, 6 aa, synthetic construct
YPGNGD

SEQ ID NO: 806 - protein, 118 aa, synthetic construct
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 807 - DNA, 354 bp, synthetic construct
  source 1..354: mol_type = other DNA
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 808 - protein, 444 aa, synthetic construct
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 809 - DNA, 1332 bp, synthetic construct
  source 1..1332: mol_type = other DNA
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 810 - protein, 15 aa, synthetic construct
RASESVEYYGTSLMQ

SEQ ID NO: 811 - protein, 7 aa, synthetic construct
AASNVES

SEQ ID NO: 812 - protein, 9 aa, synthetic construct
QQSRKDPST

SEQ ID NO: 813 - protein, 11 aa, synthetic construct
SESVEYYGTSL

SEQ ID NO: 814 - skipped

SEQ ID NO: 815 - protein, 6 aa, synthetic construct
SRKDPS

SEQ ID NO: 816 - protein, 111 aa, synthetic construct
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 817 - DNA, 333 bp, synthetic construct
  source 1..333: mol_type = other DNA
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 818 - protein, 218 aa, synthetic construct
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 819 - DNA, 654 bp, synthetic construct
  source 1..654: mol_type = other DNA
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 820 - protein, 17 aa, synthetic construct
DIYPGQGDTSYNQKFKG

SEQ ID NO: 821 - protein, 6 aa, synthetic construct
YPGQGD

SEQ ID NO: 822 - protein, 118 aa, synthetic construct
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 823 - DNA, 354 bp, synthetic construct
  source 1..354: mol_type = other DNA
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 824 - protein, 444 aa, synthetic construct
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 825 - DNA, 1332 bp, synthetic construct
  source 1..1332: mol_type = other DNA
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 826 - protein, 111 aa, synthetic construct
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 827 - DNA, 333 bp, synthetic construct
  source 1..333: mol_type = other DNA
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 828 - protein, 218 aa, synthetic construct
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 829 - DNA, 654 bp, synthetic construct
  source 1..654: mol_type = other DNA
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 830 - protein, 114 aa, synthetic construct
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 831 - protein, 108 aa, synthetic construct
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 832 - protein, 120 aa, synthetic construct
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 833 - protein, 113 aa, synthetic construct
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 834-900 - skipped (67 entries)

SEQ ID NO: 901 - protein, 121 aa, synthetic construct
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 902 - protein, 107 aa, synthetic construct
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 903 - protein, 451 aa, synthetic construct
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 904 - protein, 214 aa, synthetic construct
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 905 - DNA, 363 bp, synthetic construct
  source 1..363: mol_type = other DNA
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctcc

SEQ ID NO: 906 - DNA, 321 bp, synthetic construct
  source 1..321: mol_type = other DNA
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaag

SEQ ID NO: 907 - DNA, 1353 bp, synthetic construct
  source 1..1353: mol_type = other DNA
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctccgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 908 - DNA, 642 bp, synthetic construct
  source 1..642: mol_type = other DNA
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 909 - protein, 5 aa, synthetic construct
SYGVD

SEQ ID NO: 910 - protein, 7 aa, synthetic construct
GFSLSSY

SEQ ID NO: 911 - protein, 16 aa, synthetic construct
VIWGGGGTYYASSLMG

SEQ ID NO: 912 - protein, 5 aa, synthetic construct
WGGGG

SEQ ID NO: 913 - protein, 13 aa, synthetic construct
HAYGHDGGFAMDY

SEQ ID NO: 914 - protein, 11 aa, synthetic construct
RASESVSSNVA

SEQ ID NO: 915 - protein, 7 aa, synthetic construct
SESVSSN

SEQ ID NO: 916 - protein, 7 aa, synthetic construct
GASNRAT

SEQ ID NO: 917 - skipped

SEQ ID NO: 918 - protein, 9 aa, synthetic construct
GQSYSYPFT

SEQ ID NO: 919 - protein, 6 aa, synthetic construct
SYSYPF

SEQ ID NO: 920 - protein, 124 aa, synthetic construct
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 921 - protein, 107 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 922-1000 - skipped (79 entries)

SEQ ID NO: 1001 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1002 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 1003 - protein, 114 aa, synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 1004 - protein, 297 aa, synthetic construct
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1005 - protein, 114 aa, Homo sapiens
  VARIANT 93: note = Xaa = Glu or Lys
NWVNVISDLKKIEDLIQSMHIDATLYTESDVHPSCKVTAMKCFLLELQVISLESGDASIHDTVENLIILANNSLSSNGNVTESGCKECEELEXKNIKEFLQSFVHIVQMFINTS

SEQ ID NO: 1006 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 1007-1100 - skipped (94 entries)

SEQ ID NO: 1101 - protein, 121 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLWEVRALPSVYWGQGTLVTVSS

SEQ ID NO: 1102 - protein, 109 aa, synthetic construct
SYELTQPPSVSVAPGQTARITCGANDIGSKSVHWYQQKAGQAPVLVVSEDIIRPSGIPERISGSNSGNTATLTISRVEAGDEADYYCQVWDRDSDQYVFGTGTKVTVLG

SEQ ID NO: 1103 - protein, 447 aa, synthetic construct
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1104 - protein, 215 aa, synthetic construct
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0034259 A1
(43) Pub. Date: Jan. 30, 2025

(54) Dosage Regimens For Anti-Pd-L1 Antibodies And Uses Thereof

(71) Applicant: Novartis AG, Basel (CH)

(72) Inventors: Andrew Marc Stein, Cambridge, MA (US); Kulandayan Kasi Subramanian, Cambridge, MA (US)

(21) Appl. No.: 18/904,819

(22) Filed: Oct. 2, 2024

Related U.S. Application Data
(63) Continuation of application No. 17/046,444, filed on Oct. 9, 2020.
(60) Provisional application No. 62/657,141, filed on Apr. 13, 2018.

(51) Int. Cl. C07K 16/28; A61K 38/17; A61K 39/00; A61K 39/395; A61P 35/00; C07K 16/30

(52) U.S. Cl. CPC C07K 16/2827; A61K 38/1774; A61K 39/3955; A61P 35/00; C07K 16/2896; C07K 16/30; A61K 2039/505; C07K 2317/51; C07K 2317/515; C07K 2317/56; C07K 2317/565; C07K 2317/76

(57) ABSTRACT

Antibody molecules that specifically bind to PD-L1 are disclosed. Combination therapies comprising the anti-PD-L1 antibody molecules are also disclosed. The anti-PD-L1 antibody molecules can be used to treat, prevent and/or diagnose cancerous or infectious conditions and disorders.

### CROSS REFERENCE TO RELATED APPLICATIONS

This application is a continuation application of U.S. application Ser. No. 17/046,444, filed Oct. 9, 2020, which is the national stage entry of International Patent Application No. PCT/US2019/027175, filed on Apr. 12, 2019, and claims the benefit of U.S. Provisional Application No. 62/657,141, filed Apr. 13, 2018. The contents of the aforementioned applications are hereby incorporated by reference in their entirety.

### SEQUENCE LISTING

This application contains a Sequence Listing that has been submitted electronically as an XML file named 45607-0012002_ST26_SL.XML. The XML file, created on Sep. 25, 2024, is 404,101 bytes in size. The material in the XML file is hereby incorporated by reference in its entirety.

### BACKGROUND

The ability of T cells to mediate an immune response against an antigen requires two distinct signaling interactions (Viglietta, V. et al. (2007) Neurotherapeutics 4:666-675; Korman, A. J. et al. (2007) Adv. Immunol. 90:297-339). First, an antigen that has been arrayed on the surface of antigen-presenting cells (APC) is presented to an antigen-specific naive CD4⁺ T cell. Such presentation delivers a signal via the T cell receptor (TCR) that directs the T cell to initiate an immune response specific to the presented antigen. Second, various co-stimulatory and inhibitory signals mediated through interactions between the APC and distinct T cell surface molecules trigger the activation and proliferation of the T cells and ultimately their inhibition.

The immune system is tightly controlled by a network of costimulatory and co-inhibitory ligands and receptors. These molecules provide the second signal for T cell activation and provide a balanced network of positive and negative signals to maximize immune responses against infection, while limiting immunity to self (Wang, L. et al. (Epub Mar. 7, 2011) J. Exp. Med. 208(3):577-92; Lepenies, B. et al. (2008) Endocrine, Metabolic & Immune Disorders—Drug Targets 8:279-288). Examples of costimulatory signals include the binding between the B7.1 (CD80) and B7.2 (CD86) ligands of the APC and the CD28 and CTLA-4 receptors of the CD4⁺ T-lymphocyte (Sharpe, A. H. et al. (2002) Nature Rev. Immunol. 2:116-126; Lindley, P. S. et al. (2009) Immunol. Rev. 229:307-321). Binding of B7.1 or B7.2 to CD28 stimulates T cell activation, whereas binding of B7.1 or B7.2 to CTLA-4 inhibits such activation (Dong, C. et al. (2003) Immunolog. Res. 28(1):39-48; Greenwald, R. J. et al. (2005) Ann. Rev. Immunol. 23:515-548). CD28 is constitutively expressed on the surface of T cells (Gross, J., et al. (1992) J. Immunol. 149:380-388), whereas CTLA-4 expression is rapidly up-regulated following T-cell activation (Linsley, P. et al. (1996) Immunity 4:535-543).

Other ligands of the CD28 receptor include a group of related B7 molecules, also known as the “B7 Superfamily” (Coyle, A. J. et al. (2001) Nature Immunol. 2(3):203-209; Sharpe, A. H. et al. (2002) Nature Rev. Immunol. 2:116-126; Collins, M. et al. (2005) Genome Biol. 6:223.1-223.7; Korman, A. J. et al. (2007) Adv. Immunol. 90:297-339). Several members of the B7 Superfamily are known, including B7.1 (CD80), B7.2 (CD86), the inducible co-stimulator ligand (ICOS-L), the programmed death-1 ligand (PD-L1; B7-H1), the programmed death-2 ligand (PD-L2; B7-DC), B7-H3, B7-H4, and B7-H6 (Collins, M. et al. (2005) Genome Biol. 6:223.1-223.7).

The Programmed Death 1 (PD-1) protein is an inhibitory member of the extended CD28/CTLA-4 family of T cell regulators (Okazaki et al. (2002) Curr Opin Immunol 14: 391779-82; Bennett et al. (2003) J. Immunol. 170:711-8). Other members of the CD28 family include CD28, CTLA-4, ICOS, and BTLA. Two cell surface glycoprotein ligands for PD-1 have been identified, Program Death Ligand 1 (PD-L1) and Program Death Ligand 2 (PD-L2). PD-L1 and PD-L2 have been shown to downregulate T cell activation and cytokine secretion upon binding to PD-1 (Freeman et al. (2000) J Exp Med 192:1027-34; Latchman et al. (2001) Nat Immunol 2:261-8; Carter et al. (2002) Eur J Immunol 32:634-43; Ohigashi et al. (2005) Clin Cancer Res 11:2947-53).

PD-L1 (also known as cluster of differentiation 274 (CD274) or B7 homolog 1 (B7-H1)) is a 40 kDa type 1 transmembrane protein. PD-L1 binds to its receptor, PD-1, found on activated T cells, B cells, and myeloid cells, to modulate activation or inhibition. Both PD-L1 and PD-L2 are B7 homologs that bind to PD-1, but do not bind to CD28 or CTLA-4 (Blank et al. (2005) Cancer Immunol Immunother. 54:307-14). Binding of PD-L1 with its receptor PD-1 on T cells delivers a signal that inhibits TCR-mediated activation of IL-2 production and T cell proliferation. The mechanism involves inhibition of ZAP70 phosphorylation and its association with CD3ζ (Sheppard et al. (2004) FEBSLett. 574:37-41). PD-1 signaling attenuates PKC-θ activation loop phosphorylation resulting from TCR signaling, necessary for the activation of transcription factors NF-κB and AP-1, and for production of IL-2. PD-L1 also binds to the costimulatory molecule CD80 (B7-1), but not CD86 (B7-2) (Butte et al. (2008) Mol Immunol. 45:3567-72).

Expression of PD-L1 on the cell surface has been shown to be upregulated through IFN-γ stimulation. PD-L1 expression has been found in many cancers, including human lung, ovarian and colon carcinoma, and various myelomas, and is often associated with poor prognosis (Iwai et al. (2002) PNAS 99:12293-7; Ohigashi et al. (2005) Clin Cancer Res 11:2947-53; Okazaki et al. (2007) Intern. Immun. 19:813-24; Thompson et al. (2006) Cancer Res. 66:3381-5). PD-L1 has been suggested to play a role in tumor immunity by increasing apoptosis of antigen-specific T-cell clones (Dong et al. (2002) Nat Med 8:793-800). It has also been suggested that PD-L1 might be involved in intestinal mucosal inflammation and that inhibition of PD-L1 suppresses wasting disease associated with colitis (Kanai et al. (2003) J Immunol 171:4156-63).

Therefore, the need exits for novel therapeutic approaches that regulate PD-L1 functions and the functions of PD-L1 expressing cells, including dosage regimens and formulations for anti-PD-L1 antibody molecules to treat diseases, such as cancer.

### SUMMARY

Disclosed herein, at least in part, are antibody molecules (e.g., humanized antibody molecules) that bind to Program Death Ligand 1 (PD-L1) with high affinity and specificity. Pharmaceutical compositions and dose formulations comprising the anti-PD-L1 antibody molecules are also provided. The anti-PD-L1 antibody molecules disclosed herein can be used (alone or in combination with other therapeutic agents, procedures, or modalities) to treat or prevent disorders, such as cancerous disorders (e.g., solid tumors and hematological cancers), as well as infectious diseases (e.g., chronic infectious disorders or sepsis). Thus, methods, including dosage regimens, for treating various disorders using the anti-PD-L1 antibody molecules are disclosed herein. In certain embodiments, the anti-PD-L1 antibody molecule is administered or used at a flat or fixed dose.

Accordingly, in one aspect, the disclosure features a method of treating (e.g., inhibiting, reducing, ameliorating, or preventing) a disorder, e.g., a hyperproliferative condition or disorder (e.g., a cancer) in a subject.

In certain embodiments, the method includes administering to the subject an anti-PD-L1 antibody 25 molecule, e.g., an anti-PD-L1 antibody molecule described herein, at a dose of about 1000 mg to about 1400 mg or about 1400 mg to about 1900 mg, once every three or once every four weeks.

In certain embodiments, the anti-PD-L1 antibody molecule is administered at a dose of about 1000 mg to about 1400 mg once every three or once every four weeks. In other embodiments, the anti-PD-L1 antibody molecule is administered at a dose of about 1400 mg to about 1900 mg once every three or once every four weeks.

In some embodiments, the anti-PD-L1 antibody molecule is administered at a dose of about 1000 mg to about 1400 mg, e.g., about 1100 mg to about 1400 mg, about 1100 mg to about 1300 mg, about 1000 mg to about 1200 mg, about 1000 mg to about 1300 mg, about about 1200 mg to about 1400 mg, about 1000 mg to about 1300 mg, about about 1100 mg to about 1200 mg, or about 1200 mg to about 1300 mg, e.g., about 1000 mg, about 1100 mg, about 1200 mg, about 1300 mg, or about 1400 mg, once every three weeks. In certain embodiments, the anti-PD-L1 antibody molecule is administered at a dose of about 1100 mg to about 1300 mg, e.g., about 1200 mg, once every three weeks.

In some embodiments, the anti-PD-L1 antibody molecule is administered at a dose of about 1400 mg to about 1900 mg, e.g., about 1400 mg to about 1600 mg, about 1500 mg to about 1800 mg, about 1600 mg to about 1800 mg, about 1400 mg to about 1800 mg, about 1600 mg to about 1900 mg, about 1500 mg to about 1900 mg, about 1600 mg to about 1700 mg, about 1400 mg to about 1700 mg, or about 1500 mg to about 1700 mg, e.g., about 1400 mg, about 1500 mg, about 1600 mg, about 1700 mg, about 1800 mg, or about 1900 mg, once every four weeks. In certain embodiments, the anti-PD-L1 antibody molecule is administered at a dose of about 1500 mg to about 1700 mg, e.g., about 1600 mg, once every four weeks.

PD-L1 can exist as a membrane bound protein that is expressed on a wide range of tumors or in the sysmic circulation in a soluble form. The anti-PD-L1 antibody molecules described herein can bind to both soluble and membrane PD-L1. Without wishing to be bound by theory, it is believed that in some embodiments, administration of the anti-PD-L1 antibody molecule can increase total sPD-L1 (i.e., free sPD-L1 and sPD-L1-anti-PD-L1 antibody molecule complexes), due to binding of sPD-L1 by the anti-PD-L1 antibody molecule. In some embodiments, the total sPD-L1 concentration (e.g., serum concentration) in the subject after administration of the PD-L1 antibody molecule is increased by at least 2, 3, 4, 5, 6, 7, 8, 9, 10, 20, 50, or 100-fold, compared to the total sPD-L1 concentration (e.g., serum concentration) before administration of the PD-L1 antibody molecule.

In some embodiments, the anti-PD-L1 antibody molecule is administered at a dose or dosage schedule that results in 50% or more (e.g., 60% or more, 70% or more, 80% or more, 85% or more, 90% or more, 95% or more, 99% or more) of the soluble PD-L1 (sPD-L1) in the subject (e.g., in the blood) bound by the anti-PD-L1 antibody molecule.

In some embodiments, 85% or more of the soluble PD-L1 in a serum sample from the subject is bound by the anti-PD-L1 antibody molecule. In some embodiments, 90% or more of the soluble PD-L1 in a serum sample from the subject is bound by the anti-PD-L1 antibody molecule. In some embodiments, 95% or more of the soluble PD-L1 in a serum sample from the subject is bound by the anti-PD-L1 antibody molecule. In some embodiments, 99% or more of the soluble PD-L1 in a serum sample from the subject is bound by the anti-PD-L1 antibody molecule.

In some embodiments, binding of the anti-PD-L1 antibody molecule to soluble PD-L1 is determined in a blood sample (e.g., a serum sample or a plasma sample). In some embodiments, the binding of the anti-PD-L1 antibody molecule to soluble PD-L1 is determined in the cancer (e.g., a cancer sample). In some embodiments, the binding of the anti-PD-L1 antibody molecule to soluble PD-L1 is determined, e.g., measured in vitro (e.g., by ELISA or a cell-based assay) or in vivo (e.g., by imaging), or predicted from a PK/PD model, e.g., a PK/PD model described herein. In certain embodiments, the anti-PD-L1 antibody molecule is administered at a dose of about 1000 mg to about 1400 mg, e.g., about 1100 mg to about 1300 mg (e.g., about 1200 mg), once every three weeks. In some embodiments, the anti-PD-L1 antibody molecule is administered at a dose of about 1100 mg to about 1300 mg (e.g., about 1200 mg) once every three weeks.

In certain embodiments, the anti-PD-L1 antibody molecule is administered at a dose of about 1400 mg to about 1900 mg, e.g., about 1500 mg to about 1700 mg (e.g., about 1600 mg), once every four 10 weeks. In some embodiments, the anti-PD-L1 antibody molecule is administered at a dose of about 1500 mg to about 1700 mg (e.g., about 1600 mg) once every four weeks.

In some embodiments, the anti-PD-L1 antibody molecule is administered at a dose or dosage schedule that reduces the level of free soluble PD-L1 in the subject (e.g., in the blood), e.g., to 50% or less (e.g., 40% or less, 30% or less, 20% or less, 15% or less, 10% or less, 5% or less, or 1% or less) of a reference level of free soluble PD-L1.

In some embodiments, the level of free soluble PD-L1 is reduced to 15% or less of a reference level of free soluble PD-L1. In some embodiments, the level of free soluble PD-L1 is reduced to 10% or less of a reference level of free soluble PD-L1. In some embodiments, the level of free soluble PD-L1 is reduced to 5% or less of a reference level of free soluble PD-L1. In some embodiments, the level of free soluble PD-L1 is reduced to 1% or less of a reference level of free soluble PD-L1.

In some embodiments, the level of free soluble PD-L1 is determined in a blood sample (e.g., a serum sample or a plasma sample). In some embodiments, the level of free soluble PD-L1 is determined in the cancer (e.g., a cancer sample). In some embodiments, the level of free free soluble PD-L1 is determined, e.g., measured in vitro (e.g., by ELISA or a cell-based assay) or in vivo (e.g., by imaging), or predicted from a PK/PD model, e.g., a PK/PD model described herein. In some embodiments, the reference level of free soluble PD-L1 is the baseline level of free soluble PD-L1 in the subject, e.g., prior to administration of the anti-PD-L1 antibody molecule, e.g., in accordance with the dosage schedule.

In some embodiments, the anti-PD-L1 antibody molecule is administered at a dose of about 1000 mg to about 1400 mg, e.g., about 1100 mg to about 1300 mg (e.g., about 1200 mg), once every three weeks. In certain embodiments, the anti-PD-L1 antibody molecule is administered at a dose of about 1100 mg to about 1300 mg (e.g., about 1200 mg) once every three weeks.

In some embodiments, the anti-PD-L1 antibody molecule is administered at a dose of about 1400 mg to about 1900 mg, e.g., about 1500 mg to about 1700 mg (e.g., about 1600 mg), once every four weeks. In certain embodiments, the anti-PD-L1 antibody molecule is administered at a dose of about 1500 mg to about 1700 mg (e.g., about 1600 mg) once every four weeks.

In some embodiments, the anti-PD-L1 antibody molecule is administered at a dose or dosage schedule that results in 50% or more (e.g., 60% or more, 70% or more, 80% or more, 85% or more, 90% or more, 95% or more, 99% or more) of the PD-L1 in the subject (e.g., in the tumor) bound by the anti-PD-L1 antibody molecule. In some embodiments, 85% or more of the PD-L1 in the tumor is bound by the anti-PD-L1 antibody molecule. In some embodiments, 90% or more of the soluble PD-L1 in the tumor is bound by the anti-PD-L1 antibody molecule. In some embodiments, 95% or more of the PD-L1 in the tumor is bound by the anti-PD-L1 antibody molecule. In some embodiments, 99% or more of the PD-L1 in the tumor is bound by the anti-PD-L1 antibody molecule.

In some embodiments, the anti-PD-L1 antibody molecule is administered at a dose of about 1000 mg to about 1400 mg, e.g., about 1100 mg to about 1300 mg (e.g., about 1200 mg), once every three weeks. In certain embodiments, the anti-PD-L1 antibody molecule is administered at a dose of about 1100 mg to about 1300 mg (e.g., about 1200 mg) once every three weeks.

In some embodiments, the anti-PD-L1 antibody molecule is administered at a dose of about 1400 mg to about 1900 mg, e.g., about 1500 mg to about 1700 mg (e.g., about 1600 mg), once every four weeks. In certain embodiments, the anti-PD-L1 antibody molecule is administered at a dose of about 1500 mg to about 1700 mg (e.g., about 1600 mg) once every four weeks.

In some embodiments, the disorder is a cancer, e.g., a cancer described herein or a metastatic lesion thereof. In certain embodiments, the cancer is a solid tumor or a hematological cancer. In some embodiments, the cancer is a bone cancer, e.g., a chordoma. In some embodiments, the cancer is a skin cancer, e.g., a Merkel cell carcinoma or a melanoma, e.g., a cutaneous melanoma. In some embodiments, the cancer is a breast cancer, e.g., a metastatic breast carcinoma or a stage IV breast carcinoma, e.g., a triple negative breast cancer (TNBC)). In some embodiments, the cancer is a cervical cancer (e.g., a squamous cell carcinoma of the cervix). In some embodiments, the cancer is a colorectal cancer, e.g., a relapsed colorectal cancer or a metastatic colorectal cancer, e.g., a microsatellite unstable colorectal cancer, a microsatellite stable colorectal cancer, a mismatch repair proficient colorectal cancer, or a mismatch repair deficient colorectal cancer. In some embodiments, the cancer is an endometrial cancer. In some embodiments, the cancer is a lung cancer, e.g., a non-small cell lung cancer (NSCLC). In some embodiments, the cancer is an ovarian cancer. In some embodiments, the cancer is a liver cancer, e.g., a hepatocarcinoma, e.g., an advanced hepatocarcinoma. In some embodiments, the cancer is a thyroid cancer, e.g., an anaplastic thyroid cancer (ATC).

In some embodiments, the anti-PD-L1 antibody molecule is administered by infusion (e.g., intravenously or subcutaneously) at a dose (e.g., a flat dose) of about 1000 mg to about 1400 mg, e.g., about 1100 mg to about 1300 mg (e.g., about 1200 mg). In some embodiments, the anti-PD-L1 antibody molecule is administered by infusion (e.g., intravenously or subcutaneously) at a dose (e.g., a flat dose) of about 1400 mg to about 1900 mg, e.g., about 1500 mg to about 1700 mg (e.g., about 1600 mg). The dosing schedule (e.g., flat dosing schedule) can vary from e.g., once every three weeks to once every four weeks.

In one embodiment, the anti-PD-L1 antibody molecule is administered by infusion at a dose of about 1000 mg to about 1400 mg, e.g., about 1100 mg to about 1300 mg (e.g., about 1200 mg), once every three weeks.

In one embodiment, the anti-PD-L1 antibody molecule is administered by infusion at a dose of about 1400 mg to about 1900 mg, e.g., about 1500 mg to about 1700 mg (e.g., about 1600 mg), once every four weeks.

In another aspect, the disclosure features a method of reducing an activity (e.g., growth, survival, or viability, or all) of a hyperproliferative (e.g., a cancer) cell. The method includes contacting the cell with an anti-PD-L1 antibody molecule, e.g., an anti-PD-L1 antibody molecule described herein. The method can be performed in a subject, e.g., as part of a therapeutic protocol, e.g., at a dose of about 1000 mg to about 1400 mg, e.g., about 1100 mg to about 1300 mg (e.g., about 1200 mg) once every three weeks or once every four weeks. The method can be performed in a subject, e.g., as part of a therapeutic protocol, e.g., at a dose of about 1400 mg to about 1900 mg, e.g., about 1500 mg to about 1700 mg (e.g., about 1600 mg), of an anti-PD-L1 antibody molecule once every three weeks or once every four weeks. In certain embodiments, the dose is about 1000 mg to about 1400 mg, e.g., about 1100 mg to about 1300 mg (e.g., about 1200 mg) of an anti-PD-L1 antibody molecule once every three weeks. In other embodiments, the dose is about 1400 mg to about 1900 mg, e.g., about 1500 mg to about 1700 mg (e.g., about 1600 mg) of an anti-PD-L1 antibody molecule once every four weeks.

The cancer cell can be, e.g., a cell from a cancer described herein, such as a solid tumor, e.g., a bone cancer (e.g., a chordoma), a skin cancer (e.g., a Merkel cell carcinoma or a melanoma, e.g., a cutaneous melanoma), a breast cancer (e.g., a metastatic breast carcinoma or a stage IV breast carcinoma, e.g., a TNBC), a cervical cancer (e.g., a squamous cell carcinoma of the cervix), a colorectal cancer (e.g., a relapsed colorectal cancer or a metastatic colorectal cancer, e.g., a microsatellite unstable colorectal cancer, a microsatellite stable colorectal cancer, a mismatch repair proficient colorectal cancer, or a mismatch repair deficient colorectal cancer), an endometrial cancer, a lung cancer (e.g., a NSCLC), an ovarian cancer, a liver cancer (e.g., a hepatocellular carcinoma), or a thyroid cancer (e.g., an anaplastic thyroid cancer).

In certain embodiments, the cancer is a solid tumor. In some embodiments, the cancer is a bone cancer, e.g., a chordoma. In some embodiments, the cancer is a skin cancer, e.g., a Merkel cell carcinoma or a melanoma, e.g., a cutaneous melanoma. In some embodiments, the cancer is a breast cancer, e.g., a metastatic breast carcinoma or a stage IV breast carcinoma, e.g., a TNBC. In some embodiments, the cancer is a cervical cancer (e.g., a squamous cell carcinoma of the cervix). In some embodiments, the cancer is a colorectal cancer, e.g., a relapsed colorectal cancer or a metastatic colorectal cancer, e.g., a microsatellite unstable colorectal cancer, a microsatellite stable colorectal cancer, a mismatch repair proficient colorectal cancer, or a mismatch repair deficient colorectal cancer. In some embodiments, the cancer is an endometrial cancer. In some embodiments, the cancer is a lung cancer, e.g., a NSCLC. In some embodiments, the cancer is an ovarian cancer. In some embodiments, the cancer is a hepatocarcinoma, e.g., an advanced hepatocarcinoma. In some embodiments, the cancer is a thyroid cancer, e.g., an anaplastic thyroid cancer (ATC).

In certain embodiments of the methods disclosed herein, the method further includes determining the level of PD-L1 expression in tumor infiltrating lymphocytes (TILs) in the subject. In other embodiments, the level of PD-L1 expression is determined in a sample (e.g., a tumor biopsy) acquired from the subject (e.g., using immunohistochemistry). In certain embodiments, the anti-PD-L1 antibody molecule (e.g., an anti-PD-L1 antibody molecule as described herein) is administered when there is a detectable level, or an elevated level, of PD-L1 in the subject. The detection steps can also be used, e.g., to monitor the effectiveness of a therapeutic agent described herein. For example, the detection step can be used to monitor the effectiveness of the anti-PD-L1 antibody molecule.

In another aspect, the disclosure features a composition (e.g., one or more compositions or dosage forms), that includes an anti-PD-L1 antibody molecule (e.g., an anti-PD-L1 antibody molecule as described herein). Formulations, e.g., dosage formulations, and kits, e.g., therapeutic kits, that include an anti-PD-L1 antibody molecule (e.g., an anti-PD-L1 antibody molecule as described herein), are also described herein. In certain embodiments, the composition or formulation comprises about 1000 mg to about 1400 mg, e.g., about 1100 mg to about 1300 mg (e.g., about 1200 mg) of an anti-PD-L1 antibody molecule (e.g., an anti-PD-L1 antibody molecule as described herein). In certain embodiments, the composition or formulation comprises about 1400 mg to about 1900 mg, e.g., about 1500 mg to about 1700 mg (e.g., about 1600 mg), of an anti-PD-L1 antibody molecule (e.g., an anti-PD-L1 antibody molecule as described herein). In some embodiments, the composition or formulation is administered or used once every three weeks or once every four weeks. In one embodiment, the composition or formulation comprises about 1200 mg of an anti-PD-L1 antibody molecule (e.g., an anti-PD-L1 antibody molecule as described herein), and is administered or used once every three weeks. In one embodiment, the composition or formulation comprises about 1600 mg of an anti-PD-L1 antibody molecule (e.g., an anti-PD-L1 antibody molecule as described herein), and is administered or used once every four weeks. In certain embodiments, the composition or formulation is used to treat a cancer, e.g., a cancer disclosed herein or a metastatic lesion thereof.

Additional features or embodiments of the methods, compositions, dosage formulations, and kits described herein include one or more of the following.

### Antibody Molecules to PD-L1

In one embodiment, the anti-PD-L1 antibody molecule comprises at least one, two, three, four, five, or six complementarity determining regions (CDRs) (or collectively all of the CDRs) from a heavy and light chain variable region comprising an amino acid sequence shown in Table 3 (e.g., from the heavy and light chain variable region sequences of BAP058-Clone O or BAP058-Clone N disclosed in Table 3), or encoded by a nucleotide sequence shown in Table 3. In some embodiments, the CDRs are according to the Kabat definition (e.g., as set out in Table 3). In some embodiments, the CDRs are according to the Chothia definition (e.g., as set out in Table 3). In some embodiments, the CDRs are according to the combined CDR definitions of both Kabat and Chothia (e.g., as set out in Table 3). In one embodiment, the combination of Kabat and Chothia CDR of VH CDR1 comprises the amino acid sequence GYTFTSYWMY (SEQ ID NO: 647). In one embodiment, one or more of the CDRs (or collectively all of the CDRs) have one, two, three, four, five, six, or more changes, e.g., amino acid substitutions (e.g., conservative amino acid substitutions) or deletions, relative to an amino acid sequence shown in Table 3, or encoded by a nucleotide sequence shown in Table 3.

In one embodiment, the anti-PD-L1 antibody molecule comprises a heavy chain variable region (VH) comprising a VHCDR1 amino acid sequence of SEQ ID NO: 601, a VHCDR2 amino acid sequence of SEQ ID NO: 602, and a VHCDR3 amino acid sequence of SEQ ID NO: 603; and a light chain variable region (VL) comprising a VLCDR1 amino acid sequence of SEQ ID NO: 609, a VLCDR2 amino acid sequence of SEQ ID NO: 610, and a VLCDR3 amino acid sequence of SEQ ID NO: 611, each disclosed in Table 3.

In one embodiment, the anti-PD-L1 antibody molecule comprises a VH comprising a VHCDR1 encoded by the nucleotide sequence of SEQ ID NO: 628, a VHCDR2 encoded by the nucleotide sequence of SEQ ID NO: 629, and a VHCDR3 encoded by the nucleotide sequence of SEQ ID NO: 630; and a VL comprising a VLCDR1 encoded by the nucleotide sequence of SEQ ID NO: 633, a VLCDR2 encoded by the nucleotide sequence of SEQ ID NO: 634, and a VLCDR3 encoded by the nucleotide sequence of SEQ ID NO: 635, each disclosed in Table 3.

In one embodiment, the anti-PD-L1 antibody molecule comprises a VH comprising the amino acid sequence of SEQ ID NO: 606, or an amino acid sequence at least 85%, 90%, 95%, or 99% identical or higher to SEQ ID NO: 606. In one embodiment, the anti-PD-L1 antibody molecule comprises a VL comprising the amino acid sequence of SEQ ID NO: 616, or an amino acid sequence at least 85%, 90%, 95%, or 99% identical or higher to SEQ ID NO: 616. In one embodiment, the anti-PD-L1 antibody molecule comprises a VH comprising the amino acid sequence of SEQ ID NO: 620, or an amino acid sequence at least 85%, 90%, 95%, or 99% identical or higher to SEQ ID NO: 620. In one embodiment, the anti-PD-L1 antibody molecule comprises a VL comprising the amino acid sequence of SEQ ID NO: 624, or an amino acid sequence at least 85%, 90%, 95%, or 99% identical or higher to SEQ ID NO: 624. In one embodiment, the anti-PD-L1 antibody molecule comprises a VH comprising the amino acid sequence of SEQ ID NO: 606 and a VL comprising the amino acid sequence of SEQ ID NO: 616. In one embodiment, the anti-PD-L1 antibody molecule comprises a VH comprising the amino acid sequence of SEQ ID NO: 620 and a VL comprising the amino acid sequence of SEQ ID NO: 624.

In one embodiment, the antibody molecule comprises a VH encoded by the nucleotide sequence of SEQ ID NO: 607, or a nucleotide sequence at least 85%, 90%, 95%, or 99% identical or higher to SEQ ID NO: 607. In one embodiment, the antibody molecule comprises a VL encoded by the nucleotide sequence of SEQ ID NO: 617, or a nucleotide sequence at least 85%, 90%, 95%, or 99% identical or higher to SEQ ID NO: 617. In one embodiment, the antibody molecule comprises a VH encoded by the nucleotide sequence of SEQ ID NO: 621, or a nucleotide sequence at least 85%, 90%, 95%, or 99% identical or higher to SEQ ID NO: 621. In one embodiment, the antibody molecule comprises a VL encoded by the nucleotide sequence of SEQ ID NO: 625, or a nucleotide sequence at least 85%, 90%, 95%, or 99% identical or higher to SEQ ID NO: 625. In one embodiment, the antibody molecule comprises a VH encoded by the nucleotide sequence of SEQ ID NO: 607 and a VL encoded by the nucleotide sequence of SEQ ID NO: 617. In one embodiment, the antibody molecule comprises a VH encoded by the nucleotide sequence of SEQ ID NO: 621 and a VL encoded by the nucleotide sequence of SEQ ID NO: 625.

In one embodiment, the anti-PD-L1 antibody molecule comprises a heavy chain comprising the amino acid sequence of SEQ ID NO: 608, or an amino acid sequence at least 85%, 90%, 95%, or 99% identical or higher to SEQ ID NO: 608. In one embodiment, the anti-PD-L1 antibody molecule comprises a light chain comprising the amino acid sequence of SEQ ID NO: 618, or an amino acid sequence at least 85%, 90%, 95%, or 99% identical or higher to SEQ ID NO: 618. In one embodiment, the anti-PD-L1 antibody molecule comprises a heavy chain comprising the amino acid sequence of SEQ ID NO: 622, or an amino acid sequence at least 85%, 90%, 95%, or 99% identical or higher to SEQ ID NO: 622. In one embodiment, the anti-PD-L1 antibody molecule comprises a light chain comprising the amino acid sequence of SEQ ID NO: 626, or an amino acid sequence at least 85%, 90%, 95%, or 99% identical or higher to SEQ ID NO: 626. In one embodiment, the anti-PD-L1 antibody molecule comprises a heavy chain comprising the amino acid sequence of SEQ ID NO: 608 and a light chain comprising the amino acid sequence of SEQ ID NO: 618. In one embodiment, the anti-PD-L1 antibody molecule comprises a heavy chain comprising the amino acid sequence of SEQ ID NO: 622 and a light chain comprising the amino acid sequence of SEQ ID NO: 626.

In one embodiment, the antibody molecule comprises a heavy chain encoded by the nucleotide sequence of SEQ ID NO: 615, or a nucleotide sequence at least 85%, 90%, 95%, or 99% identical or higher to SEQ ID NO: 615. In one embodiment, the antibody molecule comprises a light chain encoded by the nucleotide sequence of SEQ ID NO: 619, or a nucleotide sequence at least 85%, 90%, 95%, or 99% identical or higher to SEQ ID NO: 619. In one embodiment, the antibody molecule comprises a heavy chain encoded by the nucleotide sequence of SEQ ID NO: 623, or a nucleotide sequence at least 85%, 90%, 95%, or 99% identical or higher to SEQ ID NO: 623. In one embodiment, the antibody molecule comprises a light chain encoded by the nucleotide sequence of SEQ ID NO: 627, or a nucleotide sequence at least 85%, 90%, 95%, or 99% identical or higher to SEQ ID NO: 627. In one embodiment, the antibody molecule comprises a heavy chain encoded by the nucleotide sequence of SEQ ID NO: 615 and a light chain encoded by the nucleotide sequence of SEQ ID NO: 619. In one embodiment, the antibody molecule comprises a heavy chain encoded by the nucleotide sequence of SEQ ID NO: 623 and a light chain encoded by the nucleotide sequence of SEQ ID NO: 627.

### Other Exemplary PD-L1 Inhibitors

In one embodiment, the anti-PD-L1 antibody molecule is Atezolizumab (Genentech/Roche), also known as MPDL3280A, RG7446, RO5541267, YW243.55.S70, or TECENTRIQ™. Atezolizumab and other anti-PD-L1 antibodies are disclosed in U.S. Pat. No. 8,217,149, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Atezolizuma, e.g., as disclosed in Table 4.

In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Avelumab, e.g., as disclosed in Table 4.

In one embodiment, the anti-PD-L1 antibody molecule is Durvalumab (MedImmune/AstraZeneca), also known as MEDI4736. Durvalumab and other anti-PD-L1 antibodies are 5 disclosed in U.S. Pat. No. 8,779,108, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Durvalumab, e.g., as disclosed in Table 4.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559, e.g., as disclosed in Table 4.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

### Formulations

The anti-PD-L1 antibody molecules described herein can be formulated into a formulation (e.g., a dose formulation or dosage form) suitable for administration (e.g., intravenous administration) to a subject as described herein. The formulation described herein can be a liquid formulation, a lyophilized formulation, or a reconstituted formulation.

In certain embodiments, the formulation is a liquid formulation. In some embodiments, the formulation (e.g., liquid formulation) comprises an anti-PD-L1 antibody molecule (e.g., an anti-PD-L1 antibody molecule described herein) and a buffering agent.

In some embodiments, the formulation (e.g., liquid formulation) comprises an anti-PD-L1 antibody molecule present at a concentration of 25 mg/mL to 250 mg/mL, e.g., 50 mg/mL to 200 mg/mL, 60 mg/mL to 180 mg/mL, 70 mg/mL to 150 mg/mL, 80 mg/mL to 120 mg/mL, 90 mg/mL to 110 mg/mL, 50 mg/mL to 150 mg/mL, 50 mg/mL to 100 mg/mL, 150 mg/mL to 200 mg/mL, or 100 mg/mL to 200 mg/mL, e.g., 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL. In certain embodiments, the anti-PD-L1 antibody molecule is present at a concentration of 80 mg/mL to 120 mg/mL, e.g., 100 mg/mL.

In some embodiments, the formulation (e.g., liquid formulation) comprises a buffering agent comprising histidine (e.g., a histidine buffer). In certain embodiments, the buffering agent (e.g., histidine buffer) is present at a concentration of 1 mg/mL to 10 mg/mL, e.g., 2 mg/mL to 8 mg/mL, 1 mg/mL to 5 mg/mL, 3 mg/mL to 7 mg/mL, 2 mg/mL to 6 mg/mL, 3 mg/mL to 8 mg/mL, 1 mg/mL to 5 mg/mL, 2 mg/mL to 7 mg/mL, 3 mg/mL to 9 mg/mL, or 1 mg/mL to 6 mg/mL, e.g., 2 mg/mL, 3 mg/mL, 4 mg/mL, 5 mg/mL, 6 mg/mL, 7 mg/mL, 8 mg/mL, or 9 mg/mL. In some embodiments, the buffering agent (e.g., histidine buffer) is present at a concentration of 2 mg/mL to 6 mg/mL, e.g., about 3 mg/mL (e.g., 3.1 mg/mL). In other embodiments, the buffering agent (e.g., a histidine buffer) or the formulation has a pH of 4 to 7, e.g., 5 to 6, e.g., 5, 5.5, or 6. In some embodiments, the buffering agent (e.g., histidine buffer) or the formulation has a pH of 5 to 6, e.g., 5.5. In certain embodiments, the buffering agent comprises a histidine buffer at a concentration of 2 mg/mL to 6 mg/mL (e.g., about 3 mg/mL) and has a pH of 5 to 6 (e.g., 5.5). In certain embodiments, the buffering agent comprises histidine and histidine-HCl.

In some embodiments, the formulation (e.g., liquid formulation) comprises an anti-PD-L1 antibody molecule present at a concentration of 80 to 120 mg/mL, e.g., 100 mg/mL; and a buffering agent that comprises a histidine buffer at a concentration of 2 mg/mL to 6 mg/mL (e.g., about 3 mg/mL), at a pH of 5 to 6 (e.g., 5.5).

In some embodiments, the formulation (e.g., liquid formulation) further comprises a carbohydrate. In certain embodiments, the carbohydrate is sucrose. In some embodiments, the carbohydrate (e.g., sucrose) is present at a concentration of 20 mg/mL to 200 mg/mL, e.g., 25 mg/mL to 180 mg/mL, 30 mg/mL to 170 mg/ml, 45 mg/mL to 140 mg/ml, 60 mg/mL to 190 mg/mL, 35 mg/mL to 165 mg/mL, 70 mg/mL to 130 mg/mL, 65 mg/mL to 145 mg/mL, 40 mg/mL to 160 mg/mL, 55 mg/mL to 165 mg/mL, 30 mg/mL to 150 mg/mL, 50 mg/mL to 175 mg/mL, or 75 mg/mL to 125 mg/mL, e.g., 30 mg/mL, 40 mg/mL, 50 mg/mL, 60 mg/mL, 70 mg/mL, 75 mg/mL, 80 mg/mL, 85 mg/mL, 90 mg/mL, 100 mg/mL, 110 mg/mL, 120 mg/mL, 130 mg/mL, 140 mg/mL, or 150 mg/mL. In some embodiments, the formulation comprises a carbohydrate or sucrose present at a concentration of 50 mg/mL to 100 mg/mL, e.g., about 75 mg/mL (e.g., 75.3 mg/mL).

In some embodiments, the formulation (e.g., liquid formulation) comprises an anti-PD-L1 antibody molecule present at a concentration of 80 to 120 mg/mL, e.g., 100 mg/mL; a buffering agent that comprises a histidine buffer at a concentration of 2 mg/mL to 6 mg/mL, e.g., about 3 mg/mL; and a carbohydrate or sucrose present at a concentration of 50 mg/mL to 100 mg/mL, e.g., about 75 mg/mL, at a pH of 5 to 6 (e.g., 5.5).

In some embodiments, the formulation (e.g., liquid formulation) further comprises a surfactant. In certain embodiments, the surfactant is polysorbate 20. In some embodiments, the surfactant or polysorbate 20) is present at a concentration of 0.1 mg/mL to 1.0 mg/mL e.g. 0.2 mg/mL to 0.9 mg/mL, 0.3 mg/mL to 0.8 mg/mL, 0.4 mg/mL to 0.9 mg/mL, 0.3 mg/mL to 0.7 mg/mL, 0.2 mg/mL to 0.8 mg/mL, 0.3 mg/mL to 0.6 mg/mL, 0.4 mg/mL to 0.7 mg/mL, 0.2 mg/mL to 0.7 mg/mL, 0.3 mg/mL to 0.9 mg/mL, 0.3 mg/mL to 0.5 mg/mL, 0.4 mg/mL to 0.8 mg/mL, or 0.2 mg/mL to 0.5 mg/mL, e.g., 0.2 mg/mL, 0.3 mg/mL, 0.4 mg/mL, 0.5 mg/mL, 0.6 mg/mL, 0.7 mg/mL, 0.8 mg/mL, or 0.9 mg/mL. In some embodiments, the formulation comprises a surfactant or polysorbate 20 present at a concentration of 0.2 mg/mL to 0.6 mg/mL, e.g., 0.4 mg/mL.

In some embodiments, the formulation (e.g., liquid formulation) comprises an anti-PD-L1 antibody molecule present at a concentration of 80 to 120 mg/mL, e.g., 100 mg/mL; a buffering agent that comprises a histidine buffer at a concentration of 2 mg/mL to 6 mg/mL, e.g., about 3 mg/mL; a carbohydrate or sucrose present at a concentration of 50 mg/mL to 100 mg/mL, e.g., about 75 mg/mL; and a surfactant or polysorbate 20 present at a concentration of 0.2 mg/mL to 0.6 mg/mL, e.g., 0.4 mg/mL, at a pH of 5 to 6 (e.g., 5.5).

In some embodiments, the formulation (e.g., liquid formulation) comprises an anti-PD-L1 antibody molecule present at a concentration of 100 mg/mL; a buffering agent that comprises a histidine buffer (e.g., histidine/histidine-HCL) at a concentration of about 3 mg/mL (e.g., 3.1 mg/mL); a carbohydrate or sucrose present at a concentration of about 75 mg/mL (e.g., 75.3 mg/mL); and a surfactant or polysorbate 20 present at a concentration of 0.4 mg/mL, at a pH of 5 to 6 (e.g., 5.5).

A formulation described herein can be stored in a container. The container used for any of the formulations described herein can include, e.g., a vial, and optionally, a stopper, a cap, or both. In certain embodiments, the vial is a glass vial, e.g., a 6R white glass vial or colorless glass vial. In other embodiments, the stopper is a rubber stopper, e.g., a grey rubber stopper. In other embodiments, the cap is a flip-off cap, e.g., an aluminum flip-off cap. In some embodiments, the container comprises a 6R white glass vial, a grey rubber stopper, and an aluminum flip-off cap. In some embodiments, the container (e.g., vial) is for a single-use container. In certain embodiments, 25 mg/mL to 250 mg/mL, e.g., 50 mg/mL to 200 mg/mL, 60 mg/mL to 180 mg/mL, 70 mg/mL to 150 mg/mL, 80 mg/mL to 120 mg/mL, 90 mg/mL to 110 mg/mL, 50 mg/mL to 150 mg/mL, 50 mg/mL to 100 mg/mL, 150 mg/mL to 200 mg/mL, or 100 mg/mL to 200 mg/mL, e.g., 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL, of the anti-PD-L1 antibody molecule, is present in the container (e.g., vial).

In another aspect, the disclosure features therapeutic kits that include the anti-PD-L1 antibody molecules, compositions, or formulations described herein, and instructions for use, e.g., in accordance with dosage regimens described herein.

### Therapeutic Use

The anti-PD-L1 antibody molecules described herein can inhibit, reduce, or neutralize one or more activities of PD-L1, resulting in blockade or reduction of an immune checkpoint. Thus, the anti-PD-L1 antibody molecules described herein can be used to treat or prevent disorders (e.g., cancer), where enhancing an immune response in a subject is desired.

Accordingly, in another aspect, a method of modulating an immune response in a subject is provided. The method comprises administering to the subject an anti-PD-L1 antibody molecule described herein in accordance with a dosage regimen described herein, alone or in combination with one or more therapeutic agents, procedures, or modalities, such that the immune response in the subject is modulated. In one embodiment, the antibody molecule enhances, stimulates, or increases the immune response in the subject. The subject can be a mammal, e.g., a primate, preferably a higher primate, e.g., a human (e.g., a patient having, or at risk of having, a disorder described herein). In one embodiment, the subject is in need of enhancing an immune response. In one embodiment, the subject has, or is at risk of, having a disorder described herein, e.g., a cancer or an infectious disorder as described herein. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

In one aspect, a method of treating (e.g., one or more of reducing, inhibiting, or delaying progression) a cancer or a tumor in a subject is provided. The method comprises administering to the subject an anti-PD-L1 antibody molecule described herein in accordance with a dosage regimen described herein, alone or in combination with one or more therapeutic agents, procedures, or modalities.

In certain embodiments, the cancer treated with the anti-PD-L1 antibody molecule, includes but is not limited to, a solid tumor, a hematological cancer (e.g., leukemia, lymphoma, myeloma, e.g., multiple myeloma), and a metastatic lesion. In one embodiment, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma, e.g., a cutaneous melanoma), pancreas, and bones (e.g., a chordoma), as well as adenocarcinomas which include malignancies such as colon cancers, rectal cancer, renal cancer (e.g., renal-cell carcinoma (clear cell or non-clear cell renal cell carcinoma), liver cancer, lung cancer (e.g., non-small cell lung cancer (squamous or non-squamous non-small cell lung cancer)), cancer of the small intestine, and cancer of the esophagus. The cancer may be at an early, intermediate, late stage, or metastatic cancer.

In one embodiment, the cancer is chosen from a lung cancer (e.g., a non-small cell lung cancer (NSCLC) (e.g., a NSCLC with squamous and/or non-squamous histology, or a NSCLC adenocarcinoma), or a small cell lung cancer (SCLC)), a skin cancer (e.g., a Merkel cell carcinoma or a melanoma (e.g., an advanced melanoma)), an ovarian cancer, a mesothelioma, a bladder cancer, a soft tissue sarcoma (e.g., a hemangiopericytoma (HPC)), a bone cancer (a bone sarcoma), a kidney cancer (e.g., a renal cancer (e.g., a renal cell carcinoma)), a liver cancer (e.g., a hepatocellular carcinoma), a cholangiocarcinoma, a sarcoma, a myelodysplastic syndrome (MDS), a prostate cancer, a breast cancer (e.g., a breast cancer that does not express one, two or all of estrogen receptor, progesterone receptor, or Her2/neu, e.g., a triple negative breast cancer), a colorectal cancer (e.g., a relapsed colorectal cancer or a metastatic colorectal cancer, e.g., a microsatellite unstable colorectal cancer, a microsatellite stable colorectal cancer, a mismatch repair proficient colorectal cancer, or a mismatch repair deficient colorectal cancer), a nasopharyngeal cancer, a duodenal cancer, an endometrial cancer, a pancreatic cancer, a head and neck cancer (e.g., head and neck squamous cell carcinoma (HNSCC)), an anal cancer, a gastro-esophageal cancer, athyroid cancer (e.g., anaplastic thyroid carcinoma), a cervical cancer (e.g., a squamous cell carcinoma of the cervix), a neuroendocrine tumor (NET) (e.g., an atypical pulmonary carcinoid tumor), a lymphoproliferative disease (e.g., a post-transplant lymphoproliferative disease), a lymphoma (e.g., T-cell lymphoma, B-cell lymphoma, or a non-Hogdkin lymphoma), a myeloma (e.g., a multiple myeloma), or a leukemia (e.g., a myeloid leukemia or a lymphoid leukemia).

In certain embodiments, the cancer is a solid tumor. In some embodiments, the cancer is brain tumor, e.g., a glioblastoma, a gliosarcoma, or a recurrent brain tumor. In some embodiments, the cancer is a pancreatic cancer, e.g., an advanced pancreatic cancer. In some embodiments, the cancer is a skin cancer, e.g., a melanoma (e.g., a stage II-IV melanoma, an HLA-A2 positive melanoma, an unresectable melanoma, or a metastatic melanoma), or a Merkel cell carcinoma. In some embodiments, the cancer is a renal cancer, e.g., a renal cell carcinoma (RCC) (e.g., a metastatic renal cell carcinoma). In some embodiments, the cancer is a breast cancer, e.g., a metastatic breast carcinoma or a stage IV breast carcinoma, e.g., a triple negative breast cancer (TNBC). In some embodiments, the cancer is a virus-associated cancer. In some embodiments, the cancer is an anal canal cancer (e.g., a squamous cell carcinoma of the anal canal). In some embodiments, the cancer is a cervical cancer (e.g., a squamous cell carcinoma of the cervix). In some embodiments, the cancer is a gastric cancer (e.g., an Epstein Barr Virus (EBV) positive gastric cancer, or a gastric or gastro-esophageal junction carcinoma). In some embodiments, the cancer is a head and neck cancer (e.g., an HPV positive and negative squamous cell cancer of the head and neck (SCCHN)). In some embodiments, the cancer is a nasopharyngeal cancer (NPC). In some embodiments, the cancer is a penile cancer (e.g., a squamous cell carcinoma of the penile). In some embodiments, the cancer is a vaginal or vulvar cancer (e.g., a squamous cell carcinoma of the vagina or vulva). In some embodiments, the cancer is a colorectal cancer, e.g., a relapsed colorectal cancer, a metastatic colorectal cancer, e.g., a microsatellite unstable colorectal cancer, a microsatellite stable colorectal cancer, a mismatch repair proficient colorectal cancer, or a mismatch repair deficient colorectal cancer. In some embodiments, the cancer is a lung cancer, e.g., a non-small cell lung cancer (NSCLC).

In certain embodiments, the cancer is a hematological cancer. In some embodiments, the cancer is a leukemia. In some embodiments, the cancer is a lymphoma, e.g., a Hodgkin lymphoma (HL) or a diffuse large B cell lymphoma (DLBCL) (e.g., a relapsed or refractory HL or DLBCL). In some embodiments, the cancer is a myeloma.

In another embodiment, the cancer is chosen from a carcinoma (e.g., advanced or metastatic carcinoma), melanoma or a lung carcinoma, e.g., a non-small cell lung carcinoma. In one embodiment, the cancer is a lung cancer, e.g., a non-small cell lung cancer or small cell lung cancer. In some embodiments, the non-small cell lung cancer is a stage I (e.g., stage Ia or Ib), stage II (e.g., stage IIa or IIb), stage III (e.g., stage IIIa or IIIb), or stage IV, non-small cell lung cancer. In one embodiment, the cancer is a melanoma, e.g., an advanced melanoma. In one embodiment, the cancer is an advanced or unresectable melanoma that does not respond to other therapies. In other embodiments, the cancer is a melanoma with a BRAF mutation (e.g., a BRAF V600 mutation). In another embodiment, the cancer is a hepatocarcinoma, e.g., an advanced hepatocarcinoma, with or without a viral infection, e.g., a chronic viral hepatitis. In another embodiment, the cancer is a prostate cancer, e.g., an advanced prostate cancer. In yet another embodiment, the cancer is a myeloma, e.g., multiple myeloma. In yet another embodiment, the cancer is a renal cancer, e.g., a renal cell carcinoma (RCC) (e.g., a metastatic RCC, a non-clear cell renal cell carcinoma (nccRCC), or clear cell renal cell carcinoma (CCRCC)).

In one embodiment, the cancer microenvironment has an elevated level of PD-L1 expression. In one embodiment, the cancer microenvironment has an elevated level of LAG-3 expression. Alternatively, or in combination, the cancer microenvironment can have increased IFNγ and/or CD8 expression.

In some embodiments, the subject has, or is identified as having, a tumor that has one or more of high PD-L1 level or expression, or as being Tumor Infiltrating Lymphocyte (TIL)+(e.g., as having an increased number of TILs), or both. In certain embodiments, the subject has, or is identified as having, a tumor that has high PD-L1 level or expression and that is TIL+. In some embodiments, the methods described herein further include identifying a subject based on having a tumor that has one or more of high PD-L1 level or expression, or as being TIL+, or both. In certain embodiments, the methods described herein further include identifying a subject based on having a tumor that has high PD-L1 level or expression and as being TIL+. In some embodiments, tumors that are TIL+ are positive for CD8 and IFNγ. In some embodiments, the subject has, or is identified as having, a high percentage of cells that are positive for one, two or more of PD-L1, CD8, and/or IFNγ. In certain embodiments, the subject has or is identified as having a high percentage of cells that are positive for all of PD-L1, CD8, and IFNγ. In some embodiments, the methods described herein further include identifying a subject based on having a high percentage of cells that are positive for one, two or more of PD-L1, CD8, and/or IFNγ. In certain embodiments, the methods described herein further include identifying a subject based on having a high percentage of cells that are positive for all of PD-L1, CD8, and IFNγ. In some embodiments, the subject has, or is identified as having, one, two or more of PD-L1, CD8, and/or IFNγ, and one or more of a lung cancer, e.g., squamous cell lung cancer or lung adenocarcinoma (e.g., an NSCLC); a head and neck cancer; a squamous cell cervical cancer; a stomach cancer; an esophageal cancer; a thyroid cancer (e.g., anaplastic thyroid carcinoma); a skin cancer (e.g., a Merkel cell carcinoma or a melanoma), a breast cancer (e.g., a TNBC), and/or a nasopharyngeal cancer (NPC). In certain embodiments, the methods described herein further describe identifying a subject based on having one, two or more of PD-L1, CD8, and/or IFNγ, and one or more of a lung cancer, e.g., squamous cell lung cancer or lung adenocarcinoma (e.g., an NSCLC); a head and neck cancer; a squamous cell cervical cancer; a stomach cancer; a thyroid cancer (e.g., anaplastic thyroid carcinoma); a skin cancer (e.g., a Merkel cell carcinoma or a melanoma), an neuroendocrine tumor, a breast cancer (e.g., a TNBC), and/or a nasopharyngeal cancer.

Methods, compositions, and formulations disclosed herein are useful for treating metastatic lesions associated with the aforementioned cancers.

In a further aspect, the disclosure provides a method of treating an infectious disease (e.g., an infectious disease described herein) in a subject, comprising administering to the subject an anti-PD-L1 antibody molecule described herein in accordance with a dosage regimen described herein.

Still further, the invention provides a method of enhancing an immune response to an antigen in a subject, comprising administering to the subject: (i) the antigen; and (ii) an anti-PD-L1 antibody molecule described herein, in accordance with a dosage regimen described herein, such that an immune response to the antigen in the subject is enhanced. The antigen can be, for example, a tumor antigen, a viral antigen, a bacterial antigen or an antigen from a pathogen.

The anti-PD-L1 antibody molecule described herein can be administered to the subject systemically (e.g., orally, parenterally, subcutaneously, intravenously, rectally, intramuscularly, intraperitoneally, intranasally, transdermally, or by inhalation or intracavitary installation), topically, or by application to mucous membranes, such as the nose, throat and bronchial tubes. In certain embodiments, the anti-PD-L1 antibody molecule is administered intravenously at a flat dose described herein.

### Combination Therapies

The anti-PD-L1 antibody molecules described herein can be used in combination with other therapeutic agents, procedures, or modalities.

In one embodiment, the methods described herein include administering to the subject a combination comprising an anti-PD-L1 antibody molecule described herein, in combination with a therapeutic agent, procedure, or modality, in an amount effective to treat or prevent a disorder. In certain embodiments, the anti-PD-L1 antibody molecule is administered or used in accordance with a dosage regimen described herein. In other embodiments, the antibody molecule is administered or used as a composition or formulation described herein.

The anti-PD-L1 antibody molecule and the therapeutic agent, procedure, or modality can be administered or used simultaneously or sequentially in any order. Any combination and sequence of the anti-PD-L1 antibody molecule and the therapeutic agent, procedure, or modality (e.g., as described herein) can be used. The antibody molecule and/or the therapeutic agent, procedure or modality can be administered or used during periods of active disorder, or during a period of remission or less active disease. The antibody molecule can be administered before, concurrently with, or after the treatment with the therapeutic agent, procedure or modality.

In certain embodiments, the anti-PD-L1 antibody molecule described herein is administered in combination with one or more of other antibody molecules, chemotherapy, other anti-cancer therapy (e.g., targeted anti-cancer therapies, gene therapy, viral therapy, RNA therapy bone marrow transplantation, nanotherapy, or oncolytic drugs), cytotoxic agents, immune-based therapies (e.g., cytokines or cell-based immune therapies), surgical procedures (e.g., lumpectomy or mastectomy), or radiation procedures, or a combination of any of the foregoing. The additional therapy may be in the form of adjuvant or neoadjuvant therapy. In some embodiments, the additional therapy is an enzymatic inhibitor (e.g., a small molecule enzymatic inhibitor) or a metastatic inhibitor. Exemplary cytotoxic agents that can be administered in combination with include antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteasome inhibitors, and radiation (e.g., local or whole body irradiation (e.g., gamma irradiation)). In other embodiments, the additional therapy is surgery or radiation, or a combination thereof. In other embodiments, the additional therapy is a therapy targeting one or more of PI3K/AKT/mTOR pathway, an HSP90 inhibitor, or a tubulin inhibitor.

In some embodiments, the anti-PD-L1 antibody described herein is administered as a monotherapy.

Alternatively, or in combination with the aforesaid combinations, the anti-PD-L1 antibody described herein can be administered or used in combination with, one or more of: an immunomodulator (e.g., an activator of a costimulatory molecule or an inhibitor of an inhibitory molecule, e.g., an immune checkpoint molecule); a vaccine, e.g., a therapeutic cancer vaccine; or other forms of cellular immunotherapy.

In certain embodiments, the anti-PD-L1 molecule described herein is administered or used in combination with a modulator of a costimulatory molecule or an inhibitory molecule, e.g., a co-inhibitory ligand or receptor.

In one embodiment, the anti-PD-L1 antibody molecule described herein is administered or used in combination with a modulator, e.g., agonist, of a costimulatory molecule. In one embodiment, the agonist of the costimulatory molecule is chosen from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3, or CD83 ligand.

In another embodiment, the anti-PD-L1 antibody molecule described herein is administered or used in combination with a GITR agonist, e.g., an anti-GITR antibody molecule.

In one embodiment, the anti-PD-L1 antibody molecule described herein is administered or used in combination with an inhibitor of an inhibitory (or immune checkpoint) molecule chosen from PD-1, PD-L1, PD-L2, CTLA-4, TIM-3, LAG-3, CEACAM (e.g., CEACAM-1, CEACAM-3, and/or CEACAM-5), VISTA, BTLA, TIGIT, LAIR1, CD160, 2B4 and/or TGF beta. In one embodiment, the inhibitor is a soluble ligand (e.g., a CTLA-4-Ig), or an antibody or antibody fragment that binds to PD-1, PD-L1, LAG-3, PD-L2, or CTLA-4.

In another embodiment, the anti-PD-L1 antibody molecule described herein is administered or used in combination with a PD-1 inhibitor, e.g., an anti-PD-1 antibody molecule. In another embodiment, the anti-PD-L1 antibody molecule described herein is administered or used in combination with a TIM-3 inhibitor, e.g., an anti-TIM-3 antibody molecule. In another embodiment, the anti-PD-L1 antibody molecule described herein is administered or used in combination with a LAG-3 inhibitor, e.g., an anti-LAG-3 antibody molecule.

In another embodiment, the anti-PD-L1 antibody molecule described herein is administered or used in combination with a chemotherapeutic agent. In certain embodiments, the chemotherapeutic agent comprises a platinum agent (e.g., carboplatin, cisplatin, oxaliplatin, or tetraplatin). In certain embodiments, the chemotherapeutic agent comprises cisplatin, permetrexed, or both. Cisplatin is also known as cisplatinum, platamin, neoplatin, cismaplat, or cis-diamminedichloridoplatinum(II) (CDDP). Permetrxed is also known as (S)-2-(4-(2-(2-amino-4-oxo-4,7-dihydro-3H-pyrrolo[2,3-d]pyrimidin-5-yl)ethyl)benzamido)pentanedioic acid. In certain embodiments, the chemotherapeutic agent comprises a nucleotide analog or precursor analog (e.g., capecitabine, azacitidine, azathioprine, cytarabine, doxifluridine, fluorouracil, gemcitabine, hydroxyurea, mercaptopurine, methotrexate, or tioguanine (thioguanine)). In certain embodiments, the chemotherapeutic agent comprises a hypomethylating agent (e.g., decitabine). In one embodiment, the chemotherapeutic agent comprises nab-paclitaxel.

Other exemplary chemotherapeutic agents that can be used in combination with the anti-PD-L1 antibody molecule include, but are not limited to, an alkylating agent (e.g., a bifunctional alkylator (e.g., cyclophosphamide, a mechlorethamine, chlorambucil, or melphalan)), a monofunctional alkylator (e.g., dacarbazine (DTIC), nitrosoureas, ortemozolomide (oral dacarbazine)), an anthracycline (e.g., daunorubicin, doxorubicin, epirubicin, idarubicin, mitoxantrone, or valrubicin), a cytoskeletal disruptor or taxane (e.g., paclitaxel, docetaxel, abraxane, or taxotere), an epothilone, a histone deacetylase inhibitor (e.g., vorinostat or romidepsin), an inhibitor of topoisomerase I (e.g., irinotecan or topotecan), an inhibitor of topoisomerase II (e.g., etoposide, teniposide, or tafluposide), a kinase inhibitor (e.g., bortezomib, erlotinib, gefitinib, imatinib, vemurafenib, or vismodegib), a peptide antibiotic (e.g., bleomycin or actinomycin), a retinoid (e.g., tretinoin, alitretinoin, or bexarotene), or a vinca alkaloid or derivative thereof (e.g., vinblastine, vincristine, vindesine, or vinorelbine).

In another embodiment, the anti-PD-L1 antibody molecule described herein is administered or used in combination with a PD-1 inhibitor (e.g., an anti-PD-1 antibody molecule) and a TIM-3 inhibitor (e.g., an anti-TIM-3 antibody molecule). In another embodiment, the anti-PD-L1 antibody molecule described herein is administered or used in combination with a PD-1 inhibitor (e.g., an anti-PD-1 antibody molecule) and a LAG-3 inhibitor (e.g., an anti-LAG-3 antibody molecule). In another embodiment, the anti-PD-L1 antibody molecule described herein is administered or used in combination with a TIM-3 inhibitor (e.g., an anti-TIM-3 antibody molecule) and a LAG-3 inhibitor (e.g., an anti-LAG-3 antibody molecule). In another embodiment, the anti-PD-L1 antibody molecule described herein is administered or used in combination with a PD-1 inhibitor (e.g., an anti-PD-1 antibody molecule) and a chemotherapeutic agent (e.g., a platinum agent (e.g., carboplatin, cisplatin, oxaliplatin, or tetraplatin) or a nucleotide analog or precursor analog (e.g., capecitabine)). In another embodiment, the anti-PD-L1 antibody molecule described herein is administered or used in combination with a CEACAM inhibitor (e.g., CEACAM-1, CEACAM-3, and/or CEACAM-5 inhibitor), e.g., an anti-CEACAM antibody molecule. In another embodiment, the anti-PD-L1 antibody molecule is administered or used in combination with a CEACAM-1 inhibitor, e.g., an anti-CEACAM-1 antibody molecule. In another embodiment, the anti-PD-L1 antibody molecule is administered or used in combination with a CEACAM-3 inhibitor, e.g., an anti-CEACAM-3 antibody molecule. In another embodiment, the anti-PD-L1 antibody molecule is administered or used in combination with a CEACAM-5 inhibitor, e.g., an anti-CEACAM-5 antibody molecule.

The combination of antibody molecules disclosed herein can be administered separately, e.g., as separate antibody molecules, or linked, e.g., as a bispecific or trispecific antibody molecule. In one embodiment, a bispecific antibody that includes an anti-PD-L1 antibody molecule and an anti-PD-1, anti-CEACAM (e.g., anti-CEACAM-1, CEACAM-3, and/or anti-CEACAM-5), anti-LAG-3, or anti-TIM-3 antibody molecule, is administered. In certain embodiments, the combination of antibodies disclosed herein is used to treat a cancer, e.g., a cancer as described herein (e.g., a solid tumor or a hematologic malignancy).

In another embodiment, the anti-PD-L1 antibody molecule is administered or used in combination with an anti-PD-1 antibody molecule, e.g., to treat a brain cancer (e.g., a glioblastoma), a melanoma, a renal cancer (e.g., a renal cell carcinoma), a virus-associated cancer (e.g., an anal canal cancer, a cervical cancer, a gastric cancer, a head and neck cancer, a nasopharyngeal cancer (NPC), a penile cancer, or a vaginal or vulvar cancer), a colorectal cancer, or a lung cancer (e.g., a non-small cell lung cancer (NSCLC)). In certain embodiments, the anti-PD-L1 antibody molecule is administered or used in combination with an anti-PD-1 antibody molecule, e.g., to treat a breast cancer, e.g., a triple negative breast cancer (TNBC).

In another embodiment, the anti-PD-L1 antibody molecule is administered or used in combination with a chemotherapeutic agent (e.g., gemcitabine, paclitaxel), e.g., to treat a pancreatic cancer or a breast cancer.

In another embodiment, the anti-PD-L1 antibody molecule is administered or used in combination with a chemotherapeutic agent (e.g., a platinum agent (e.g., carboplatin, cisplatin, oxaliplatin, or tetraplatin) or a nucleotide analog or precursor analog (e.g., capecitabine)), e.g., to treat a breast cancer, e.g., a TNBC. In certain embodiments, the anti-PD-L1 antibody molecule is administered or used in combination with an anti-PD-1 antibody molecule and a chemotherapeutic agent (e.g., a platinum agent (e.g., carboplatin, cisplatin, oxaliplatin, or tetraplatin) or a nucleotide analog or precursor analog (e.g., capecitabine)), e.g., to treat a breast cancer, e.g., a TNBC. In other embodiments, the anti-PD-L1 antibody molecule is administered or used in combination with a cytokine. The cytokine can be administered as a fusion molecule to the anti-PD-L1 antibody molecule, or as separate compositions. In other embodiments, the anti-PD-L1 antibody molecule is administered or used in combination with one, two, three, or more cytokines, e.g., as a fusion molecule or as separate compositions. In one embodiment, the cytokine is an interleukin (IL) chosen from one, two, three or more of IL-1, IL-2, IL-12, IL-15, or IL-21. In one embodiment, a bispecific antibody molecule has a first binding specificity to a first target (e.g., to PD-L1), a second binding specificity to a second target (e.g., PD-1, TIM-3, or LAG-3), and is optionally linked to an interleukin (e.g., IL-12) domain e.g., full length IL-12 or a portion thereof. In certain embodiments, the combination of anti-PD-L1 antibody molecule and the cytokine described herein is used to treat a cancer, e.g., a cancer as described herein (e.g., a solid tumor).

In other embodiments, the anti-PD-L1 antibody molecule is administered or used in combination with an antibody specific against an HLA C, e.g., an antibody specific to Killer-cell Immunoglobulin-like Receptors (also referred to herein as an “anti-KIR antibody”). In certain embodiments, the combination of anti-PD-L1 antibody molecule and anti-KIR antibody is used to treat a cancer, e.g., a cancer as described herein (e.g., a solid tumor, e.g., an advanced solid tumor).

In other embodiments, the anti-PD-L1 antibody molecule is administered or used in combination with a cellular immunotherapy (e.g., PROVENGE® (e.g., Sipuleucel-T)), and optionally in combination with cyclophosphamide. In certain embodiments, the combination of anti-PD-L1 antibody molecule, PROVENGE®, and/or cyclophosphamide is used to treat a cancer, e.g., a cancer as described herein (e.g., a prostate cancer, e.g., an advanced prostate cancer).

In other embodiments, the anti-PD-L1 antibody molecule is administered or used in combination with a vaccine, e.g., a cancer vaccine, (e.g., a dendritic cell renal carcinoma (DC-RCC) vaccine). In one embodiment, the vaccine is peptide-based, DNA-based, RNA-based, or antigen-based, or a combination thereof. In embodiments, the vaccine comprises one or more peptides, nucleic acids (e.g., DNA or RNA), antigens, or a combination thereof. In certain embodiments, the combination of the anti-PD-L1 antibody molecule and the DC-RCC vaccine is used to treat a cancer, e.g., a cancer as described herein (e.g., a renal carcinoma, e.g., metastatic renal cell carcinoma (RCC) or clear cell renal cell carcinoma (CCRCC)).

In other embodiments, the anti-PD-L1 antibody molecule is administered or used in combination with an adjuvant.

In other embodiments, the anti-PD-L1 antibody molecule is administered or used in combination with chemotherapy, and/or immunotherapy. For example, the anti-PD-L1 antibody molecule can be used to treat a myeloma, alone or in combination with one or more of chemotherapy or other anti-cancer agents (e.g., thalidomide analogs, e.g., lenalidomide), an anti-PD-1 antibody molecule, tumor antigen-pulsed dendritic cells, fusions (e.g., electrofusions) of tumor cells and dendritic cells, or vaccination with immunoglobulin idiotype produced by malignant plasma cells. In other embodiments, the anti-PD-L1 antibody molecule is administered or used in combination with an anti-PD-1 antibody molecule to treat a myeloma, e.g., a multiple myeloma.

In other embodiments, the anti-PD-L1 antibody molecule is administered or used in combination with chemotherapy to treat a lung cancer, e.g., non-small cell lung cancer. In other embodiments, the anti-PD-L1 antibody molecule is administered or used with standard lung, e.g., NSCLC, chemotherapy, e.g., platinum doublet therapy, to treat lung cancer. In other embodiments, the anti-PD-L1 antibody molecule is administered or used in combination with an indoleamine-pyrrole 2,3-dioxygenase (IDO) inhibitor (e.g., (4E)-4-[(3-chloro-4-fluoroanilino)-nitrosomethylidene]-1,2,5-oxadiazol-3-amine (also known as INCB24360), indoximod (1-methyl-D-tryptophan), or α-cyclohexyl-5H-Imidazo[5,1-a]isoindole-5-ethanol (also known as NLG919)) in a subject with advanced or metastatic cancer (e.g., a patient with metastatic and recurrent NSCL cancer).

In yet other embodiments, In other embodiments, the anti-PD-L1 antibody molecule is administered or used in combination with one or more of: an immune-based strategy (e.g., interleukin-2 or interferon-α), a targeting agent (e.g., a VEGF inhibitor such as a monoclonal antibody to VEGF); a VEGF tyrosine kinase inhibitor, such as sunitinib, sorafenib, axitinib, and pazopanib; an RNAi inhibitor; or an inhibitor of a downstream mediator of VEGF signaling, e.g., an inhibitor of the mammalian target of rapamycin (mTOR), e.g., everolimus and temsirolimus. Any of such combinations can be used to treat a renal cancer, e.g., renal cell carcinoma (RCC) (e.g., clear cell renal cell carcinoma (CCRCC), a non-clear cell renal cell carcinoma (nccRCC), or metastatic RCC), or a liver cancer (e.g., a hepatocellular carcinoma).

In other embodiments, the anti-PD-L1 antibody molecule is administered or used in combination with a MEK inhibitor (e.g., a MEK inhibitor as described herein). In some embodiments, the combination of the anti-PD-L1 antibody molecule and the MEK inhibitor is used to treat a cancer (e.g., a cancer described herein). In some embodiments, the cancer treated with the combination is chosen from a melanoma, a colorectal cancer, a non-small cell lung cancer, an ovarian cancer, a breast cancer, a prostate cancer, a pancreatic cancer, a hematological malignancy, or a renal cell carcinoma. In certain embodiments, the cancer includes a BRAF mutation (e.g., a BRAF V600E mutation), a BRAF wildtype, a KRAS wildtype or an activating KRAS mutation. The cancer may be at an early, intermediate or late stage.

In other embodiments, the anti-PD-L1 antibody molecule is administered or used in combination with one, two, or all of a chemotherapeutic agent (e.g., a platinum agent (e.g., carboplatin, oxaliplatin, cisplatin, or tetraplatin)) or a nucleotide analog or precursor analog (e.g., capecitabine)), leucovorin, or 5-FU (e.g., a FOLFOX co-treatment). Alternatively or in combination, the combination further includes a VEGF inhibitor (e.g., a VEGF inhibitor as disclosed herein). In some embodiments, the combination of the anti-PD-L1 antibody molecule, the FOLFOX co-treatment, and the VEGF inhibitor is used to treat a cancer (e.g., a cancer described herein). In some embodiments, the cancer treated with the combination is chosen from a melanoma, a colorectal cancer, a non-small cell lung cancer, an ovarian cancer, a breast cancer, a prostate cancer, a pancreatic cancer, a hematological malignancy, or a renal cell carcinoma. The cancer may be at an early, intermediate, or late stage.

In other embodiments, the anti-PD-L1 antibody molecule is administered or used with a tyrosine kinase inhibitor (e.g., axitinib) to treat renal cell carcinoma and other solid tumors.

In other embodiments, the anti-PD-L1 antibody molecule is administered or used with a 4-1BB receptor targeting agent (e.g., an antibody that stimulates signaling through 4-1BB (CD-137), e.g., PF-2566). In other embodiments, the anti-PD-L1 antibody molecule is administered or used in combination with a tyrosine kinase inhibitor (e.g., axitinib) and a 4-1BB receptor targeting agent.

The anti-PD-L1 antibody molecule can be bound to a substance, e.g., a cytotoxic agent or moiety (e.g., a therapeutic drug; a compound emitting radiation; molecules of plant, fungal, or bacterial origin; or a biological protein (e.g., a protein toxin) or particle (e.g., a recombinant viral particle, e.g., via a viral coat protein)). For example, the antibody can be coupled to a radioactive isotope such as an α-, β-, or γ-emitter, or a β- and γ-emitter.

### Immunomodulators

The anti-PD-L1 antibody molecules described herein can be used in combination with one or more immunomodulators.

In certain embodiments, the immunomodulator is an inhibitor of an immune checkpoint molecule. In one embodiment, the immunomodulator is an inhibitor of PD-1, LAG-3, PD-L2, CTLA-4, TIM-3, CEACAM (e.g., CEACAM-1, -3 and/or -5), VISTA, BTLA, TIGIT, LAIR1, CD160, 2B4 and/or TGF beta. In one embodiment, the inhibitor of an immune checkpoint molecule inhibits PD-1, LAG-3, TIM-3, CEACAM (e.g., CEACAM-1, -3 and/or -5), CTLA-4, or any combination thereof.

Inhibition of an inhibitory molecule can be performed at the DNA, RNA, or protein level. In embodiments, an inhibitory nucleic acid (e.g., a dsRNA, siRNA, or shRNA), can be used to inhibit expression of an inhibitory molecule. In other embodiments, the inhibitor of an inhibitory molecule is, a polypeptide e.g., a soluble ligand (e.g., PD-1-Ig or CTLA-4 Ig), or an antibody molecule that binds to the inhibitory molecule; e.g., an antibody molecule that binds to PD-1, LAG-3, PD-L2, CEACAM (e.g., CEACAM-1, -3 and/or -5), CTLA-4, TIM-3, VISTA, BTLA, TIGIT, LAIR1, CD160, 2B4, and/or TGF beta, or a combination thereof.

In certain embodiments, the anti-PD-L1 antibody molecule is in the form of a bispecific or multispecific antibody molecule. In one embodiment, the bispecific antibody molecule has a first binding specificity to PD-L1 and a second binding specificity, e.g., a second binding specificity to PD-1, LAG-3, CEACAM (e.g., CEACAM-1, -3 and/or -5), TIM-3, or PD-L2. In one embodiment, the bispecific antibody molecule binds to PD-L1 and PD-1. In one embodiment, the bispecific antibody molecule binds to PD-L1 and LAG-3. In another embodiment, the bispecific antibody molecule binds to PD-L1 and TIM-3. In another embodiment, the bispecific antibody molecule binds to PD-L1 and PD-L2. In another embodiment, the bispecific antibody molecule binds to PD-L1 and CEACAM (e.g., CEACAM-1, -3 and/or -5). In another embodiment, the bispecific antibody molecule binds to PD-L1 and CEACAM-1. In still another embodiment, the bispecific antibody molecule binds to PD-L1 and CEACAM-3. In yet another embodiment, the bispecific antibody molecule binds to PD-L1 and CEACAM-5.

In other embodiments, the anti-PD-L1 antibody molecule is used in combination with a bispecific or multispecific antibody molecule. In some embodiments, the bispecific antibody molecule binds to PD-1 or PD-L1. In some embodiments, the bispecific antibody molecule binds to PD-1 and PD-L2. In some embodiments, the bispecific antibody molecule binds to CEACAM (e.g., CEACAM-1, -3 and/or -5) and LAG-3.

Any combination of the aforesaid molecules can be made in a multispecific antibody molecule, e.g., a trispecific antibody that includes a first binding specificity to PD-L1, and a second and third binding specificities to two or more of PD-1, LAG-3, CEACAM (e.g., CEACAM-1, -3 and/or -5), TIM-3, or PD-L2.

In certain embodiments, the immunomodulator is an inhibitor of PD-1, e.g., human PD-1. In one embodiment, the inhibitor of PD-1 is an antibody molecule to PD-1 (e.g., an anti-PD-1 antibody molecule as described herein).

The combination of the PD-1 inhibitor with the anti-PD-L1 antibody molecule can further include one or more additional immunomodulators, e.g., in combination with an inhibitor of TIM-3, CEACAM (e.g., CEACAM-1, -3 and/or -5), or CTLA-4. In one embodiment, the inhibitor of PD-1 (e.g., the anti-PD-1 antibody molecule) is administered in combination with the anti-PD-L1 antibody molecule and a TIM-3 inhibitor (e.g., an anti-TIM-3 antibody molecule). In another embodiment, the inhibitor of PD-1 (e.g., the anti-PD-1 antibody molecule) is administered in combination with the anti-PD-L1 antibody molecule and a CEACAM inhibitor (e.g., CEACAM-1, -3 and/or -5 inhibitor), e.g., an anti-CEACAM antibody molecule. In another embodiment, the inhibitor of PD-1 (e.g., the anti-PD-1 antibody molecule) is administered in combination with the anti-PD-L1 antibody molecule and a CEACAM-1 inhibitor (e.g., an anti-CEACAM-1 antibody molecule). In another embodiment, the inhibitor of PD-1 (e.g., the anti-PD-1 antibody molecule) is administered in combination with the anti-PD-L1 antibody molecule and a CEACAM-1 inhibitor (e.g., an anti-CEACAM-3 antibody molecule). In another embodiment, the inhibitor of PD-1 (e.g., the anti-PD-1 antibody molecule) is administered in combination with the anti-PD-L1 antibody molecule and a CEACAM-5 inhibitor (e.g., an anti-CEACAM-5 antibody molecule). In yet other embodiments, the inhibitor of PD-1 (e.g., the anti-PD-1 antibody molecule) is administered in combination with the anti-PD-L1 antibody molecule and a TIM-3 inhibitor (e.g., an anti-TIM-3 antibody molecule). Other combinations of immunomodulators with the anti-PD-L1 antibody molecule and a PD-1 inhibitor including, e.g., one or more of PD-L2, CTLA-4, LAG-3, CEACAM (e.g., CEACAM-1, -3 and/or -5), VISTA, BTLA, TIGIT, LAIR1, CD160, 2B4, and/or TGF beta) are also within the present invention. Any of the antibody molecules known in the art or disclosed herein can be used in the aforesaid combinations of inhibitors of checkpoint molecule.

In other embodiments, the immunomodulator is an inhibitor of CEACAM (e.g., CEACAM-1, -3 and/or -5), e.g., human CEACAM (e.g., CEACAM-1, -3 and/or -5). In one embodiment, the immunomodulator is an inhibitor of CEACAM-1, e.g., human CEACAM-1. In another embodiment, the immunomodulator is an inhibitor of CEACAM-3, e.g., human CEACAM-3. In another embodiment, the immunomodulator is an inhibitor of CEACAM-5, e.g., human CEACAM-5. In one embodiment, the inhibitor of CEACAM (e.g., CEACAM-1, -3 and/or -5) is an antibody molecule to CEACAM (e.g., CEACAM-1, -3 and/or -5). The combination of the CEACAM (e.g., CEACAM-1, -3 and/or -5) inhibitor and the anti-PD-L1 antibody molecule can further include one or more additional immunomodulators, e.g., in combination with an inhibitor of TIM-3, PD-1, LAG-3, or CTLA-4.

In other embodiments, the immunomodulator is an inhibitor of TIM-3, e.g., human TIM-3. In one embodiment, the inhibitor of TIM-3 is an antibody molecule to TIM-3. The combination of the TIM-3 inhibitor and the anti-PD-L1 antibody molecule can further include one or more additional immunomodulators, e.g., in combination with an inhibitor of CEACAM (e.g., CEACAM-1, -3 and/or -5), PD-1, LAG-3, or CTLA-4.

In certain embodiments, the immunomodulator used in the combinations disclosed herein (e.g., in combination with a therapeutic agent chosen from an antigen-presentation combination) is an activator or agonist of a costimulatory molecule. In one embodiment, the agonist of the costimulatory molecule is chosen from an agonist (e.g., an agonistic antibody or antigen-binding fragment thereof, or a soluble fusion) of OX40, CD2, CD27, CD28, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3, or CD83 ligand.

In other embodiments, the immunomodulator is a GITR agonist. In one embodiment, the GITR agonist is an antibody molecule to GITR. The anti-GITR antibody molecule and the anti-PD-L1 antibody molecule may be in the form of separate antibody composition, or as a bispecific antibody molecule. The combination of the GITR agonist with the anti-PD-L1 antibody molecule can further include one or more additional immunomodulators, e.g., in combination with an inhibitor of PD-1, LAG-3, CTLA-4, CEACAM (e.g., CEACAM-1, -3 and/or -5), or TIM-3. In some embodiments, the anti-GITR antibody molecule is a bispecific antibody that binds to GITR and PD-1, LAG-3, CTLA-4, CEACAM (e.g., CEACAM-1, -3 and/or -5), or TIM-3. In other embodiments, a GITR agonist can be administered in combination with one or more additional activators of costimulatory molecules, e.g., an agonist of OX40, CD2, CD27, CD28, CDS, ICAM-1, LFA-1 (CD11a/CD18), ICOS (CD278), 4-1BB (CD137), CD30, CD40, BAFFR, HVEM, CD7, LIGHT, NKG2C, SLAMF7, NKp80, CD160, B7-H3, or CD83 ligand.

In other embodiments, the immunomodulator is an OX40 agonist. In one embodiment, the OX40 agonist is an antibody molecule to OX40. The OX40 antibody molecule and the anti-PD-L1 antibody molecule may be in the form of separate antibody composition, or as a bispecific antibody molecule. The combination of the OX40 agonist with the anti-PD-L1 antibody molecule can further include one or more additional immunomodulators, e.g., in combination with an inhibitor of PD-1, LAG-3, CTLA-4, CEACAM (e.g., CEACAM-1, -3, and/or -5), or TIM-3. In some embodiments, the anti-OX40 antibody molecule is a bispecific antibody that binds to OX40 and PD-1, LAG-3, CTLA-4, CEACAM (e.g., CEACAM-1, -3 and/or -5), or TIM-3. In other embodiments, the OX40 agonist can be administered in combination with other costimulatory molecule, e.g., an agonist of GITR, CD2, CD27, CD28, CDS, ICAM-1, LFA-1 (CD11a/CD18), ICOS (CD278), 4-1BB (CD137), CD30, CD40, BAFFR, HVEM, CD7, LIGHT, NKG2C, SLAMF7, NKp80, CD160, B7-H3, or CD83 ligand.

It is noted that only exemplary combinations of inhibitors of checkpoint inhibitors or agonists of costimulatory molecules are provided herein. Additional combinations of these agents are within the scope of the present invention.

### Biomarkers

In certain embodiments, any of the methods disclosed herein further includes evaluating or monitoring the effectiveness of a therapy (e.g., a monotherapy or a combination therapy) described herein, in a subject (e.g., a subject having a cancer, e.g., a cancer described herein). The method includes acquiring a value of effectiveness to the therapy, wherein said value is indicative of the effectiveness of the therapy.

In embodiments, the value of effectiveness to the therapy comprises a measure of one, two, three, four, five, six, seven, eight, nine, or more (e.g., all) of the following:
(i) a parameter of a tumor infiltrating lymphocyte (TIL) phenotype;
(ii) a parameter of a myeloid cell population;
(iii) a parameter of a surface expression marker;
(iv) a parameter of a biomarker of an immunologic response;
(v) a parameter of a systemic cytokine modulation;
(vi) a parameter of circulating free DNA (cfDNA);
(vii) a parameter of systemic immune-modulation;
(viii) a parameter of microbiome;
(ix) a parameter of a marker of activation in a circulating immune cell; or
(x) a parameter of a circulating cytokine.

In some embodiments, the parameter of a TIL phenotype comprises the level or activity of one, two, three, four, or more (e.g., all) of Hematoxylin and eosin (H&E) staining for TIL counts, CD8, FOXP3, CD4, or CD3, in the subject, e.g., in a sample from the subject (e.g., a tumor sample).

In some embodiments, the parameter of a myeloid cell population comprises the level or activity of one or both of CD68 or CD163, in the subject, e.g., in a sample from the subject (e.g., a tumor sample).

In some embodiments, the parameter of a surface expression marker comprises the level or activity of one, two, three, or more (e.g., all) of PD-L1, TIM-3, PD-1, or LAG-3, in the subject, e.g., in a sample from the subject (e.g., a tumor sample). In certain embodiments, the level of PD-L1, TIM-3, PD-1, or LAG-3 is determined by immunohistochemistry (IHC). In certain embodiments, the level of PD-L1 is determined.

In some embodiments, the parameter of a biomarker of an immunologic response comprises the level or sequence of one or more nucleic acid-based markers, in the subject, e.g., in a sample from the subject (e.g., a tumor sample).

In some embodiments, the parameter of systemic cytokine modulation comprises the level or activity of one, two, three, four, five, six, seven, eight, or more (e.g., all) of IL-18, IFN-γ, ITAC (CXCL11), IL-6, IL-10, IL-4, IL-17, IL-15, or TGF-beta, in the subject, e.g., in a sample from the subject (e.g., a blood sample, e.g., a plasma sample).

In some embodiments, the parameter of cfDNA comprises the sequence or level of one or more circulating tumor DNA (cfDNA) molecules, in the subject, e.g., in a sample from the subject (e.g., a blood sample, e.g., a plasma sample).

In some embodiments, the parameter of systemic immune-modulation comprises phenotypic characterization of an activated immune cell, e.g., a CD3-expressing cell, a CD8-expressing cell, or both, in the subject, e.g., in a sample from the subject (e.g., a blood sample, e.g., a PBMC sample).

In some embodiments, the parameter of microbiome comprises the sequence or expression level of one or more genes in the microbiome, in the subject, e.g., in a sample from the subject (e.g., a stool sample).

In some embodiments, the parameter of a marker of activation in a circulating immune cell comprises the level or activity of one, two, three, four, five or more (e.g., all) of circulating CD8+, HLA-DR+Ki67+, T cells, IFN-γ, IL-18, or CXCL11 (IFN-γ induced CCK) expressing cells, in a sample (e.g., a blood sample, e.g., a plasma sample).

In some embodiments, the parameter of a circulating cytokine comprises the level or activity of IL-6, in the subject, e.g., in a sample from the subject (e.g., a blood sample, e.g., a plasma sample).

In some embodiments of any of the methods disclosed herein, the therapy comprises a combination of an anti-PD-L1 antibody molecule described herein and a second inhibitor of an immune checkpoint molecule, e.g., an inhibitor of PD-1 (e.g., an anti-PD-1 antibody molecule) or an inhibitor of LAG-3 (e.g., an anti-LAG-3 antibody molecule).

In some embodiments of any of the methods disclosed herein, the measure of one or more of (i)-(x) is obtained from a sample acquired from the subject. In some embodiments, the sample is chosen from a tumor sample, a blood sample (e.g., a plasma sample or a PBMC sample), or a stool sample.

In some embodiments of any of the methods disclosed herein, the subject is evaluated prior to receiving, during, or after receiving, the therapy.

In some embodiments of any of the methods disclosed herein, the measure of one or more of (i)-(x) evaluates a profile for one or more of gene expression, flow cytometry or protein expression.

In some embodiments of any of the methods disclosed herein, the presence of an increased level or activity of one, two, three, four, five, or more (e.g., all) of circulating CD8+, HLA-DR+Ki67+, T cells, IFN-γ, IL-18, or CXCL11 (IFN-γ induced CCK) expressing cells, and/or the presence of an decreased level or activity of IL-6, in the subject or sample, is a positive predictor of the effectiveness of the therapy.

Alternatively, or in combination with the methods disclosed herein, responsive to said value, performing one, two, three, four or more (e.g., all) of:
(i) administering to the subject the therapy;
(ii) administered an altered dosing of the therapy;
(iii) altering the schedule or time course of the therapy;
(iv) administering to the subject an additional agent (e.g., a therapeutic agent described herein) in combination with the therapy; or
(v) administering to the subject an alternative therapy.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1B depict a graph of mean (±SD) serum concentration-time plots for the anti-PD-L1 antibody molecule FAZ053 after single (cycle 1) and multiple administrations (cycle 3 or 5) at doses of 80 mg, 240 mg, 800 mg, 1200 mg, or 1600 mg every 3 weeks (FIG. 1A) or 800 mg, 1200 mg, or 1600 mg every 6 weeks (FIG. 1).

FIG. 2 depicts a graph of serum concentration-time plots of total soluble PD-L1 (sPD-L1) for subjects administered FAZ053 at doses of 80 mg, 240 mg, 800 mg, 1200 mg, or 1600 mg every 3 weeks or 800, 1200, or 1600 mg every 6 weeks.

FIG. 3 depicts a graph of predicted PD-L1 receptor occupancy in a tumor after administration of FAZ053 at doses of 80 mg, 240 mg, 800 mg, 1200 mg, or 1600 mg at the end of Cycle 1 (week 3). The box shows the interquantile range (IQR) and the line extends to the furthest point that is no more than 1.5*IQR from the box.

FIG. 4 depicts a graph of best % change for anti-tumor activity from baseline by dose category for subjects with a variety of cancer types administered FAZ053 at doses of 80 mg, 240 mg, 800 mg, or 1600 mg every 3 weeks.

FIG. 5 depicts a graph of best % change for anti-tumor activity from baseline by dose category for subjects with a variety of cancer types administered FAZ053 at doses of 800 mg or 1600 mg every 6 weeks.

### DETAILED DESCRIPTION

Programmed Death Ligand 1 (PD-L1) is a ligand for the immunoinhibitory receptor Programmed Death 1 (PD-1). Binding of PD-L1 to PD-1 leads to the inhibition of T cell receptor-mediated lymphocyte proliferation and cytokine secretion, which enhances antitumor immunity. PD-L1 is expressed on activated T cells, dendritic cells, NK cells, macrophages, B cells, monocytes, and vascular endothelium cells. Many tumor infiltrating T lymphocytes predominantly express PD-1 compared to T lymphocytes in normal tissues and peripheral blood T lymphocytes, indicating that up-regulation of PD-1 on tumor-reactive T cells can contribute to impaired antitumor immune responses. Thus, PD-L1 signaling may lead to attenuation of T cell activation and evasion of immune surveillance.

Accordingly, disclosed herein are, at least in part, are antibody molecules (e.g., humanized antibody molecules) that bind PD-L1 with high affinity and specificity. Pharmaceutical compositions and dose formulations comprising the anti-PD-L1 antibody molecules are also provided. The anti-PD-L1 antibody molecules disclosed herein can be used (alone or in combination with other therapeutic agents, procedures, or modalities) to treat or prevent disorders, such as cancerous disorders (e.g., solid tumors and hematological cancers), as well as infectious diseases (e.g., chronic infectious disorders or sepsis). Thus, methods, including dosage regimens, for treating various disorders using the anti-PD-L1 antibody molecules are disclosed herein. In certain embodiments, the anti-PD-L1 antibody molecule is administered or used at a flat or fixed dose. In some embodiments, the anti-PD-L 1 antibody is administered as a monotherapy. In other embodiments, the anti-PD-L1 antibody is administered in combination with other therapeutic agents.

### Definitions

Additional terms are defined below and throughout the application.

As used herein, the articles “a” and “an” refer to one or to more than one (e.g., to at least one) of the grammatical object of the article.

The term “or” is used herein to mean, and is used interchangeably with, the term “and/or,” unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

By “a combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. In will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In embodiments, the additional therapeutic agent is administered at a therapeutic or lower-than therapeutic dose. In certain embodiments, the concentration of the second therapeutic agent that is required to achieve inhibition, e.g., growth inhibition, is lower when the second therapeutic agent is administered in combination with the first therapeutic agent, e.g., the anti-PD-L1 antibody molecule, than when the second therapeutic agent is administered individually. In certain embodiments, the concentration of the first therapeutic agent that is required to achieve inhibition, e.g., growth inhibition, is lower when the first therapeutic agent is administered in combination with the second therapeutic agent than when the first therapeutic agent is administered individually. In certain embodiments, in a combination therapy, the concentration of the second therapeutic agent that is required to achieve inhibition, e.g., growth inhibition, is lower than the therapeutic dose of the second therapeutic agent as a monotherapy, e.g., 10-20%, 20-30%, 30-40%, 40-50%, 50-60%, 60-70%, 70-80%, or 80-90% lower. In certain embodiments, in a combination therapy, the concentration of the first therapeutic agent that is required to achieve inhibition, e.g., growth inhibition, is lower than the therapeutic dose of the first therapeutic agent as a monotherapy, e.g., 10-20%, 20-30%, 30-40%, 40-50%, 50-60%, 60-70%, 70-80%, or 80-90% lower.

The term “inhibition,” “inhibitor,” or “antagonist” includes a reduction in a certain parameter, e.g., an activity, of a given molecule, e.g., an immune checkpoint inhibitor. For example, inhibition of an activity, e.g., a PD-L1 activity, of at least 5%, 10%, 20%, 30%, 40%, or more is included by this term. Thus, inhibition need not be 100%.

The term “activation,” “activator,” or “agonist” includes an increase in a certain parameter, e.g., an activity, of a given molecule, e.g., a costimulatory molecule. For example, increase of an activity, e.g., a costimulatory activity, of at least 5%, 10%, 25%, 50%, 75%, or more is included by this term.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place.

The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, solid tumors, e.g., lung cancer, breast cancer, prostate cancer, ovarian cancer, cervical cancer, skin cancer, bone cancer, pancreatic cancer, colorectal cancer, renal cancer, liver cancer, and brain cancer, and hematologic malignancies, e.g., lymphoma and leukemia, and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The term “antigen presenting cell” or “APC” refers to an immune system cell, such as an accessory cell (e.g., a B-cell, a dendritic cell, and the like), that displays a foreign antigen complexed with major histocompatibility complexes (MHCs) on its surface. T-cells may recognize these complexes using their T-cell receptors (TCRs). APCs process antigens and present them to T-cells.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to, an MHC class I molecule, TNF receptor proteins, immunoglobulin-like proteins, cytokine receptors, integrins, signalling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, CD19a, and a ligand that specifically binds with CD83.

“Immune effector cell,” or “effector cell” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

“Immune effector” or “effector” “function” or “response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

As used herein, the terms “treat,” “treatment,” and “treating” refer to the reduction or amelioration of the progression, severity and/or duration of a disorder, e.g., a proliferative disorder, or the amelioration of one or more symptoms (preferably, one or more discernible symptoms) of the disorder resulting from the administration of one or more therapies. In specific embodiments, the terms “treat,” “treatment,” and “treating” refer to the amelioration of at least one measurable physical parameter of a proliferative disorder, such as growth of a tumor, not necessarily discernible by the patient. In other embodiments the terms “treat,” “treatment,” and “treating” refer to the inhibition of the progression of a proliferative disorder, either physically by, e.g., stabilization of a discernible symptom, physiologically by, e.g., stabilization of a physical parameter, or both. In other embodiments the terms “treat,” “treatment,” and “treating” refer to the reduction or stabilization of tumor size or cancerous cell count.

The compositions, formulations, and methods of the present invention encompass polypeptides and nucleic acids having the sequences specified, or sequences substantially identical or similar thereto, e.g., sequences at least 85%, 90%, or 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence, such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence, such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98%, or 99% identity to a reference sequence, e.g., a sequence provided herein.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to a nucleic acid (SEQ ID NO: 1) molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof, amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a nonnatural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

### Dosage Regimens

The PD-L1 inhibitors, e.g., anti-PD-L1 antibody molecules described herein, can be administered according to a dosage regimen described herein to treat (e.g., inhibit, reduce, ameliorate, or prevent) a disorder, e.g., a hyperproliferative condition or disorder (e.g., a cancer) in a subject. In certain embodiments, the anti-PD-L1 antibody molecule is administered to the subject at a dose of about 20 mg to about 2000 mg, e.g., once every two, three, four, six, or eight weeks.

In some aspect, the disclosure features a method of treating a cancer in a subject, the method comprising administering to the subject an anti-PD-L1 antibody molecule (e.g., an anti-PD-L1 antibody molecule described herein) at a dose or dosage schedule described herein.

In some embodiments, the dosage regimen (e.g., 1200 mg once every three weeks) is chosen based on good overall safety, sPDL1 binding (Total sPDL1) and exploratory modeling that predicted >99% receptor occupancy in the tumor. Without wishing to be bound by theory, it is believed that in some embodiments although the lower dose of 800 mg once every three weeks is predicted to achieve similar receptor occupancy, a 1200 mg once every three weeks dose is used to increase the likelihood of tumor penetration of the anti-PD-L1 antibody molecule and/or to overcome a possible higher burden of sPDL1 (antigen sink) in the subject (e.g., due to the variability in total sPDL1).

In some embodiments, the dosage regimen (e.g., 1600 mg once every four weeks) is chosen based on the same expected steady state average PK concentration (Cave) as the 1200 mg once every three weeks regimen. Average steady state concentration (Cave) for the anti-PD-L1 antibody molecule can be calculated as Dose/(CL*τ) where i is the dosing frequency and CL is the intrinsic clearance of the anti-PD-L1 antibody molecule. Based on this formula, Cave=1200 mg/(CL*3 weeks)=1600 mg/(CL*4 weeks). Without wishing to be bound by theory, it is believed that in some embodiments, doses up to 1600 mg once every three weeks are safe and generally well tolerated in patients.

In certain embodiments, the dosage regimen is chosen based on a PK/PD and receptor occupancy based approach. Without wishing to be bound by theory, it is believed that in some embodiments, the dosage regimen allows for flexibility in adjusting the administration regimen to enhance patient convenience, e.g., dosing schedule for combination drugs.

In certain embodiments, the anti-PD-L1 antibody molecule (e.g., an anti-PD-L1 antibody molecule described herein) is administered at a dose greater than or equal to about 800 mg once every three weeks or greater than or equal to about 1600 mg once every six weeks. Without wishing to be bound by theory, it is believed that in some embodiments, a dose greater than or equal to about 800 mg once every three weeks or greater than or equal to about 1600 mg once every six weeks results in sustained binding of soluble PD-L1 throughout the dosing interval. In some embodiments, the anti-PD-L1 antibody is administered at a dose or dosage regimen that results in occupancy of PD-L1 in the subject. In certain embodiments, a dose greater than or equal to 800 mg (e.g., 1200 mg) once every three weeks of the anti-PD-L1 antibody results in occupancy of PD-L1 (e.g., 99% receptor occupancy of PD-L1) in the subject.

In some embodiments, the dose or dosage regimen is selected based on having the same expected steady state average PK concentration (Cave) as the anti-PD-L1 antibody when administered at a dose that results in occupancy of PD-L1 (e.g., a dose greater than or equal to 800 mg (e.g., 1200 mg) once every three weeks). In certain embodiments, a dose of about 1500 mg to about 1700 mg (e.g., about 1600 mg) once every four weeks has the same expected Cave as the anti-PD-L1 antibody when administered at a dose greater than or equal to 800 mg (e.g., 1200 mg) once every three weeks.

In some embodiments, the anti-PD-L1 antibody molecule is administered at a dose or dosage schedule that results in binding, e.g., saturation, of soluble PD-L1 in the subject. In some embodiments, the anti-PD-L1 antibody molecule is administered at a dose or dosage schedule that results in at least 50%, 60%, 70%, 80%, 85%, 90%, 95%, 98%, or 99% binding, e.g., saturation, of soluble PD-L1 in the subject, e.g., within 1, 2, 3, 4, 5, 6, 7, 8, 9, 10, 11, or 12, 24, 36, or 48 weeks of administration. In some embodiments, the anti-PD-L1 antibody molecule is administered at a dose or dosage schedule that results in sustained binding, e.g., sustained saturation, of soluble PD-L1 in the subject, e.g., for 1, 2, 3, 4, 5, 6, 7, 8, 9, or more weeks of administration.

In some embodiments, the anti-PD-L1 antibody molecule is administered at a dose or dosage schedule that results in binding, e.g., occupancy, of PD-L1 in a tumor in the subject. In some embodiments, the anti-PD-L1 antibody molecule is administered at a dose or dosage schedule that results in at least 50%, 60%, 70%, 80%, 90%, 95%, 98%, or 99% binding, e.g., occupancy, of PD-L1 in a tumor in the subject, e.g., within 1, 2, 3, 4, 5, 6, 7, 8, 9, 10, 11, 12, 24, 36, or 48 weeks of administration. In some embodiments, the anti-PD-L1 antibody molecule is administered at a dose or dosage schedule that results in sustained binding, e.g., sustained occupancy, of PD-L1 in the subject, e.g., for 1, 2, 3, 4, 5, 6, 7, 8, 9, or more weeks of administration.

In other embodiments, the anti-PD-L1 antibody molecule is administered at a dose or dosage schedule that results in at least 50%, 60%, 70%, 80%, 85%, 90%, 95%, 98%, or 99% binding, e.g., saturation, of soluble PD-L1 in the subject; and that results in at least 50%, 60%, 70%, 80%, 85%, 90%, 95%, 98%, or 99% binding, e.g., occupancy, of PD-L1 in a tumor in the subject. In some embodiments, the saturation and/or occupancy occurs, e.g., within 1, 2, 3, 4, 5, 6, 7, 8, 9, 10, 11, 12, 24, 36, or 48 weeks of administration. In some embodiments, the saturation and/or occupancy occurs, e.g., for 1, 2, 3, 4, 5, 6, 7, 8, 9, or more weeks of administration.

In some embodiments, the anti-PD-L1 antibody molecule is administered at a dose or dosage schedule that results in 50% or more (e.g., 60% or more, 70% or more, 75% or more, 80% or more, 85% or more, 90% or more, 95% or more, 99% or more) of the soluble PD-L1 in the subject (e.g., blood) bound by the anti-PD-L1 antibody molecule. In some embodiments, the binding of the anti-PD-L1 antibody molecule to soluble PD-L1 is determined in a blood sample (e.g., a serum sample or a plasma sample). In some embodiments, the binding of the anti-PD-L1 antibody molecule to soluble PD-L1 is determined, e.g., measured in vitro (e.g., by ELISA or a cell-based assay) or in vivo (e.g., by imaging), or predicted from a PK/PD model, e.g., a PK/PD model described herein.

In some embodiments, 50% or more of the soluble PD-L1 in a serum sample from the subject is bound by the anti-PD-L1 antibody molecule. In some embodiments, 60% or more of the soluble PD-L1 in a serum sample from the subject is bound by the anti-PD-L1 antibody molecule. In some embodiments, 70% or more of the soluble PD-L1 in a serum sample from the subject is bound by the anti-PD-L1 antibody molecule. In some embodiments, 80% or more of the soluble PD-L1 in a serum sample from the subject is bound by the anti-PD-L1 antibody molecule. In some embodiments, 90% or more of the soluble PD-L1 in a serum sample from the subject is bound by the anti-PD-L1 antibody molecule. In some embodiments, 95% or more of the soluble PD-L1 in a serum sample from the subject is bound by the anti-PD-L1 antibody molecule. In some embodiments, 99% or more of the soluble PD-L1 in a serum sample from the subject is bound by the anti-PD-L1 antibody molecule.

In some embodiments, the anti-PD-L1 antibody molecule is administered at a dose or dosage schedule that reduces the level of free soluble PD-L1 in the subject (e.g., in the blood), e.g., to 40% or less (e.g., 50% or less, 40% or less, 30% or less, 20% or less, 15% or less, 10% or less, 5% or less, or 1% or less) of a reference level of free soluble PD-L1.

In some embodiments, the level of free soluble PD-L1 is determined in a blood sample (e.g., a serum sample or a plasma sample). In some embodiments, the reference level of free soluble PD-L1 is the baseline level of free soluble PD-L1 in the subject, e.g., prior to administration of the anti-PD-L1 antibody molecule, e.g., in accordance with the dosage schedule. In some embodiments, the level of free soluble PD-L1 is determined, e.g., measured in vitro (e.g., by ELISA or a cell-based assay) or in vivo (e.g., by imaging), or predicted from a PK/PD model, e.g., a PK/PD model described herein.

In some embodiments, the level of free soluble PD-L1 is reduced to 20% or less of a reference level of free soluble PD-L1 in a serum sample from the subject. In some embodiments, the level of free soluble PD-L1 is reduced to 10% or less of a reference level of free soluble PD-L1 in a serum sample from the subject. In some embodiments, the level of free soluble PD-L1 is reduced to 5% or less of a reference level of free soluble PD-L1 in a serum sample from the subject. In some embodiments, the level of free soluble PD-L1 is reduced to 1% or less of a reference level of free soluble PD-L1 in a serum sample from the subject.

In some embodiments, the anti-PD-L1 antibody molecule is administered at a dose of about 20 mg to about 2000 mg, about 15 mg to about 1600 mg, about 20 mg to about 1400 mg, about 25 mg to about 1200 mg, about 40 mg to about 1800 mg, about 60 mg to about 1600 mg, about 80 mg to about 1400 mg, about 100 mg to about 1200 mg, about 120 mg to about 1000 mg, about 140 mg to about 800 mg, about 160 mg to about 600 mg, about 180 mg to about 400 mg, about 200 mg to about 300 mg, about 220 mg to about 260 mg, about 40 mg to about 1600 mg, about 40 mg to about 1200 mg, 40 mg to about 1000 mg, 40 mg to about 800 mg, about 40 mg to about 600 mg, about 40 mg to about 400 mg, about 40 mg to about 200 mg, about 40 mg to about 100 mg, about 40 mg to about 80 mg, about 1600 mg to about 1800 mg, about 1200 mg to about 1800 mg, about 1000 mg to about 1800 mg, about 800 mg to about 1800 mg, about 600 mg to about 1800 mg, about 400 mg to about 1800 mg, about 200 mg to about 1800 mg, about 100 mg to about 1800 mg, or about 80 to about 1800 mg, e.g., once every three weeks, once every four weeks, or once every six weeks.

In some embodiments, the anti-PD-L1 antibody molecule is administered at a dose of about 40 mg to about 120 mg, 60 mg to about 100 mg, about 70 mg to about 90 mg, about 60 mg to about 80 mg, about 80 mg to about 100 mg, e.g., about 40 mg, about 50 mg, about 60 mg, about 70 mg, about 80 mg, about 90 mg, about 100 mg, about 110 mg, or about 120 mg, e.g., once every three weeks, once every four weeks, or once every six weeks. In certain embodiments, the anti-PD-L1 antibody molecule is administered at a dose of about 60 mg to about 100 mg, e.g., about 80 mg, once every three weeks. In other embodiments, the anti-PD-L1 antibody molecule is administered at a dose of about 60 mg to about 100 mg, e.g., about 80 mg, once every four weeks. In other embodiments, the anti-PD-L1 antibody molecule is administered at a dose of about 60 mg to about 100 mg, e.g., about 80 mg, once every six weeks.

In some embodiments, the anti-PD-L1 antibody molecule is administered at a dose of about 200 mg to about 300 mg, about 220 mg to about 280 mg, about 230 mg and 250 mg, about 200 mg to about 240 mg, about 240 mg to about 260 mg, e.g., about 200 mg, about 220 mg, about 240 mg, about 260 mg, about 280 mg, or about 300 mg, e.g., once every three weeks, once every four weeks, or once every six weeks. In certain embodiments, the anti-PD-L1 antibody molecule is administered at a dose of about 220 mg to about 260 mg, e.g., about 240 mg, once every three weeks. In certain embodiments, the anti-PD-L1 antibody molecule is administered at a dose of about 220 mg to about 260 mg, e.g., about 240 mg, once every four weeks. In certain embodiments, the anti-PD-L1 antibody molecule is administered at a dose of about 220 mg to about 260 mg, e.g., about 240 mg, once every six weeks.

In some embodiments, the anti-PD-L1 antibody molecule is administered at a dose of about 500 mg to about 1000 mg, about 550 mg to about 950 mg, about 600 mg to about 900 mg, about 650 mg to about 925, about 700 mg to about 900 mg, e.g., about 500 mg, about 600 mg, about 700 mg, about 725 mg, about 750 mg, about 800 mg, about 825 mg, about 850 mg, about 900 mg, or about 1000 mg, e.g., once every three weeks, once every four weeks, or once every six weeks. In certain embodiments, the anti-PD-L1 antibody molecule is administered at a dose of about 700 mg to about 900 mg, e.g., about 800 mg, once every three weeks. In other embodiments, the anti-PD-L1 antibody molecule is administered at a dose of about 700 mg to about 900 mg, e.g., about 800 mg, once every four weeks. In other embodiments, the anti-PD-L1 antibody molecule is administered at a dose of about 700 mg to about 900 mg, e.g., about 800 mg, once every six weeks.

In some embodiments, the anti-PD-L1 antibody molecule is administered at a dose of about 900 mg to about 1400 mg, about 1000 mg to about 1400 mg, about 1100 mg to about 1300 mg, about 1000 mg to about 1200 mg, about 1200 mg to about 1400 mg, e.g., about 900 mg, about 1000 mg, about 1100 mg, about 1200 mg, about 1300 mg, or about 1400 mg, e.g., once every three weeks, once every four weeks, or once every six weeks. In certain embodiments, the anti-PD-L1 antibody molecule is administered at a dose of about 1000 mg to about 1400 mg, e.g., about 1200 mg, once every three weeks. In other embodiments, the anti-PD-L1 antibody molecule is administered at a dose of about 1000 mg to about 1400 mg, e.g., about 1200 mg, once every four weeks. In other embodiments, the anti-PD-L1 antibody molecule is administered at a dose of about 1000 mg to about 1400 mg, e.g., about 1200 mg, once every six weeks.

In some embodiments, the anti-PD-L1 antibody molecule is administered at a dose of about 1300 mg to about 1900 mg, about 1400 mg to about 1800 mg, about 1500 mg to about 1700 mg, about 1400 mg to about 1700 mg, about 1500 mg to about 1800 mg, e.g., about 1300 mg, about 1400 mg, about 1500 mg, about 1600 mg, about 1700 mg, about 1800 mg, or about 1900 mg, e.g., once every three weeks, once every four weeks, or once every six weeks. In certain embodiments, the anti-PD-L1 antibody molecule is administered at a dose of about 1400 mg to about 1800 mg, e.g., about 1600 mg, once every three weeks. In other embodiments, the anti-PD-L1 antibody molecule is administered at a dose of about 1400 mg to about 1800 mg, e.g., about 1200 mg, once every four weeks. In other embodiments, the anti-PD-L1 antibody molecule is administered at a dose of about 1400 mg to about 1800 mg, e.g., about 1200 mg, once every six weeks.

In some embodiments, the anti-PD-L1 antibody molecule is administered at a dose of about 2000 mg or less, about 1900 mg or less, about 1800 mg or less, about 1700 mg or less, about 1600 mg or less, about 1500 mg or less, about 1400 mg or less, about 1300 mg or less, about 1200 mg or less, about 1100 mg or less, about 1000 mg or less, about 900 mg or less, about 800 mg or less, about 700 mg or less, about 600 mg or less, about 500 mg or less, about 400 mg or less, about 300 mg or less, about 250 mg or less, about 200 mg or less, about 150 mg or less, about 100 mg or less, or about 80 mg or less, once every three weeks, once every four weeks, or once every six weeks.

In some embodiments, the disorder is a cancer, e.g., a cancer described herein. In certain embodiments, the cancer is a solid tumor. In some embodiments, the cancer is an ovarian cancer. In other embodiments, the cancer is a lung cancer, e.g., a small cell lung cancer (SCLC) or a non-small cell lung cancer (NSCLC). In other embodiments, the cancer is a mesothelioma. In other embodiments, the cancer is a skin cancer, e.g., a Merkel cell carcinoma or a melanoma. In other embodiments, the cancer is a kidney cancer, e.g., a renal cell carcinoma. In other embodiments, the cancer is a bladder cancer. In other embodiments, the cancer is a soft tissue sarcoma, e.g., a hemangiopericytoma (HPC). In other embodiments, the cancer is a bone cancer, e.g., a bone sarcoma. In other embodiments, the cancer is a colorectal cancer. In other embodiments, the cancer is a pancreatic cancer. In other embodiments, the cancer is a nasopharyngeal cancer. In other embodiments, the cancer is a breast cancer. In other embodiments, the cancer is a duodenal cancer. In other embodiments, the cancer is an endometrial cancer. In other embodiments, the cancer is an adenocarcinoma, e.g., an unknown adenocarcinoma. In other embodiments, the cancer is a liver cancer, e.g., a hepatocellular carcinoma. In other embodiments, the cancer is a cholangiocarcinoma. In other embodiments, the cancer is a sarcoma. In certain embodiments, the cancer is a myelodysplastic syndrome (MDS) (e.g., a high risk MDS). In other embodiments, the cancer is a leukemia (e.g., an acute myeloid leukemia (AML), e.g., a relapsed or refractory AML or a de novo AML). In other embodiments, the cancer is a lymphoma. In other embodiments, the cancer is a myeloma. In other embodiments, the cancer is an MSI-high cancer. In some embodiments, the cancer is a metastatic cancer. In other embodiments, the cancer is an advanced cancer. In other embodiments, the cancer is a relapsed or refractory cancer.

In one embodiment, the cancer is a Merkel cell carcinoma. In other embodiments, the cancer is a melanoma. In other embodiments, the cancer is a breast cancer, e.g., a triple negative breast cancer (TNBC) or a HER2-negative breast cancer. In other embodiments, the cancer is a renal cell carcinoma (e.g., a clear cell renal cell carcinoma (CCRCC) or a non-clear cell renal cell carcinoma (nccRCC)). In other embodiments, the cancer is a thyroid cancer, e.g., an anaplastic thyroid carcinoma (ATC). In other embodiments, the cancer is a neuroendocrine tumor (NET), e.g., an atypical pulmonary carcinoid tumor or an NET in pancreas, gastrointestinal (GI) tract, or lung. In certain embodiments, the cancer is a non-small cell lung cancer (NSCLC) (e.g., a squamous NSCLC or a non-squamous NSCLC). In certain embodiments, the cancer is a fallopian tube cancer. In certain embodiments, the cancer is a microsatellite instability-high colorectal cancer (MSI-high CRC) or a microsatellite stable colorectal cancer (MSS CRC).

### Antibody Molecules

Disclosed herein methods, compositions, and formulations that include an inhibitor of PD-L1, e.g., an antibody molecule that binds to a mammalian, e.g., human, PD-L1. For example, the antibody molecule binds specifically to an epitope, e.g., linear or conformational epitope, (e.g., an epitope as described herein) on PD-L1.

As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full length antibody which has an immunoglobulin Fc region). In an embodiment, an antibody molecule comprises a full length antibody, or a full length immunoglobulin chain. In an embodiment, an antibody molecule comprises an antigen binding or functional fragment of a full length antibody, or a full length immunoglobulin chain. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule.

In an embodiment, an antibody molecule is a monospecific antibody molecule and binds a single epitope. For example, a monospecific antibody molecule can have a plurality of immunoglobulin variable domain sequences, each of which binds the same epitope.

In an embodiment, an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap. In an embodiment the first and second epitopes do not overlap. In an embodiment, the first and second epitopes are on different antigens, e.g., the different proteins (or different subunits of a multimeric protein). In an embodiment, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody molecule comprises a half antibody having binding specificity for a first epitope and a half antibody having binding specificity for a second epitope. In an embodiment, a bispecific antibody molecule comprises a half antibody, or fragment thereof, having binding specificity for a first epitope and a half antibody, or fragment thereof, having binding specificity for a second epitope. In an embodiment, a bispecific antibody molecule comprises a scFv, or fragment thereof, have binding specificity for a first epitope and a scFv, or fragment thereof, have binding specificity for a second epitope. In an embodiment, the first epitope is located on PD-L1 and the second epitope is located on PD-1, TIM-3, CEACAM (e.g., CEACAM-1 and/or CEACAM-5), LAG-3, or PD-L2.

Protocols for generating multi-specific (e.g., bispecific or trispecific) or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002/004587A1, US2002/076406A1, US2002/103345A1, US2003/207346A1, US2003/211078A1, US2004/219643A1, US2004/220388A1, US2004/242847A1, US2005/003403A1, US2005/004352A1, US2005/069552A1, US2005/079170A1, US2005/100543A1, US2005/136049A1, US2005/136051A1, US2005/163782A1, US2005/266425A1, US2006/083747A1, US2006/120960A1, US2006/204493A1, US2006/263367A1, US2007/004909A1, US2007/087381A1, US2007/128150A1, US2007/141049A1, US2007/154901A1, US2007/274985A1, US2008/050370A1, US2008/069820A1, US2008/152645A1, US2008/171855A1, US2008/241884A1, US2008/254512A1, US2008/260738A1, US2009/130106A1, US2009/148905A1, US2009/155275A1, US2009/162359A1, US2009/162360A1, US2009/175851A1, US2009/175867A1, US2009/232811A1, US2009/234105A1, US2009/263392A1, US2009/274649A1, EP346087A2, WO00/06605A2, WO02/072635A2, WO04/081051A1, WO06/020258A2, WO2007/044887A2, WO2007/095338A2, WO2007/137760A2, WO2008/119353A1, WO2009/021754A2, WO2009/068630A1, WO91/03493A1, WO93/23537A1, WO94/09131A1, WO94/12625A2, WO95/09917A1, WO96/37621A2, WO99/64460A1. The contents of the above-referenced applications are incorporated herein by reference in their entireties.

In other embodiments, the anti-PD-L1 antibody molecule (e.g., a monospecific, bispecific, or multispecific antibody molecule) is covalently linked, e.g., fused, to another partner e.g., a protein e.g., one, two or more cytokines, e.g., as a fusion molecule for example a fusion protein. In other embodiments, the fusion molecule comprises one or more proteins, e.g., one, two or more cytokines. In one embodiment, the cytokine is an interleukin (IL) chosen from one, two, three or more of IL-1, IL-2, IL-12, IL-15, or IL-21. In one embodiment, a bispecific antibody molecule has a first binding specificity to a first target (e.g., to PD-L1), a second binding specificity to a second target (e.g., PD-1 or TIM-3), and is optionally linked to an interleukin (e.g., IL-12) domain e.g., full length IL-12 or a portion thereof.

A “fusion protein” and a “fusion polypeptide” refer to a polypeptide having at least two portions covalently linked together, where each of the portions is a polypeptide having a different property. The property may be a biological property, such as activity in vitro or in vivo. The property can also be simple chemical or physical property, such as binding to a target molecule, catalysis of a reaction, etc. The two portions can be linked directly by a single peptide bond or through a peptide linker, but are in reading frame with each other.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment, an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL, and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, and HCDR3) and three CDRs in each light chain variable region (LCDR1, LCDR2, and LCDR3).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

Generally, unless specifically indicated, the anti-PD-L1 antibody molecules can include any combination of one or more Kabat CDRs and/or Chothia hypervariable loops. In one embodiment, the following definitions are used for the anti-PD-L1 antibody molecules: HCDR1 according to the combined CDR definitions of both Kabat and Chothia, and HCCDRs 2-3 and LCCDRs 1-3 according the CDR definition of Kabat. Under all definitions, each VH and VL typically includes three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to the PD-L1 polypeptide, or an epitope thereof. With respect to proteins (or protein mimetics), the antigen-binding site typically includes one or more loops (of at least four amino acids or amino acid mimics) that form an interface that binds to the PD-L1 polypeptide. Typically, the antigen-binding site of an antibody molecule includes at least one or two CDRs and/or hypervariable loops, or more typically at least three, four, five or six CDRs and/or hypervariable loops.

The terms “compete” or “cross-compete” are used interchangeably herein to refer to the ability of an antibody molecule to interfere with binding of an anti-PD-L1 antibody molecule, e.g., an anti-PD-L1 antibody molecule provided herein, to a target, e.g., human PD-L1. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, for example, a FACS assay, an ELISA, or BIACORE assay. In some embodiments, a competition binding assay is a quantitative competition assay. In some embodiments, a first anti-PD-L1 antibody molecule is said to compete for binding to the target with a second anti-PD-L1 antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see e.g., Saleh et al., Cancer Immunol. Immunother. 32:180-190 (1990)) and also because of potential allergic reactions (see e.g., LoBuglio et al., Hybridoma, 5:5117-5123 (1986)).

The antibody molecule can be a polyclonal or a monoclonal antibody. In other embodiments, the antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio Technology 9:1370-1372; Hay et al. (1992) Hum Antibody Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to PD-L1. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (see e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody molecule of the invention may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, 0-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecule can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody molecules may be conjugated to another molecular entity, typically a label or a therapeutic (e.g., a cytotoxic or cytostatic) agent or moiety. Radioactive isotopes can be used in diagnostic or therapeutic applications.

The invention provides radiolabeled antibody molecules and methods of labeling the same. In one embodiment, a method of labeling an antibody molecule is disclosed. The method includes contacting an antibody molecule, with a chelating agent, to thereby produce a conjugated antibody.

As is discussed above, the antibody molecule can be conjugated to a therapeutic agent. Therapeutically active radioisotopes have already been mentioned. Examples of other therapeutic agents include taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see e.g., U.S. Pat. No. 5,208,020), CC-1065 (see e.g., U.S. Pat. Nos. 5,475,092, 5,585,499, 5,846, 545), and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In one aspect, the disclosure provides a method of providing a target binding molecule that specifically binds to a target disclosed herein, e.g., PD-L1. For example, the target binding molecule is an antibody molecule. The method includes: providing a target protein that comprises at least a portion of non-human protein, the portion being homologous to (at least 70, 75, 80, 85, 87, 90, 92, 94, 95, 96, 97, 98% identical to) a corresponding portion of a human target protein, but differing by at least one amino acid (e.g., at least one, two, three, four, five, six, seven, eight, or nine amino acids); obtaining an antibody molecule that specifically binds to the antigen; and evaluating efficacy of the binding agent in modulating activity of the target protein. The method can further include administering the binding agent (e.g., antibody molecule) or a derivative (e.g., a humanized antibody molecule) to a human subject.

This disclosure provides an isolated nucleic acid molecule encoding the above antibody molecule, vectors and host cells thereof. The nucleic acid molecule includes but is not limited to RNA, genomic DNA and cDNA.

### Exemplary PD-L1 Inhibitors

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety.

In one embodiment, the anti-PD-L1 antibody molecule comprises at least one, two, three, four, five, or six complementarity determining regions (CDRs) (or collectively all of the CDRs) from a heavy and light chain variable region comprising an amino acid sequence shown in Table 3 (e.g., from the heavy and light chain variable region sequences of BAP058-Clone O or BAP058-Clone N disclosed in Table 3), or encoded by a nucleotide sequence shown in Table 3. In some embodiments, the CDRs are according to the Kabat definition (e.g., as set out in Table 3). In some embodiments, the CDRs are according to the Chothia definition (e.g., as set out in Table 3). In some embodiments, the CDRs are according to the combined CDR definitions of both Kabat and Chothia (e.g., as set out in Table 3). In one embodiment, the combination of Kabat and Chothia CDR of VH CDR1 comprises the amino acid sequence GYTFTSYWMY (SEQ ID NO: 647). In one embodiment, one or more of the CDRs (or collectively all of the CDRs) have one, two, three, four, five, six, or more changes, e.g., amino acid substitutions (e.g., conservative amino acid substitutions) or deletions, relative to an amino acid sequence shown in Table 3, or encoded by a nucleotide sequence shown in Table 3.

In one embodiment, the anti-PD-L1 antibody molecule comprises a heavy chain variable region (VH) comprising a VHCDR1 amino acid sequence of SEQ ID NO: 601, a VHCDR2 amino acid sequence of SEQ ID NO: 602, and a VHCDR3 amino acid sequence of SEQ ID NO: 603; and a light chain variable region (VL) comprising a VLCDR1 amino acid sequence of SEQ ID NO: 609, a VLCDR2 amino acid sequence of SEQ ID NO: 610, and a VLCDR3 amino acid sequence of SEQ ID NO: 611, each disclosed in Table 3.

In one embodiment, the anti-PD-L1 antibody molecule comprises a VH comprising a VHCDR1 encoded by the nucleotide sequence of SEQ ID NO: 628, a VHCDR2 encoded by the nucleotide sequence of SEQ ID NO: 629, and a VHCDR3 encoded by the nucleotide sequence of SEQ ID NO: 630; and a VL comprising a VLCDR1 encoded by the nucleotide sequence of SEQ ID NO: 633, a VLCDR2 encoded by the nucleotide sequence of SEQ ID NO: 634, and a VLCDR3 encoded by the nucleotide sequence of SEQ ID NO: 635, each disclosed in Table 3.

In one embodiment, the anti-PD-L1 antibody molecule comprises a VH comprising the amino acid sequence of SEQ ID NO: 606, or an amino acid sequence at least 85%, 90%, 95%, or 99% identical or higher to SEQ ID NO: 606. In one embodiment, the anti-PD-L1 antibody molecule comprises a VL comprising the amino acid sequence of SEQ ID NO: 616, or an amino acid sequence at least 85%, 90%, 95%, or 99% identical or higher to SEQ ID NO: 616. In one embodiment, the anti-PD-L1 antibody molecule comprises a VH comprising the amino acid sequence of SEQ ID NO: 620, or an amino acid sequence at least 85%, 90%, 95%, or 99% identical or higher to SEQ ID NO: 620. In one embodiment, the anti-PD-L1 antibody molecule comprises a VL comprising the amino acid sequence of SEQ ID NO: 624, or an amino acid sequence at least 85%, 90%, 95%, or 99% identical or higher to SEQ ID NO: 624. In one embodiment, the anti-PD-L1 antibody molecule comprises a VH comprising the amino acid sequence of SEQ ID NO: 606 and a VL comprising the amino acid sequence of SEQ ID NO: 616. In one embodiment, the anti-PD-L1 antibody molecule comprises a VH comprising the amino acid sequence of SEQ ID NO: 620 and a VL comprising the amino acid sequence of SEQ ID NO: 624.

In one embodiment, the antibody molecule comprises a VH encoded by the nucleotide sequence of SEQ ID NO: 607, or a nucleotide sequence at least 85%, 90%, 95%, or 99% identical or higher to SEQ ID NO: 607. In one embodiment, the antibody molecule comprises a VL encoded by the nucleotide sequence of SEQ ID NO: 617, or a nucleotide sequence at least 85%, 90%, 95%, or 99% identical or higher to SEQ ID NO: 617. In one embodiment, the antibody molecule comprises a VH encoded by the nucleotide sequence of SEQ ID NO: 621, or a nucleotide sequence at least 85%, 90%, 95%, or 99% identical or higher to SEQ ID NO: 621. In one embodiment, the antibody molecule comprises a VL encoded by the nucleotide sequence of SEQ ID NO: 625, or a nucleotide sequence at least 85%, 90%, 95%, or 99% identical or higher to SEQ ID NO: 625. In one embodiment, the antibody molecule comprises a VH encoded by the nucleotide sequence of SEQ ID NO: 607 and a VL encoded by the nucleotide sequence of SEQ ID NO: 617. In one embodiment, the antibody molecule comprises a VH encoded by the nucleotide sequence of SEQ ID NO: 621 and a VL encoded by the nucleotide sequence of SEQ ID NO: 625.

In one embodiment, the anti-PD-L1 antibody molecule comprises a heavy chain comprising the amino acid sequence of SEQ ID NO: 608, or an amino acid sequence at least 85%, 90%, 95%, or 99% identical or higher to SEQ ID NO: 608. In one embodiment, the anti-PD-L1 antibody molecule comprises a light chain comprising the amino acid sequence of SEQ ID NO: 618, or an amino acid sequence at least 85%, 90%, 95%, or 99% identical or higher to SEQ ID NO: 618. In one embodiment, the anti-PD-L1 antibody molecule comprises a heavy chain comprising the amino acid sequence of SEQ ID NO: 622, or an amino acid sequence at least 85%, 90%, 95%, or 99% identical or higher to SEQ ID NO: 622. In one embodiment, the anti-PD-L1 antibody molecule comprises a light chain comprising the amino acid sequence of SEQ ID NO: 626, or an amino acid sequence at least 85%, 90%, 95%, or 99% identical or higher to SEQ ID NO: 626. In one embodiment, the anti-PD-L1 antibody molecule comprises a heavy chain comprising the amino acid sequence of SEQ ID NO: 608 and a light chain comprising the amino acid sequence of SEQ ID NO: 618. In one embodiment, the anti-PD-L1 antibody molecule comprises a heavy chain comprising the amino acid sequence of SEQ ID NO: 622 and a light chain comprising the amino acid sequence of SEQ ID NO: 626.

In one embodiment, the antibody molecule comprises a heavy chain encoded by the nucleotide sequence of SEQ ID NO: 615, or a nucleotide sequence at least 85%, 90%, 95%, or 99% identical or higher to SEQ ID NO: 615. In one embodiment, the antibody molecule comprises a light chain encoded by the nucleotide sequence of SEQ ID NO: 619, or a nucleotide sequence at least 85%, 90%, 95%, or 99% identical or higher to SEQ ID NO: 619. In one embodiment, the antibody molecule comprises a heavy chain encoded by the nucleotide sequence of SEQ ID NO: 623, or a nucleotide sequence at least 85%, 90%, 95%, or 99% identical or higher to SEQ ID NO: 623. In one embodiment, the antibody molecule comprises a light chain encoded by the nucleotide sequence of SEQ ID NO: 627, or a nucleotide sequence at least 85%, 90%, 95%, or 99% identical or higher to SEQ ID NO: 627. In one embodiment, the antibody molecule comprises a heavy chain encoded by the nucleotide sequence of SEQ ID NO: 615 and a light chain encoded by the nucleotide sequence of SEQ ID NO: 619. In one embodiment, the antibody molecule comprises a heavy chain encoded by the nucleotide sequence of SEQ ID NO: 623 and a light chain encoded by the nucleotide sequence of SEQ ID NO: 627.

The antibody molecules described herein can be made by vectors, host cells, and methods described in US 2016/0108123, incorporated by reference in its entirety.

TABLE 3
Amino acid and nucleotide sequences of
exemplary anti-PD-L1 antibody molecules

| BAP058-Clone O HC | | |
|---|---|---|
| SEQ ID NO: 601 (Kabat) | HCDR1 | SYWMY |
| SEQ ID NO: 602 (Kabat) | HCDR2 | RIDPNSGSTKYNEKFKN |
| SEQ ID NO: 603 (Kabat) | HCDR3 | DYRKGLYAMDY |
| SEQ ID NO: 604 | HCDR1 | GYTFTSY |
| (Chothia) | | |
| SEQ ID NO: 605 | HCDR2 | DPNSGS |
| (Chothia) | | |
| SEQ ID NO: 603 | HCDR3 | DYRKGLYAMDY |
| (Chothia) | | |
| SEQ ID NO: 606 | VH | EVQLVQSGAEVKKPGATVKISCKVSGYTFTSYWMYWVRQARGQ |
| | | RLEWIGRIDPNSGSTKYNEKFKNRFTISRDNSKNTLYLQMNSLRA |
| | | EDTAVYYCARDYRKGLYAMDYWGQGTTVTVSS |
| SEQ ID NO: 607 | DNA VH | GAAGTGCAGCTGGTGCAGTCAGGCGCCGAAGTGAAGAAACCC |
| | | GGCGCTACCGTGAAGATTAGCTGTAAAGTCTCAGGCTACACCT |
| | | TCACTAGCTACTGGATGTACTGGGTCCGACAGGCTAGAGGGCA |
| | | AAGACTGGAGTGGATCGGTAGAATCGACCCTAATAGCGGCTC |
| | | TACTAAGTATAACGAGAAGTTTAAGAATAGGTTCACTATTAGT |
| | | AGGGATAACTCTAAGAACACCCTGTACCTGCAGATGAATAGC |
| | | CTGAGAGCCGAGGACACCGCCGTCTACTACTGCGCTAGAGACT |
| | | ATAGAAAGGGCCTGTACGCTATGGACTACTGGGGTCAAGGCA |
| | | CTACCGTGACCGTGTCTTCA |
| SEQ ID NO: 608 | Heavy | EVQLVQSGAEVKKPGATVKISCKVSGYTFTSYWMYWVRQARGQ |
| | chain | RLEWIGRIDPNSGSTKYNEKFKNRFTISRDNSKNTLYLQMNSLRA |
| | | EDTAVYYCARDYRKGLYAMDYWGQGTTVTVSSASTKGPSVFPL |
| | | APCSRSTSESTAALGCLVKDYFPEPVTVSWNSGALTSGVHTFPAV |
| | | LQSSGLYSLSSVVTVPSSSLGTKTYTCNVDHKPSNTKVDKRVESK |
| | | YGPPCPPCPAPEFLGGPSVFLFPPKPKDTLMISRTPEVTCVVVDVS |
| | | QEDPEVQFNWYVDGVEVHNAKTKPREEQFNSTYRVVSVLTVLH |
| | | QDWLNGKEYKCKVSNKGLPSSIEKTISKAKGQPREPQVYTLPPSQ |
| | | EEMTKNQVSLTCLVKGFYPSDIAVEWESNGQPENNYKTTPPVLD |
| | | SDGSFFLYSRLTVDKSRWQEGNVFSCSVMHEALHNHYTQKSLSL |
| | | SLG |
| SEQ ID NO: 615 | DNA | GAAGTGCAGCTGGTGCAGTCAGGCGCCGAAGTGAAGAAACCC |
| | heavy | GGCGCTACCGTGAAGATTAGCTGTAAAGTCTCAGGCTACACCT |
| | chain | TCACTAGCTACTGGATGTACTGGGTCCGACAGGCTAGAGGGCA |
| | | AAGACTGGAGTGGATCGGTAGAATCGACCCTAATAGCGGCTC |
| | | TACTAAGTATAACGAGAAGTTTAAGAATAGGTTCACTATTAGT |
| | | AGGGATAACTCTAAGAACACCCTGTACCTGCAGATGAATAGC |
| | | CTGAGAGCCGAGGACACCGCCGTCTACTACTGCGCTAGAGACT |
| | | ATAGAAAGGGCCTGTACGCTATGGACTACTGGGGTCAAGGCA |
| | | CTACCGTGACCGTGTCTTCAGCTAGCACTAAGGGCCCGTCCGT |
| | | GTTCCCCCTGGCACCTTGTAGCCGGAGCACTAGCGAATCCACC |
| | | GCTGCCCTCGGCTGCCTGGTCAAGGATTACTTCCCGGAGCCCG |
| | | TGACCGTGTCCTGGAACAGCGGAGCCCTGACCTCCGGAGTGCA |
| | | CACCTTCCCCGCTGTGCTGCAGAGCTCCGGGCTGTACTCGCTG |
| | | TCGTCGGTGGTCACGGTGCCTTCATCTAGCCTGGGTACCAAGA |
| | | CCTACACTTGCAACGTGGACCACAAGCCTTCCAACACTAAGGT |
| | | GGACAAGCGCGTCGAATCGAAGTACGGCCCACCGTGCCCGCC |
| | | TTGTCCCGCGCCGGAGTTCCTCGGCGGTCCCTCGGTCTTTCTGT |
| | | TCCCACCGAAGCCCAAGGACACTTTGATGATTTCCCGCACCCC |
| | | TGAAGTGACATGCGTGGTCGTGGACGTGTCACAGGAAGATCC |
| | | GGAGGTGCAGTTCAATTGGTACGTGGATGGCGTCGAGGTGCA |
| | | CAACGCCAAAACCAAGCCGAGGGAGGAGCAGTTCAACTCCAC |
| | | TTACCGCGTCGTGTCCGTGCTGACGGTGCTGCATCAGGACTGG |
| | | CTGAACGGGAAGGAGTACAAGTGCAAAGTGTCCAACAAGGGA |
| | | CTTCCTAGCTCAATCGAAAAGACCATCTCGAAAGCCAAGGGA |
| | | CAGCCCCGGGAACCCCAAGTGTATACCCTGCCACCGAGCCAG |
| | | GAAGAAATGACTAAGAACCAAGTCTCATTGACTTGCCTTGTGA |
| | | AGGGCTTCTACCCATCGGATATCGCCGTGGAATGGGAGTCCAA |
| | | CGGCCAGCCGGAAAACAACTACAAGACCACCCCTCCGGTGCT |
| | | GGACTCAGACGGATCCTTCTTCCTCTACTCGCGGCTGACCGTG |
| | | GATAAGAGCAGATGGCAGGAGGGAAATGTGTTCAGCTGTTCT |
| | | GTGATGCATGAAGCCCTGCACAACCACTACACTCAGAAGTCCC |
| | | TGTCCCTCTCCCTGGGA |
| BAP058-Clone O LC | | |
| SEQ ID NO: 609 (Kabat) | LCDR1 | KASQDVGTAVA |
| SEQ ID NO: 610 (Kabat) | LCDR2 | WASTRHT |
| SEQ ID NO: 611 (Kabat) | LCDR3 | QQYNSYPLT |
| SEQ ID NO: 612 | LCDR1 | SQDVGTA |
| (Chothia) | | |
| SEQ ID NO: 613 | LCDR2 | WAS |
| (Chothia) | | |
| SEQ ID NO: 614 | LCDR3 | YNSYPL |
| (Chothia) | | |
| SEQ ID NO: 616 | VL | AIQLTQSPSSLSASVGDRVTITCKASQDVGTAVAWYLQKPGQSPQ |
| | | LLIYWASTRHTGVPSRFSGSGSGTDFTFTISSLEAEDAATYYCQQY |
| | | NSYPLTFGQGTKVEIK |
| SEQ ID NO: 617 | DNA VL | GCTATTCAGCTGACTCAGTCACCTAGTAGCCTGAGCGCTAGTG |
| | | TGGGCGATAGAGTGACTATCACCTGTAAAGCCTCTCAGGACGT |
| | | GGGCACCGCCGTGGCCTGGTATCTGCAGAAGCCTGGTCAATCA |
| | | CCTCAGCTGCTGATCTACTGGGCCTCTACTAGACACACCGGCG |
| | | TGCCCTCTAGGTTTAGCGGTAGCGGTAGTGGCACCGACTTCAC |
| | | CTTCACTATCTCTTCACTGGAAGCCGAGGACGCCGCTACCTAC |
| | | TACTGTCAGCAGTATAATAGCTACCCCCTGACCTTCGGTCAAG |
| | | GCACTAAGGTCGAGATTAAG |
| SEQ ID NO: 618 | Light | AIQLTQSPSSLSASVGDRVTITCKASQDVGTAVAWYLQKPGQSPQ |
| | chain | LLIYWASTRHTGVPSRFSGSGSGTDFTFTISSLEAEDAATYYCQQY |
| | | NSYPLTFGQGTKVEIKRTVAAPSVFIFPPSDEQLKSGTASVVCLLN |
| | | NFYPREAKVQWKVDNALQSGNSQESVTEQDSKDSTYSLSSTLTL |
| | | SKADYEKHKVYACEVTHQGLSSPVTKSFNRGEC |
| SEQ ID NO: 619 | DNA | GCTATTCAGCTGACTCAGTCACCTAGTAGCCTGAGCGCTAGTG |
| | light | TGGGCGATAGAGTGACTATCACCTGTAAAGCCTCTCAGGACGT |
| | chain | GGGCACCGCCGTGGCCTGGTATCTGCAGAAGCCTGGTCAATCA |
| | | CCTCAGCTGCTGATCTACTGGGCCTCTACTAGACACACCGGCG |
| | | TGCCCTCTAGGTTTAGCGGTAGCGGTAGTGGCACCGACTTCAC |
| | | CTTCACTATCTCTTCACTGGAAGCCGAGGACGCCGCTACCTAC |
| | | TACTGTCAGCAGTATAATAGCTACCCCCTGACCTTCGGTCAAG |
| | | GCACTAAGGTCGAGATTAAGCGTACGGTGGCCGCTCCCAGCGT |
| | | GTTCATCTTCCCCCCCAGCGACGAGCAGCTGAAGAGCGGCACC |
| | | GCCAGCGTGGTGTGCCTGCTGAACAACTTCTACCCCCGGGAGG |
| | | CCAAGGTGCAGTGGAAGGTGGACAACGCCCTGCAGAGCGGCA |
| | | ACAGCCAGGAGAGCGTCACCGAGCAGGACAGCAAGGACTCCA |
| | | CCTACAGCCTGAGCAGCACCCTGACCCTGAGCAAGGCCGACT |
| | | ACGAGAAGCATAAGGTGTACGCCTGCGAGGTGACCCACCAGG |
| | | GCCTGTCCAGCCCCGTGACCAAGAGCTTCAACAGGGGCGAGT |
| | | GC |
| BAP058-Clone N HC | | |
| SEQ ID NO: 601 (Kabat) | HCDR1 | SYWMY |
| SEQ ID NO: 602 (Kabat) | HCDR2 | RIDPNSGSTKYNEKFKN |
| SEQ ID NO: 603 (Kabat) | HCDR3 | DYRKGLYAMDY |
| SEQ ID NO: 604 | HCDR1 | GYTFTSY |
| (Chothia) | | |
| SEQ ID NO: 605 | HCDR2 | DPNSGS |
| (Chothia) | | |
| SEQ ID NO: 603 | HCDR3 | DYRKGLYAMDY |
| (Chothia) | | |
| SEQ ID NO: 620 | VH | EVQLVQSGAEVKKPGATVKISCKVSGYTFTSYWMYWVRQATGQ |
| | | GLEWMGRIDPNSGSTKYNEKFKNRVTITADKSTSTAYMELSSLRS |
| | | EDTAVYYCARDYRKGLYAMDYWGQGTTVTVSS |
| SEQ ID NO: 621 | DNA VH | GAAGTGCAGCTGGTGCAGTCAGGCGCCGAAGTGAAGAAACCC |
| | | GGCGCTACCGTGAAGATTAGCTGTAAAGTCTCAGGCTACACCT |
| | | TCACTAGCTACTGGATGTACTGGGTCCGACAGGCTACCGGTCA |
| | | AGGCCTGGAGTGGATGGGTAGAATCGACCCTAATAGCGGCTC |
| | | TACTAAGTATAACGAGAAGTTTAAGAATAGAGTGACTATCACC |
| | | GCCGATAAGTCTACTAGCACCGCCTATATGGAACTGTCTAGCC |
| | | TGAGATCAGAGGACACCGCCGTCTACTACTGCGCTAGAGACTA |
| | | TAGAAAGGGCCTGTACGCTATGGACTACTGGGGTCAAGGCAC |
| | | TACCGTGACCGTGTCTTCA |
| SEQ ID NO: 622 | Heavy | EVQLVQSGAEVKKPGATVKISCKVSGYTFTSYWMYWVRQATGQ |
| | chain | GLEWMGRIDPNSGSTKYNEKFKNRVTITADKSTSTAYMELSSLRS |
| | | EDTAVYYCARDYRKGLYAMDYWGQGTTVTVSSASTKGPSVFPL |
| | | APCSRSTSESTAALGCLVKDYFPEPVTVSWNSGALTSGVHTFPAV |
| | | LQSSGLYSLSSVVTVPSSSLGTKTYTCNVDHKPSNTKVDKRVESK |
| | | YGPPCPPCPAPEFLGGPSVFLFPPKPKDTLMISRTPEVTCVVVDVS |
| | | QEDPEVQFNWYVDGVEVHNAKTKPREEQFNSTYRVVSVLTVLH |
| | | QDWLNGKEYKCKVSNKGLPSSIEKTISKAKGQPREPQVYTLPPSQ |
| | | EEMTKNQVSLTCLVKGFYPSDIAVEWESNGQPENNYKTTPPVLD |
| | | SDGSFFLYSRLTVDKSRWQEGNVFSCSVMHEALHNHYTQKSLSL |
| | | SLG |
| SEQ ID NO: 623 | DNA | GAAGTGCAGCTGGTGCAGTCAGGCGCCGAAGTGAAGAAACCC |
| | heavy | GGCGCTACCGTGAAGATTAGCTGTAAAGTCTCAGGCTACACCT |
| | chain | TCACTAGCTACTGGATGTACTGGGTCCGACAGGCTACCGGTCA |
| | | AGGCCTGGAGTGGATGGGTAGAATCGACCCTAATAGCGGCTC |
| | | TACTAAGTATAACGAGAAGTTTAAGAATAGAGTGACTATCACC |
| | | GCCGATAAGTCTACTAGCACCGCCTATATGGAACTGTCTAGCC |
| | | TGAGATCAGAGGACACCGCCGTCTACTACTGCGCTAGAGACTA |
| | | TAGAAAGGGCCTGTACGCTATGGACTACTGGGGTCAAGGCAC |
| | | TACCGTGACCGTGTCTTCAGCTAGCACTAAGGGCCCGTCCGTG |
| | | TTCCCCCTGGCACCTTGTAGCCGGAGCACTAGCGAATCCACCG |
| | | CTGCCCTCGGCTGCCTGGTCAAGGATTACTTCCCGGAGCCCGT |
| | | GACCGTGTCCTGGAACAGCGGAGCCCTGACCTCCGGAGTGCA |
| | | CACCTTCCCCGCTGTGCTGCAGAGCTCCGGGCTGTACTCGCTG |
| | | TCGTCGGTGGTCACGGTGCCTTCATCTAGCCTGGGTACCAAGA |
| | | CCTACACTTGCAACGTGGACCACAAGCCTTCCAACACTAAGGT |
| | | GGACAAGCGCGTCGAATCGAAGTACGGCCCACCGTGCCCGCC |
| | | TTGTCCCGCGCCGGAGTTCCTCGGCGGTCCCTCGGTCTTTCTGT |
| | | TCCCACCGAAGCCCAAGGACACTTTGATGATTTCCCGCACCCC |
| | | TGAAGTGACATGCGTGGTCGTGGACGTGTCACAGGAAGATCC |
| | | GGAGGTGCAGTTCAATTGGTACGTGGATGGCGTCGAGGTGCA |
| | | CAACGCCAAAACCAAGCCGAGGGAGGAGCAGTTCAACTCCAC |
| | | TTACCGCGTCGTGTCCGTGCTGACGGTGCTGCATCAGGACTGG |
| | | CTGAACGGGAAGGAGTACAAGTGCAAAGTGTCCAACAAGGGA |
| | | CTTCCTAGCTCAATCGAAAAGACCATCTCGAAAGCCAAGGGA |
| | | CAGCCCCGGGAACCCCAAGTGTATACCCTGCCACCGAGCCAG |
| | | GAAGAAATGACTAAGAACCAAGTCTCATTGACTTGCCTTGTGA |
| | | AGGGCTTCTACCCATCGGATATCGCCGTGGAATGGGAGTCCAA |
| | | CGGCCAGCCGGAAAACAACTACAAGACCACCCCTCCGGTGCT |
| | | GGACTCAGACGGATCCTTCTTCCTCTACTCGCGGCTGACCGTG |
| | | GATAAGAGCAGATGGCAGGAGGGAAATGTGTTCAGCTGTTCT |
| | | GTGATGCATGAAGCCCTGCACAACCACTACACTCAGAAGTCCC |
| | | TGTCCCTCTCCCTGGGA |
| BAP058-Clone N LC | | |
| SEQ ID NO: 609 (Kabat) | LCDR1 | KASQDVGTAVA |
| SEQ ID NO: 610 (Kabat) | LCDR2 | WASTRHT |
| SEQ ID NO: 611 (Kabat) | LCDR3 | QQYNSYPLT |
| SEQ ID NO: 612 | LCDR1 | SQDVGTA |
| (Chothia) | | |
| SEQ ID NO: 613 | LCDR2 | WAS |
| (Chothia) | | |
| SEQ ID NO: 614 | LCDR3 | YNSYPL |
| (Chothia) | | |
| SEQ ID NO: 624 | VL | DVVMTQSPLSLPVTLGQPASISCKASQDVGTAVAWYQQKPGQAP |
| | | RLLIYWASTRHTGVPSRFSGSGSGTEFTLTISSLQPDDFATYYCQQ |
| | | YNSYPLTFGQGTKVEIK |
| SEQ ID NO: 625 | DNA | GACGTCGTGATGACTCAGTCACCCCTGAGCCTGCCCGTGACCC |
| | VL | TGGGGCAGCCCGCCTCTATTAGCTGTAAAGCCTCTCAGGACGT |
| | | GGGCACCGCCGTGGCCTGGTATCAGCAGAAGCCAGGGCAAGC |
| | | CCCTAGACTGCTGATCTACTGGGCCTCTACTAGACACACCGGC |
| | | GTGCCCTCTAGGTTTAGCGGTAGCGGTAGTGGCACCGAGTTCA |
| | | CCCTGACTATCTCTTCACTGCAGCCCGACGACTTCGCTACCTAC |
| | | TACTGTCAGCAGTATAATAGCTACCCCCTGACCTTCGGTCAAG |
| | | GCACTAAGGTCGAGATTAAG |
| SEQ ID NO: 626 | Light | DVVMTQSPLSLPVTLGQPASISCKASQDVGTAVAWYQQKPGQAP |
| | chain | RLLIYWASTRHTGVPSRFSGSGSGTEFTLTISSLQPDDFATYYCQQ |
| | | YNSYPLTFGQGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| SEQ ID NO: 627 | DNA | GACGTCGTGATGACTCAGTCACCCCTGAGCCTGCCCGTGACCC |
| | light | TGGGGCAGCCCGCCTCTATTAGCTGTAAAGCCTCTCAGGACGT |
| | chain | GGGCACCGCCGTGGCCTGGTATCAGCAGAAGCCAGGGCAAGC |
| | | CCCTAGACTGCTGATCTACTGGGCCTCTACTAGACACACCGGC |
| | | GTGCCCTCTAGGTTTAGCGGTAGCGGTAGTGGCACCGAGTTCA |
| | | CCCTGACTATCTCTTCACTGCAGCCCGACGACTTCGCTACCTAC |
| | | TACTGTCAGCAGTATAATAGCTACCCCCTGACCTTCGGTCAAG |
| | | GCACTAAGGTCGAGATTAAGCGTACGGTGGCCGCTCCCAGCGT |
| | | GTTCATCTTCCCCCCCAGCGACGAGCAGCTGAAGAGCGGCACC |
| | | GCCAGCGTGGTGTGCCTGCTGAACAACTTCTACCCCCGGGAGG |
| | | CCAAGGTGCAGTGGAAGGTGGACAACGCCCTGCAGAGCGGCA |
| | | ACAGCCAGGAGAGCGTCACCGAGCAGGACAGCAAGGACTCCA |
| | | CCTACAGCCTGAGCAGCACCCTGACCCTGAGCAAGGCCGACT |
| | | ACGAGAAGCATAAGGTGTACGCCTGCGAGGTGACCCACCAGG |
| | | GCCTGTCCAGCCCCGTGACCAAGAGCTTCAACAGGGGCGAGT |
| | | GC |
| BAP058-Clone O HC | | |
| SEQ ID NO: 628 (Kabat) | HCDR1 | AGCTACTGGATGTAC |
| SEQ ID NO: 629 (Kabat) | HCDR2 | AGAATCGACCCTAATAGCGGCTCTACTAAGTATAACGAGAAG |
| | | TTTAAGAAT |
| SEQ ID NO: 630 (Kabat) | HCDR3 | GACTATAGAAAGGGCCTGTACGCTATGGACTAC |
| SEQ ID NO: 631 | HCDR1 | GGCTACACCTTCACTAGCTAC |
| (Chothia) | | |
| SEQ ID NO: 632 | HCDR2 | GACCCTAATAGCGGCTCT |
| (Chothia) | | |
| SEQ ID NO: 630 | HCDR3 | GACTATAGAAAGGGCCTGTACGCTATGGACTAC |
| (Chothia) | | |
| BAP058-Clone O LC | | |
| SEQ ID NO: 633 (Kabat) | LCDR1 | AAAGCCTCTCAGGACGTGGGCACCGCCGTGGCC |
| SEQ ID NO: 634 (Kabat) | LCDR2 | TGGGCCTCTACTAGACACACC |
| SEQ ID NO: 635 (Kabat) | LCDR3 | CAGCAGTATAATAGCTACCCCCTGACC |
| SEQ ID NO: 636 | LCDR1 | TCTCAGGACGTGGGCACCGCC |
| (Chothia) | | |
| SEQ ID NO: 637 | LCDR2 | TGGGCCTCT |
| (Chothia) | | |
| SEQ ID NO: 638 | LCDR3 | TATAATAGCTACCCCCTG |
| (Chothia) | | |
| BAP058-Clone N HC | | |
| SEQ ID NO: 628 (Kabat) | HCDR1 | AGCTACTGGATGTAC |
| SEQ ID NO: 629 (Kabat) | HCDR2 | AGAATCGACCCTAATAGCGGCTCTACTAAGTATAACGAGAAG |
| | | TTTAAGAAT |
| SEQ ID NO: 630 (Kabat) | HCDR3 | GACTATAGAAAGGGCCTGTACGCTATGGACTAC |
| SEQ ID NO: 631 | HCDR1 | GGCTACACCTTCACTAGCTAC |
| (Chothia) | | |
| SEQ ID NO: 632 | HCDR2 | GACCCTAATAGCGGCTCT |
| (Chothia) | | |
| SEQ ID NO: 630 | HCDR3 | GACTATAGAAAGGGCCTGTACGCTATGGACTAC |
| (Chothia) | | |
| BAP058-Clone N LC | | |
| SEQ ID NO: 633 (Kabat) | LCDR1 | AAAGCCTCTCAGGACGTGGGCACCGCCGTGGCC |
| SEQ ID NO: 634 (Kabat) | LCDR2 | TGGGCCTCTACTAGACACACC |
| SEQ ID NO: 635 (Kabat) | LCDR3 | CAGCAGTATAATAGCTACCCCCTGACC |
| SEQ ID NO: 636 | LCDR1 | TCTCAGGACGTGGGCACCGCC |
| (Chothia) | | |
| SEQ ID NO: 637 | LCDR2 | TGGGCCTCT |
| (Chothia) | | |
| SEQ ID NO: 638 | LCDR3 | TATAATAGCTACCCCCTG |
| (Chothia) | | |

### Other Exemplary PD-L1 Inhibitors

In one embodiment, the anti-PD-L1 antibody molecule is Atezolizumab (Genentech/Roche), also known as MPDL3280A, RG7446, R05541267, YW243.55.S70, or TECENTRIQ™. Atezolizumab and other anti-PD-L1 antibodies are disclosed in U.S. Pat. No. 8,217,149, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Atezolizuma, e.g., as disclosed in Table 4.

In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Avelumab, e.g., as disclosed in Table 4.

In one embodiment, the anti-PD-L1 antibody molecule is Durvalumab (MedImmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Durvalumab, e.g., as disclosed in Table 4.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559, e.g., as disclosed in Table 4.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

TABLE 4
Amino acid sequences of other exemplary anti-PD-L1 antibody molecules

| | | |
|---|---|---|
| Atezolizumab | | |
| SEQ ID NO: 639 | Heavy | EVQLVESGGGLVQPGGSLRLSCAASGFTFSDSWIHWVRQAPGKGLEWVAWI |
| | chain | SPYGGSTYYADSVKGRFTISADTSKNTAYLQMNSLRAEDTAVYYCARRHWP |
| | | GGFDYWGQGTLVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLH |
| | | QDWLNGKEYKCKVSNKALPAPIEKTISKAKGQPREPQVYTLPPSREEMTKNQ |
| | | VSLTCLVKGFYPSDIAVEWESNGQPENNYKTTPPVLDSDGSFFLYSKLTVDKS |
| | | RWQQGNVFSCSVMHEALHNHYTQKSLSLSPGK |
| SEQ ID NO: 640 | Light | DIQMTQSPSSLSASVGDRVTITCRASQDVSTAVAWYQQKPGKAPKLLIYSASF |
| | chain | LYSGVPSRFSGSGSGTDFTLTISSLQPEDFATYYCQQYLYHPATFGQGTKVEIK |
| | | RTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNR |
| | | GEC |
| Avelumab | | |
| SEQ ID NO: 641 | Heavy | EVQLLESGGGLVQPGGSLRLSCAASGFTFSSYIMMWVRQAPGKGLEWVSSIY |
| | chain | PSGGITFYADTVKGRFTISRDNSKNTLYLQMNSLRAEDTAVYYCARIKLGTV |
| | | TTVDYWGQGTLVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLH |
| | | QDWLNGKEYKCKVSNKALPAPIEKTISKAKGQPREPQVYTLPPSRDELTKNQ |
| | | VSLTCLVKGFYPSDIAVEWESNGQPENNYKTTPPVLDSDGSFFLYSKLTVDKS |
| | | RWQQGNVFSCSVMHEALHNHYTQKSLSLSPGK |
| SEQ ID NO: 642 | Light | QSALTQPASVSGSPGQSITISCTGTSSDVGGYNYVSWYQQHPGKAPKLMIYD |
| | chain | VSNRPSGVSNRFSGSKSGNTASLTISGLQAEDEADYYCSSYTSSSTRVFGTGT |
| | | KVTVLGQPKANPTVTLFPPSSEELQANKATLVCLISDFYPGAVTVAWKADGS |
| | | PVKAGVETTKPSKQSNNKYAASSYLSLTPEQWKSHRSYSCQVTHEGSTVEKT |
| | | VAPTECS |
| Durvalumab | | |
| SEQ ID NO: 643 | Heavy | EVQLVESGGGLVQPGGSLRLSCAASGFTFSRYWMSWVRQAPGKGLEWVANI |
| | chain | KQDGSEKYYVDSVKGRFTISRDNAKNSLYLQMNSLRAEDTAVYYCAREGG |
| | | WFGELAFDYWGQGTLVTVSSASTKGPSVFPLAPSSKSTSGGTAALGCLVKDY |
| | | FPEPVTVSWNSGALTSGVHTFPAVLQSSGLYSLSSVVTVPSSSLGTQTYICNV |
| | | NHKPSNTKVDKRVEPKSCDKTHTCPPCPAPEFEGGPSVFLFPPKPKDTLMISR |
| | | TPEVTCVVVDVSHEDPEVKFNWYVDGVEVHNAKTKPREEQYNSTYRVVSV |
| | | LTVLHQDWLNGKEYKCKVSNKALPASIEKTISKAKGQPREPQVYTLPPSREE |
| | | MTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGK |
| SEQ ID NO: 644 | Light | SRATGIPDRFSGSGSGTDFTLTISRLEPEDFAVYYCQQYGSLPWTFGQGTKVEI |
| | chain | KRTVAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKSFNR |
| | | GEC |
| BMS-936559 | | |
| SEQ ID NO: 645 | VH | QVQLVQSGAEVKKPGSSVKVSCKTSGDTFSTYAISWVRQAPGQGLEWMGGII |
| | | PIFGKAHYAQKFQGRVTITADESTSTAYMELSSLRSEDTAVYFCARKFHFVSG |
| | | SPFGMDVWGQGTTVTVSS |
| SEQ ID NO: 646 | VL | EIVLTQSPATLSLSPGERATLSCRASQSVSSYLAWYQQKPGQAPRLLIYDASN |
| | | RATGIPARFSGSGSGTDFTLTISSLEPEDFAVYYCQQRSNWPTFGQGTKVEIK |

### PD-1 Inhibitors

In certain embodiments, the anti-PD-L1 antibody molecule described herein is administered in combination with a PD-1 inhibitor. In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHRI210 (Incyte), or AMP-224 (Amplimmune).

### Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 1 (e.g., from the heavy and light chain variable region sequences of BAP049-Clone-E or BAP049-Clone-B disclosed in Table 1), or encoded by a nucleotide sequence shown in Table 1. In some embodiments, the CDRs are according to the Kabat definition (e.g., as set out in Table 1). In some embodiments, the CDRs are according to the Chothia definition (e.g., as set out in Table 1). In some embodiments, the CDRs are according to the combined CDR definitions of both Kabat and Chothia (e.g., as set out in Table 1). In one embodiment, the combination of Kabat and Chothia CDR of VH CDR1 comprises the amino acid sequence GYTFTTYWMH (SEQ ID NO: 541). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 1, or encoded by a nucleotide sequence shown in Table 1.

In one embodiment, the anti-PD-1 antibody molecule comprises a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 1.

In one embodiment, the antibody molecule comprises a VH comprising a VHCDR1 encoded by the nucleotide sequence of SEQ ID NO: 524, a VHCDR2 encoded by the nucleotide sequence of SEQ ID NO: 525, and a VHCDR3 encoded by the nucleotide sequence of SEQ ID NO: 526; and a VL comprising a VLCDR1 encoded by the nucleotide sequence of SEQ ID NO: 529, a VLCDR2 encoded by the nucleotide sequence of SEQ ID NO: 530, and a VLCDR3 encoded by the nucleotide sequence of SEQ ID NO: 531, each disclosed in Table 1.

In one embodiment, the anti-PD-1 antibody molecule comprises a VH comprising the amino acid sequence of SEQ ID NO: 506, or an amino acid sequence at least 85%, 90%, 95%, or 99% identical or higher to SEQ ID NO: 506. In one embodiment, the anti-PD-1 antibody molecule comprises a VL comprising the amino acid sequence of SEQ ID NO: 520, or an amino acid sequence at least 85%, 90%, 95%, or 99% identical or higher to SEQ ID NO: 520. In one embodiment, the anti-PD-1 antibody molecule comprises a VL comprising the amino acid sequence of SEQ ID NO: 516, or an amino acid sequence at least 85%, 90%, 95%, or 99% identical or higher to SEQ ID NO: 516. In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

In one embodiment, the antibody molecule comprises a VH encoded by the nucleotide sequence of SEQ ID NO: 507, or a nucleotide sequence at least 85%, 90%, 95%, or 99% identical or higher to SEQ ID NO: 507. In one embodiment, the antibody molecule comprises a VL encoded by the nucleotide sequence of SEQ ID NO: 521 or 517, or a nucleotide sequence at least 85%, 90%, 95%, or 99% identical or higher to SEQ ID NO: 521 or 517. In one embodiment, the antibody molecule comprises a VH encoded by the nucleotide sequence of SEQ ID NO: 507 and a VL encoded by the nucleotide sequence of SEQ ID NO: 521 or 517.

In one embodiment, the anti-PD-1 antibody molecule comprises a heavy chain comprising the amino acid sequence of SEQ ID NO: 508, or an amino acid sequence at least 85%, 90%, 95%, or 99% identical or higher to SEQ ID NO: 508. In one embodiment, the anti-PD-1 antibody molecule comprises a light chain comprising the amino acid sequence of SEQ ID NO: 522, or an amino acid sequence at least 85%, 90%, 95%, or 99% identical or higher to SEQ ID NO: 522. In one embodiment, the anti-PD-1 antibody molecule comprises a light chain comprising the amino acid sequence of SEQ ID NO: 518, or an amino acid sequence at least 85%, 90%, 95%, or 99% identical or higher to SEQ ID NO: 518. In one embodiment, the anti-PD-1 antibody molecule comprises a heavy chain comprising the amino acid sequence of SEQ ID NO: 508 and a light chain comprising the amino acid sequence of SEQ ID NO: 522. In one embodiment, the anti-PD-1 antibody molecule comprises a heavy chain comprising the amino acid sequence of SEQ ID NO: 508 and a light chain comprising the amino acid sequence of SEQ ID NO: 518.

In one embodiment, the antibody molecule comprises a heavy chain encoded by the nucleotide sequence of SEQ ID NO: 509, or a nucleotide sequence at least 85%, 90%, 95%, or 99% identical or higher to SEQ ID NO: 509. In one embodiment, the antibody molecule comprises a light chain encoded by the nucleotide sequence of SEQ ID NO: 523 or 519, or a nucleotide sequence at least 85%, 90%, 95%, or 99% identical or higher to SEQ ID NO: 523 or 519. In one embodiment, the antibody molecule comprises a heavy chain encoded by the nucleotide sequence of SEQ ID NO: 509 and a light chain encoded by the nucleotide sequence of SEQ ID NO: 523 or 519.

The antibody molecules described herein can be made by vectors, host cells, and methods described in US 2015/0210769, incorporated by reference in its entirety.

TABLE 1
Amino acid and nucleotide sequences of exemplary anti-PD-1 antibody molecules

| BAP049-Clone-B HC | | |
|---|---|---|
| SEQ ID NO: 501 (Kabat) | HCDR1 | TYWMH |
| SEQ ID NO: 502 (Kabat) | HCDR2 | NIYPGTGGSNFDEKFKN |
| SEQ ID NO: 503 (Kabat) | HCDR3 | WTTGTGAY |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQATGQG |
| | | LEWMGNIYPGTGGSNFDEKFKNRVTITADKSTSTAYMELSSLRSE |
| | | DTAVYYCTRWTTGTGAYWGQGTTVTVSS |
| SEQ ID NO: 507 | DNA VH | GAGGTGCAGCTGGTGCAGTCAGGCGCCGAAGTGAAGAAGCCCG |
| | | GCGAGTCACTGAGAATTAGCTGTAAAGGTTCAGGCTACACCTT |
| | | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTGA |
| | | GATCAGAGGACACCGCCGTCTACTACTGCACTAGGTGGACTAC |
| | | CGGCACAGGCGCCTACTGGGGTCAAGGCACTACCGTGACCGTG |
| | | TCTAGC |
| SEQ ID NO: 508 | Heavy | EVQLVQSGAEVKKPGESLRISCKGSGYTFTTYWMHWVRQATGQG |
| | chain | LEWMGNIYPGTGGSNFDEKFKNRVTITADKSTSTAYMELSSLRSE |
| | | DTAVYYCTRWTTGTGAYWGQGTTVTVSSASTKGPSVFPLAPCSRS |
| | | TSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQF |
| | | NWYVDGVEVHNAKTKPREEQFNSTYRVVSVLTVLHQDWLNGKE |
| | | YKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRL |
| | | TVDKSRWQEGNVFSCSVMHEALHNHYTQKSLSLSLG |
| SEQ ID NO: 509 | DNA | GAGGTGCAGCTGGTGCAGTCAGGCGCCGAAGTGAAGAAGCCCG |
| | heavy | GCGAGTCACTGAGAATTAGCTGTAAAGGTTCAGGCTACACCTT |
| | chain | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTGA |
| | | GATCAGAGGACACCGCCGTCTACTACTGCACTAGGTGGACTAC |
| | | CGGCACAGGCGCCTACTGGGGTCAAGGCACTACCGTGACCGTG |
| | | TCTAGCGCTAGCACTAAGGGCCCGTCCGTGTTCCCCCTGGCACC |
| | | TTGTAGCCGGAGCACTAGCGAATCCACCGCTGCCCTCGGCTGCC |
| | | TGGTCAAGGATTACTTCCCGGAGCCCGTGACCGTGTCCTGGAAC |
| | | AGCGGAGCCCTGACCTCCGGAGTGCACACCTTCCCCGCTGTGCT |
| | | GCAGAGCTCCGGGCTGTACTCGCTGTCGTCGGTGGTCACGGTGC |
| | | CTTCATCTAGCCTGGGTACCAAGACCTACACTTGCAACGTGGAC |
| | | CACAAGCCTTCCAACACTAAGGTGGACAAGCGCGTCGAATCGA |
| | | AGTACGGCCCACCGTGCCCGCCTTGTCCCGCGCCGGAGTTCCTC |
| | | GGCGGTCCCTCGGTCTTTCTGTTCCCACCGAAGCCCAAGGACAC |
| | | TTTGATGATTTCCCGCACCCCTGAAGTGACATGCGTGGTCGTGG |
| | | ACGTGTCACAGGAAGATCCGGAGGTGCAGTTCAATTGGTACGT |
| | | GGATGGCGTCGAGGTGCACAACGCCAAAACCAAGCCGAGGGA |
| | | GGAGCAGTTCAACTCCACTTACCGCGTCGTGTCCGTGCTGACGG |
| | | TGCTGCATCAGGACTGGCTGAACGGGAAGGAGTACAAGTGCAA |
| | | AGTGTCCAACAAGGGACTTCCTAGCTCAATCGAAAAGACCATC |
| | | TCGAAAGCCAAGGGACAGCCCCGGGAACCCCAAGTGTATACCC |
| | | TGCCACCGAGCCAGGAAGAAATGACTAAGAACCAAGTCTCATT |
| | | GACTTGCCTTGTGAAGGGCTTCTACCCATCGGATATCGCCGTGG |
| | | AATGGGAGTCCAACGGCCAGCCGGAAAACAACTACAAGACCA |
| | | CCCCTCCGGTGCTGGACTCAGACGGATCCTTCTTCCTCTACTCG |
| | | CGGCTGACCGTGGATAAGAGCAGATGGCAGGAGGGAAATGTGT |
| | | TCAGCTGTTCTGTGATGCATGAAGCCCTGCACAACCACTACACT |
| | | CAGAAGTCCCTGTCCCTCTCCCTGGGA |
| BAP049-Clone-B LC | | |
| SEQ ID NO: 510 (Kabat) | LCDR1 | KSSQSLLDSGNQKNFLT |
| SEQ ID NO: 511 (Kabat) | LCDR2 | WASTRES |
| SEQ ID NO: 512 (Kabat) | LCDR3 | QNDYSYPYT |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 513 | LCDR3 | DYSYPY |
| (Chothia) | | |
| SEQ ID NO: 516 | VL | EIVLTQSPATLSLSPGERATLSCKSSQSLLDSGNQKNFLTWYQQKP |
| | | GKAPKLLIYWASTRESGVPSRFSGSGSGTDFTFTISSLQPEDIATYY |
| | | CQNDYSYPYTFGQGTKVEIK |
| SEQ ID NO: 517 | DNA VL | GAGATCGTCCTGACTCAGTCACCCGCTACCCTGAGCCTGAGCCC |
| | | TGGCGAGCGGGCTACACTGAGCTGTAAATCTAGTCAGTCACTG |
| | | CTGGATAGCGGTAATCAGAAGAACTTCCTGACCTGGTATCAGC |
| | | AGAAGCCCGGTAAAGCCCCTAAGCTGCTGATCTACTGGGCCTC |
| | | TACTAGAGAATCAGGCGTGCCCTCTAGGTTTAGCGGTAGCGGT |
| | | AGTGGCACCGACTTCACCTTCACTATCTCTAGCCTGCAGCCCGA |
| | | GGATATCGCTACCTACTACTGTCAGAACGACTATAGCTACCCCT |
| | | ACACCTTCGGTCAAGGCACTAAGGTCGAGATTAAG |
| SEQ ID NO: 518 | Light | EIVLTQSPATLSLSPGERATLSCKSSQSLLDSGNQKNFLTWYQQKP |
| | chain | GKAPKLLIYWASTRESGVPSRFSGSGSGTDFTFTISSLQPEDIATYY |
| | | CQNDYSYPYTFGQGTKVEIKRTVAAPSVFIFPPSDEQLKSGTASVV |
| | | CLLNNFYPREAKVQWKVDNALQSGNSQESVTEQDSKDSTYSLSST |
| | | LTLSKADYEKHKVYACEVTHQGLSSPVTKSFNRGEC |
| SEQ ID NO: 519 | DNA | GAGATCGTCCTGACTCAGTCACCCGCTACCCTGAGCCTGAGCCC |
| | light | TGGCGAGCGGGCTACACTGAGCTGTAAATCTAGTCAGTCACTG |
| | chain | CTGGATAGCGGTAATCAGAAGAACTTCCTGACCTGGTATCAGC |
| | | AGAAGCCCGGTAAAGCCCCTAAGCTGCTGATCTACTGGGCCTC |
| | | TACTAGAGAATCAGGCGTGCCCTCTAGGTTTAGCGGTAGCGGT |
| | | AGTGGCACCGACTTCACCTTCACTATCTCTAGCCTGCAGCCCGA |
| | | GGATATCGCTACCTACTACTGTCAGAACGACTATAGCTACCCCT |
| | | ACACCTTCGGTCAAGGCACTAAGGTCGAGATTAAGCGTACGGT |
| | | GGCCGCTCCCAGCGTGTTCATCTTCCCCCCCAGCGACGAGCAGC |
| | | TGAAGAGCGGCACCGCCAGCGTGGTGTGCCTGCTGAACAACTT |
| | | CTACCCCCGGGAGGCCAAGGTGCAGTGGAAGGTGGACAACGCC |
| | | CTGCAGAGCGGCAACAGCCAGGAGAGCGTCACCGAGCAGGAC |
| | | AGCAAGGACTCCACCTACAGCCTGAGCAGCACCCTGACCCTGA |
| | | GCAAGGCCGACTACGAGAAGCATAAGGTGTACGCCTGCGAGGT |
| | | GACCCACCAGGGCCTGTCCAGCCCCGTGACCAAGAGCTTCAAC |
| | | AGGGGCGAGTGC |
| BAP049-Clone-E HC | | |
| SEQ ID NO: 501 (Kabat) | HCDR1 | TYWMH |
| SEQ ID NO: 502 (Kabat) | HCDR2 | NIYPGTGGSNFDEKFKN |
| SEQ ID NO: 503 (Kabat) | HCDR3 | WTTGTGAY |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQATGQG |
| | | LEWMGNIYPGTGGSNFDEKFKNRVTITADKSTSTAYMELSSLRSE |
| | | DTAVYYCTRWTTGTGAYWGQGTTVTVSS |
| SEQ ID NO: 507 | DNA VH | GAGGTGCAGCTGGTGCAGTCAGGCGCCGAAGTGAAGAAGCCCG |
| | | GCGAGTCACTGAGAATTAGCTGTAAAGGTTCAGGCTACACCTT |
| | | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTGA |
| | | GATCAGAGGACACCGCCGTCTACTACTGCACTAGGTGGACTAC |
| | | CGGCACAGGCGCCTACTGGGGTCAAGGCACTACCGTGACCGTG |
| | | TCTAGC |
| SEQ ID NO: 508 | Heavy | EVQLVQSGAEVKKPGESLRISCKGSGYTFTTYWMHWVRQATGQG |
| | chain | LEWMGNIYPGTGGSNFDEKFKNRVTITADKSTSTAYMELSSLRSE |
| | | DTAVYYCTRWTTGTGAYWGQGTTVTVSSASTKGPSVFPLAPCSRS |
| | | TSESTAALGCLVKDYFPEPVTVSWNSGALTSGVHTFPAVLQSSGL |
| | | YSLSSVVTVPSSSLGTKTYTCNVDHKPSNTKVDKRVESKYGPPCPP |
| | | CPAPEFLGGPSVFLFPPKPKDTLMISRTPEVTCVVVDVSQEDPEVQF |
| | | NWYVDGVEVHNAKTKPREEQFNSTYRVVSVLTVLHQDWLNGKE |
| | | YKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRL |
| | | TVDKSRWQEGNVFSCSVMHEALHNHYTQKSLSLSLG |
| SEQ ID NO: 509 | DNA | GAGGTGCAGCTGGTGCAGTCAGGCGCCGAAGTGAAGAAGCCCG |
| | heavy | GCGAGTCACTGAGAATTAGCTGTAAAGGTTCAGGCTACACCTT |
| | chain | CACTACCTACTGGATGCACTGGGTCCGCCAGGCTACCGGTCAA |
| | | GGCCTCGAGTGGATGGGTAATATCTACCCCGGCACCGGCGGCT |
| | | CTAACTTCGACGAGAAGTTTAAGAATAGAGTGACTATCACCGC |
| | | CGATAAGTCTACTAGCACCGCCTATATGGAACTGTCTAGCCTGA |
| | | GATCAGAGGACACCGCCGTCTACTACTGCACTAGGTGGACTAC |
| | | CGGCACAGGCGCCTACTGGGGTCAAGGCACTACCGTGACCGTG |
| | | TCTAGCGCTAGCACTAAGGGCCCGTCCGTGTTCCCCCTGGCACC |
| | | TTGTAGCCGGAGCACTAGCGAATCCACCGCTGCCCTCGGCTGCC |
| | | TGGTCAAGGATTACTTCCCGGAGCCCGTGACCGTGTCCTGGAAC |
| | | AGCGGAGCCCTGACCTCCGGAGTGCACACCTTCCCCGCTGTGCT |
| | | GCAGAGCTCCGGGCTGTACTCGCTGTCGTCGGTGGTCACGGTGC |
| | | CTTCATCTAGCCTGGGTACCAAGACCTACACTTGCAACGTGGAC |
| | | CACAAGCCTTCCAACACTAAGGTGGACAAGCGCGTCGAATCGA |
| | | AGTACGGCCCACCGTGCCCGCCTTGTCCCGCGCCGGAGTTCCTC |
| | | GGCGGTCCCTCGGTCTTTCTGTTCCCACCGAAGCCCAAGGACAC |
| | | TTTGATGATTTCCCGCACCCCTGAAGTGACATGCGTGGTCGTGG |
| | | ACGTGTCACAGGAAGATCCGGAGGTGCAGTTCAATTGGTACGT |
| | | GGATGGCGTCGAGGTGCACAACGCCAAAACCAAGCCGAGGGA |
| | | GGAGCAGTTCAACTCCACTTACCGCGTCGTGTCCGTGCTGACGG |
| | | TGCTGCATCAGGACTGGCTGAACGGGAAGGAGTACAAGTGCAA |
| | | AGTGTCCAACAAGGGACTTCCTAGCTCAATCGAAAAGACCATC |
| | | TCGAAAGCCAAGGGACAGCCCCGGGAACCCCAAGTGTATACCC |
| | | TGCCACCGAGCCAGGAAGAAATGACTAAGAACCAAGTCTCATT |
| | | GACTTGCCTTGTGAAGGGCTTCTACCCATCGGATATCGCCGTGG |
| | | AATGGGAGTCCAACGGCCAGCCGGAAAACAACTACAAGACCA |
| | | CCCCTCCGGTGCTGGACTCAGACGGATCCTTCTTCCTCTACTCG |
| | | CGGCTGACCGTGGATAAGAGCAGATGGCAGGAGGGAAATGTGT |
| | | TCAGCTGTTCTGTGATGCATGAAGCCCTGCACAACCACTACACT |
| | | CAGAAGTCCCTGTCCCTCTCCCTGGGA |
| BAP049-Clone-E LC | | |
| SEQ ID NO: 510 (Kabat) | LCDR1 | KSSQSLLDSGNQKNFLT |
| SEQ ID NO: 511 (Kabat) | LCDR2 | WASTRES |
| SEQ ID NO: 512 (Kabat) | LCDR3 | QNDYSYPYT |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 520 | VL | EIVLTQSPATLSLSPGERATLSCKSSQSLLDSGNQKNFLTWYQQKP |
| | | GQAPRLLIYWASTRESGVPSRFSGSGSGTDFTFTISSLEAEDAATYY |
| | | CQNDYSYPYTFGQGTKVEIK |
| SEQ ID NO: 521 | DNA VL | GAGATCGTCCTGACTCAGTCACCCGCTACCCTGAGCCTGAGCCC |
| | | TGGCGAGCGGGCTACACTGAGCTGTAAATCTAGTCAGTCACTG |
| | | CTGGATAGCGGTAATCAGAAGAACTTCCTGACCTGGTATCAGC |
| | | AGAAGCCCGGTCAAGCCCCTAGACTGCTGATCTACTGGGCCTCT |
| | | ACTAGAGAATCAGGCGTGCCCTCTAGGTTTAGCGGTAGCGGTA |
| | | GTGGCACCGACTTCACCTTCACTATCTCTAGCCTGGAAGCCGAG |
| | | GACGCCGCTACCTACTACTGTCAGAACGACTATAGCTACCCCTA |
| | | CACCTTCGGTCAAGGCACTAAGGTCGAGATTAAG |
| SEQ ID NO: 522 | Light | EIVLTQSPATLSLSPGERATLSCKSSQSLLDSGNQKNFLTWYQQKP |
| | chain | GQAPRLLIYWASTRESGVPSRFSGSGSGTDFTFTISSLEAEDAATYY |
| | | CQNDYSYPYTFGQGTKVEIKRTVAAPSVFIFPPSDEQLKSGTASVV |
| | | CLLNNFYPREAKVQWKVDNALQSGNSQESVTEQDSKDSTYSLSST |
| | | LTLSKADYEKHKVYACEVTHQGLSSPVTKSFNRGEC |
| SEQ ID NO: 523 | DNA | GAGATCGTCCTGACTCAGTCACCCGCTACCCTGAGCCTGAGCCC |
| | light | TGGCGAGCGGGCTACACTGAGCTGTAAATCTAGTCAGTCACTG |
| | chain | CTGGATAGCGGTAATCAGAAGAACTTCCTGACCTGGTATCAGC |
| | | AGAAGCCCGGTCAAGCCCCTAGACTGCTGATCTACTGGGCCTCT |
| | | ACTAGAGAATCAGGCGTGCCCTCTAGGTTTAGCGGTAGCGGTA |
| | | GTGGCACCGACTTCACCTTCACTATCTCTAGCCTGGAAGCCGAG |
| | | GACGCCGCTACCTACTACTGTCAGAACGACTATAGCTACCCCTA |
| | | CACCTTCGGTCAAGGCACTAAGGTCGAGATTAAGCGTACGGTG |
| | | GCCGCTCCCAGCGTGTTCATCTTCCCCCCCAGCGACGAGCAGCT |
| | | GAAGAGCGGCACCGCCAGCGTGGTGTGCCTGCTGAACAACTTC |
| | | TACCCCCGGGAGGCCAAGGTGCAGTGGAAGGTGGACAACGCCC |
| | | TGCAGAGCGGCAACAGCCAGGAGAGCGTCACCGAGCAGGACA |
| | | GCAAGGACTCCACCTACAGCCTGAGCAGCACCCTGACCCTGAG |
| | | CAAGGCCGACTACGAGAAGCATAAGGTGTACGCCTGCGAGGTG |
| | | ACCCACCAGGGCCTGTCCAGCCCCGTGACCAAGAGCTTCAACA |
| | | GGGGCGAGTGC |
| BAP049-Clone-B HC | | |
| SEQ ID NO: 524 (Kabat) | HCDR1 | ACCTACTGGATGCAC |
| SEQ ID NO: 525 (Kabat) | HCDR2 | AATATCTACCCCGGCACCGGCGGCTCTAACTTCGACGAGAAGT |
| | | TTAAGAAT |
| SEQ ID NO: 526 (Kabat) | HCDR3 | TGGACTACCGGCACAGGCGCCTAC |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-B LC | | |
| SEQ ID NO: 529 (Kabat) | LCDR1 | AAATCTAGTCAGTCACTGCTGGATAGCGGTAATCAGAAGAACT |
| | | TCCTGACC |
| SEQ ID NO: 530 (Kabat | LCDR2 | TGGGCCTCTACTAGAGAATCA |
| SEQ ID NO: 531 (Kabat) | LCDR3 | CAGAACGACTATAGCTACCCCTACACC |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |
| BAP049-Clone-E HC | | |
| SEQ ID NO: 524 (Kabat) | HCDR1 | ACCTACTGGATGCAC |
| SEQ ID NO: 525 (Kabat) | HCDR2 | AATATCTACCCCGGCACCGGCGGCTCTAACTTCGACGAGAAGT |
| | | TTAAGAAT |
| SEQ ID NO: 526 (Kabat) | HCDR3 | TGGACTACCGGCACAGGCGCCTAC |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-E LC | | |
| SEQ ID NO: 529 (Kabat) | LCDR1 | AAATCTAGTCAGTCACTGCTGGATAGCGGTAATCAGAAGAACT |
| | | TCCTGACC |
| SEQ ID NO: 530 (Kabat) | LCDR2 | TGGGCCTCTACTAGAGAATCA |
| SEQ ID NO: 531 (Kabat) | LCDR3 | CAGAACGACTATAGCTACCCCTACACC |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |

### Other Exemplary PD-1 Inhibitors

In one embodiment, the anti-PD-1 antibody molecule is Nivolumab (Bristol-Myers Squibb), also known as MDX-1106, MDX-1106-04, ONO-4538, BMS-936558, or OPDIVO®. Nivolumab (clone 5C4) and other anti-PD-1 antibodies are disclosed in U.S. Pat. No. 8,008,449 and WO 2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 2.

In one embodiment, the anti-PD-1 antibody molecule is Pembrolizumab (Merck & Co), also known as Lambrolizumab, MK-3475, MK03475, SCH-900475, or KEYTRUDAR. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 2.

In one embodiment, the anti-PD-1 antibody molecule is Pidilizumab (CureTech), also known as CT-011. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 2.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

TABLE 2
Amino acid sequences of other exemplary anti-PD-1 antibody molecules

| | | |
|---|---|---|
| Nivolumab | | |
| SEQ ID | Heavy | QVQLVESGGGVVQPGRSLRLDCKASGITFSNSGMHWVRQAPGKGLEWVAV |
| NO: 535 | chain | IWYDGSKRYYADSVKGRFTISRDNSKNTLFLQMNSLRAEDTAVYYCATND |
| | | DYWGQGTLVTVSSASTKGPSVFPLAPCSRSTSESTAALGCLVKDYFPEPV |
| | | TVSWNSGALTSGVHTFPAVLQSSGLYSLSSVVTVPSSSLGTKTYTCNVDH |
| | | KPSNTKVDKRVESKYGPPCPPCPAPEFLGGPSVFLFPPKPKDTLMISRTP |
| | | EVTCVVVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPSQEE |
| | | MTKNQVSLTCLVKGFYPSDIAVEWESNGQPENNYKTTPPVLDSDGSFFLY |
| | | SRLTVDKSRWQEGNVFSCSVMHEALHNHYTQKSLSLSLGK |
| SEQ ID | Light | EIVLTQSPATLSLSPGERATLSCRASQSVSSYLAWYQQKPGQAPRLLIYD |
| NO: 536 | chain | ASNRATGIPARFSGSGSGTDFrLTISSLEPEDFAVYYCQQSSNWPRTFGQ |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |
| Pembrolizumab | | |
| SEQ ID | Heavy | QVQLVQSGVEVKKPGASVKVSCKASGYTFTNYYMYWVRQAPGQGLEWMG |
| NO: 537 | chain | GINPSNGGTNFNEKFKNRVTLTTDSSTTTAYMELKSLQFDDTAVYYCARR |
| | | DYRFDMGFDYWGQGTTVTVSSASTKGPSVFPLAPCSRSTSESTAALGCLV |
| | | KDYFPEPVTVSWNSGALTSGVHTFPAVLQSSGLYSLSSVVTVPSSSLGTK |
| | | TYTCNVDHKPSNTKVDKRVESKYGPPCPPCPAPEFLGGPSVFLFPPKPKD |
| | | TLMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQVY |
| | | TLPPSQEEMTKNQVSLTCLVKGFYPSDIAVEWESNGQPENNYKTTPPVLD |
| | | SDGSFFLYSRLTVDKSRWQEGNVFSCSVMHEALHNHYTQKSLSLSLGK |
| SEQ ID | Light | EIVLTQSPATLSLSPGERAILSCRASKGVSTSGYSYLHWYQQKPGQAPRL |
| NO: 538 | chain | LIYLASYLESGVPARFSGSGSGTDFTLTISSLEPEDFAVYYCQHSRDLPL |
| | | TFGGGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVYACEV |
| | | THQGLSSPVTKSFNRGEC |
| Pidilizumab | | |
| SEQ ID | Heavy | QVQLVQSGSELKKPGASVKISCKASGYTFTNYGMNWVRQAPGQGLQWMGW |
| NO: 539 | chain | INTDSGESTYAEEFKGRFVFSLDTSVNTAYLQITSLTAEDTGMYFCVRVG |
| | | YDALDYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKITPPVLDS |
| | | DGSFFLYSKLTVDKSRWQQGNVFSCSVMHEALHNHYTQKSLSLSPGK |
| SEQ ID | Light | EIVLTQSPSSLSASVGDRVTITCSARSSVSYYIHWFQQKPGKAPKLWIYR |
| NO: 540 | chain | TSNLASGVPSRFSGSGSGTSYCLTINSLQPEDFATYYCQQRSSFPLTFGG |
| | | GTKLEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |

### LAG-3 Inhibitors

In certain embodiments, the anti-PD-L1 antibody molecule described herein is administered in combination with a LAG-3 inhibitor. In some embodiments, the LAG-3 inhibitor is chosen from LAG525 (Novartis), BMS-986016 (Bristol-Myers Squibb), or TSR-033 (Tesaro).

### Exemplary LAG-3 Inhibitors

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises at least one, two, three, four, five or six complementarity determining regions (CDRs) (or collectively all of the CDRs) from a heavy and light chain variable region comprising an amino acid sequence shown in Table 5 (e.g., from the heavy and light chain variable region sequences of BAP050-Clone I or BAP050-Clone J disclosed in Table 5), or encoded by a nucleotide sequence shown in Table 5. In some embodiments, the CDRs are according to the Kabat definition (e.g., as set out in Table 5). In some embodiments, the CDRs are according to the Chothia definition (e.g., as set out in Table 5). In some embodiments, the CDRs are according to the combined CDR definitions of both Kabat and Chothia (e.g., as set out in Table 5). In one embodiment, the combination of Kabat and Chothia CDR of VH CDR1 comprises the amino acid sequence GFTLTNYGMN (SEQ ID NO: 766). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 5, or encoded by a nucleotide sequence shown in Table 5.

In one embodiment, the anti-LAG-3 antibody molecule comprises a heavy chain variable region (VH) comprising a VHCDR1 amino acid sequence of SEQ ID NO: 701, a VHCDR2 amino acid sequence of SEQ ID NO: 702, and a VHCDR3 amino acid sequence of SEQ ID NO: 703; and a light chain variable region (VL) comprising a VLCDR1 amino acid sequence of SEQ ID NO: 710, a VLCDR2 amino acid sequence of SEQ ID NO: 711, and a VLCDR3 amino acid sequence of SEQ ID NO: 712, each disclosed in Table 5.

In one embodiment, the anti-LAG-3 antibody molecule comprises a VH comprising a VHCDR1 encoded by the nucleotide sequence of SEQ ID NO: 736 or 737, a VHCDR2 encoded by the nucleotide sequence of SEQ ID NO: 738 or 739, and a VHCDR3 encoded by the nucleotide sequence of SEQ ID NO: 740 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 5. In one embodiment, the anti-LAG-3 antibody molecule comprises a VH comprising a VHCDR1 encoded by the nucleotide sequence of SEQ ID NO: 758 or 737, a VHCDR2 encoded by the nucleotide sequence of SEQ ID NO: 759 or 739, and a VHCDR3 encoded by the nucleotide sequence of SEQ ID NO: 760 or 741; and a VL comprising a VLCDR1 encoded by the nucleotide sequence of SEQ ID NO: 746 or 747, a VLCDR2 encoded by the nucleotide sequence of SEQ ID NO: 748 or 749, and a VLCDR3 encoded by the nucleotide sequence of SEQ ID NO: 750 or 751, each disclosed in Table 5.

In one embodiment, the anti-LAG-3 antibody molecule comprises a VH comprising the amino acid sequence of SEQ ID NO: 706, or an amino acid sequence at least 85%, 90%, 95%, or 99% identical or higher to SEQ ID NO: 706. In one embodiment, the anti-LAG-3 antibody molecule comprises a VL comprising the amino acid sequence of SEQ ID NO: 718, or an amino acid sequence at least 85%, 90%, 95%, or 99% identical or higher to SEQ ID NO: 718. In one embodiment, the anti-LAG-3 antibody molecule comprises a VH comprising the amino acid sequence of SEQ ID NO: 724, or an amino acid sequence at least 85%, 90%, 95%, or 99% identical or higher to SEQ ID NO: 724. In one embodiment, the anti-LAG-3 antibody molecule comprises a VL comprising the amino acid sequence of SEQ ID NO: 730, or an amino acid sequence at least 85%, 90%, 95%, or 99% identical or higher to SEQ ID NO: 730. In one embodiment, the anti-LAG-3 antibody molecule comprises a VH comprising the amino acid sequence of SEQ ID NO: 706 and a VL comprising the amino acid sequence of SEQ ID NO: 718. In one embodiment, the anti-LAG-3 antibody molecule comprises a VH comprising the amino acid sequence of SEQ ID NO: 724 and a VL comprising the amino acid sequence of SEQ ID NO: 730.

In one embodiment, the antibody molecule comprises a VH encoded by the nucleotide sequence of SEQ ID NO: 707 or 708, or a nucleotide sequence at least 85%, 90%, 95%, or 99% identical or higher to SEQ ID NO: 707 or 708. In one embodiment, the antibody molecule comprises a VL encoded by the nucleotide sequence of SEQ ID NO: 719 or 720, or a nucleotide sequence at least 85%, 90%, 95%, or 99% identical or higher to SEQ ID NO: 719 or 720. In one embodiment, the antibody molecule comprises a VH encoded by the nucleotide sequence of SEQ ID NO: 725 or 726, or a nucleotide sequence at least 85%, 90%, 95%, or 99% identical or higher to SEQ ID NO: 725 or 726. In one embodiment, the antibody molecule comprises a VL encoded by the nucleotide sequence of SEQ ID NO: 731 or 732, or a nucleotide sequence at least 85%, 90%, 95%, or 99% identical or higher to SEQ ID NO: 731 or 732. In one embodiment, the antibody molecule comprises a VH encoded by the nucleotide sequence of SEQ ID NO: 707 or 708 and a VL encoded by the nucleotide sequence of SEQ ID NO: 719 or 720. In one embodiment, the antibody molecule comprises a VH encoded by the nucleotide sequence of SEQ ID NO: 725 or 726 and a VL encoded by the nucleotide sequence of SEQ ID NO: 731 or 732.

In one embodiment, the anti-LAG-3 antibody molecule comprises a heavy chain comprising the amino acid sequence of SEQ ID NO: 709, or an amino acid sequence at least 85%, 90%, 95%, or 99% identical or higher to SEQ ID NO: 709. In one embodiment, the anti-LAG-3 antibody molecule comprises a light chain comprising the amino acid sequence of SEQ ID NO: 721, or an amino acid sequence at least 85%, 90%, 95%, or 99% identical or higher to SEQ ID NO: 721. In one embodiment, the anti-LAG-3 antibody molecule comprises a heavy chain comprising the amino acid sequence of SEQ ID NO: 727, or an amino acid sequence at least 85%, 90%, 95%, or 99% identical or higher to SEQ ID NO: 727. In one embodiment, the anti-LAG-3 antibody molecule comprises a light chain comprising the amino acid sequence of SEQ ID NO: 733, or an amino acid sequence at least 85%, 90%, 95%, or 99% identical or higher to SEQ ID NO: 733. In one embodiment, the anti-LAG-3 antibody molecule comprises a heavy chain comprising the amino acid sequence of SEQ ID NO: 709 and a light chain comprising the amino acid sequence of SEQ ID NO: 721. In one embodiment, the anti-LAG-3 antibody molecule comprises a heavy chain comprising the amino acid sequence of SEQ ID NO: 727 and a light chain comprising the amino acid sequence of SEQ ID NO: 733.

In one embodiment, the antibody molecule comprises a heavy chain encoded by the nucleotide sequence of SEQ ID NO: 716 or 717, or a nucleotide sequence at least 85%, 90%, 95%, or 99% identical or higher to SEQ ID NO: 716 or 717. In one embodiment, the antibody molecule comprises a light chain encoded by the nucleotide sequence of SEQ ID NO: 722 or 723, or a nucleotide sequence at least 85%, 90%, 95%, or 99% identical or higher to SEQ ID NO: 722 or 723. In one embodiment, the antibody molecule comprises a heavy chain encoded by the nucleotide sequence of SEQ ID NO: 728 or 729, or a nucleotide sequence at least 85%, 90%, 95%, or 99% identical or higher to SEQ ID NO: 728 or 729. In one embodiment, the antibody molecule comprises a light chain encoded by the nucleotide sequence of SEQ ID NO: 734 or 735, or a nucleotide sequence at least 85%, 90%, 95%, or 99% identical or higher to SEQ ID NO: 734 or 735. In one embodiment, the antibody molecule comprises a heavy chain encoded by the nucleotide sequence of SEQ ID NO: 716 or 717 and a light chain encoded by the nucleotide sequence of SEQ ID NO: 722 or 723. In one embodiment, the antibody molecule comprises a heavy chain encoded by the nucleotide sequence of SEQ ID NO: 728 or 729 and a light chain encoded by the nucleotide sequence of SEQ ID NO: 734 or 735.

The antibody molecules described herein can be made by vectors, host cells, and methods described in US 2015/0259420, incorporated by reference in its entirety.

TABLE 5
Amino acid and nucleotide sequences of exemplary anti-LAG-3 antibody molecules

| | | |
|---|---|---|
| BAP050-Clone I HC | | |
| SEQ ID NO: 701(Kabat) | HCDR1 | NYGMN |
| SEQ ID NO: 702 (Kabat) | HCDR2 | WINTDTGEPTYADDFKG |
| SEQ ID NO: 703 (Kabat) | HCDR3 | NPPYYYGTNNAEAMDY |
| SEQ ID NO: 704 | HCDRI | GFTLTNY |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO: 706 | VH | QVQLVQSGAEVKKPGASVKVSCKASGFTLTNYGMNWVRQARGQ |
| | | RLEWIGWINTDTGEPTYADDEKGRFVFSLDTSVSTAYLQISSL |
| | | KAEDTAVYYCARNPPYYYGTNNAEAMDYWGQGTTVTVSS |
| SEQ ID NO: 707 | DNA VH | CAAGTGCAGCTGGTGCAGTCGGGAGCCGAAGTGAAGAAGCCTG |
| | | GAGCCTCGGTGAAGGTGTCGTGCAAGGCATCCGGATTCACCCT |
| | | CACCAATTACGGGATGAACTGGGTCAGACAGGCCCGGGGTCAA |
| | | CGGCTGGAGTGGATCGGATGGATTAACACCGACACCGGGGAGC |
| | | CTACCTACGCGGACGATTTCAAGGGACGGTTCGTGTTCTCCCT |
| | | CGACACCTCCGTGTCCACCGCCTACCTCCAAATCTCCTCACTG |
| | | AAAGCGGAGGACACCGCCGTGTACTATTGCGCGAGGAACCCGC |
| | | CCTACTACTACGGAACCAACAACGCCGAAGCCATGGACTACTG |
| | | GGGCCAGGGCACCACTGTGACTGTGTCCAGC |
| SEQ ID NO: 708 | DNA VH | CAGGTGCAGCTGGTGCAGTCTGGCGCCGAAGTGAAGAAACCTG |
| | | GCGCCTCCGTGAAGGTGTCCTGCAAGGCCTCTGGCITCACCCT |
| | | GACCAACTACGGCATGAACTGGGTGCGACAGGCCAGGGGCCAG |
| | | CGGCTGGAATGGATCGGCTGGATCAACACCGACACCGGCGAGC |
| | | CTACCTACGCCGACGACTTCAAGGGCAGATTCGTGTTCTCCCT |
| | | GGACACCTCCGTGTCCACCGCCTACCTGCAGATCTCCAGCCTG |
| | | AAGGCCGAGGATACCGCCGTGTACTACTGCGCCCGGAACCCCC |
| | | CTTACTACTACGGCACCAACAACGCCGAGGCCATGGACTATTG |
| | | GGGCCAGGGCACCACCGTGACCGIGTCCTCT |
| SEQ ID NO: 709 | Heavy | QVQLVQSGAEVKKPGASVKVSCKASGFTLTNYGMNWVRQARGQ |
| | chain | RLEWIGWINTDTGEPTYADDEKGREVESLDTSVSTAYLQISSL |
| | | KAEDTAVYYCARNPPYYYGTNNAEAMDYWGQGTTVTVSSASTK |
| | | GPSVFPLAPCSRSTSESTAALGCLVKDYTPEPVTVSWNSGALT |
| | | SGVHTFPAVLQSSGLYSLSSVVTVPSSSLGTKTYTCNVDHKPS |
| | | NTKVDKRVESKYGPPCPPCPAPEFLGGPSVFLFPPKPKDTLMI |
| | | SRTPEVTCWVDVSQEDPEVQFNWYVDGVEVHXAKTKPREEQFN |
| | | STYRVVSVLTVLHQDWLNGKEYKCKVSNKGLPSSIEKTISKAK |
| | | GQPREPQVYTLPPSQEEMTKNQVSLTCLVKGFYPSDIAVEWES |
| | | NGQPENNYKTTPPVLDSDGSFFLYSRLTVDKSRWQEGNVFSCS |
| | | VMHEALHNHYTQKSLSLSLG |
| SEQ ID NO: 716 | DNA | CAAGTGCAGCTGGTGCAGTCGGGAGCCGAAGTGAAGAAGCCTG |
| | heavy | GAGCCTCGGTGAAGGTGTCGTGCAAGGCATCCGGATTCACCCT |
| | chain | CACCAATTACGGGATGAACTGGGTCAGACAGGCCCGGGGTCAA |
| | | CGGCTGGAGTGGATCGGATGGATTAACACCGACACCGGGGAGC |
| | | CTACCTACGCGGACGATTTCAAGGGACGGTTCGTGTTCTCCCT |
| | | CGACACCTCCGTGTCCACCGCCTACCTCCAAATCTCCTCACTG |
| | | AAAGCGGAGGACACCGCCGTGTACTATTGCGCGAGGAACCCGC |
| | | CCTACTACTACGGAACCAACAACGCCGAAGCCATGGACTACTG |
| | | GGGCCAGGGCACCACTGTGACTGTGTCCAGCGCGTCCACTAAG |
| | | GGCCCGTCCGTGTTCCCCCTGGCACCTTGTAGCCGGAGCACTA |
| | | GCGAATCCACCGCTGCCCTCGGCTGCCTGGTCAAGGATTACTT |
| | | GCCCGAGCCCGTGACCGTGTCCTGGAACAGCGGAGCCCTGACC |
| | | TCCGGAGTGCACACCTTCCCCGCTGTGCTGCAGAGCTCCGGGC |
| | | TGTACTCGCTGTCGTCGGTGGTCACGGTGCCTTCATCTAGCCT |
| | | GGGTACCAAGACCTACACTTGCAACGTGGACCACAAGCCTTCC |
| | | AACACT |
| | | AAGGTGGACAAGCGCGTCGAATCGAAGTACGGCCCACCGTGCC |
| | | CGCCTTGTCCCGCGCCGGAGTTCCTCGGCGGTCCCTCGGTCTT |
| | | TCTGTTCCCACCGAAGCCCAAGGACACTTTGATGATTTCCCGC |
| | | ACCCCTGAAGTGACATGCGTGGTCGTGGACGTGTCACAGGAAG |
| | | ATCCGGAGGTGCAGTTCAATTGGTACGTGGATGGCGTCGAGGT |
| | | GCACAACGCCAAAACCAAGCCGAGGGAGGAGCAGTTCAACTCC |
| | | ACTTACCGCGTCGTGTCCGTGCTGACGGTGCTGCATCAGGACT |
| | | GGCTGAACGGGAAGGAGTACAAGTGCAAAGTGTCCAACAAGGG |
| | | ACTTCCTAGCTCAATCGAAAAGACCATCTCGAAAGCCAAGGGA |
| | | CAGCCCCGGGAACCCCAAGTGTATACCCTGCCACCGAGCCAGG |
| | | AAGAAATGACTAAGAACCAAGTCTCATTGACTTGCCTTGTGAA |
| | | GGGCTTCTACCCATCGGATATCGCCGTGGAATGGGAGTCCAAC |
| | | GGCCAGCCGGAAAACAACTACAAGACCACCCCTCCGGTGCTGG |
| | | ACTCAGACGGATCCTTCTTCCTCTACTCGCGGCTGACCGTGGA |
| | | TAAGAGCAGATGGCAGGAGGGAAATGTGTTCAGCTGTTCTGTG |
| | | ATGCATGAAGCCCTGCACAACCACTACACTCAGAAGTCCCTGT |
| | | CCCTCTCCCTGGGA |
| SEQ ID NO: 717 | DNA | CAGGTGCAGCTGGTGCAGTCTGGCGCCGAAGTGAAGAAACCTG |
| | heavy | GCGCCTCCGTGAAGGTGTCCTGCAAGGCCTCTGGCTTCACCCT |
| | chain | GACCAACTACGGCATGAACTGGGTGCGACAGGCCAGGGGCCAG |
| | | CGGCTGGAATGGATCGGCTGGATCAACACCGACACCGGCGAGC |
| | | CTACCTACGCCGACGACTTCAAGGGCAGATTCGTGTTCTCCCT |
| | | GGACACCTCCGTGTCCACCGCCTACCTGCAGATCTCCAGCCTG |
| | | AAGGCCGAGGATACCGCCGTGTACTACTGCGCCCGGAACCCCC |
| | | CTTACTACTACGGCACCAACAACGCCGAGGCCATGGACTATTG |
| | | GGGCCAGGGCACCACCGTGACCGTGTCCTCTGCTTCTACCAAG |
| | | GGGCCCAGCGTGTTCCCCCTGGCCCCCTGCTCCAGAAGCACCA |
| | | GCGAGAGCACAGCCGCCCTGGGCTGCCTGGTGAAGGACTACTT |
| | | CCCCGAGCCCGTGACCGTGTCCTGGAACAGCGGAGCCCTGACC |
| | | AGCGGCGTGCACACCTTCCCCGCCGTGCTGCAGAGCAGCGGCC |
| | | TGTACAGCCTGAGCAGCGTGGTGACCGTGCCCAGCAGCAGCCT |
| | | GGGCACCAAGACCTACACCTGTAACGTGGACCACAAGCCCAGC |
| | | AACACCAAGGTGGACAAGAGGGTGGAGAGCAAGTACGGCCCAC |
| | | CCTGCCCCCCCTGCCCAGCCCCCGAGTTCCTGGGCGGACCCAG |
| | | CGTGTTCCTGTTCCCCCCCAAGCCCAAGGACACCCTGATGATC |
| | | AGCAGAACCCCCGAGGTGACCTGTGTGGTGGTGGACGTGTCCC |
| | | AGGAGGACCCCGAGGTCCAGTTCAACTGGTACGTGGACGGCGT |
| | | GGAGGTGCACAACGCCAAGACCAAGCCCAGAGAGGAGCAGTTT |
| | | AACAGCACCTACCGGGTGGTGTCCGTGCTGACCGTGCTGCACC |
| | | AGGACTGGCTGAACGGCAAAGAGTACAAGTGTAAGGTCTCCAA |
| | | CAAGGGCCTGCCAAGCAGCATCGAAAAGACCATCAGCAAGGCC |
| | | AAGGGCCAGCCTAGAGAGCCCCAGGTCTACACCCTGCCACCCA |
| | | GCCAAGAGGAGATGACCAAGAACCAGGTGTCCCTGACCTGTCT |
| | | GGTGAAGGGCTTCTACCCAAGCGACATCGCCGTGGAGTGGGAG |
| | | AGCAACGGCCAGCCCGAGAACAACTACAAGACCACCCCCCCAG |
| | | TGCTGGACAGCGACGGCAGCTTCTTCCTGTACAGCAGGCTGAC |
| | | CGTGGACAAGTCCAGATGGCAGGAGGGCAACGTCTTTAGCTGC |
| | | TCCGTGATGCACGAGGCCCTGCACAACCACTACACCCAGAAGA |
| | | GCCTGAGCCTGTCCCTGGGC |
| BAP050-Clone I LC | | |
| SEQ ID NO: 710 (Kabat) | LCDR1 | SSSQDISNYLN |
| SEQ ID NO: 711 (Kabat) | LCDR2 | YTSTLHL |
| SEQ ID NO: 712 (Kabat) | LCDR3 | QQYYNLPWT |
| SEQ ID NO: 713 | LCDR1 | SQDISNY |
| (Chothia) | | |
| SEQ ID NO: 714 (Chothia) | LCDR2 | YTS |
| SEQ ID NO: 715 (Chothia) | LCDR3 | YYNLPW |
| SEQ ID NO: 718 | VL | DIQMTQSPSSLSASVGDRVTITCSSSQDISNYLXWYLQK |
| | | PGQSPQLLIYYTSTLHLGVPSRFSGSGSGTEFTLTISSL |
| | | QPDDFATYYCQQYYNLPWTFGQGTKVEIK |
| | DNA VL | GATATTCAGATGACTCAGTCACCTAGTAGCCTGAGCGCT |
| | | AGTGTGGGCGATAGAGTGACTATCACCTGTAGCTCTAGT |
| | | CAGGATATCTCTAACTACCTGAACTGGTATCTGCAGAAG |
| | | CCCGGTCAATCACCTCAGCTGCTGATCTACTACACTAGC |
| | | ACCCTGCACCTGGGCGTGCCCTCTAGGTTTAGCGGTAGC |
| | | GGTAGTGGCACCGAGTTCACCCTGACTATCTCTAGCCTG |
| | | CAGCCCGACGACTTCGCTACCTACTACTGTCAGCAGTAC |
| | | TATAACCTGCCCTGGACCTTCGGTCAAGGCACTAAGGTC |
| | | GAGATTAAG |
| SEQ ID NO: 720 | DNA VL | GACATCCAGATGACCCAGTCCCCCTCCAGCCTGTCTGCTT |
| | | CCGTGGGCGACAGAGTGACCATCACCTGTTCCTCCAGCCA |
| | | GGACATCTCCAACTACCTGAACTGGTATCTGCAGAAGCCC |
| | | GGCCAGTCCCCTCAGCTGCTGATCTACTACACCTCCACCC |
| | | TGCACCTGGGCGTGCCCTCCAGATTTTCCGGCTCTGGCTC |
| | | TGGCACCGAGTTTACCCTGACCATCAGCTCCCTGCAGCCC |
| | | GACGACTTCGCCACCTACTACTGCCAGCAGTACTACAACC |
| | | TGCCCTGGACCTTCGGCCAGGGCACCAAGGTGGAAATCAA |
| | | G |
| SEQ ID NO: 721 | Light | DIQMTQSPSSLSASVGDRVTITCSSSQDISNYLXWYLQKP |
| | chain | GQSPQLLIYYTSTLHLGVPSRFSGSGSGTEFTLTISSLQP |
| | | DDFATYYCQQYYXLPWTFGQGTKVEIKRTVAAPSVFIFPP |
| | | SDEQLKSGTASVVCLLNNFYPREAKVQWKVDXALQSGNSQ |
| | | ESVTEQDSKDSTYSLSSTLTLSKADYEKHKVYACEVTHQG |
| | | LSSPVTKSFNRGEC |
| SEQ ID NO: 722 | DNA | GATATTCAGATGACTCAGTCACCTAGTAGCCTGAGCGCTA |
| | light | GTGTGGGCGATAGAGTGACTATCACCTGTAGCTCTAGTCA |
| | chain | GGATATCTCTAACTACCTGAACTGGTATCTGCAGAAGCCC |
| | | GGTCAATCACCTCAGCTGCTGATCTACTACACTAGCACCC |
| | | TGCACCTGGGCGTGCCCTCTAGGTTTAGCGGTAGCGGTAG |
| | | TGGCACCGAGTTCACCCTGACTATCTCTAGCCTGCAGCCC |
| | | GACGACTTCGCTACCTACTACTGTCAGCAGTACTATAACC |
| | | TGCCCTGGACCTTCGGTCAAGGCACTAAGGTCGAGATTAA |
| | | GCGTACGGTGGCCGCTCCCAGCGTGTTCATCTTCCCCCCC |
| | | AGCGACGAGCAGCTGAAGAGCGGCACCGCCAGCGTGGTGT |
| | | GCCTGCTGAACAACTTCTACCCCCGGGAGGCCAAGGTGCA |
| | | GTGGAAGGTGGACAACGCCCTGCAGAGCGGCAACAGCCAG |
| | | GAGAGCGTCACCGAGCAGGACAGCAAGGACTCCACCTACA |
| | | GCCTGAGCAGCACCCTGACCCTGAGCAAGGCCGACTACGA |
| | | GAAGCATAAGGTGTACGCCTGCGAGGTGACCCACCAGGGC |
| | | CTGTCCAGCCCCGTGACCAAGAGCTTCAACAGGGGCGAG |
| | | TGC |
| SEQ ID NO: 723 | DNA | GACATCCAGATGACCCAGTCCCCCTCCAGCCTGTCTGCTTC |
| | light | CGTGGGCGACAGAGTGACCATCACCTGTTCCTCCAGCCAGG |
| | chain | ACATCTCCAACTACCTGAACTGGTATCTGCAGAAGCCCGGC |
| | | CAGTCCCCTCAGCTGCTGATCTACTACACCTCCACCCTGCA |
| | | CCTGGGCGTGCCCTCCAGATTTTCCGGCTCTGGCTCTGGCA |
| | | CCGAGTTTACCCTGACCATCAGCTCCCTGCAGCCCGACGAC |
| | | TTCGCCACCTACTACTGCCAGCAGTACTACAACCTGCCCTG |
| | | GACCTTCGGCCAGGGCACCAAGGTGGAAATCAAGCGTACGG |
| | | TGGCCGCTCCCAGCGTGTTCATCTTCCCCCCAAGCGACGAG |
| | | CAGCTGAAGAGCGGCACCGCCAGCGTGGTGTGTCTGCTGAA |
| | | CAACTTCTACCCCAGGGAGGCCAAGGTGCAGTGGAAGGTGG |
| | | ACAACGCCCTGCAGAGCGGCAACAGCCAGGAGAGCGTCACC |
| | | GAGCAGGACAGCAAGGACTCCACCTACAGCCTGAGCAGCAC |
| | | CCTGACCCTGAGCAAGGCCGACTACGAGAA |
| | | GCACAAGGTGTACGCCTGTGAGGTGACCCACCAGGGCCTGT |
| | | CCAGCCCCGTGACCAAGAGCTTCAACAGGGGCGAGTGC |
| BAP050-Clone J HC | | |
| SEQ ID NO: 701 (Kabat) | HCDR1 | NYGMN |
| SEQ ID NO: 702 (Kabat) | HCDR2 | WINTDTGEPTYADDFKG |
| SEQ ID NO: 703 (Kabat) | HCDR3 | NPPYYYGTNNAEAMDY |
| SEQ ID NO: 704 | HCDR1 | GFTLTNY |
| (Chothia) | | |
| SEQ ID NO: 705 | HCDR2 | NTDTGE |
| (Chothia) | | |
| SEQ ID NO: 703 | HCDR3 | NPPYYYGTNNAEAMDY |
| (Chothia) | | |
| SEQ ID NO: 724 | VH | QVQLVQSGAEVKKPGASVKVSCKASGFTLTNYGMNWVRQA |
| | | PGQGLEWMGWINTDTGEPTYADDFKGRFVFSLDTSVSTAY |
| | | LQISSLKAEDTAVYYCARNPPYYYGTNNAEAMDYWGQGTT |
| | | VTVSS |
| SEQ ID NO: 725 | DNA VH | CAGGTGCAGCTGGTGCAGTCAGGCGCCGAAGTGAAGAAA |
| | | CCCGGCGCTAGTGTGAAAGTCAGCTGTAAAGCTAGTGGC |
| | | TTCACCCTGACTAACTACGGGATGAACTGGGTCCGCCAG |
| | | GCCCCAGGTCAAGGCCTCGAGTGGATGGGCTGGATTAAC |
| | | ACCGACACCGGCGAGCCTACCTACGCCGACGACTTTAAG |
| | | GGCAGATTCGTGTTTAGCCTGGACACTAGTGTGTCTACC |
| | | GCCTACCTGCAGATCTCTAGCCTGAAGGCCGAGGACACC |
| | | GCCGTCTACTACTGCGCTAGAAACCCCCCCTACTACTAC |
| | | GGCACTAACAACGCCGAGGCTATGGACTACTGGGGTCAA |
| | | GGCACTACCGTGACCGTGTCTAGC |
| SEQ ID NO: 726 | DNA VH | CAGGTGCAGCTGGTGCAGTCTGGCGCCGAAGTGAAGAAA |
| | | CCTGGCGCCTCCGTGAAGGTGTCCTGCAAGGCCTCTGGC |
| | | TTCACCCTGACCAACTACGGCATGAACTGGGTGCGACAG |
| | | GCCCCTGGACAGGGCCTGGAATGGATGGGCTGGATCAAC |
| | | ACCGACACCGGCGAGCCTACCTACGCCGACGACTTCAAG |
| | | GGCAGATTCGTGTTCTCCCTGGACACCTCCGTGTCCACC |
| | | GCCTACCTGCAGATCTCCAGCCTGAAGGCCGAGGATACC |
| | | GCCGTGTACTACTGCGCCCGGAACCCCCCTTACTACTAC |
| | | GGCACCAACAACGCCGAGGCCATGGACTATTGGGGCCAG |
| | | GGCACCACCGTGACCGTGTCCTCT |
| SEQ ID NO: 727 | Heavy | QVQLVQSGAEVKKPGASVKVSCKASGFTLTNYGMNWVRQ |
| | chain | APGQGLEWMGWINTDTGEPTYADDFKGRFVESLDTSVST |
| | | AYLQISSLKAEDTAVYYCARNPPYYYGTNNAEAMDYWGQ |
| | | GTTVTVSSASTKGPSVFPLAPCSRSTSESTAALGCLVKD |
| | | YFPEPVTVSWNSGALTSGVHTEPAVLQSSGLYSLSSVVT |
| | | VPSSSLGTKTYTCNVDHKPSNTKVDKRVESKYGPPCPPC |
| | | PAPEFLGGPSVFLFPPKPKDTLMISRTPEVTCVVVDVSQ |
| | | EDPEVQFNWYVDGVEVHNAKTKPREEQFNSTYRVVSVLT |
| | | VLHQDWLNGKEYKCKVSNKGLPSSIEKTISKAKGQPREP |
| | | QVYTLPPSQEEMTKNQVSLTCLVKGFYPSDIAVEWESNG |
| | | QPENNYKTTPPVLDSDGSFFLYSRLTVDKSRWQEGNVFS |
| | | CSVMHEALHNHYTQKSLSLSLG |
| SEQ ID NO: 728 | DNA | CAGGTGCAGCTGGTGCAGTCAGGCGCCGAAGTGAAGAAA |
| | heavy | CCCGGCGCTAGTGTGAAAGTCAGCTGTAAAGCTAGTGGC |
| | chain | TTCACCCTGACTAACTACGGGATGAACTGGGTCCGCCAG |
| | | GCCCCAGGTCAAGGCCTCGAGTGGATGGGCTGGATTAAC |
| | | ACCGACACCGGCGAGCCTACCTACGCCGACGACTTTAAG |
| | | GGCAGATTCGTGTTTAGCCTGGACACTAGTGTGTCTACC |
| | | GCCTACCTGCAGATCTCTAGCCTGAAGGCCGAGGACACC |
| | | GCCGTCTACTACTGCGCTAGAAACCCCCCCTACTACTAC |
| | | GGCACTAACAACGCCGAGGCTATGGACTACTGGGGTCAA |
| | | GGCACTACCGTGACCGTGTCTAGCGCTAGCACTAAGGGC |
| | | CCGTCCGTGTTCCCCCTGGCACCTTGTAGCCGGAGCACT |
| | | AGCGA |
| | | ATCCACCGCTGCCCTCGGCTGCCTGGTCAAGGATTACTTCC |
| | | CGGAGCCCGTGACCGTGTCCTGGAACAGCGGAGCCCTGACCT |
| | | CCGGAGTGCACACCTTCCCCGCTGTGCTGCAGAGCTCCGGGC |
| | | TGTACTCGCTGTCGTCGGTGGTCACGGTGCCTTCATCTAGCC |
| | | TGGGTACCAAGACCTACACTTGCAACGTGGACCACAAGCCTT |
| | | CCAACACTAAGGTGGACAAGCGCGTCGAATCGAAGTACGGCC |
| | | CACCGTGCCCGCCTTGTCCCGCGCCGGAGTTCCTCGGCGGTC |
| | | CCTCGGTCTTTCTGTTCCCACCGAAGCCCAAGGACACTTTGA |
| | | TGATTTCCCGCACCCCTGAAGTGACATGCGTGGTCGTGGACG |
| | | TGTCACAGGAAGATCCGGAGGTGCAGTTCAATTGGTACGTGG |
| | | ATGGCGTCGAGGTGCACAACGCCAAAACCAAGCCGAGGGAGG |
| | | AGCAGTTCAACTCCACTTACCGCGTCGTGTCCGTGCTGACGG |
| | | TGCTGCATCAGGACTGGCTGAACGGGAAGGAGTACAAGTGCA |
| | | AAGTGTCCAACAAGGGACTTCCTAGCTCAATCGAAAAGACCA |
| | | TCTCGAAAGCCAAGGGACAGCCCCGGGAACCCCAAGTGTATA |
| | | CCCTGCCACCGAGCCAGGAAGAAATGACTAAGAACCAAGTCT |
| | | CATTGACTTGCCTTGTGAAGCTGCTTCTACCCATCGGATATC |
| | | GCCGTGGAATGGGAGTCCAACGGCCAGCCGGAAAACAACTAC |
| | | AAGACCACCCCTCCGGTGCTGGACTCAGACGGATCCTTCTTC |
| | | CTCTACTCGCGGCTGACCGTGGATAAGAGCAGATGGCAGGAG |
| | | GGAAATGTGTTCAGCTGTTCTGTGATGCATGAAGCCCTGCAC |
| | | AACCACTACACTCAGAAGTCCCTGTCCCTCTCCCTGGGA |
| SEQ ID NO: 729 | DNA | CAGGTGCAGCTGGTGCAGTCTGGCGCCGAAGTGAAGAAACC |
| | heavy | TGGCGCCTCCGTGAAGGTGTCCTGCAAGGCCTCTGGCTTCA |
| | chain | CCCTGACCAACTACGGCATGAACTGGGTGCGACAGGCCCCT |
| | | GGACAGGGCCTGGAATGGATGGGCTGGATCAACACCGACAC |
| | | CGGCGAGCCTACCTACGCCGACGACTTCAAGGGCAGATTCG |
| | | TGTTCTCCCTGGACACCTCCGTGTCCACCGCCTACCTGCAG |
| | | ATCTCCAGCCTGAAGGCCGAGGATACCGCCGTGTACTACTG |
| | | CGCCCGGAACCCCCCTTACTACTACGGCACCAACAACGCCG |
| | | AGGCCATGGACTATTGGGGCCAGGGCACCACCGTGACCGTG |
| | | TCCTCTGCTTCTACCAAGGGGCCCAGCGTGTTCCCCCTGGC |
| | | CCCCTGCTCCAGAAGCACCAGCGAGAGCACAGCCGCCCTGG |
| | | GCTGCCTGGTGAAGGACTACTTCCCCGAGCCCGTGACCGTG |
| | | TCCTGGAACAGCGGAGCCCTGACCAGCGGCGTGCACACCTT |
| | | CCCCGCCGTGCTGCAGAGCAGCGGCCTGTACAGCCTGAGCA |
| | | GCGTGGTGACCGTGCCCAGCAGCAGCCTGGGCACCAAGACC |
| | | TACACCTGTAACGTGGACCACAAGCCCAGCAACACCAAGGT |
| | | GGACAAGAGGGTGGAGAGCAAGTACGGCCCACCCTGCCCCC |
| | | CCTGCCCAGCCCCCGAGTTCCTGGGCGGACCCAGCGTGTTC |
| | | CTGTTCCCCCCCAAGCCCAAGGACACCCTGATGATCAGCAG |
| | | AACCCCCGAGGTGACCTGTGTGGTGGTGGACGTGTCCCAGG |
| | | AGGACCCCGAGGTCCAGTTCAACTGGTACGTGGACGGCGTG |
| | | GAGGTGCACAACGCCAAGACCAAGCCCAGAGAGGAGCAGTT |
| | | TAACAGCACCTACCGGGTGGTGTCCGTGCTGACCGTGCTGC |
| | | ACCAGGACTGGCTGAACGGCAAAGAGTACAAGTGTAAGGTC |
| | | TCCAACAAGGGCCTGCCAAGCAGCATCGAAAAGACCATCAG |
| | | CAAGGCCAAGGGCCAGCCTAGAGAGCCCCAGGTCTACACCC |
| | | TGCCACCCAGCCAAGAGGAGATGACCAAGAACCAGGTGTCC |
| | | CTGACCTGTCTGGTGAAGGGCTTCTACCCAAGCGACATCGC |
| | | CGTGGAGTGGGAGAGCAACGGCCAGCCCGAGAACAACTACA |
| | | AGACCACCCCCCCAGTGCTGGACAGCGACGGCAGCTTdTCC |
| | | TGTACAGCAGGCTGACCGTGGACAAGTCCAGATGGCAGGAG |
| | | GGCAACGTCTTTAGCTGCTCCGTGATGCACGAGGCCCTGCA |
| | | CAACCACTACACCCAGAAGAGCCTGAGCCTGTCCCTGGGC |
| BAP050-Clone J LC | | |
| SEQ ID NO: 710 (Kabat) | LCDR1 | SSSQDISNYLN |
| SEQ ID NO: 711 (Kabat) | LCDR2 | YTSTLHL |
| SEQ ID NO: 712 (Kabat) | LCDR3 | QQYYNLPWT |
| SEQ ID NO: 713 | LCDR1 | SQDISNY |
| (Chothia) | | |
| SEQ ID NO: 714 | LCDR2 | YTS |
| (Chothia) | | |
| SEQ ID NO: 715 | LCDR3 | YYNLPW |
| (Chothia) | | |
| SEQ ID NO: 730 | VL | DIQVITQSPSSLSASVGDRVTITCSSSQDISNYLNWYQQKPGK |
| | | APKLLIYYTSTLHLGIPPRFSGSGYGTDFTLTINNIESEDAAY |
| | | YFCQQYYNLPWTFGQGTKVEIK |
| SEQ ID NO: 731 | DNA VL | GATATTCAGATGACTCAGTCACCTAGTAGCCTGAGCGCTAGTGT |
| | | GGGCGATAGAGTGACTATCACCTGTAGCTCTAGTCAGGATATCT |
| | | CTAACTACCTGAACTGGTATCAGCAGAAGCCCGGTAAAGCCCC |
| | | TAAGCTGCTGATCTACTACACTAGCACCCTGCACCTGGGAATCC |
| | | CCCCTAGGTTTAGCGGTAGCGGCTACGGCACCGACTTCACCCTG |
| | | ACTATTAACAATATCGAGTCAGAGGACGCCGCCTACTACTTCTG |
| | | TCAGCAGTACTATAACCTGCCCTGGACCTTCGGTCAAGGCACTA |
| | | AGGTCGAGATTAAG |
| SEQ ID NO: 732 | DNA VL | GACATCCAGATGACCCAGTCCCCCTCCAGCCTGTCTGCTTCCGT |
| | | GGGCGACAGAGTGACCATCACCTGTTCCTCCAGCCAGGACATC |
| | | TCCAACTACCTGAACTGGTATCAGCAGAAGCCCGGCAAGGCCC |
| | | CCAAGCTGCTGATCTACTACACCTCCACCCTGCACCTGGGCATC |
| | | CCCCCTAGATTCTCCGGCTCTGGCTACGGCACCGACTTCACCCT |
| | | GACCATCAACAACATCGAGTCCGAGGACGCCGCCTACTACTTC |
| | | TGCCAGCAGTACTACAACCTGCCCTGGACCTTCGGCCAGGGCA |
| | | CCAAGGTGGAAATCAAG |
| SEQ ID NO: 733 | Light | DIQMTQSPSSLSASVGDRVTITCSSSQDISNYLNWYQQKPGKA |
| | chain | PKLLIYYTSTLHLGIPPRFSGSGYGTDFTLTINNIESEDAAYY |
| | | FCQQYYNLPWTFGQGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| SEQ ID NO: 734 | DNA light | GATATTCAGATGACTCAGTCACCTAGTAGCCTGAGCGCTAGTGT |
| | chain | GGGCGATAGAGTGACTATCACCTGTAGCTCTAGTCAGGATATCT |
| | | CTAACTACCTGAACTGGTATCAGCAGAAGCCCGGTAAAGCCCC |
| | | TAAGCTGCTGATCTACTACACTAGCACCCTGCACCTGGGAATCC |
| | | CCCCTAGGTTTAGCGGTAGCGGCTACGGCACCGACTTCACCCTG |
| | | ACTATTAACAATATCGAGTCAGAGGACGCCGCCTACTACTTCTG |
| | | TCAGCAGTACTATAACCTGCCCTGGACCTTCGGTCAAGGCACTA |
| | | AGGTCGAGATTAAGCGTACGGTGGCCGCTCCCAGCGTGTTCAT |
| | | CTTCCCCCCCAGCGACGAGCAGCTGAAGAGCGGCACCGCCAGC |
| | | GTGGTGTGCCTGCTGAACAACTTCTACCCCCGGGAGGCCAAGG |
| | | TGCAGTGGAAGGTGGACAACGCCCTGCAGAGCGGCAACAGCCA |
| | | GGAGAGCGTCACCGAGCAGGACAGCAAGGACTCCACCTACAGC |
| | | CTGAGCAGCACCCTGACCCTGAGCAAGGCCGACTACGAGAAGC |
| | | ATAAGGTGTACGCCTGCGAGGTGACCCACCAGGGCCTGTCCAG |
| | | CCCCGTGACCAAGAGCTTCAACAGGGGCGAGTGC |
| SEQ ID NO: 735 | DNA light | GACATCCAGATGACCCAGTCCCCCTCCAGCCTGTCTGCTTCCGT |
| | chain | GGGCGACAGAGTGACCATCACCTGTTCCTCCAGCCAGGACATC |
| | | TCCAACTACCTGAACTGGTATCAGCAGAAGCCCGGCAAGGCCC |
| | | CCAAGCTGCTGATCTACTACACCTCCACCCTGCACCTGGGCATC |
| | | CCCCCTAGATTCTCCGGCTCTGGCTACGGCACCGACTTCACCCT |
| | | GACCATCAACAACATCGAGTCCGAGGACGCCGCCTACTACTTC |
| | | TGCCAGCAGTACTACAACCTGCCCTGGACCTTCGGCCAGGGCA |
| | | CCAAGGTGGAAATCAAGCGTACGGTGGCCGCTCCCAGCGTGTT |
| | | CATCTTCCCCCCAAGCGACGAGCAGCTGAAGAGCGGCACCGCC |
| | | AGCGTGGTGTGTCTGCTGAACAACTTCTACCCCAGGGAGGCCA |
| | | AGGTGCAGTGGAAGGTGGACAACGCCCTGCAGAGCGGCAACA |
| | | GCCAGGAGAGCGTCACCGAGCAGGACAGCAAGGACTCCACCTA |
| | | CAGCCTGAGCAGCACCCTGACCCTGAGCAAGGCCGACTACGAG |
| | | AAGCACAAGGTGTACGCCTGTGAGGTGACCCACCAGGGCCTGT |
| | | CCAGCCCCGTGACCAAGAGCTTCAACAGGGGCGAGTGC |
| BAP050-Clone I HC | | |
| SEQ ID NO: 736 (Kabat) | HCDR1 | AATTACGGGATGAAC |
| SEQ ID NO: 737 (Kabat) | HCDR1 | AACTACGGCATGAAC |
| SEQ ID NO: 738 (Kabat) | HCDR2 | TGGATTAACACCGACACCGGGGAGCCTACCTACGCGGACGATT |
| | | TCAAGGGA |
| SEQ ID NO: 739 (Kabat) | HCDR2 | TGGATCAACACCGACACCGGCGAGCCTACCTACGCCGACGACT |
| | | TCAAGGGC |
| SEQ ID NO: 740 (Kabat) | HCDR3 | AACCCGCCCTACTACTACGGAACCAACAACGCCGAAGCCATGG |
| | | ACTAC |
| SEQ ID NO: 741 (Kabat) | HCDR3 | AACCCCCCTTACTACTACGGCACCAACAACGCCGAGGCCAT |
| | | GGACTAT |
| SEQ ID NO: 742 | HCDR1 | GGATTCACCCTCACCAATTAC |
| (Chothia) | | |
| SEQ ID NO: 743 | HCDR1 | GGCTTCACCCTGACCAACTAC |
| (Chothia) | | |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 740 | HCDR3 | AACCCGCCCTACTACTACGGAACCAACAACGCCGAAGCCATGG |
| (Chothia) | | ACTAC |
| SEQ ID NO: 741 | HCDR3 | AACCCCCCTTACTACTACGGCACCAACAACGCCGAGGCCATGG |
| (Chothia) | | ACTAT |
| BAP050-Clone I LC | | |
| SEQ ID NO: 746 (Kabat) | LCDR1 | AGCTCTAGTCAGGATATCTCTAACTACCTGAAC |
| SEQ ID NO: 747 (Kabat) | LCDR1 | TCCTCCAGCCAGGACATCTCCAACTACCTGAAC |
| SEQ ID NO: 748 (Kabat) | LCDR2 | TACACTAGCACCCTGCACCTG |
| SEQ ID NO: 749 (Kabat) | LCDR2 | TACACCTCCACCCTGCACCTG |
| SEQ ID NO: 750 (Kabat) | LCDR3 | CAGCAGTACTATAACCTGCCCTGGACC |
| SEQ ID NO: 751 (Kabat) | LCDR3 | CAGCAGTACTACAACCTGCCCTGGACC |
| SEQ ID NO: 752 | LCDR1 | AGTCAGGATATCTCTAACTAC |
| (Chothia) | | |
| SEQ ID NO: 753 | LCDR1 | AGCCAGGACATCTCCAACTAC |
| (Chothia) | | |
| SEQ ID NO: 754 | LCDR2 | TACACTAGC |
| (Chothia) | | |
| SEQ ID NO: 755 | LCDR2 | TACACCTCC |
| (Chothia) | | |
| SEQ ID NO: 756 | LCDR3 | TACTATAACCTGCCCTGG |
| (Chothia) | | |
| SEQ ID NO: 757 | LCDR3 | TACTACAACCTGCCCTGG |
| (Chothia) | | |
| BAP050-Clone J HC | | |
| SEQ ID NO: 758 (Kabat) | HCDR1 | AACTACGGGATGAAC |
| SEQ ID NO: 737 (Kabat) | HCDR1 | AACTACGGCATGAAC |
| SEQ ID NO: 759 (Kabat) | HCDR2 | TGGATTAACACCGACACCGGCGAGCCTACCTACGCCGACGACT |
| | | TTAAGGGC |
| SEQ ID NO: 739 (Kabat) | HCDR2 | TGGATCAACACCGACACCGGCGAGCCTACCTACGCCGACGACT |
| | | TCAAGGGC |
| SEQ ID NO: 760 (Kabat) | HCDR3 | AACCCCCCCTACTACTACGGCACTAACAACGCCGAGGCTATGG |
| | | ACTAC |
| SEQ ID NO: 741 (Kabat) | HCDR3 | AACCCCCCTTACTACTACGGCACCAACAACGCCGAGGCCATGG |
| | | ACTAT |
| SEQ ID NO: 761 | HCDR1 | GGCTTCACCCTGACTAACTAC |
| (Chothia) | | |
| SEQ ID NO: 743 | HCDR1 | GGCTTCACCCTGACCAACTAC |
| (Chothia) | | |
| SEQ ID NO: 744 | HCDR2 | AACACCGACACCGGGGAG |
| (Chothia) | | |
| SEQ ID NO: 745 | HCDR2 | AACACCGACACCGGCGAG |
| (Chothia) | | |
| SEQ ID NO: 760 | HCDR3 | AACCCCCCCTACTACTACGGCACTAACAACGCCGAGGCTATGG |
| (Chothia) | | ACTAC |
| SEQ ID NO: 741 | HCDR3 | AACCCCCCTTACTACTACGGCACCAACAACGCCGAGGCCATGG |
| (Chothia) | | ACTAT |
| BAP050-Clone J LC | | |
| SEQ ID NO: 746 (Kabat) | LCDR1 | AGCTCTAGTCAGGATATCTCTAACTACCTGAAC |
| SEQ ID NO: 747 (Kabat) | LCDR1 | TCCTCCAGCCAGGACATCTCCAACTACCTGAAC |
| SEQ ID NO: 748 (Kabat) | LCDR2 | TACACTAGCACCCTGCACCTG |
| SEQ ID NO: 749 (Kabat) | LCDR2 | TACACCTCCACCCTGCACCTG |
| SEQ ID NO: 750 (Kabat) | LCDR3 | CAGCAGTACTATAACCTGCCCTGGACC |
| SEQID NO: 751 (Kabat) | LCDR3 | CAGCAGTACTACAACCTGCCCTGGACC |
| SEQ ID NO: 752 | LCDR1 | AGTCAGGATATCTCTAACTAC |
| (Chothia) | | |
| SEQ ID NO: 753 | LCDR1 | AGCCAGGACATCTCCAACTAC |
| (Chothia) | | |
| SEQ ID NO: 754 | LCDR2 | TACACTAGC |
| (Chothia) | | |
| SEQ ID NO: 755 | LCDR2 | TACACCTCC |
| (Chothia) | | |
| SEQ ID NO: 756 | LCDR3 | TACTATAACCTGCCCTGG |
| (Chothia) | | |
| SEQ ID NO: 757 | LCDR3 | TACTACAACCTGCCCTGG |
| (Chothia) | | |

### Other Exemplary LAG-3 Inhibitors

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 6.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 6. In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein. In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE 6
Amino acid sequences of other exemplary anti-LAG-3 antibody molecules

| | | |
|---|---|---|
| BMS-986016 | | |
| SEQ ID | Heavy | QVQLQQWGAGLLKPSETLSLTCAVYGGSFSDYYWNWIRQPPGKGLEWI |
| NO: 762 | chain | GEINHRGSTNSNPSLKSRVTLSLDTSKNQFSLKLRSVTAADTAVYYC |
| | | AFGYSDYEYNWFDPWGQGTLVTVSSASTKGPSVFPLAPCSRSTSESTA |
| | | ALGCLVKDYFPEPVTVSWNSGALTSGVHTFPAVLQSSGLYSLSSVVTV |
| | | PSSSLGTKTYTCNVDHKPSNTKVDKRVESKYGPPCPPCPAPEFLGGPS |
| | | VFLFPPKPKDTLMISRTPEVTCVVVDVSQEDPEVQFNWYVDGVEVHNA |
| | | KTKPREEQFNSTYRVVSVLTVLHQDWLNGKEYKCKVSNKGLPSSIEKT |
| | | ISKAKGQPREPQVYTLPPSQEEMTKNQVSLTCLVKGFYPSDIAVEWES |
| | | NGQPENNYKTTPPVLDSDGSFFLYSRLTVDKSRWQEGNVFSCSVMHE |
| | | ALHNHYTQKSLSLSLGK |
| SEQ ID | Light | EIVLTQSPATLSLSPGERATLSCRASQSISSYLAWYQQKPGQAPRLLIY |
| NO: 763 | chain | DASNRATGIPARFSGSGSGTDFTLTISSLEPEDFAVYYCQQRSNWPLTF |
| | | GQGTNLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACE |
| | | VTHQGLSSPVTKSFNRGEC |
| IMP731 | | |
| SEQ ID | Heavy | QVQLKESGPGLVAPSQSLSITCTVSGFSLTAYGVNWVRQPPGKGLEWL |
| NO: 764 | chain | GMIWDDGSTDYNSALKSRLSISKDNSKSQVFLKMNSLQTDDTARYYC |
| | | AREGDVAFDYWGQGTTLTVSSASTKGPSVFPLAPSSKSTSGGTAALGC |
| | | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEA |
| | | LHNHYTQKSLSLSPGK |
| SEQ ID | Heavy | DIVMTQSPSSLAVSVGQKVTMSCKSSQSLLNGSNQKNYLAWYQQKPG |
| NO: 765 | chain | QSPKLLVYFASTRDSGVPDRFIGSGSGTDFTLTISSVQAEDLADYFCL |
| | | QHFGTPPTFGGGTKLEIKRTVAAPSVFIFPPSDEQLKSGTASVVCLLN |
| | | NFYPREAKVQWKVDNALQSGNSQESVTEQDSKDSTYSLSSTLTLSKADYE |

### TIM-3 Inhibitors

In certain embodiments, the anti-PD-L1 antibody molecule described herein is administered in combination with a TIM-3 inhibitor. In some embodiments, the TIM-3 inhibitor is MGB453 (Novartis) or TSR-022 (Tesaro).

### Exemplary TIM-3 Inhibitors

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 7 (e.g., from the heavy and light chain variable region sequences of ABTIM3-hum 11 or ABTIM3-hum03 disclosed in Table 7), or encoded by a nucleotide sequence shown in Table 7. In some embodiments, the CDRs are according to the Kabat definition (e.g., as set out in Table 7). In some embodiments, the CDRs are according to the Chothia definition (e.g., as set out in Table 7). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 7, or encoded by a nucleotide sequence shown in Table 7.

In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 802, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 7. In one embodiment, the anti-TIM-3 antibody molecule comprises a heavy chain variable region (VH) comprising a VHCDR1 amino acid sequence of SEQ ID NO: 801, a VHCDR2 amino acid sequence of SEQ ID NO: 820, and a VHCDR3 amino acid sequence of SEQ ID NO: 803; and a light chain variable region (VL) comprising a VLCDR1 amino acid sequence of SEQ ID NO: 810, a VLCDR2 amino acid sequence of SEQ ID NO: 811, and a VLCDR3 amino acid sequence of SEQ ID NO: 812, each disclosed in Table 7.

In one embodiment, the anti-TIM-3 antibody molecule comprises a VH comprising the amino acid sequence of SEQ ID NO: 806, or an amino acid sequence at least 85%, 90%, 95%, or 99% identical or higher to SEQ ID NO: 806. In one embodiment, the anti-TIM-3 antibody molecule comprises a VL comprising the amino acid sequence of SEQ ID NO: 816, or an amino acid sequence at least 85%, 90%, 95%, or 99% identical or higher to SEQ ID NO: 816. In one embodiment, the anti-TIM-3 antibody molecule comprises a VH comprising the amino acid sequence of SEQ ID NO: 822, or an amino acid sequence at least 85%, 90%, 95%, or 99% identical or higher to SEQ ID NO: 822. In one embodiment, the anti-TIM-3 antibody molecule comprises a VL comprising the amino acid sequence of SEQ ID NO: 826, or an amino acid sequence at least 85%, 90%, 95%, or 99% identical or higher to SEQ ID NO: 826. In one embodiment, the anti-TIM-3 antibody molecule comprises a VH comprising the amino acid sequence of SEQ ID NO: 806 and a VL comprising the amino acid sequence of SEQ ID NO: 816. In one embodiment, the anti-TIM-3 antibody molecule comprises a VH comprising the amino acid sequence of SEQ ID NO: 822 and a VL comprising the amino acid sequence of SEQ ID NO: 826.

In one embodiment, the antibody molecule comprises a VH encoded by the nucleotide sequence of SEQ ID NO: 807, or a nucleotide sequence at least 85%, 90%, 95%, or 99% identical or higher to SEQ ID NO: 807. In one embodiment, the antibody molecule comprises a VL encoded by the nucleotide sequence of SEQ ID NO: 817, or a nucleotide sequence at least 85%, 90%, 95%, or 99% identical or higher to SEQ ID NO: 817. In one embodiment, the antibody molecule comprises a VH encoded by the nucleotide sequence of SEQ ID NO: 823, or a nucleotide sequence at least 85%, 90%, 95%, or 99% identical or higher to SEQ ID NO: 823. In one embodiment, the antibody molecule comprises a VL encoded by the nucleotide sequence of SEQ ID NO: 827, or a nucleotide sequence at least 85%, 90%, 95%, or 99% identical or higher to SEQ ID NO: 827. In one embodiment, the antibody molecule comprises a VH encoded by the nucleotide sequence of SEQ ID NO: 807 and a VL encoded by the nucleotide sequence of SEQ ID NO: 817. In one embodiment, the antibody molecule comprises a VH encoded by the nucleotide sequence of SEQ ID NO: 823 and a VL encoded by the nucleotide sequence of SEQ ID NO: 827.

In one embodiment, the anti-TIM-3 antibody molecule comprises a heavy chain comprising the amino acid sequence of SEQ ID NO: 808, or an amino acid sequence at least 85%, 90%, 95%, or 99% identical or higher to SEQ ID NO: 808. In one embodiment, the anti-TIM-3 antibody molecule comprises a light chain comprising the amino acid sequence of SEQ ID NO: 818, or an amino acid sequence at least 85%, 90%, 95%, or 99% identical or higher to SEQ ID NO: 818. In one embodiment, the anti-TIM-3 antibody molecule comprises a heavy chain comprising the amino acid sequence of SEQ ID NO: 824, or an amino acid sequence at least 85%, 90%, 95%, or 99% identical or higher to SEQ ID NO: 824. In one embodiment, the anti-TIM-3 antibody molecule comprises a light chain comprising the amino acid sequence of SEQ ID NO: 828, or an amino acid sequence at least 85%, 90%, 95%, or 99% identical or higher to SEQ ID NO: 828. In one embodiment, the anti-TIM-3 antibody molecule comprises a heavy chain comprising the amino acid sequence of SEQ ID NO: 808 and a light chain comprising the amino acid sequence of SEQ ID NO: 818. In one embodiment, the anti-TIM-3 antibody molecule comprises a heavy chain comprising the amino acid sequence of SEQ ID NO: 824 and a light chain comprising the amino acid sequence of SEQ ID NO: 828.

In one embodiment, the antibody molecule comprises a heavy chain encoded by the nucleotide sequence of SEQ ID NO: 809, or a nucleotide sequence at least 85%, 90%, 95%, or 99% identical or higher to SEQ ID NO: 809. In one embodiment, the antibody molecule comprises a light chain encoded by the nucleotide sequence of SEQ ID NO: 819, or a nucleotide sequence at least 85%, 90%, 95%, or 99% identical or higher to SEQ ID NO: 819. In one embodiment, the antibody molecule comprises a heavy chain encoded by the nucleotide sequence of SEQ ID NO: 825, or a nucleotide sequence at least 85%, 90%, 95%, or 99% identical or higher to SEQ ID NO: 825. In one embodiment, the antibody molecule comprises a light chain encoded by the nucleotide sequence of SEQ ID NO: 829, or a nucleotide sequence at least 85%, 90%, 95%, or 99% identical or higher to SEQ ID NO: 829. In one embodiment, the antibody molecule comprises a heavy chain encoded by the nucleotide sequence of SEQ ID NO: 809 and a light chain encoded by the nucleotide sequence of SEQ ID NO: 819. In one embodiment, the antibody molecule comprises a heavy chain encoded by the nucleotide sequence of SEQ ID NO: 825 and a light chain encoded by the nucleotide sequence of SEQ ID NO: 829.

The antibody molecules described herein can be made by vectors, host cells, and methods described in US 2015/0218274, incorporated by reference in its entirety.

TABLE 7
Amino acid and nucleotide sequences of exemplary anti-TIM-3 antibody molecules

| | | |
|---|---|---|
| ABTIM3-hum11 | | |
| SEQ ID NO: 801 (Kabat) | HCDR1 | SYNMH |
| SEQ ID NO: 802 (Kabat) | HCDR2 | DIYPGNGDTSYNQKFKG |
| SEQ ID NO: 803 (Kabat) | HCDR3 | VGGAFPMDY |
| SEQ ID NO: 804 (Chothia) | HCDR1 | GYTFTSY |
| SEQ ID NO: 805 (Chothia) | HCDR2 | YPGNGD |
| SEQ ID NO: 803 (Chothia) | HCDR3 | VGGAFPMDY |
| SEQ ID NO: 806 | VH | QVQLVQSGAEVKKPGSSVKVSCKASGYTFTSYNMHWVRQAPG |
| | | QGLEWMGDIYPGNGDTSYNQKIKGRVT1TADKSTSTVYMELSS |
| | | LRSEDTAVYYCARVGGAFPMDYWGQGTTVTVSS |
| SEQ ID NO: 807 | DNA VH | CAGGTGCAGCTGGTGCAGTCAGGCGCCGAAGTGAAGAAACC |
| | | CGGCTCTAGCGTGAAAGTTTCTTGTAAAGCTAGTGGCTACAC |
| | | CTTCACTAGCTATAATATGCACTGGGTTCGCCAGGCCCCAGG |
| | | GCAAGGCCTCGAGTGGATGGGCGATATCTACCCCGGGAACGG |
| | | CGACACTAGTTATAATCAGAAGTTTAAGGGTAGAGTCACTAT |
| | | CACCGCCGATAACTCTACTAGCACCGTCTATATGGAACTGAG |
| | | TTCCCTGAGGTCTGAGGACACCGCCGTCTACTACTGCGCTAG |
| | | AGTGGGCGGAGCCTTCCCTATGGACTACTGGGGTCAAGGCAC |
| | | TACCGTGACCGTGTCTAGC |
| SEQ ID NO: 808 | Heavy chain | QVQLVQSGAEVKKPGSSVKVSCKASGYTFTSYNMHWVRQAPG |
| | | QGLEWMGDIYPGNGDTSYNQKFKGRVTITADKSTSTVYMELSS |
| | | LRSEDTAVYYCARVGGAFPMDYWGQGTTVTVSSASTKGPSVFP |
| | | LAPCSRSTSESTAALGCLVKDYFPEPVTVSWNSGALTSGVHTFP |
| | | AVLQSSGLYSLSSVVTVPSSSLGTKTYTCNVDHKPSNTKVDKRV |
| | | ESKYGPPCPPCPAPEFLGGPSVFLFPPKPKDTLMISRTPEVTCVV |
| | | VDVSQEDPEVQFNWYVDGVEVHNAKTKPREEQFNSTYRVVSVLT |
| | | VLHQDWLNGKEYKCKVSNKGLPSSlEKIISKAKGQPREPQVYIL |
| | | PPSQEEMTKNQVSLTCLVKGFYPSDIAVEWESNGQPENNYKTTP |
| | | PVLDSDGSFFLYSRLTVDKSRWQEGNVFSCSVMHEALHNHYTQ |
| | | KSLSLSLG |
| SEQ ID NO: 809 | DNA heavy | CAGGTGCAGCTGGTGCAGTCAGGCGCCGAAGTGAAGAAACC |
| | chain | CGGCTCTAGCGTGAAAGTTTCTTGTAAAGCTAGTGGCTACAC |
| | | CTTCACTAGCTATAATATGCACTGGGTTCGCCAGGCCCCAGG |
| | | GCAAGGCCTCGAGTGGATGGGCGATATCTACCCCGGGAACGG |
| | | CGACACTAGTTATAATCAGAAGTTTAAGGGTAGAGTCACTAT |
| | | CACCGCCGATAAGTCTACTAGCACCGTCTATATGGAACTGAG |
| | | TTCCCTGAGGTCTGAGGACACCGCCGTCTACTACTGCGCTAG |
| | | AGTGGGCGGAGCCTTCCCTATGGACTACTGGGGTCAAGGCAC |
| | | TACCGTGACCGTGTCTAGCGCTAGCACTAAGGGCCCGTCCGT |
| | | GTTCCCCCTGGCACCTTGTAGCCGGAGCACTAGCGAATCCAC |
| | | CGCTGCCCTCGGCTGCCTGGTCAAGGATTACTTCCCGGAGCC |
| | | CGTGACCGTGTCCTGGAACAGCGGAGCCCTGACCTCCGGAGT |
| | | GCACACCTTCCCCGCTGTGCTGCAGAGCTCCGGGCTGTACTC |
| | | GCTGTCGTCGGTGGTCACGGTGCCTTCATCTAGCCTGGGTACC |
| | | AAGACCTACACTTGCAACGTGGACCACAAGCCTTCCAACACT |
| | | AAGGTGGACAAGCGCGTCGAATCGAAGTACGGCCCACCGTG |
| | | CCCGCCTTGTCCCGCGCCGGAGTTCCTCGGCGGTCCCTCGGTC |
| | | TTTCTGTTCCCACCGAAGCCCAAGGACACTTTGATGATTTCCC |
| | | GCACCCCTGAAGTGACATGCGTGGTCGTGGACGTGTCACAGG |
| | | AAGATCCGGAGGTGCAGTTCAATTGGTACGTGGATGGCGTCG |
| | | AGGTGCACAACGCCAAAACCAAGCCGAGGGAGGAGCAGTTC |
| | | AACTCCACTTACCGCGTCGTGTCCGTGCTGACGGTGCTGCATC |
| | | AGGACTGGCTGAACGGGAAGGAGTACAAGTGCAAAGTGTCC |
| | | AACAAGGGACTTCCTAGCTCAATCGAAAAGACCATCTCGAAA |
| | | GCCAAGGGACAGCCCCGGGAACCCCAAGTGTATACCCTGCCA |
| | | CCGAGCCAGGAAGAAATGACTAAGAACCAAGTCTCATTGACT |
| | | TGCCTTGTGAAGGGCTCTACCCATCGGATATCGCCGTGGAA |
| | | TGGGAGTCCAACGGCCAGCCGGAAAACAACTACAAGACCAC |
| | | CCCTCCGGTGCTGGACTCAGACGGATCCTTCTTCCTCTACT |
| | | CGCGGCTGACCGTGGATAAGAGCAGATGGCAGGAGGGAAAT |
| | | GTGTTCAGCTGTTCTGTGATGCATGAAGCCCTGCACAACCA |
| | | CTACACTCAGAAGTCCCTGTCCCTCTCCCTGGGA |
| SEQ ID NO: 810 (Kabat) | LCDR1 | RASESVEYYGTSLMQ |
| SEQ ID NO: 811 (Kabat) | LCDR2 | AASNVES |
| SEQ ID NO: 812 (Kabat) | LCDR3 | QQSRKDPST |
| SEQ ID NO: 813 (Chothia) | LCDR1 | SESVEYYGTSL |
| SEQ ID NO: 814 (Chothia) | LCDR2 | AAS |
| SEQ ID NO: 815 (Chothia) | LCDR3 | SRKDPS |
| SEQ ID NO: 816 | VL | AIQLTQSPSSLSASVGDRVTITCRASESVEYYGTSLMQWYQQKP |
| | | GKAPKLLIYAASNVESGVPSRESGSGSGTDFTLTISSLQPEDFA |
| | | TYFCQQSRKDPSTFGGGTKVEIK |
| SEQ ID NO: 817 | DNA VL | GCTATTCAGCTGACTCAGTCACCTAGTAGCCTGAGCGCTAGT |
| | | GTGGGCGATAGAGTGACTATCACCTGTAGAGCTAGTGAATCA |
| | | GTCGAGTACTACGGCACTAGCCTGATGCAGTGGTATCAGCAG |
| | | AAGCCCGGGAAAGCCCCTAAGCTGCTGATCTACGCCGCCTCT |
| | | AACGTGGAATCAGGCGTGCCCTCTAGGTTTAGCGGTAGCGGT |
| | | AGTGGCACCGACTTCACCCTGACTATCTCTAGCCTGCAGCCC |
| | | GAGGACTTCGCTACCTACTTCTGTCAGCAGTCTAGGAAGGAC |
| | | CCTAGCACCTTCGGCGGAGGCACTAAGGTCGAGATTAAG |
| SEQ ID NO: 818 | Light chain | AIQLTQSPSSLSASVGDRVTITCRASESVEYYGTSLMQWYQQKP |
| | | GKAPKLLIYAASNVESGVPSRFSGSGSGTDFTLTISSLQPEDFA |
| | | TYFCQQSRKDPSTFGGGTKVEIKRTVAAPSVFIFPPSDEQLKSG |
| | | TASVVCLLNNFYPREAKVQWKVDNALQSGXSQESVTEQDSKDST |
| | | YSLSSTLTLSKADYEKHKVYACEVTHQGLSSPVTKSFNRGEC |
| SEQ ID NO: 819 | DNA | GCTATTCAGCTGACTCAGTCACCTAGTAGCCTGAGCGCTAGT |
| | Light | GTGGGCGATAGAGTGACTATCACCTGTAGAGCTAGTGAATCA |
| | chain | GTCGAGTACTACGGCACTAGCCTGATGCAGTGGTATCAGCAG |
| | | AAGCCCGGGAAAGCCCCTAAGCTGCTGATCTACGCCGCCTCT |
| | | AACGTGGAATCAGGCGTGCCCTCTAGGTTTAGCGGTAGCGGT |
| | | AGTGGCACCGACTTCACCCTGACTATCTCTAGCCTGCAGCCC |
| | | GAGGACTTCGCTACCTACTTCTGTCAGCAGTCTAGGAAGGAC |
| | | CCTAGCACCTTCGGCGGAGGCACTAAGGTCGAGATTAAGCGT |
| | | ACGGTGGCCGCTCCCAGCGTGTTCATCTTCCCCCCCAGCGAC |
| | | GAGCAGCTGAAGAGCGGCACCGCCAGCGTGGTGTGCCTGCTG |
| | | AACAACTTCTACCCCCGGGAGGCCAAGGTGCAGTGGAAGGTG |
| | | GACAACGCCCTGCAGAGCGGCAACAGCCAGGAGAGCGTCAC |
| | | CGAGCAGGACAGCAAGGACTCCACCTACAGCCTGAGCAGCA |
| | | CCCTGACCCTGAGCAAGGCCGACTACGAGAAGCATAAGGTG |
| | | TACGCCTGCGAGGTGACCCACCAGGGCCTGTCCAGCCCCGT |
| | | GACCAAGAGCTTCAACAGGGGCGAGTGC |
| ABTIM3-hum03 | | |
| SEQ ID NO: 801 (Kabat) | HCDR1 | SYNMH |
| SEQ ID NO: 820 (Kabat) | HCDR2 | D1YPGQGDTSYNQKFKG |
| SEQ ID NO: 803 (Kabat) | HCDR3 | VGGAFPMDY |
| SEQ ID NO: 804 (Chothia) | HCDR1 | GYTFTSY |
| SEQ ID NO: 821 (Chothia) | HCDR2 | YPGNGD |
| SEQ ID NO: 803 (Chothia) | HCDR3 | VGGAFPMDY |
| SEQ ID NO: 822 | VH | QVQLVQSGAEVKKPGASVKVSCKASGYTFTSYNMHWVRQA |
| | | PGQGLEWIGDIYPGQGDTSYNQKFKGRATMTADKSTSTVY |
| | | MELSSLRSEDTAVYYCARVGGAFPMDYWGQGTLVTVSS |
| SEQ ID NO: 823 | DNA VH | CAGGTGCAGCTGGTGCAGTCAGGCGCCGAAGTGAAGAAACC |
| | | CGGCGCTAGTGTGAAAGTTAGCTGTAAAGCTAOTGGCTATAC |
| | | TTTCACTTCTTATAATATGCACTGGGTCCGCCAGGCCCCAGGT |
| | | CAAGGCCTCGAGTGGATCGGCGATATCTACCCCGGTCAAGGC |
| | | GACACTTCCTATAATCAGAAGTTTAAGGGTAGAGCTACTATG |
| | | ACCGCCGATAAGTCTACTTCTACCGTCTATATGGAACTGAGTT |
| | | CCCTGAGGTCTGAGGACACCGCCGTCTACTACTGCGCTAGAG |
| | | TGGGCGGAGCCTTCCCAATGGACTACTGGGGTCAAGGCACCC |
| | | TGGTCACCGTGTCTAGC |
| SEQ ID NO: 824 | Heavy chain | QVQLVQSGAEVKKPGASVKVSCKASGYTFTSYNMHWVRQAPG |
| | | QGLEWIGDIYPGQGDTSYNQKFKGRATMTADKSTSTVYMELSS |
| | | LRSEDTAVYYCARVGGAFPMDYWGQGTLVTVSSASTKGPSVFP |
| | | LAPCSRSTSESTAALGCLVKDYFPEPVTVSWNSGALTSGVHTFP |
| | | AVLQSSGLYSLSSVVTVPSSSLGTKTYTCNVDHKPSNTKVDKRV |
| | | ESKYGPPCPPCPAPEILGGPSVFLIPPKPKDTLMISRTPEVTCVV |
| | | VDVSQEDPEVQFNWYVDGVEVHNAKTKPREEQFNSTYRVVSVLT |
| | | VLHQDWLNGKEYKCKVSNKGLPSSIEKTISKAKGQPREPQVYTL |
| | | PPSQEEMTKNQVSLTCLVKGFYPSDIAVEWESNGQPENNYKTTP |
| | | PVLDSDGSFFLYSRLTVDKSRWQEGNVFSCSVMHEALHNHYTQ |
| | | KSLSLSLG |
| SEQ ID NO: 825 | DNA heavy | CAGGTGCAGCTGGTGCAGTCAGGCGCCGAAGTGAAGAAACC |
| | chain | CGGCGCTAGTGTGAAAGTTAGCTGTAAAGCTAGTGGCTATAC |
| | | TTTCACTTCTTATAATATGCACTGGGTCCGCCAGGCCCCAGGT |
| | | CAAGGCCTCGAGTGGATCGGCGATATCTACCCCGGTCAAGGC |
| | | GACACTTCCTATAATCAGAATTTAAGGGTAGAGCTACTATG |
| | | ACCGCCGATAAGTCTACTTCTACCGTCTATATGGAACTGAGTT |
| | | CCCTGAGGTCTGAGGACACCGCCGTCTACTACTGCGCTAGAG |
| | | TGGGCGGAGCCTTCCCAATGGACTACTGGGGTCAAGGCACCC |
| | | TGGTCACCGTGTCTAGCGCTAGCACTAAGGGCCCGTCCGTGT |
| | | TCCCCCTGGCACCTTGTAGCCGGAGCACTAGCGAATCCACCG |
| | | CTGCCCTCGGCTGCCTGGTCAAGGATTACTTCCCGGAGCCCGT |
| | | GACCGTGTCCTGGAACAGCGGAGCCCTGACCTCCGGAGTGCA |
| | | CACCTTCCCCGCTGTGCTGCAGAGCTCCGGGCTGTACTCGCTG |
| | | TCGTCGGTGGTCACGGTGCCTTCATCTAGCCTGGGTACCAAG |
| | | ACCTACACTTGCAACGTGGACCACAAGCCTTCCAACACTAAG |
| | | GTGGACAAGCGCGTCGAATCGAAGTACGGCCCACCGTGCCCG |
| | | CCTTGTCCCGCGCCGGAGTTCCTCGGCGGTCCCTCGGTCTTTC |
| | | TGTTCCCACCGAAGCCCAAGGACACTTTGATGATTTCCCGCA |
| | | CCCCTGAAGTGACATGCGTGGTCGTGGACGTGTCACAGGAAG |
| | | ATCCGGAGGTGCAGTTCAATTGGTACGTGGATGGCGTCGAGG |
| | | TGCACAACGCCAAAACCAAGCCGAGGGAGGAGCAGTTCAAC |
| | | TCCACTTACCGCGTCGTGTCCGTGCTGACGGTGCTGCATCAGG |
| | | ACTGGCTGAACGGGAAGGAGTACAAGTGCAAAGTGTCCAAC |
| | | AAGGGACTTCCTAGCTCAATCGAAAAGACCATCTCGAAAGCC |
| | | AAGGGACAGCCCCGGGAACCCCAAGTGTATACCCTGCCACCG |
| | | AGCCAGGAAGAAATGACTAAGAACCAAGTCTCATTGACTTGC |
| | | CTTGTGAAGGGCTTCTACCCATCGGATATCGCCGTGGAATGG |
| | | GAGTCCAACGGCCAGCCGGAAAACAACTACAAGACCACCCC |
| | | TCCGGTGCTGGACTCAGACGGATCCTTCTTCCTCTACTCGCGG |
| | | CTGACCGTGGATAAGAGCAGATGGCAGGAGGGAAATGTGTT |
| | | CAGCTGTTCTGTGATGCATGAAGCCCTGCACAACCACTACAC |
| | | TCAGAAGTCCCTGTCCCTCTCCCTGGGA |
| SEQ ID NO: 810 (Kabat) | LCDR1 | RASESVEYYGTSLMQ |
| SEQ ID NO: 811 (Kabat) | LCDR2 | AASNVES |
| SEQ ID NO: 812 (Kabat) | LCDR3 | QQSRKDPST |
| SEQ ID NO: 813 (Chothia) | LCDR1 | SESVEYYGTSL |
| SEQ ID NO: 814 (Chothia) | LCDR2 | AAS |
| SEQ ID NO: 815 (Chothia) | LCDR3 | SRKDPS |
| SEQ ID NO: 826 | VL | DIVLTQSPDSLAVSLGERATINCRASESVEYYGTSLMQWYQQK |
| | | PGQPPKLLIYAASNVESGVPDRFSGSGSGTDFTLTISSLQAED |
| | | VAVYYCQQSRKDPTFGGGTKVEIK |
| SEQ ID NO: 827 | DNA VL | GATATCGTCCTGACTCAGTCACCCGATAGCCTGGCCGTCAGC |
| | | CTGGGCGAGCGGGCTACTATTAACTGTAGAGCTAGTGAATCA |
| | | GTCGAGTACTACGGCACTAGCCTGATGCAGTGGTATCAGCAG |
| | | AAGCCCGGTCAACCCCCTAAGCTGCTGATCTACGCCGCCTCT |
| | | AACGTGGAATCAGGCGTGCCCGATAGGTTTAGCGGTAGCGGT |
| | | AGTGGCACCGACTTCACCCTGACTATTAGTAGCCTGCAGGCC |
| | | GAGGACGTGGCCGTCTACTACTGTCAGCAGTCTAGGAAGGAC |
| | | CCTAGCACCTTCGGCGGAGGCACTAAGGTCGAGATTAAG |
| SEQ ID NO: 828 | Light chain | DIVLTQSPDSLAVSLGERATINCRASESVEYYGTSLMQWYQQ |
| | | KPGQPPKLLIYAASNVESGVPDRFSGSGSGTDFTLTISSLQA |
| | | EDVAVYYCQQSRKDPSTFGGGTKVEIKRTVAAPSVFIFPPSD |
| | | EQLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| SEQ ID NO: 829 | DNA | GATATCGTCCTGACTCAGTCACCCGATAGCCTGGCCGTCAGC |
| | light chain | CTGGGCGAGCGGGCTACTATTAACTGTAGAGCTAGTGAATCA |
| | | GTCGAGTACTACGGCACTAGCCTGATGCAGTGGTATCAGCAG |
| | | AAGCCCGGTCAACCCCCTAAGCTGCTGATCTACGCCGCCTCT |
| | | AACGTGGAATCAGGCGTGCCCGATAGGTTTAGCGGTAGCGGT |
| | | AGTGGCACCGACTTCACCCTGACTATTAGTAGCCTGCAGGCC |
| | | GAGGACGTGGCCGTCTACTACTGTCAGCAGTCTAGGAAGGAC |
| | | CCTAGCACCTTCGGCGGAGGCACTAAGGTCGAGATTAAGCGT |
| | | ACGGTGGCCGCTCCCAGCGTGTTCATCTTCCCCCCCAGCGAC |
| | | GAGCAGCTGAAGAGCGGCACCGCCAOCGTGGTGTGCCTGCTG |
| | | AACAACTTCTACCCCCGGGAGGCCAAGGTGCAGTGGAAGGTG |
| | | GACAACGCCCTGCAGAGCGGCAACAGCCAGGAGAGCGTCAC |
| | | CGAGCAGGACAGCAAGGACTCCACCTACAGCCTGAGCAGCA |
| | | CCCTGACCCTGAGCAAGGCCGACTACGAGAAGCATAAGGTGT |
| | | ACGCCTGCGAGGTGACCCACCAGGGCCTGTCCAGCCCCGTGA |
| | | CCAAGAGCTTCAACAGGGGCGAGTGC |

### Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 8. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 8
Amino acid sequences of other
exemplary anti-TIM-3 antibody molecules

| | | |
|---|---|---|
| APE5137 | | |
| SEQ ID | VH | EVQLLESGGGLVQPGGSLRL |
| NO: 830 | | SCAAASGITFSSYDMSWVRQ |
| | | APGKGLDWVSTISGGGTYTY |
| | | YQDSVKGRFTISRDNSKNTL |
| | | YLQMNSLRAEDTAVYYCASM |
| | | DYWGQGTTVTVSSA |
| SEQ ID | VL | DIQMTQSPSSLSASVGDRVT |
| NO: 831 | | ITCRASQSIRRYLNWYHQKP |
| | | GKAPKLLIYGASTLQSGVPS |
| | | RFSGSGSGTDFTLTISSLQP |
| | | EDFAVYYCQQSHSAPLTFGG |
| | | GTKVEIKR |
| APE5121 | | |
| SEQ ID | VH | EVQVLESGGGLVQPGGSLRL |
| NO: 832 | | YCVASGFTFSGSYAMSWVRQ |
| | | APGKGLEWVSAISGSGGSTY |
| | | YADSVKGRFTISRDNSKNTL |
| | | YLQMNSLRAEDTAVYYCAKK |
| | | YYVGPADYWGQGTLVTVSSG |
| SEQ ID | VL | DIVMTQSPDSLAVSLGERAT |
| NO: 833 | | INCKSSQSVLYSSNNKNYLA |
| | | WYQHKPGQPPKLL1YWASTR |
| | | ESGVPDRFSGSGSGTDFTLT |
| | | ISSLQAEDVAVYYCQQYYSS |
| | | PLTFGGGTKIEVK |

### GITR Agonists

In certain embodiments, the anti-PD-L1 antibody molecule described herein is administered in combination with a GITR agonist. In some embodiments, the GITR agonist is GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

### Exemplary GITR Agonists

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety.

In one embodiment, the anti-GITR antibody molecule comprises at least one, two, three, four, five or six complementarity determining regions (CDRs) (or collectively all of the CDRs) from a heavy and light chain variable region comprising an amino acid sequence shown in Table 9 (e.g., from the heavy and light chain variable region sequences of MAB7 disclosed in Table 9), or encoded by a nucleotide sequence shown in Table 9. In some embodiments, the CDRs are according to the Kabat definition (e.g., as set out in Table 9). In some embodiments, the CDRs are according to the Chothia definition (e.g., as set out in Table 9). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 9, or encoded by a nucleotide sequence shown in Table 9.

In one embodiment, the anti-GITR antibody molecule comprises a heavy chain variable region (VH) comprising a VHCDR1 amino acid sequence of SEQ ID NO: 909, a VHCDR2 amino acid sequence of SEQ ID NO: 911, and a VHCDR3 amino acid sequence of SEQ ID NO: 913; and a light chain variable region (VL) comprising a VLCDR1 amino acid sequence of SEQ ID NO: 914, a VLCDR2 amino acid sequence of SEQ ID NO: 916, and a VLCDR3 amino acid sequence of SEQ ID NO: 918, each disclosed in Table 9.

In one embodiment, the anti-GITR antibody molecule comprises a VH comprising the amino acid sequence of SEQ ID NO: 901, or an amino acid sequence at least 85%, 90%, 95%, or 99% identical or higher to SEQ ID NO: 901. In one embodiment, the anti-GITR antibody molecule comprises a VL comprising the amino acid sequence of SEQ ID NO: 902, or an amino acid sequence at least 85%, 90%, 95%, or 99% identical or higher to SEQ ID NO: 902. In one embodiment, the anti-GITR antibody molecule comprises a VH comprising the amino acid sequence of SEQ ID NO: 901 and a VL comprising the amino acid sequence of SEQ ID NO: 902.

In one embodiment, the antibody molecule comprises a VH encoded by the nucleotide sequence of SEQ ID NO: 905, or a nucleotide sequence at least 85%, 90%, 95%, or 99% identical or higher to SEQ ID NO: 905. In one embodiment, the antibody molecule comprises a VL encoded by the nucleotide sequence of SEQ ID NO: 906, or a nucleotide sequence at least 85%, 90%, 95%, or 99% identical or higher to SEQ ID NO: 906. In one embodiment, the antibody molecule comprises a VH encoded by the nucleotide sequence of SEQ ID NO: 905 and a VL encoded by the nucleotide sequence of SEQ ID NO: 906.

In one embodiment, the anti-GITR antibody molecule comprises a heavy chain comprising the amino acid sequence of SEQ ID NO: 903, or an amino acid sequence at least 85%, 90%, 95%, or 99% identical or higher to SEQ ID NO: 903. In one embodiment, the anti-GITR antibody molecule comprises a light chain comprising the amino acid sequence of SEQ ID NO: 904, or an amino acid sequence at least 85%, 90%, 95%, or 99% identical or higher to SEQ ID NO: 904. In one embodiment, the anti-GITR antibody molecule comprises a heavy chain comprising the amino acid sequence of SEQ ID NO: 903 and a light chain comprising the amino acid sequence of SEQ ID NO: 904.

In one embodiment, the antibody molecule comprises a heavy chain encoded by the nucleotide sequence of SEQ ID NO: 907, or a nucleotide sequence at least 85% , 90%, 95%, or 99% identical or higher to SEQ ID NO: 907. In one embodiment, the antibody molecule comprises a light chain encoded by the nucleotide sequence of SEQ ID NO: 908, or a nucleotide sequence at least 85% , 90%, 95%, or 99% identical or higher to SEQ ID NO: 908. In one embodiment, the antibody molecule comprises a heavy chain encoded by the nucleotide sequence of SEQ ID NO: 907 and a light chain encoded by the nucleotide sequence of SEQ ID NO: 908.

The antibody molecules described herein can be made by vectors, host cells, and methods described in WO 2016/057846, incorporated by reference in its entirety.

TABLE 9
Amino acid and nucleotide sequences of exemplary anti-GITR antibody molecule

| | | |
|---|---|---|
| MAB7 | | |
| SEQ ID NO: 901 | VH | EVQLVESGGGLVQSGGSLRLSCAASGFSLSSYGVDWVRQA |
| | | PGKGLEWVGVIWGGGGTYYASSLMGRFTISRDNSKNTLYL |
| | | QMNSLRAEDTAVYYCARHAYGHDGGFAMDYWGQGTLVTVS |
| | | S |
| SEQ ID NO: 902 | VL | EIVMTQSPATLSVSPGERATLSCRASESVSSNVAWYQQRP |
| | | GQAPRLLIYGASNRATGIPARFSGSGSGTDFTLTISRLEP |
| | | EDFAVYYCGQSYSYPITFGQGTKLEIK |
| SEQ ID NO: 903 | Heavy | EVQLVESGGGLVQSGGSLRLSCAASGFSLSSYGVDWVRQA |
| | Chain | PGKGLEWVGVIWGGGGTYYASSLMGRFTISRDNSKNTLYL |
| | | QMNSLRAEDTAVYYCARHAYGHDGGFAMDYWGQGTLVTVS |
| | | SASTKGPSVFPLAPSSKSTSGGTAALGCLVKDYFPEPVTV |
| | | SWNSGALTSGVHTFPAVLQSSGLYSLSSVVTVPSSSLGTQ |
| | | TYICNVNHKPSNTKVDKRVEPKSCDKTHTCPPCPAPELLG |
| | | GPSVFLFPPKPKDTLMISRTPEVTCVVVDVSHEDPEVKFN |
| | | WYVDGVEVHNAKTKPREEQYNSTYRVVSVLTVLHQDWLNG |
| | | KEYKCKVSNKALPAPIEKTISKAKGQPREPQVYTLPPSRE |
| | | EMTKNQVSLTCLVKGFYSDIAVEWESNGQPENNYKTTPPV |
| | | LDSDGSFFLYSKLTVDKSRWQQGNVFSCSVMHEALHNHY |
| | | TQKSLSLSPGK |
| SEQ ID NO: 904 | Light | EIVMTQSPATLSVSPGERATLSCRASESVSSNVAWYQQRP |
| | Chain | GQAPRLLIYGASNRATGIPARFSGSGSGTDFTLTISRLEP |
| | | EDFAVYYCGQSYSYPFTFGQGTKLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| SEQ ID NO: 905 | DNA VH | GAGGTGCAGCTGGTGGAATCTGGCGGCGGACTGGTGCAGT |
| | | CCGGCGGCTCTCTGAGACTGTCTTGCGCTGCCTCCGGCTT |
| | | CTCCCTGTCCTCTTACGGCGTGGACTGGGTGCGACAGGCC |
| | | CCTGGCAAGGGCCTGGAATGGGTGGGAGTGATCTGGGGC |
| | | GGAGGCGGCACCTACTACGCCTCTTCCCTGATGGGCCGGT |
| | | TCACCATCTCCCGGGACAACTCCAAGAACACCCTGTACCT |
| | | GCAGATGAACTCCCTGCGGGCCGAGGACACCGCCGTGTAC |
| | | TACTGCGCCAGACACGCCTACGGCCACGACGGCGGCrrCG |
| | | CCATGGATTATTGGGGCCAGGGCACCCTGGTGACAGTGTC |
| | | CTCC |
| SEQ ID NO: 906 | DNA VL | GAGATCGTGATGACCCAGTCCCCCGCCACCCTGTCTGTGT |
| | | C1CCCGGCGAGAGAGCCACCCTGAGCTGCAGAGCCTCCGA |
| | | GTCCGTGTCCTCCAACGTGGCCTGGTATCAGCAGAGACCT |
| | | GGTCAGGCCCCTCGGCTGCTGATCTACGGCGCCTCTAACC |
| | | GGGCCACCGGCATCCCTGCCAGATTCTCCGGCTCCGGCAG |
| | | CGGCACCGACTTCACCCTGACCATCTCCCGGCTGGAACCC |
| | | GAGGACTTCGCCGTGTACTACTGCGGCCAGTCCTACTCAT |
| | | ACCCCTTCACCTTCGGCCAGGGCACCAAGCTGGAAATCAA |
| | | CT |
| SEQ ID NO: 907 | DNA | GAGGTGCAGCTGGTGGAATCTGGCGGCGGACTGGTGCAGT |
| | Heavy | CCGGCGGCTCTCTGAGACTGTCTTGCGCTGCCTCCGGCTT |
| | Chain | CTCCCTGTCCTCTTACGGCGTGGACTGGGTGCGACAGGCC |
| | | CCTGGCAAGGGCCTGGAATGGGTGGGAGTGATCTGGGGC |
| | | GGAGGCGGCACCTACTACGCCTCTTCCCTGATGGGCCGGT |
| | | TCACCATCTCCCGGGACAACTCCAAGAACACCCTGTACCT |
| | | GCAGATGAACTCCCTGCGGGCCGAGGACACCGCCGTGTAC |
| | | TACTGCGCCAGACACGCCTACGGCCACGACGGCGGCTTCG |
| | | CCATGGATTATTGGGGCCAGGGCACCCTGGTGACACTGTC |
| | | CTCCGCTAGCACCAAGGGCCCAAGTGTGTTTCCCCTGGCC |
| | | CCCAGCAGCAAGTCTACTTCCGGCGGAACTGCTGCCCTGG |
| | | GTTGCCTGGTGAAGGACTACTTCCCCGAGCCCGTGACAGT |
| | | GTCCTGGAACTCTGGGGCTCTGACTTCCGGCGTGCACACC |
| | | TTCCCCGCCGTGCTGCAGAGCAGCGGCCTGTACAGCCTGA |
| | | GCAGCGTGGTGACAGTGCCCTCCAGCTCTCTGGGAACCCA |
| | | GACCTATATCTGCAACGTGAACCACAAGCCCAGCAACACC |
| | | AAGGTGGACAAGAGAGTGGAGCCCAAGAGCTGCGACAAG |
| | | ACCCACACCTGCCCCCCCTGCCCAGCTCCAGAACTGCTGG |
| | | GAGGGCCTTCCGTGTTCCTGTTCCCCCCCAAGCCCAAGGA |
| | | CACCCTGATGATCAGCAGGACCCCCGAGGTGACCTGCGTG |
| | | GTGGTGGACGTGTCCCACGAGGACCCAGAGGTGAAGTTC |
| | | AACTGGTACGTGGACGGCGTGGAGGTGCACAACGCCAAG |
| | | ACCAAGCCCAGAGAGGAGCAGTACAACAGCACCTACAGG |
| | | GTGGTGTCCGTGCTGACCGTGCTGCACCAGGACTGGCTGA |
| | | ACGGCAAAGAATACAAGTGCAAAGTCTCCAACAAGGCCC |
| | | TGCCAGCCCCAATCGAAAAGACAATCAGCAAGGCCAAGG |
| | | GCCAGCCACGGGAGCCCCAGGTGTACACCCTGCCCCCCAG |
| | | CCGGGAGGAGATGACCAAGAACCAGGTGTCCCTGACCTG |
| | | TCTGGTGAAGGGCTTCTACCCCAGCGATATCGCCGTGGAG |
| | | TGGGAGAGCAACGGCCAGCCCGAGAACAACTACAAGACC |
| | | ACCCCCCCAGTGCTGGACAGCGACGGCAGCTTCTTCCTGT |
| | | ACAGCAAGCTGACCGTGGACAAGTCCAGGTGGCAGCAGG |
| | | GCAACGTGTTCAGCTGCAGCGTGATGCACGAGGCCCTGCA |
| | | CAACCACTACACCCAGAAGTCCCTGAGCCTGAGCCCCGGC |
| | | AAG |
| SEQ ID NO: 908 | DNA | GAGATCGTGATGACCCAGTCCCCCGCCACCCTGTCTGTGT |
| | Light | CTCCCGGCGAGAGAGCCACCCTGAGCTGCAGAGCCTCCGA |
| | Chain | GTCCGTGTCCTCCAACGTGGCCTGGTATCAGCAGAGACCT |
| | | GGTCAGGCCCCTCGGCTGCTGATCTACGGCGCCTCTAACC |
| | | GGGCCACCGGCATCCCTGCCAGATTCTCCGGCTCCGGCAG |
| | | CGGCACCGACTTCACCCrGACCATCTCCCGGCTGGAACCC |
| | | GAGGACTTCGCCGTGTACTACTGCGGCCAGTCCTACTCAT |
| | | ACCCCTTCACCTTCGGCCAGGGCACCAAGCTGGAAATCAA |
| | | GCGTACGGTGGCCGCTCCCAGCGTGTTCATCTTCCCCCCC |
| | | AGCGACGAGCAGCTGAAGAGCGGCACCGCCAGCGTGGTGT |
| | | GCCTGCTGAACAACTTCTACCCCCGGGAGGCCAAGGTGC |
| | | AGTGGAAGGTGGACAACGCCCTGCAGAGCGGCAACAGCC |
| | | AGGAGAGCGTCACCGAGCAGGACAGCAAGGACTCCACCT |
| | | ACAGCCTGAGCAGCACCCTGACCCTGAGCAAGGCCGACT |
| | | ACGAGAAGCATAAGGTGTACGCCTGCGAGGTGACCCACC |
| | | AGGGCCTGTCCAGCCCCGTGACCAAGAGCTTCAACAGGG |
| | | GCGAGTGC |
| SEQ ID NO: 909 (KABAT) | HCDR1 | SYGVD |
| SEQ ID NO: 910 (CHOTHIA) | HCDR1 | GFSLSSY |
| SEQ ID NO: 911 (KABAT) | HCDR2 | VIWGGGGTYYASSLMG |
| SEQ ID NO: 912 (CHOTHIA) | HCDR2 | WGGGG |
| SEQ ID NO: 913 (KABAT) | HCDR3 | HAYGHDGGFAMDY |
| SEQ ID NO: 913 (CHOTHIA) | HCDR3 | HAYGHDGGFAMDY |
| SEQ ID NO: 914 (KABAT) | LCDR1 | RASESVSSNVA |
| SEQ ID NO: 915 (CHOTHIA) | LCDRI | SESVSSN |
| SEQ ID NO: 916 (KABAT) | LCDR2 | GASNRAT |
| SEQ ID NO: 917 (CHOTHIA) | LCDR2 | GAS |
| SEQ ID NO: 918 (KABAT) | LCDR3 | GQSYSYPIT |
| SEQ ID NO: 919 (CHOTHIA) | LCDR3 | SYSYPF |

### Other Exemplary GITR Agonists

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table 10.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5): 1108-1118, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of MK-4166 or MK-1248.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of TRX518.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of INCAGN1876.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of AMG 228.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety. In one embodiment, the GITR agonist comprises one or more of the CDR sequences (or collectively all of the CDR sequences), the heavy chain or light chain variable region sequence, or the heavy chain or light chain sequence of INBRX-110.

In one embodiment, the GITR agonist (e.g., a fusion protein) is MEDI 1873 (MedImmune), also known as MEDI1873. MEDI 1873 and other GITR agonists are disclosed, e.g., in US 2017/0073386, WO 2017/025610, and Ross et al. Cancer Res 2016; 76(14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE 10
Amino acid sequence of other exemplary
anti-GITR antibody molecules

| | | |
|---|---|---|
| BMS-986156 | | |
| SEQ ID | VH | QVQLVESGGGVV |
| NO: 920 | | QPGRSLRLSCAA |
| | | SGETESSYGMHW |
| | | VRQAPGKGLEWV |
| | | AVIWYEGSNKYY |
| | | ADSVKGRFTISR |
| | | DNSKNTLYLQMN |
| | | SLRAEDTAVYYC |
| | | ARGGSMVRGDYY |
| | | YGMDVWGQGTTV |
| | | TVSS |
| SEQ ID | VL | AIQLTQSPSSLS |
| NO: 921 | | ASVGDRVTITCR |
| | | ASQGISSALAWY |
| | | QQKPGKAPKLLI |
| | | YDASSLESGVPS |
| | | RFSGSGSGTDFT |
| | | LTISSLQPEDFA |
| | | TYYCQQFNSYPY |
| | | TFGQGTKLEIK |

### IL15/IL-15Ra Complexes

In certain embodiments, the anti-PD-L1 antibody molecule described herein is administered in combination with an IL-15/IL-15Ra complex. In some embodiments, the IL-15/IL-15Ra complex is chosen from NIZ985 (Novartis), ATL-803 (Altor) or CYP0150 (Cytune).

### Exemplary IL-15/IL-]5Ra Complexes

In one embodiment, the IL-15/IL-15Ra complex comprises human IL-15 complexed with a soluble form of human IL-15Ra. The complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the composition comprises an amino acid sequence of SEQ ID NO: 1001 in Table 11 and the soluble form of human IL-15Ra comprises an amino acid sequence of SEQ ID NO: 1002 in Table 11, as described in WO 2014/066527, incorporated by reference in its entirety. The molecules described herein can be made by vectors, host cells, and methods described in WO 2007/084342, incorporated by reference in its entirety.

TABLE 11
Amino acid and nucleotide sequences of
exemplary IL-15/IL-15Ra complexes
NIZ985

| | | |
|---|---|---|
| SEQ ID NO: | Human | NWVNVISDLKKIEDLIQSMH |
| 1001 | IL-15 | IDATLYTESDVHPSCKVTAM |
| | | KCFLLELQVISLESGDASIH |
| | | DTVENLIILANNSLSSNGNV |
| | | TESGCKECEELEEKNIKEFL |
| | | QSFVHIVQMFINTS |
| SEQ ID NO: | Human | ITCPPPMSVEHADIWVKSYS |
| 1002 | Soluble | LYSRERYICNSGFKRKAGTS |
| | IL-15Ra | SLTECVLNKATNVAHWTTPS |
| | | LKCIRDPALVHQRPAPPSTV |
| | | TTAGVTPQPESLSPSGKEPA |
| | | ASSPSSNNTAATTAAIVPGS |
| | | QLVIPSKSPSTGTTEISSHE |
| | | SSHGTPSQTTAKNWELTASA |
| | | SHQPPGVYPQG |

### Other Exemplary IL-15/IL-15Ra Complexes

In one embodiment, the IL-15/IL-15Ra complex is ALT-803, an IL-15/IL-15Ra Fc fusion protein (IL-15N72D:IL-15RaSu/Fc soluble complex). ALT-803 is disclosed in WO 2008/143794, incorporated by reference in its entirety. In one embodiment, the IL-15/IL-15Ra Fc fusion protein comprises the sequences as disclosed in Table 12.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is disclosed in WO 2007/04606 and WO 2012/175222, incorporated by reference in their entirety. In one embodiment, the IL-15/IL-15Ra sushi domain fusion comprises the sequences as disclosed in Table 12.

TABLE 12
Amino acid sequences of other
exemplary IL-15/IL-15Ra complexes
ALT-803 (Altor)

| | | |
|---|---|---|
| SEQ ID NO: | IL-15N72D | NWVNVISDLKKIE |
| 1003 | | DLIQSMHIDATLY |
| | | TESDVHPSCKVTA |
| | | MKCFLLELQVISL |
| | | ESGDASIHDTVEN |
| | | LIILANDSLSSNG |
| | | NVTESGCKECEEL |
| | | EEKNIKEFLQSFV |
| | | HIVQMFINTS |
| SEQ ID NO: | IL-15RaSu/Fc | ITCPPPMSVEHAD |
| 1004 | | IWVKSYSLYSRER |
| | | YICNSGFKRKAGT |
| | | SSLTECVLNKATN |
| | | VAHWTTPSLKCIR |
| | | EPKSCDKTHTCPP |
| | | CPAPELLGGPSVF |
| | | LFPPKPKDTLMIS |
| | | RTPEVTCVVVDVS |
| | | HEDPEVKFNWYVD |
| | | GVEVHNAKTKPRE |
| | | EQYNSTYRVVSVL |
| | | TVLHQDWLNGKEY |
| | | KCKVSNKALPAPI |
| | | EKTISKAKGQPRE |
| | | PQVYTLPPSRDEL |
| | | TKNQVSLTCLVKG |
| | | FYPSDIAVEWESN |
| | | GQPENNYKTTPPV |
| | | LDSDGSFFLYSKL |
| | | TVDKSRWQQGNVF |
| | | SCSVMHEALHNHY |
| | | TQKSLSLSPGK |

IL-15/IL-15Ra sushi domain fusion (Cytune)

| | | |
|---|---|---|
| SEQ ID | Human | NWVNVISDLKKIE |
| NO: 1005 | IL-15 | DLIQSMHIDATLY |
| | | TESDVHPSCKVTA |
| | | MKCFLLELQVISL |
| | | ESGDASIHDTVEN |
| | | LIILANNSLSSNG |
| | | NVTESGCKECEEL |
| | | EXKNIKEFLQSFV |
| | | HIVQMFINTS |
| | | Where X is E or K |
| SEQ ID | Human | ITCPPPMSVEHAD |
| NO: 1006 | IL-15Ra | IWVKSYSLYSRER |
| | sushi and | YICNSGFKRKAGT |
| | hinge | SSLTECVLNKATN |
| | domains | VAHWTTPSLKCIR |
| | | DPALVHQRPAPP |

TGFβ inhibitors

In certain embodiments, the anti-PD-L1 antibody molecule described herein is administered in combination with a transforming growth factor beta (TGFβ) inhibitor. In some embodiments, the TGFβ inhibitor is chosen from XOMA 089 (Novartis) or fresolimumab (Sanofi-Aventis).

Exemplary TGFβ inhibitors

In some embodiments, the TGFβ inhibitor comprises XOMA 089, or a compound disclosed in International Application Publication No. WO 2012/167143, which is incorporated by reference in its entirety. TGFβ is also known as TGF-β, TGFb, or TGF-beta, and is used interchangeably herein.

XOMA 089 is also known as XPA.42.089. XOMA 089 is a fully human monoclonal antibody that specifically binds and neutralizes TGF-beta 1 and 2 ligands.

The heavy chain variable region of XOMA 089 comprises the amino acid sequence of:

(SEQ ID NO: 1101)
QVQLVQSGAEVKKPGSSVKVSCKASGGTFSSYA
ISWVRQAPGQGLEWMGGIIPIFGTANYAQKFQG
RVTITADESTSTAYMELSSLRSEDTAVYYCARG
LWEVRALPSVYWGQGTLVTVSS
(disclosed as SEQ ID NO: 6 in
WO 2012/167143).

The light chain variable region of XOMA 089 comprises the amino acid sequence of:

(SEQ ID NO: 1102)
SYELTQPPSVSVAPGQTARITCGANDIGSKSVHWYQQKAGQA
PVLVVSEDIIRPSGIPERISGSNSGNTATLTISRVEAGDEAD
YYCQVWDRDSDQYVFGTGTKVTVLG
(disclosed as SEQ ID NO: 8 in WO 2012/167143).

XOMA 089 binds with high affinity to the human TGF-β isoforms. Generally, XOMA 089 binds with high affinity to TGF-β1 and TGF-β2, and to a lesser extent to TGF-β3. In Biacore assays, the K_{D} of XOMA 089 on human TGF-β is 14.6 pM for TGF-β1, 67.3 pM for TGF-β2, and 948 pM for TGF-β3. Given the high affinity binding to all three TGF-β isoforms, in certain embodiments, XOMA 089 is expected to bind to TGF-β 1, 2 and 3 at a dose of XOMA 089 as described herein. XOMA 089 cross-reacts with rodent and cynomolgus monkey TGF-β and shows functional activity in vitro and in vivo, making rodent and cynomolgus monkey relevant species for toxicology studies.

In one embodiment, the TGF-β inhibitor (e.g., XOMA 089) is administered at a dose between 0.1 mg/kg and 20 mg/kg, e.g., between 0.1 mg/kg and 15 mg/kg, between 0.1 mg/kg and 12 mg/kg, between 0.3 mg/kg and 6 mg/kg, between 1 mg/kg and 3 mg/kg, between 0.1 mg/kg and 1 mg/kg, between 0.1 mg/kg and 0.5 mg/kg, between 0.1 mg/kg and 0.3 mg/kg, between 0.3 mg/kg and 3 mg/kg, between 0.3 mg/kg and 1 mg/kg, between 3 mg/kg and 6 mg/kg, or between 6 mg/kg and 12 mg/kg, e.g., at a dose of about 0.1 mg/kg, 0.3 mg/kg, 0.5 mg/kg, 1 mg/kg, 3 mg/kg, 6 mg/kg, 12 mg/kg, or 15 mg/kg, e.g., once every week, once every two weeks, once every three weeks, once every four weeks, or once every six weeks.

In one embodiment, the TGF-β inhibitor (e.g., XOMA 089) is administered at a dose between 0.1 mg/kg and 15 mg/kg (e.g., between 0.3 mg/kg and 12 mg/kg or between 1 mg/kg and 6 mg, e.g., about 0.1 mg/kg, 0.3 mg/kg, 1 mg/kg, 3 mg/kg, 6 mg/kg, 12 mg/kg, or 15 mg/kg), e.g., once every three weeks. For example, the TGF-β inhibitor (e.g., XOMA 089) can be administered at a dose between 0.1 mg/kg and 1 mg/kg (e.g., between 0.1 mg/kg and 1 mg/kg, e.g., 0.3 mg/kg), e.g., once every three weeks. In one embodiment, the TGF-β inhibitor (e.g., XOMA 089) is administered intravenously.

### Other Exemplary TGFβ Inhibitors

In some embodiments, the TGFβ inhibitor comprises fresolimumab (CAS Registry Number: 948564-73-6). Fresolimumab is also known as GC1008. Fresolimumab is a human monoclonal antibody that binds to and inhibits TGF-beta isoforms 1, 2 and 3.

The heavy chain of fresolimumab comprises the amino acid sequence of:

(SEQ ID NO: 1103)
QVQLVQSGAEVKKPGSSVKVSCKASGYTFSSN
VISWVRQAPGQGLEWMGGVIPIVDIANYAQRF
KGRVTITADESTSTTYMELSSLRSEDTAVYYC
ASTLGLVLDAMDYWGQGTLVTVSSASTKGPSV
FPLAPCSRSTSESTAALGCLVKDYFPEPVTVS
WNSGALTSGVHTFPAVLQSSGLYSLSSVVTVP
SSSLGTKTYTCNVDHKPSNTKVDKRVESKYGP
PCPSCPAPEFLGGPSVFLFPPKPKDTLMISRT
PEVTCVVVDVSQEDPEVQFNWYVDGVEVHNAK
TKPREEQFNSTYRVVSVLTVLHQDWLNGKEYK
CKVSNKGLPSSIEKTISKAKGQPREPQVYTLP
PSQEEMTKNQVSLTCLVKGFYPSDIAVEWESN
GQPENNYKTTPPVLDSDGSFFLYSRLTVDKSR
WQEGNVFSCSVMHEALHNHYTQKSLSLSLGK.

The light chain of fresolimumab comprises the amino acid sequence of:

(SEQ ID NO: 1104)
ETVLTQSPGTLSLSPGERATLSCRASQSLGSS
YLAWYQQKPGQAPRLLIYGASSRAPGIPDRFS
GSGSGTDFTLTISRLEPEDFAVYYCQQYADSP
ITFGQGTRLEIKRTVAAPSVFIFPPSDEQLKS
GTASVVCLLNNFYPREAKVQWKVDNALQSGNS
QESVTEQDSKDSTYSLSSTLTLSKADYEKHKV
YACEVTHQGLSSPVTKSFNRGEC.

Fresolimumab is disclosed, e.g., in International Application Publication No. WO 2006/086469, and U.S. Pat. Nos. 8,383,780 and 8,591,901, which are incorporated by reference in their entirety.

### Pharmaceutical Compositions, Formulations, and Kits

In another aspect, the disclosure provides compositions, e.g., pharmaceutically acceptable compositions, which include an anti-PD-L1 antibody molecule described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions described herein may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical preferred compositions are in the form of injectable or infusible solutions. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (e.g., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

An anti-PD-L1 antibody molecule or a composition described herein can be formulated into a formulation (e.g., a dose formulation or dosage form) suitable for administration (e.g., intravenous administration) to a subject as described herein. The formulation described herein can be a liquid formulation, a lyophilized formulation, or a reconstituted formulation.

In certain embodiments, the formulation is a liquid formulation. In some embodiments, the formulation (e.g., liquid formulation) comprises an anti-PD-L1 antibody molecule (e.g., an anti-PD-L1 antibody molecule described herein) and a buffering agent.

In some embodiments, the formulation (e.g., liquid formulation) comprises an anti-PD-L1 antibody molecule present at a concentration of 25 mg/mL to 250 mg/mL, e.g., 50 mg/mL to 200 mg/mL, 60 mg/mL to 180 mg/mL, 70 mg/mL to 150 mg/mL, 80 mg/mL to 120 mg/mL, 90 mg/mL to 110 mg/mL, 50 mg/mL to 150 mg/mL, 50 mg/mL to 100 mg/mL, 150 mg/mL to 200 mg/mL, or 100 mg/mL to 200 mg/mL, e.g., 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL. In certain embodiments, the anti-PD-L1 antibody molecule is present at a concentration of 80 mg/mL to 120 mg/mL, e.g., 100 mg/mL.

In some embodiments, the formulation (e.g., liquid formulation) comprises a buffering agent comprising histidine (e.g., a histidine buffer). In certain embodiments, the buffering agent (e.g., histidine buffer) is present at a concentration of 1 mg/mL to 10 mg/mL, e.g., 2 mg/mL to 8 mg/mL, 1 mg/mL to 5 mg/mL, 3 mg/mL to 7 mg/mL, 2 mg/mL to 6 mg/mL, 3 mg/mL to 8 mg/mL, 1 mg/mL to 5 mg/mL, 2 mg/mL to 7 mg/mL, 3 mg/mL to 9 mg/mL, or 1 mg/mL to 6 mg/mL, e.g., 2 mg/mL, 3 mg/mL, 4 mg/mL, 5 mg/mL, 6 mg/mL, 7 mg/mL, 8 mg/mL, or 9 mg/mL. In some embodiments, the buffering agent (e.g., histidine buffer) is present at a concentration of 2 mg/mL to 6 mg/mL, e.g., about 3 mg/mL (e.g., 3.1 mg/mL). In other embodiments, the buffering agent (e.g., a histidine buffer) or the formulation has a pH of 4 to 7, e.g., 5 to 6, e.g., 5, 5.5, or 6. In some embodiments, the buffering agent (e.g., histidine buffer) or the formulation has a pH of 5 to 6, e.g., 5.5. In certain embodiments, the buffering agent comprises a histidine buffer at a concentration of 2 mg/mL to 6 mg/mL (e.g., about 3 mg/mL) and has a pH of 5 to 6 (e.g., 5.5). In certain embodiments, the buffering agent comprises histidine and histidine-HCl.

In some embodiments, the formulation (e.g., liquid formulation) comprises an anti-PD-L1 antibody molecule present at a concentration of 80 to 120 mg/mL, e.g., 100 mg/mL; and a buffering agent that comprises a histidine buffer at a concentration of 2 mg/mL to 6 mg/mL (e.g., about 3 mg/mL), at a pH of 5 to 6 (e.g., 5.5).

In some embodiments, the formulation (e.g., liquid formulation) further comprises a carbohydrate. In certain embodiments, the carbohydrate is sucrose. In some embodiments, the carbohydrate (e.g., sucrose) is present at a concentration of 20 mg/mL to 200 mg/mL, e.g., 25 mg/mL to 180 mg/mL, 30 mg/mL to 170 mg/ml, 45 mg/mL to 140 mg/ml, 60 mg/mL to 190 mg/mL, 35 mg/mL to 165 mg/mL, 70 mg/mL to 130 mg/mL, 65 mg/mL to 145 mg/mL, 40 mg/mL to 160 mg/mL, 55 mg/mL to 165 mg/mL, 30 mg/mL to 150 mg/mL, 50 mg/mL to 175 mg/mL, or 75 mg/mL to 125 mg/mL, e.g., 30 mg/mL, 40 mg/mL, 50 mg/mL, 60 mg/mL, 70 mg/mL, 75 mg/mL, 80 mg/mL, 85 mg/mL, 90 mg/mL, 100 mg/mL, 110 mg/mL, 120 mg/mL, 130 mg/mL, 140 mg/mL, or 150 mg/mL. In some embodiments, the formulation comprises a carbohydrate or sucrose present at a concentration of 50 mg/mL to 100 mg/mL, e.g., about 75 mg/mL (e.g., 75.3 mg/mL).

In some embodiments, the formulation (e.g., liquid formulation) comprises an anti-PD-L1 antibody molecule present at a concentration of 80 to 120 mg/mL, e.g., 100 mg/mL; a buffering agent that comprises a histidine buffer at a concentration of 2 mg/mL to 6 mg/mL, e.g., about 3 mg/mL; and a carbohydrate or sucrose present at a concentration of 50 mg/mL to 100 mg/mL, e.g., about 75 mg/mL, at a pH of 5 to 6 (e.g., 5.5).

In some embodiments, the formulation (e.g., liquid formulation) further comprises a surfactant. In certain embodiments, the surfactant is polysorbate 20. In some embodiments, the surfactant or polysorbate 20) is present at a concentration of 0.1 mg/mL to 1.0 mg/mL e.g. 0.2 mg/mL to 0.9 mg/mL, 0.3 mg/mL to 0.8 mg/mL, 0.4 mg/mL to 0.9 mg/mL, 0.3 mg/mL to 0.7 mg/mL, 0.2 mg/mL to 0.8 mg/mL, 0.3 mg/mL to 0.6 mg/mL, 0.4 mg/mL to 0.7 mg/mL, 0.2 mg/mL to 0.7 mg/mL, 0.3 mg/mL to 0.9 mg/mL, 0.3 mg/mL to 0.5 mg/mL, 0.4 mg/mL to 0.8 mg/mL, or 0.2 mg/mL to 0.5 mg/mL, e.g., 0.2 mg/mL, 0.3 mg/mL, 0.4 mg/mL, 0.5 mg/mL, 0.6 mg/mL, 0.7 mg/mL, 0.8 mg/mL, or 0.9 mg/mL. In some embodiments, the formulation comprises a surfactant or polysorbate 20 present at a concentration of 0.2 mg/mL to 0.6 mg/mL, e.g., 0.4 mg/mL.

In some embodiments, the formulation (e.g., liquid formulation) comprises an anti-PD-L1 antibody molecule present at a concentration of 80 to 120 mg/mL, e.g., 100 mg/mL; a buffering agent that comprises a histidine buffer at a concentration of 2 mg/mL to 6 mg/mL, e.g., about 3 mg/mL; a carbohydrate or sucrose present at a concentration of 50 mg/mL to 100 mg/mL, e.g., about 75 mg/mL; and a surfactant or polysorbate 20 present at a concentration of 0.2 mg/mL to 0.6 mg/mL, e.g., 0.4 mg/mL, at a pH of 5 to 6 (e.g., 5.5).

In some embodiments, the formulation (e.g., liquid formulation) comprises an anti-PD-L1 antibody molecule present at a concentration of 100 mg/mL; a buffering agent that comprises a histidine buffer (e.g., histidine/histidine-HCL) at a concentration of about 3 mg/mL (e.g., 3.1 mg/mL); a carbohydrate or sucrose present at a concentration of about 75 mg/mL (e.g., 75.3 mg/mL); and a surfactant or polysorbate 20 present at a concentration of 0.4 mg/mL, at a pH of 5 to 6 (e.g., 5.5).

In some embodiments, the liquid formulation is prepared by diluting a formulation comprising an anti-PD-L1 antibody molecule described herein. For example, a drug substance formulation can be diluted with a solution comprising one or more excipients (e.g., concentrated excipients). In some embodiments, the solution comprises one, two, or all of histidine, sucrose, or polysorbate 20. In certain embodiments, the solution comprises the same excipient(s) as the drug substance formulation. Exemplary excipients include, but are not limited to, an amino acid (e.g., histidine), a carbohydrate (e.g., sucrose), or a surfactant (e.g., polysorbate 20). In certain embodiments, the liquid formulation is not a reconstituted lyophilized formulation. In other embodiments, the liquid formulation is a reconstituted lyophilized formulation. In some embodiments, the formulation is stored as a liquid. In other embodiments, the formulation is prepared as a liquid and then is dried, e.g., by lyophilization or spray-drying, prior to storage.

In certain embodiments, 0.5 mL to 10 mL (e.g., 0.5 mL to 8 mL, 1 mL to 6 mL, or 2 mL to 5 mL, e.g., 1 mL, 1.2 mL, 1.5 mL, 2 mL, 3 mL, 4 mL, 4.5 mL, or 5 mL) of the liquid formulation is filled per container (e.g., vial). In other embodiments, the liquid formulation is filled into a container (e.g., vial) such that an extractable volume of at least 1 mL (e.g., at least 1.2 mL, at least 1.5 mL, at least 2 mL, at least 3 mL, at least 4 mL, or at least 5 mL) of the liquid formulation can be withdrawn per container (e.g., vial). In certain embodiments, the liquid formulation is extracted from the container (e.g., vial) without diluting at a clinical site. In certain embodiments, the liquid formulation is diluted from a drug substance formulation and extracted from the container (e.g., vial) at a clinical site. In certain embodiments, the formulation (e.g., liquid formulation) is injected to an infusion bag, e.g., within 1 hour (e.g., within 45 minutes, 30 minutes, or 15 minutes) before the infusion starts to the patient.

A formulation described herein can be stored in a container. The container used for any of the formulations described herein can include, e.g., a vial, and optionally, a stopper, a cap, or both. In certain embodiments, the vial is a glass vial, e.g., a 6R white glass vial or colorless glass vial. In other embodiments, the stopper is a rubber stopper, e.g., a grey rubber stopper. In other embodiments, the cap is a flip-off cap, e.g., an aluminum flip-off cap. In some embodiments, the container comprises a 6R white glass vial, a grey rubber stopper, and an aluminum flip-off cap. In some embodiments, the container (e.g., vial) is for a single-use container. In certain embodiments, 25 mg/mL to 250 mg/mL, e.g., 50 mg/mL to 200 mg/mL, 60 mg/mL to 180 mg/mL, 70 mg/mL to 150 mg/mL, 80 mg/mL to 120 mg/mL, 90 mg/mL to 110 mg/mL, 50 mg/mL to 150 mg/mL, 50 mg/mL to 100 mg/mL, 150 mg/mL to 200 mg/mL, or 100 mg/mL to 200 mg/mL, e.g., 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL, of the anti-PD-L1 antibody molecule, is present in the container (e.g., vial).

In some embodiments, the formulation is a lyophilized formulation. In certain embodiments, the lyophilized formulation is lyophilized or dried from a liquid formulation comprising an anti-PD-L1 antibody molecule described herein. For example, 1 to 5 mL, e.g., 1 to 2 mL, of a liquid formulation can be filled per container (e.g., vial) and lyophilized.

In some embodiments, the formulation is a reconstituted formulation. In certain embodiments, the reconstituted formulation is reconstituted from a lyophilized formulation comprising an anti-PD-L1 antibody molecule described herein. For example, a reconstituted formulation can be prepared by dissolving a lyophilized formulation in a diluent such that the protein is dispersed in the reconstituted formulation. In some embodiments, the lyophilized formulation is reconstituted with 1 mL to 5 mL, e.g., 1 mL to 2 mL, e.g., 1.2 mL, of water or buffer for injection. In certain embodiments, the lyophilized formulation is reconstituted with 1 mL to 2 mL of water for injection, e.g., at a clinical site.

In some embodiments, the reconstituted formulation comprises an anti-PD-L1 antibody molecule (e.g., an anti-PD-L1 antibody molecule described herein) and a buffering agent.

In some embodiments, the reconstituted formulation comprises an anti-PD-L1 antibody molecule present at a concentration of 25 mg/mL to 250 mg/mL, e.g., 50 mg/mL to 200 mg/mL, 60 mg/mL to 180 mg/mL, 70 mg/mL to 150 mg/mL, 80 mg/mL to 120 mg/mL, 90 mg/mL to 110 mg/mL, 50 mg/mL to 150 mg/mL, 50 mg/mL to 100 mg/mL, 150 mg/mL to 200 mg/mL, or 100 mg/mL to 200 mg/mL, e.g., 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL. In certain embodiments, the anti-PD-L1 antibody molecule is present at a concentration of 80 mg/mL to 120 mg/mL, e.g., 100 mg/mL.

In some embodiments, the reconstituted formulation comprises a buffering agent comprising histidine (e.g., a histidine buffer). In certain embodiments, the buffering agent (e.g., histidine buffer) is present at a concentration of 1 mg/mL to 10 mg/mL, e.g., 2 mg/mL to 8 mg/mL, 1 mg/mL to 5 mg/mL, 3 mg/mL to 7 mg/mL, 2 mg/mL to 6 mg/mL, 3 mg/mL to 8 mg/mL, 1 mg/mL to 5 mg/mL, 2 mg/mL to 7 mg/mL, 3 mg/mL to 9 mg/mL, or 1 mg/mL to 6 mg/mL, e.g., 2 mg/mL, 3 mg/mL, 4 mg/mL, 5 mg/mL, 6 mg/mL, 7 mg/mL, 8 mg/mL, or 9 mg/mL. In some embodiments, the buffering agent (e.g., histidine buffer) is present at a concentration of 2 mg/mL to 6 mg/mL, e.g., about 3 mg/mL (e.g., 3.1 mg/mL). In other embodiments, the buffering agent (e.g., a histidine buffer) has a pH of 4 to 7, e.g., 5 to 6, e.g., 5, 5.5, or 6. In some embodiments, the buffering agent (e.g., histidine buffer) has a pH of 5 to 6, e.g., 5.5. In certain embodiments, the buffering agent comprises a histidine buffer at a concentration of 15 mM to 25 mM (e.g., 20 mM) and has a pH of 5 to 6 (e.g., 5.5). In certain embodiments, the buffering agent comprises histidine and histidine-HCl.

In some embodiments, the reconstituted formulation comprises an anti-PD-L1 antibody molecule present at a concentration of 80 to 120 mg/mL, e.g., 100 mg/mL; and a buffering agent that comprises a histidine buffer at a concentration of 2 mg/mL to 6 mg/mL (e.g., about 3 mg/mL) and has a pH of 5 to 6 (e.g., 5.5).

In some embodiments, the reconstituted formulation further comprises a carbohydrate. In certain embodiments, the carbohydrate is sucrose. In some embodiments, the carbohydrate (e.g., sucrose) is present at a concentration of 20 mg/mL to 200 mg/mL, e.g., 25 mg/mL to 180 mg/mL, 30 mg/mL to 170 mg/ml, 45 mg/mL to 140 mg/ml, 60 mg/mL to 190 mg/mL, 35 mg/mL to 165 mg/mL, 70 mg/mL to 130 mg/mL, 65 mg/mL to 145 mg/mL, 40 mg/mL to 160 mg/mL, 55 mg/mL to 165 mg/mL, 30 mg/mL to 150 mg/mL, 50 mg/mL to 175 mg/mL, or 75 mg/mL to 125 mg/mL, e.g., 30 mg/mL, 40 mg/mL, 50 mg/mL, 60 mg/mL, 70 mg/mL, 75 mg/mL, 80 mg/mL, 85 mg/mL, 90 mg/mL, 100 mg/mL, 110 mg/mL, 120 mg/mL, 130 mg/mL, 140 mg/mL, or 150 mg/mL. In some embodiments, the formulation comprises a carbohydrate or sucrose present at a concentration of 50 mg/mL to 100 mg/mL, e.g., about 75 mg/mL (e.g., 75.3 mg/mL).

In some embodiments, the reconstituted formulation comprises an anti-PD-L1 antibody molecule present at a concentration of 80 to 120 mg/mL, e.g., 100 mg/mL; a buffering agent that comprises a histidine buffer at a concentration of 2 mg/mL to 6 mg/mL, e.g., about 3 mg/mL and has a pH of 5 to 6 (e.g., 5.5); and a carbohydrate or sucrose present at a concentration of 50 mg/mL to 100 mg/mL, e.g., about 75 mg/mL.

In some embodiments, the reconstituted formulation further comprises a surfactant. In certain embodiments, the surfactant is polysorbate 20. In some embodiments, the surfactant or polysorbate 20) is present at a concentration of 0.1 mg/mL to 1.0 mg/mL e.g. 0.2 mg/mL to 0.9 mg/mL, 0.3 mg/mL to 0.8 mg/mL, 0.4 mg/mL to 0.9 mg/mL, 0.3 mg/mL to 0.7 mg/mL, 0.2 mg/mL to 0.8 mg/mL, 0.3 mg/mL to 0.6 mg/mL, 0.4 mg/mL to 0.7 mg/mL, 0.2 mg/mL to 0.7 mg/mL, 0.3 mg/mL to 0.9 mg/mL, 0.3 mg/mL to 0.5 mg/mL, 0.4 mg/mL to 0.8 mg/mL, or 0.2 mg/mL to 0.5 mg/mL, e.g., 0.2 mg/mL, 0.3 mg/mL, 0.4 mg/mL, 0.5 mg/mL, 0.6 mg/mL, 0.7 mg/mL, 0.8 mg/mL, or 0.9 mg/mL. In some embodiments, the formulation comprises a surfactant or polysorbate 20 present at a concentration of 0.2 mg/mL to 0.6 mg/mL, e.g., 0.4 mg/mL).

In some embodiments, the reconstituted formulation comprises an anti-PD-L1 antibody molecule present at a concentration of 80 to 120 mg/mL, e.g., 100 mg/mL; a buffering agent that comprises a histidine buffer at a concentration of 2 mg/mL to 6 mg/mL, e.g., about 3 mg/mL and has a pH of 5 to 6 (e.g., 5.5); a carbohydrate or sucrose present at a concentration of 50 mg/mL to 100 mg/mL, e.g., about 75 mg/mL; and a surfactant or polysorbate 20 present at a concentration of 0.2 mg/mL to 0.6 mg/mL, e.g., 0.4 mg/mL.

In some embodiments, the reconstituted formulation comprises an anti-PD-L1 antibody molecule present at a concentration of 100 mg/mL; a buffering agent that comprises a histidine buffer (e.g., histidine/histidine-HCL) at a concentration of about 3 mg/mL (e.g., 3.1 mg/mL) and has a pH of 5.5; a carbohydrate or sucrose present at a concentration of about 75 mg/mL (e.g., 75.3 mg/mL); and a surfactant or polysorbate 20 present at a concentration of 0.4 mg/mL.

In some embodiments, the formulation is reconstituted such that an extractable volume of at least 1 mL (e.g., at least 1.2 mL, 1.5 mL, 2 mL, 2.5 mL, or 3 mL) of the reconstituted formulation can be withdrawn from the container (e.g., vial) containing the reconstituted formulation. In certain embodiments, the formulation is reconstituted and/or extracted from the container (e.g., vial) at a clinical site. In certain embodiments, the formulation (e.g., reconstituted formulation) is injected to an infusion bag, e.g., within 1 hour (e.g., within 45 minutes, 30 minutes, or 15 minutes) before the infusion starts to the patient.

Other exemplary buffering agents that can be used in the formulation described herein include, but are not limited to, an arginine buffer, a citrate buffer, or a phosphate buffer. Other exemplary carbohydrates that can be used in the formulation described herein include, but are not limited to, trehalose, mannitol, sorbitol, or a combination thereof. The formulation described herein may also contain a tonicity agent, e.g., sodium chloride, and/or a stabilizing agent, e.g., an amino acid (e.g., glycine, arginine, methionine, or a combination thereof).

The antibody molecules can be administered by a variety of methods known in the art, although for many therapeutic applications, the preferred route/mode of administration is intravenous injection or infusion. For example, the antibody molecules can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the antibody molecules can be administered by intravenous infusion at a rate of less than 10 mg/min; preferably less than or equal to 5 mg/min to reach a dose of about 1 to 100 mg/m 2, preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody molecule can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody molecule is 50 mg to 2000 mg, typically 80 mg to 1800 mg. In certain embodiments, the anti-PD-L1 antibody molecule is administered by infusion (e.g., subcutaneously or intravenously) at a dose (e.g., a flat dose) of about 60 mg to about 100 mg (e.g., about 80 mg), about 200 mg to about 300 mg (e.g., about 240 mg), about 700 mg to about 900 mg (e.g., about 800 mg), about 1000 mg to about 1400 mg (e.g., about 1200 mg), or about 1400 mg to about 1800 mg (e.g., about 1600 mg). The dosing schedule (e.g., flat dosing schedule) can vary from e.g., once a week to once every two weeks, once every three weeks, once every four weeks, once every five weeks, or once every six weeks.

In one embodiment, the anti-PD-L1 antibody molecule is administered at a dose from about 60 mg to 100 mg (e.g., about 80 mg) once every three weeks, once every four weeks, or once every six weeks.

In one embodiment, the anti-PD-L1 antibody molecule is administered at a dose from about 200 mg to about 300 mg (e.g., about 240 mg) once every three weeks, once every four weeks, or once every six weeks.

In one embodiment, the anti-PD-L1 antibody molecule is administered at a dose from about 700 mg to about 900 mg (e.g., about 800 mg) once every three weeks, once every four weeks, or once every six weeks.

In one embodiment, the anti-PD-L1 antibody molecule is administered at a dose from about 1000 mg to about 1400 mg (e.g., about 1200 mg) once every two weeks or once every four weeks.

In one embodiment, the anti-PD-L1 antibody molecule is administered at a dose from about 1400 mg to about 1800 mg (e.g., about 1600 mg) once every two weeks or once every four weeks.

In one embodiment, the anti-PD-L1 antibody molecule is administered at a dose about 80 mg once every three weeks.

In one embodiment, the anti-PD-L1 antibody molecule is administered at a dose about 240 mg once every three weeks.

In one embodiment, the anti-PD-L1 antibody molecule is administered at a dose about 800 mg once every three weeks, once every four weeks, or once every six weeks.

In one embodiment, the anti-PD-L1 antibody molecule is administered at a dose about 1200 mg once every three weeks, once every four weeks, or once every six weeks.

In one embodiment, the anti-PD-L1 antibody molecule is administered at a dose about 1600 mg once every three weeks, once every four weeks, or once every six weeks.

While not wishing to be bound by theory, in some embodiments, flat or fixed dosing can be beneficial to patients, for example, to save drug supply and to reduce pharmacy errors.

The antibody molecule can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the infusion rate of about 110 to 130 mg/m² achieves a level of about 3 mg/kg. In other embodiments, the antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the antibody is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the modified antibody or antibody fragment may vary according to factors such as the disease state, age, sex, and weight of the individual, and the ability of the antibody or antibody portion to elicit a desired response in the individual. A therapeutically effective amount is also one in which any toxic or detrimental effects of the modified antibody or antibody fragment is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the disclosure is a kit comprising an anti-PD-L1 antibody molecule, composition, or formulation described herein. The kit can include one or more other elements including: instructions for use (e.g., in accordance a dosage regimen described herein); other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

### Use of Anti-PD-L1 Antibody Molecules

The anti-PD-L1 antibody molecules described herein can be used to modify an immune response in a subject. In some embodiments, the immune response is enhanced, stimulated or up-regulated. In certain embodiments, the immune response is inhibited, reduced, or down-regulated. For example, these antibody molecules can be administered to cells in culture, e.g. in vitro or ex vivo, or in a subject, e.g., in vivo, to treat, prevent, and/or diagnose a variety of disorders, such as cancers, immune disorders, and infectious diseases.

As used herein, the term “subject” is intended to include human and non-human animals. In some embodiments, the subject is a human subject, e.g., a human patient having a disorder or condition characterized by abnormal PD-L1 functioning. Generally, the subject has at least some PD-L1 protein, including the PD-L1 epitope that is bound by the antibody molecule, e.g., a high enough level of the protein and epitope to support antibody binding to PD-L1. The term “non-human animals” includes mammals and non-mammals, such as non-human primates. In some embodiments, the subject is a human. In some embodiments, the subject is a human patient in need of enhancement of an immune response. The methods and compositions described herein are suitable for treating human patients having a disorder that can be treated by modulating (e.g., augmenting or inhibiting) an immune response.

In certain embodiments, the subject has not been treated with a PD-1/PD-L1 therapy prior to receiving the anti-PD-L1 antibody molecule. In other embodiments, the subject has been treated with a with a PD-1/PD-L1 therapy prior to receiving the anti-PD-L1 antibody molecule. In certain embodiments, the subject has been identified as having PD-L1 expression in tumor infiltrating lymphocytes. In other embodiments, the subject does not have detectable level of PD-L1 expression in tumor infiltrating lymphocytes.

### Methods of Treating Cancer

In one aspect, the disclosure relates to treatment of a subject in vivo using an anti-PD-L1 antibody molecule (e.g., an anti-PD-L1 antibody molecule described herein), or a composition or formulation comprising an anti-PD-L1 antibody molecule (e.g., a composition or formulation described herein) such that growth of cancerous tumors is inhibited or reduced.

In certain embodiments, the anti-PD-L1 antibody molecule is administered in an amount effective to treat a cancer or a metastatic lesion thereof. In some embodiments, the anti-PD-L1 antibody molecule is administered at a dose from about 20 mg to about 2000 mg once every three weeks, once every four weeks, or once every six weeks.

For example, the anti-PD-L1 antibody molecule can be administered at a dose from about 40 mg to about 2000 mg, about 300 mg to about 1800 mg, about 200 mg to about 1600 mg, about 300 mg to about 1400 mg, about 600 to about 1700 mg, about 700 mg to about 1900 mg, or about 400 mg to about 1500 mg, once every three weeks, once every four weeks, or once every six weeks.

In one embodiment, the anti-PD-L1 antibody molecule is administered at a dose from about 60 mg to 100 mg (e.g., about 80 mg) once every three weeks.

In one embodiment, the anti-PD-L1 antibody molecule is administered at a dose from about 200 mg to about 300 mg (e.g., about 240 mg) once every three weeks.

In one embodiment, the anti-PD-L1 antibody molecule is administered at a dose from about 700 mg to about 900 mg (e.g., about 800 mg) once every three weeks, once every four weeks, or once every six weeks.

In one embodiment, the anti-PD-L1 antibody molecule is administered at a dose of about 1000 mg to about 1400 mg (e.g., about 1200 mg) once every three weeks, once every four weeks, or once every six weeks.

In one embodiment, the anti-PD-L1 antibody molecule is administered at a dose from about 1400 mg to about 1800 mg (e.g., about 1600 mg) once every three weeks, once every four weeks, or once every six weeks.

In one embodiment, the anti-PD-L1 antibody molecule is administered at a dose about 80 mg once every three weeks, once every four weeks, or once every six weeks.

In one embodiment, the anti-PD-L1 antibody molecule is administered at a dose about 240 mg once every three weeks, once every four weeks, or once every six weeks.

In one embodiment, the anti-PD-L1 antibody molecule is administered at a dose about 800 mg once every three weeks, once every four weeks, or once every six weeks.

In one embodiment, the anti-PD-L1 antibody molecule is administered at a dose about 1200 mg once every three weeks, once every four weeks, or once every six weeks.

In one embodiment, the anti-PD-L1 antibody molecule is administered at a dose about 1600 mg once every three weeks, once every four weeks, or once every six weeks.

An anti-PD-L1 antibody, or a composition or formulation comprising an anti-PD-L1 antibody molecule, may be used alone to inhibit the growth of cancerous tumors. Alternatively, an anti-PD-L1 antibody, or a composition or formulation comprising an anti-PD-L1 antibody molecule, may be used in combination with one or more of: a standard of care treatment (e.g., for cancers or infectious disorders), another antibody or antigen-binding fragment thereof, an immunomodulator (e.g., an activator of a costimulatory molecule or an inhibitor of an inhibitory molecule); a vaccine, e.g., a therapeutic cancer vaccine; or other forms of cellular immunotherapy, as described herein.

Accordingly, in one embodiment, the disclosure provides a method of inhibiting growth of tumor cells in a subject, comprising administering to the subject a therapeutically effective amount of an anti-PD-L1 antibody molecule described herein, e.g., in accordance with a dosage regimen described herein. In an embodiment, the anti-PD-L1 antibody molecule is administered in the form of a composition or formulation described herein.

In one embodiment, the method is suitable for the treatment of cancer in vivo. To achieve antigen-specific enhancement of immunity, the anti-PD-L1 antibody molecule can be administered together with an antigen of interest. When an anti-PD-L1 antibody is administered in combination with one or more agents, the combination can be administered in either order or simultaneously.

In another aspect, a method of treating a subject, e.g., reducing or ameliorating, a hyperproliferative condition or disorder (e.g., a cancer), e.g., solid tumor, a hematological cancer, soft tissue tumor, or a metastatic lesion, in a subject is provided. The method includes administering to the subject an anti-PD-L1 antibody molecule, or a composition or formulation comprising an anti-PD-L1 antibody molecule, as disclosed herein, in accordance with a dosage regimen disclosed herein.

As used herein, the term “cancer” is meant to include all types of cancerous growths or oncogenic processes, metastatic tissues or malignantly transformed cells, tissues, or organs, irrespective of histopathological type or stage of invasiveness. Examples of cancerous disorders include, but are not limited to, solid tumors, hematological cancers, soft tissue tumors, and metastatic lesions. Examples of solid tumors include malignancies, e.g., sarcomas, and carcinomas (including adenocarcinomas and squamous cell carcinomas), of the various organ systems, such as those affecting liver, lung, breast, lymphoid, gastrointestinal (e.g., colon), genitourinary tract (e.g., renal, urothelial, or bladder cells), prostate, CNS (e.g., brain, neural, or glial cells), skin, pancreas, and pharynx. Adenocarcinomas include malignancies, such as most colon cancers, rectal cancer, renal-cell carcinoma, liver cancer, non-small cell carcinoma of the lung, cancer of the small intestine, and cancer of the esophagus. Squamous cell carcinomas include malignancies, e.g., in the lung, esophagus, skin, head and neck region, oral cavity, anus, and cervix. In one embodiment, the cancer is a melanoma, e.g., an advanced stage melanoma. Metastatic lesions of the aforementioned cancers can also be treated or prevented using the methods and compositions of the invention.

Exemplary cancers whose growth can be inhibited using the antibodies molecules, compositions, or formulations, as disclosed herein, include cancers typically responsive to immunotherapy. Non-limiting examples of typical cancers for treatment include, e.g., bone cancer (e.g., a chordoma), skin cancer (e.g., a Merkel cell carcinoma or a melanoma, e.g., a cutaneous melanoma), breast cancer (e.g., a metastatic breast carcinoma or a stage IV breast carcinoma, e.g., a triple negative breast cancer (TNBC)), cervical cancer (e.g., a squamous cell carcinoma of the cervix), colorectal cancer (e.g., relapsed colorectal cancer or metastatic colorectal cancer, e.g., a microsatellite unstable colorectal cancer, a microsatellite stable colorectal cancer, a mismatch repair proficient colorectal cancer, or a mismatch repair deficient colorectal cancer), endometrial cancer, lung cancer (e.g., a non-small cell lung cancer (NSCLC)), ovarian cancer, or liver cancer (e.g., a hepatocellular carcinoma).

Examples of other cancers that can be treated include, but are not limited to, basal cell carcinoma, biliary tract cancer; bladder cancer; brain and central nervous system (CNS) cancer; primary CNS lymphoma; neoplasm of the CNS; breast cancer; bone cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia (including acute myeloid leukemia, chronic myeloid leukemia, acute lymphoblastic leukemia, chronic lymphocytic leukemia, chronic, or acute leukemia); liver cancer; lung cancer (e.g., small cell and non-small cell); renal cancer (e.g., clear cell carcinoma); lymphoma including Hodgkin's and non-Hodgkin's lymphoma; lymphocytic lymphoma; melanoma (e.g., cutaneous or intraocular malignant melanoma); myeloma; neuroblastoma; oral cavity cancer (e.g., lip, tongue, mouth, and pharynx); ovarian cancer; pancreatic cancer; prostate cancer (e.g., hormone refractory prostate adenocarcinoma); retinoblastoma; rhabdomyosarcoma; rectal cancer; cancer of the respiratory system; sarcoma; skin cancer; stomach cancer; testicular cancer; thyroid cancer; uterine cancer; cancer of the urinary system, hepatocarcinoma, cancer of the anal region, carcinoma of the fallopian tubes, carcinoma of the vagina, carcinoma of the vulva, cancer of the small intestine, cancer of the endocrine system, cancer of the parathyroid gland, cancer of the adrenal gland, sarcoma of soft tissue, cancer of the urethra, cancer of the penis, solid tumors of childhood, spinal axis tumor, brain stem glioma, pituitary adenoma, Kaposi's sarcoma, epidermoid cancer, squamous cell cancer, T-cell lymphoma, environmentally induced cancers including those induced by asbestos, other carcinomas and sarcomas, and combinations of the aforementioned cancers. Additionally, refractory or recurrent malignancies can be treated using the anti-PD-L1 antibody molecules described herein.

In some embodiments, the disorder is a cancer, e.g., a cancer described herein. In certain embodiments, the cancer is a solid tumor. In some embodiments, the cancer is a bone cancer, e.g., a chordoma. In some embodiments, the cancer is a skin cancer, e.g., a melanoma (e.g., a cutaneous melanoma, a stage II-IV melanoma, an HLA-A2 positive melanoma, an unresectable melanoma, or a metastatic melanoma), or a Merkel cell carcinoma. In some embodiments, the cancer is a breast cancer, e.g., a metastatic breast carcinoma or a stage IV breast carcinoma, e.g., a TNBC. In some embodiments, the cancer is a cervical cancer (e.g., a squamous cell carcinoma of the cervix). In some embodiments, the cancer is a colorectal cancer, e.g., a relapsed colorectal cancer or a metastatic colorectal cancer, e.g., a microsatellite unstable colorectal cancer, a microsatellite stable colorectal cancer, a mismatch repair proficient colorectal cancer, or a mismatch repair deficient colorectal cancer. In some embodiments, the cancer is an endometrial cancer. In some embodiments, the cancer is a lung cancer, e.g., a NSCLC. In some embodiments, the cancer is an ovarian cancer. In some embodiments, the cancer is a hepatocarcinoma, e.g., an advanced hepatocarcinoma. In some embodiments, the cancer is brain tumor, e.g., a glioblastoma, a gliosarcoma, or a recurrent brain tumor. In some embodiments, the cancer is a pancreatic cancer, e.g., an advanced pancreatic cancer. In some embodiments, the cancer is a renal cancer, e.g., a renal cell carcinoma (RCC) (e.g., a metastatic renal cell carcinoma) or a treatment-naïve metastatic kidney cancer. In some embodiments, the cancer is a virus-associated cancer. In some embodiments, the cancer is an anal canal cancer (e.g., a squamous cell carcinoma of the anal canal). In some embodiments, the cancer is a gastric cancer (e.g., an Epstein Barr Virus (EBV) positive gastric cancer, or a gastric or gastro-esophageal junction carcinoma). In some embodiments, the cancer is a head and neck cancer (e.g., an HPV positive and negative squamous cell cancer of the head and neck (SCCHN)). In some embodiments, the cancer is a nasopharyngeal cancer (NPC). In some embodiments, the cancer is a penile cancer (e.g., a squamous cell carcinoma of the penile). In some embodiments, the cancer is a vaginal or vulvar cancer (e.g., a squamous cell carcinoma of the vagina or vulva). In some embodiments, the cancer is a colorectal cancer, e.g., a relapsed colorectal cancer, a metastatic colorectal cancer, e.g., a microsatellite unstable colorectal cancer, a microsatellite stable colorectal cancer, a mismatch repair proficient colorectal cancer, or a mismatch repair deficient colorectal cancer. In certain embodiments, the cancer is a hematological cancer. In some embodiments, the cancer is a leukemia. In some embodiments, the cancer is a lymphoma, e.g., a Hodgkin lymphoma (HL) or a diffuse large B cell lymphoma (DLBCL) (e.g., a relapsed or refractory HL or DLBCL). In some embodiments, the cancer is a myeloma. In some embodiments, the cancer is an MSI-high cancer. In some embodiments, the cancer is a metastatic cancer. In other embodiments, the cancer is an advanced cancer. In other embodiments, the cancer is a relapsed or refractory cancer.

In one embodiment, the cancer is a Merkel cell carcinoma. In other embodiments, the cancer is a melanoma. In other embodiments, the cancer is a breast cancer, e.g., a triple negative breast cancer (TNBC) or a HER2-negative breast cancer. In other embodiments, the cancer is a renal cell carcinoma (e.g., a clear cell renal cell carcinoma (CCRCC) or a non-clear cell renal cell carcinoma (nccRCC)). In other embodiments, the cancer is a thyroid cancer, e.g., an anaplastic thyroid carcinoma (ATC). In other embodiments, the cancer is a neuroendocrine tumor (NET), e.g., an atypical pulmonary carcinoid tumor or an NET in pancreas, gastrointestinal (GI) tract, or lung. In certain embodiments, the cancer is a non-small cell lung cancer (NSCLC) (e.g., a squamous NSCLC or a non-squamous NSCLC). In certain embodiments, the cancer is a fallopian tube cancer. In certain embodiments, the cancer is a microsatellite instability-high colorectal cancer (MSI-high CRC) or a microsatellite stable colorectal cancer (MSS CRC).

In other embodiments, the cancer is a hematological malignancy or cancer including, but is not limited, to a leukemia or a lymphoma. For example, an anti-PD-L1 antibody molecule can be used to treat cancers and malignancies including, but not limited to, e.g., an acute leukemia, e.g., B-cell acute lymphoid leukemia (“BALL”), T-cell acute lymphoid leukemia (“TALL”), acute lymphoid leukemia (ALL); a chronic leukemia, e.g., chronic myelogenous leukemia (CML), chronic lymphocytic leukemia (CLL); an additional hematologic cancer or hematologic condition, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenström macroglobulinemia, and “preleukemia” which are a diverse collection of hematological conditions united by ineffective production (or dysplasia) of myeloid blood cells, and the like.

As used herein, the term “subject” is intended to include human and non-human animals. In some embodiments, the subject is a human subject, e.g., a human patient having a disorder or condition characterized by abnormal PD-L1 functioning. Generally, the subject has at least some PD-L1 protein, including the PD-L1 epitope that is bound by the antibody molecule, e.g., a high enough level of the protein and epitope to support antibody binding to PD-L1. The term “non-human animals” includes mammals and non-mammals, such as non-human primates. In some embodiments, the subject is a human. In some embodiments, the subject is a human patient in need of enhancement of an immune response. The methods and compositions described herein are suitable for treating human patients having a disorder that can be treated by modulating (e.g., augmenting or inhibiting) an immune response.

In some embodiments, the anti-PD-L1 antibody molecule, or the composition or formulation comprising the anti-PD-L1 antibody molecule, is administered as a single agent. In other embodiments, the anti-PD-L1 antibody molecule, or the composition or formulation comprising the anti-PD-L1 antibody molecule, is administered in combination with a second therapeutic agent or modality, e.g., a PD-1 inhibitor or a LAG-3 inhibitor. In some embodiments, the PD-1 inhibitor is an anti-PD-1 antibody molecule, e.g., an anti-PD-1 antibody described herein. In some embodiments, the LAG-3 inhibitor is an anti-LAG-3 antibody molecule, e.g., an anti-LAG-3 antibody molecule described herein.

In some embodiments, the cancer is a solid tumor. In certain embodiments, the cancer is chosen from a bone cancer (e.g., a chordoma), a skin cancer (e.g., a Merkel cell carcinoma or a melanoma, e.g., a cutaneous melanoma), a breast cancer (e.g., a metastatic breast carcinoma or a stage IV breast carcinoma, e.g., a TNBC), a cervical cancer (e.g., a squamous cell carcinoma of the cervix), a colorectal cancer (e.g., a relapsed colorectal cancer or a metastatic colorectal cancer, e.g., a microsatellite unstable colorectal cancer, a microsatellite stable colorectal cancer, a mismatch repair proficient colorectal cancer, or a mismatch repair deficient colorectal cancer), an endometrial cancer, a lung cancer (e.g., a NSCLC), an ovarian cancer, or a liver cancer (e.g., a hepatocellular carcinoma).

In some embodiments, the anti-PD-L1 antibody molecule, or the composition or formulation comprising the anti-PD-L1 antibody molecule, is administered as a single agent to treat the solid tumor (e.g., a solid tumor described herein). In other embodiments, the anti-PD-L1 antibody molecule, or the composition or formulation comprising the anti-PD-L 1 antibody molecule, is administered in combination with a second therapeutic agent or modality, e.g., a PD-1 inhibitor, to treat the solid tumor. In some embodiments, the PD-1 inhibitor is an anti-PD-1 antibody molecule, e.g., an anti-PD-1 antibody described herein. In certain embodiments, the anti-PD-1 antibody molecule is PDR001. In certain embodiments, the anti-PD-1 antibody molecule is REGN2810. In certain embodiments, the anti-PD-1 antibody molecule is Nivolumab. In certain embodiments, the anti-PD-1 antibody molecule is Pembrolizumab. In certain embodiments, the anti-PD-1 antibody molecule is Pidilizumab. In certain embodiments, the anti-PD-1 antibody molecule is MEDI0680. In certain embodiments, the anti-PD-1 antibody molecule is TSR-042. In certain embodiments, the anti-PD-1 antibody molecule is PF-06801591. In certain embodiments, the anti-PD-1 antibody molecule is BGB-A317. In certain embodiments, the anti-PD-1 antibody molecule is BGB-108. In certain embodiments, the anti-PD-1 antibody molecule is INCSHR1210. In certain embodiments, the anti-PD-1 antibody molecule is AMP-224.

Methods and compositions disclosed herein are useful for treating metastatic lesions associated with the aforementioned cancers.

In some embodiments, the method further comprises determining whether a tumor sample is positive for one or more of PD-L1, CD8, and IFN-γ, and if the tumor sample is positive for one or more, e.g., two, or all three, of the markers, then administering to the patient a therapeutically effective amount of an anti-PD-L1 antibody molecule, optionally in combination with one or more other immunomodulators or anti-cancer agents, as described herein.

In other embodiments, the anti-PD-L1 antibody molecule is used to treat a cancer that is characterized by microsatellite instability-high (MSI-H) or mismatch repair deficient (dMMR). The identification of MSI-H or dMMR tumor status for patients can be determined using, e.g., polymerase chain reaction (PCR) tests for MSI-H status or immunohistochemistry (IHC) tests for dMMR. Methods for identification of MSI-H or dMMR tumor status are described, e.g., in Ryan et al. Crit Rev Oncol Hematol. 2017; 116:38-57; Dietmaier and Hofstadter. Lab Invest 2001, 81:1453-1456; Kawakami et al. Curr Treat Options Oncol. 2015; 16(7): 30).

The combination therapies described herein can include a composition of the present invention co-formulated with, and/or co-administered with, one or more additional therapeutic agents, e.g., one or more anti-cancer agents, cytotoxic or cytostatic agents, hormone treatment, vaccines, and/or other immunotherapies. In other embodiments, the antibody molecules are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

The methods, compositions and combinations described herein (e.g., anti-PD-L1 antibodies and methods of using them) can be used in combination with other agents or therapeutic modalities, e.g., a second therapeutic agent chosen from one or more of the agents listed in Table 6 of WO 2016/061142, the contents of which are incorporated by reference in its entirety. In one embodiment, the methods described herein include administering to the subject an anti-PD-L1 antibody molecule as described in WO 2016/061142 (optionally in combination with one or more inhibitors of PD-1, PD-L1, TIM-3, CEACAM (e.g., CEACAM-1 and/or CEACAM-5), or CTLA-4)), further include administration of a second therapeutic agent chosen from one or more of the agents listed in Table 6 of WO 2016/061142, in an amount effective to treat or prevent a disorder, e.g., a disorder as described herein, e.g., a cancer. When administered in combination, the anti-PD-L1 antibody molecule, the additional agent (e.g., second or third agent), or all, can be administered in an amount or dose that is higher, lower, or the same as the amount or dosage of each agent used individually, e.g., as a monotherapy. In certain embodiments, the administered amount or dosage of the anti-PD-L1 antibody molecule, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the anti-PD-L1 antibody molecule, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy.

In other embodiments, the additional therapeutic agent is chosen from one or more of the agents listed in Table 6 of WO 2016/061142. In some embodiments, the additional therapeutic agent is chosen from one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or a 17alpha-Hydroxylase/C17-20 Lyase inhibitor); 5) an iron chelating agent; 6) an aromatase inhibitor; 7) an inhibitor of p53, e.g., an inhibitor of a p53/Mdm2 interaction; 8) an apoptosis inducer; 9) an angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) a fibroblast growth factor receptor 2 (FGFR2)/fibroblast growth factor receptor 4 (FGFR4) inhibitor; 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) a BCR-ABL kinase inhibitor; 25) an FGFR inhibitor; 26) an inhibitor of CYP11B2; 27) a HDM2 inhibitor, e.g., an inhibitor of the HDM2-p53 interaction; 28) an inhibitor of a tyrosine kinase; 29) an inhibitor of c-MET; 30) an inhibitor of JAK; 31) an inhibitor of DAC; 32) an inhibitor of 1 I0-hydroxylase; 33) an inhibitor of IAP; 34) an inhibitor of PIM kinase; 35) an inhibitor of Porcupine; 36) an inhibitor of BRAF, e.g., BRAF V600E or wild-type BRAF; 37) an inhibitor of HER3; 38) an inhibitor of MEK; or 39) an inhibitor of a lipid kinase, e.g., as described in Table 6 of WO 2016/061142.

Additional embodiments of combination therapies comprising an anti-PD-L1 antibody molecule described herein are described in WO2016/061142, which is incorporated by reference in its entirety.

### Methods of Treating Infectious Diseases

Disclosed herein are methods of treating infectious diseases using an anti-PD-L1 antibody molecule (e.g., an anti-PD-L1 antibody molecule described herein), or a composition or formulation comprising an anti-PD-L1 antibody molecule (e.g., a composition or formulation described herein). In certain embodiments, the antibody molecule, composition, or formulation is administered to a subject in accordance with a dosage regimen described herein.

In certain embodiments, the anti-PD-L1 antibody molecule is administered in an amount effective to treat an infectious disease or a symptom thereof. In some embodiments, the anti-PD-L1 antibody molecule is administered at a dose from about 20 mg to about 2000 mg once every three weeks, once every four weeks, or once every six weeks.

For example, the anti-PD-L1 antibody molecule can be administered at a dose from about 2000 mg, about 300 mg to about 1800 mg, about 200 mg to about 1600 mg, about 300 mg to about 1400 mg, about 600 to about 1700 mg, about 700 mg to about 1900 mg, or about 400 mg to about 1500 mg once every three weeks, once every four weeks, or once every six weeks. In one embodiment, the anti-PD-L1 antibody molecule is administered at a dose from about 60 mg to 100 mg (e.g., about 80 mg) once every three weeks. In one embodiment, the anti-PD-L1 antibody molecule is administered at a dose from about 200 mg to about 300 mg (e.g., about 240 mg) once every three weeks. In one embodiment, the anti-PD-L1 antibody molecule is administered at a dose from about 700 mg to about 900 mg (e.g., about 800 mg) once every three weeks, once every four weeks, or once every six weeks. In one embodiment, the anti-PD-L1 antibody molecule is administered at a dose of about 1000 mg to about 1400 mg (e.g., about 1200 mg) once every three weeks, once every four weeks, or once every six weeks. In one embodiment, the anti-PD-L1 antibody molecule is administered at a dose from about 1400 mg to about 1900 mg (e.g., about 1600 mg) once every three weeks, once every four weeks, or once every six weeks.

Certain methods described herein are used to treat subjects that have been exposed to particular toxins or pathogens. Without wishing to be bound by theory, it is believed that in some embodiments, anti-PD-L1 antibodies can stimulate NK cell mediated killing of target cells and can enhance IFN-gamma secretion and proliferation of CD4+ T cells. Accordingly, in certain embodiments, the anti-PD-L1 antibody molecules, compositions, and formulations described herein are suitable for use in stimulating an immune response against an infectious agent. Accordingly, another aspect of the invention provides a method of treating an infectious disease in a subject comprising administering to the subject an anti-PD-L1 antibody molecule, or a composition or formulation comprising an anti-PD-L1 antibody molecule, e.g., in accordance with a dosage regimen described herein, such that the subject is treated for the infectious disease. In the treatment of infection (e.g., acute and/or chronic), administration of the anti-PD-L1 antibody molecules can be combined with conventional treatments in addition to or in lieu of stimulating natural host immune defenses to infection. Natural host immune defenses to infection include, but are not limited to inflammation, fever, antibody-mediated host defense, T-lymphocyte-mediated host defenses, including lymphokine secretion and cytotoxic T-cells (especially during viral infection), complement mediated lysis and opsonization (facilitated phagocytosis), and phagocytosis. The ability of the anti-PD-L1 antibody molecules to reactivate dysfunctional T-cells would be useful to treat chronic infections, in particular those in which cell-mediated immunity is important for complete recovery.

Similar to its application to tumors as discussed in the previous section, the anti-PD-L1 antibody molecules, compositions, and formulations described herein can be used alone, or in combination with a second therapeutic agent or modality, or as an adjuvant, in combination with a vaccine, to stimulate an immune response to a pathogen or toxin. Examples of pathogens for which this therapeutic approach may be particularly useful, include pathogens for which there is currently no effective vaccine, or pathogens for which conventional vaccines are less than completely effective. These include, but are not limited to HIV, Hepatitis (A, B, & C), Influenza, Herpes, Giardia, Malaria, Leishmania, Staphylococcus aureus, Pseudomonas aeruginosa. Anti-PD-L1 antibody molecule therapy is also useful against established infections by agents, such as HIV, that present altered antigens over the course of the infections.

Accordingly, in some embodiments, an anti-PD-L1 antibody molecule, composition, or formulation described herein is used to treat a subject that has an infection or is at risk of having an infection. An infection refers to, e.g., a disease or condition attributable to the presence in a host of a foreign organism or agent that reproduces within the host. Infections typically involve breach of a normal mucosal or other tissue barrier by an infectious organism or agent. A subject that has an infection is a subject having objectively measurable infectious organisms or agents present in the subject's body. A subject at risk of having an infection is a subject that is predisposed to develop an infection. Such a subject can include, for example, a subject with a known or suspected exposure to an infectious organism or agent. A subject at risk of having an infection also can include a subject with a condition associated with impaired ability to mount an immune response to an infectious organism or agent, e.g., a subject with a congenital or acquired immunodeficiency, a subject undergoing radiation therapy or chemotherapy, a subject with a burn injury, a subject with a traumatic injury, a subject undergoing surgery or other invasive medical or dental procedure.

Infections are broadly classified as bacterial, viral, fungal, or parasitic based on the category of infectious organism or agent involved. Other less common types of infection include, e.g., infections involving rickettsiae, mycoplasmas, and agents causing scrapie, bovine spongiform encephalopthy (BSE), and prion diseases (e.g., kuru and Creutzfeldt-Jacob disease). Examples of bacteria, viruses, fungi, and parasites which cause infection are well known in the art. An infection can be acute, sub-acute, chronic, or latent, and it can be localized or systemic. Furthermore, an infection can be predominantly intracellular or extracellular during at least one phase of the infectious organism's or agent's life cycle in the host.

### Viruses

In certain embodiments, the anti-PD-L1 antibody molecule, composition, or formulation described herein is used to treat a viral infection or a disease associated with a virus.

Examples of viruses that have been found to cause infections in humans include but are not limited to: Retroviridae (e.g., human immunodeficiency viruses, such as HIV-1 (also referred to as HTLV-III), HIV-2, LAV or HTLV-III/LAV, or HIV-III, and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=enterally transmitted; class 2=parenterally transmitted (i.e., Hepatitis C); Norwalk and related viruses, and astroviruses). Some examples of pathogenic viruses causing infections treatable by methods herein include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

For infections resulting from viral causes, the anti-PD-L1 antibody molecules can be combined by application simultaneous with, prior to or subsequent to application of standard therapies for treating viral infections. Such standard therapies vary depending upon type of virus, although in almost all cases, administration of human serum containing antibodies (e.g., IgA, IgG) specific to the virus can be effective.

Some examples of pathogenic viruses causing infections treatable by methods include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, comovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus, arboviral encephalitis virus, and ebolaviruses (e.g., BDBV, EBOV, RESTV, SUDV and TAFV).

In one embodiment, the infection is an influenza infection. Influenza infection can result in fever, cough, myalgia, headache and malaise, which often occur in seasonal epidemics. Influenza is also associated with a number of postinfectious disorders, such as encephalitis, myopericarditis, Goodpasture's syndrome, and Reye's syndrome. Influenza infection also suppresses normal pulmonary antibacterial defenses, such that patients recovering from influenza have an increased risk of developing bacterial pneumonia. Influenza viral surface proteins show marked antigenic variation, resulting from mutation and recombination. Thus, cytolytic T lymphocytes are the host's primary vehicle for the elimination of virus after infection. Influenza is classified into three primary types: A, B and C. Influenza A is unique in that it infects both humans and many other animals (e.g., pigs, horses, birds and seals) and is the principal cause of pandemic influenza. Also, when a cell is infected by two different influenza A strains, the segmented RNA genomes of two parental virus types mix during replication to create a hybrid replicant, resulting in new epidemic strains. Influenza B does not replicate in animals and thus has less genetic variation and influenza C has only a single serotype.

Most conventional therapies are palliatives of the symptoms resulting from infection, while the host's immune response actually clears the disease. However, certain strains (e.g., influenza A) can cause more serious illness and death. Influenza A may be treated both clinically and prophylactically by the administration of the cyclic amines inhibitors amantadine and rimantadine, which inhibit viral replication. However, the clinical utility of these drugs is limited due to the relatively high incidence of adverse reactions, their narrow anti-viral spectrum (influenza A only), and the propensity of the virus to become resistant. The administration of serum IgG antibody to the major influenza surface proteins, hemagglutinin and neuraminidase can prevent pulmonary infection, whereas mucosal IgA is required to prevent infection of the upper respiratory tract and trachea. The most effective current treatment for influenza is vaccination with the administration of virus inactivated with formalin or β-propiolactone.

In another embodiment, the infection is a hepatitis infection, e.g., a Hepatitis B or C infection.

Hepatitis B virus (HB-V) is the most infectious known bloodborne pathogen. It is a major cause of acute and chronic heptatis and hepatic carcinoma, as well as life-long, chronic infection. Following infection, the virus replicates in hepatocytes, which also then shed the surface antigen HBsAg. The detection of excessive levels of HBsAg in serum is used a standard method for diagnosing a hepatitis B infection. An acute infection may resolve or it can develop into a chronic persistent infection. Current treatments for chronic HBV include α-interferon, which increases the expression of class I human leukocyte antigen (HLA) on the surface of hepatocytes, thereby facilitating their recognition by cytotoxic T lymphocytes. Additionally, the nucleoside analogs ganciclovir, famciclovir and lamivudine have also shown some efficacy in the treatment of HBV infection in clinical trials. Additional treatments for HBV include pegylated α-interferon, adenfovir, entecavir and telbivudine. While passive immunity can be conferred through parental administration of anti-HBsAg serum antibodies, vaccination with inactivated or recombinant HBsAg also confers resistance to infection. The anti-PD-L1 antibody molecules may be combined with conventional treatments for hepatitis B infections for therapeutic advantage.

Hepatitis C virus (HC-V) infection may lead to a chronic form of hepatitis, resulting in cirrosis. While symptoms are similar to infections resulting from Hepatitis B, in distinct contrast to HB-V, infected hosts can be asymptomatic for 10-20 years. The anti-PD-L1 antibody molecule can be administered as a monotherapy, or combined with the standard of care for hepatitis C infection. For example, the anti-PD-L1 antibody molecule can be administered with one or more of Sovaldi (sofosbuvir) Olysio (simeprevir), plus ribavirin or pegylated interferon. Although regimens that include Incivek (telaprevir) or Victrelis (boceprevir) plus ribavirin and pegylated interferon are also approved, they are associated with increased side effects and longer duration of treatment and are therefore not considered preferred regimens.

Conventional treatment for HC-V infection includes the administration of a combination of α-interferon and ribavirin. A promising potential therapy for HC-V infection is the protease inhibitor telaprevir (VX-960). Additional treatments include: anti-PD-1 antibody (MDX-1106, Medarex), bavituximab (an antibody that binds anionic phospholipid phosphatidylserine in a B2-glycoprotein I dependent manner, Peregrine Pharmaceuticals), anti-HPV viral coat protein E2 antibod(y)(ies) (e.g., ATL 6865-Ab68+Ab65, XTL Pharmaceuticals) and Civacir® (polyclonal anti-HCV human immune globulin). The anti-PD-L1 antibodies of the invention may be combined with one or more of these treatments for hepatitis C infections for therapeutic advantage. Protease, polymerase and NS5A inhibitors which may be used in combination with the anti-PD-L1 antibody molecules to specifically treat Hepatitis C infection include those described in US 2013/0045202, incorporated herein by reference.

In another embodiment, the infection is a measles virus. After an incubation of 9-11 days, hosts infected with the measles virus develop fever, cough, coryza and conjunctivitis. Within 1-2 days, an erythematous, maculopapular rash develop, which quickly spreads over the entire body. Because infection also suppresses cellular immunity, the host is at greater risk for developing bacterial superinfections, including otitis media, pneumonia and postinfectious encephalomyelitis. Acute infection is associated with significant morbidity and mortality, especially in malnourished adolescents.

Treatment for measles includes the passive administration of pooled human IgG, which can prevent infection in non-immune subjects, even if given up to one week after exposure. However, prior immunization with live, attenuated virus is the most effective treatment and prevents disease in more than 95% of those immunized. As there is one serotype of this virus, a single immunization or infection typically results in protection for life from subsequent infection.

In a small proportion of infected hosts, measles can develop into SSPE, which is a chronic progressive neurologic disorder resulting from a persistent infection of the central nervous system. SSPE is caused by clonal variants of measles virus with defects that interfere with virion assembly and budding. For these patients, reactivation of T-cells with the anti-PD-L1 antibody molecules so as to facilitate viral clearance would be desirable.

In another embodiment, the infection is HIV. HIV attacks CD4⁺ cells, including T-lymphocytes, monocyte-macrophages, follicular dendritic cells and Langerhan's cells, and CD4⁺ helper/inducer cells are depleted. As a result, the host acquires a severe defect in cell-mediated immunity. Infection with HIV results in AIDS in at least 50% of individuals, and is transmitted via sexual contact, administration of infected blood or blood products, artificial insemination with infected semen, exposure to blood-containing needles or syringes and transmission from an infected mother to infant during childbirth.

A host infected with HIV may be asymptomatic, or may develop an acute illness that resembling mononucleosis—fever, headache, sore throat, malaise and rash. Symptoms can progress to progressive immune dysfunction, including persistent fever, night sweats, weight loss, unexplained diarrhea, eczema, psoriasis, seborrheic dermatitis, herpes zoster, oral candidiasis and oral hairy leukoplakia. Opportunistic infections by a host of parasites are common in patients whose infections develop into AIDS.

Treatments for HIV include antiviral therapies including nucleoside analogs, zidovudine (AST) either alone or in combination with didanosine or zalcitabine, dideoxyinosine, dideoxycytidine, lamidvudine, stavudine; reverse transcriptive inhibitors such as delavirdine, nevirapine, loviride, and proteinase inhibitors such as saquinavir, ritonavir, indinavir and nelfinavir. The anti-PD-L1 antibody molecules may be combined with conventional treatments for HIV infections for therapeutic advantage.

In another embodiment, the infection is a Cytomegalovirus (CMV). CMV infection is often associated with persistent, latent and recurrent infection. CMV infects and remains latent in monocytes and granulocyte-monocyte progenitor cells. The clinical symptoms of CMV include mononucleosis-like symptoms (i.e., fever, swollen glands, malaise), and a tendancy to develop allergic skin rashes to antibiotics. The virus is spread by direct contact. The virus is shed in the urine, saliva, semen and to a lesser extent in other body fluids. Transmission can also occur from an infected mother to her fetus or newborn and by blood transfusion and organ transplants. CMV infection results in general impairment of cellular immunity, characterized by impaired blastogenic responses to nonspecific mitogens and specific CMV antigens, diminished cytotoxic ability and elevation of CD8 lymphocyte number of CD4⁺ lymphocytes.

Treatments of CMV infection include the anti-virals ganciclovir, foscamet and cidovir, but these druges are typically only prescribed in immunocompromised patients. The anti-PD-L1 antibody molecules may be combined with conventional treatments for cytomegalovirus infections for therapeutic advantage.

In another embodiment, the infection is Epstein-Barr virus (EBV). EBV can establish persistent and latent infections and primarily attacks B cells. Infection with EBV results in the clinical condition of infectious mononucleosis, which includes fever, sore throat, often with exudate, generalized lymphadenopathy and splenomegaly. Hepatitis is also present, which can develop into jaundice.

While typical treatments for EBV infections are palliative of symptoms, EBV is associated with the development of certain cancers such as Burkitt's lymphoma and nasopharyngeal cancer. Thus, clearance of viral infection before these complications result would be of great benefit. The anti-PD-L1 antibody molecules may be combined with conventional treatments for Epstein-Barr virus infections for therapeutic advantage.

In another embodiment, the infection is Herpes simplex virus (HSV). HSV is transmitted by direct contact with an infected host. A direct infection may be asymptomatic, but typically result in blisters containing infectious particles. The disease manifests as cycles of active periods of disease, in which lesions appear and disappear as the viral latently infect the nerve ganglion for subsequent outbreaks. Lesions may be on the face, genitals, eyes and/or hands. In some case, an infection can also cause encephalitis.

Treatments for herpes infections are directed primarily to resolving the symptomatic outbreaks, and include systemic antiviral medicines such as: acyclovir (e.g., Zovirax®), valaciclovir, famciclovir, penciclovir, and topical medications such as docosanol (Abreva®), tromantadine and zilactin. The clearance of latent infections of herpes would be of great clinical benefit. The anti-PD-L1 antibody molecules may be combined with conventional treatments for herpes virus infections for therapeutic advantage.

In another embodiment, the infection is Human T-lymphotrophic virus (HTLV-1, HTLV-2). HTLV is transmitted via sexual contact, breast feeding or exposure to contaminated blood. The virus activates a subset of T_{H} cells called Th1 cells, resulting in their overproliferation and overproduction of Th1 related cytokines (e.g., IFN-γ and TNF-α). This in turn results in a suppression of Th2 lymphocytes and reduction of Th2 cytokine production (e.g., IL-4, IL-5, IL-10 and IL-13), causing a reduction in the ability of an infected host to mount an adequate immune response to invading organisms requiring a Th2-dependent response for clearance (e.g., parasitic infections, production of mucosal and humoral antibodies).

HTLV infections cause lead to opportunistic infections resulting in bronchiectasis, dermatitis and superinfections with Staphylococcus spp. and Strongyloides spp. resulting in death from polymicrobial sepsis. HTLV infection can also lead directly to adult T-cell leukemia/lymphoma and progressive demyelinating upper motor neuron disease known as HAM/TSP. The clearance of HTLV latent infections would be of great clinical benefit. The anti-PD-L1 antibody molecules may be combined with conventional treatments for HTLV infections for therapeutic advantage.

In another embodiment, the infection is Human papilloma virus (HPV). HPV primarily affects keratinocytes and occurs in two forms: cutaneous and genital. Transmission is believed to occur through direct contact and/or sexual activity. Both cutaneous and genital HPV infection can result in warts and latent infections and sometimes recurring infections, which are controlled by host immunity which controls the symptoms and blocks the appearance of warts, but leaves the host capable of transmitting the infection to others.

Infection with HPV can also lead to certain cancers, such as cervical, anal, vulvar, penile and oropharynial cancer. There are no known cures for HPV infection, but current treatment is topical application of Imiquimod, which stimulates the immune system to attack the affected area. The clearance of HPV latent infections would be of great clinical benefit. The anti-PD-L1 antibodies of the invention may be combined with conventional treatments for HPV infections for therapeutic advantage.

In another embodiment, the infection is Ebola virus (EBOV). EBOV is one of five known viruses within the Ebola virus genus. EBOV causes severe and often fatal hemorrhagic fever in humans and mammals, known as Ebola virus disease (EVD). Transmission occurs through contact with blood, secretions, organs, or other boldily fluids of infected patients. Currently, there is no proven treatment or vaccine.

### Bacteria

In certain embodiments, the anti-PD-L1 antibody molecule, composition, or formulation described herein is used to treat a bacterial infection or a disease associated with a bacterium.

Bacteria include both Gram negative and Gram positive bacteria. Examples of Gram positive bacteria include, but are not limited to Pasteurella species, Staphylococci species, and Streptococcus species. Examples of Gram negative bacteria include, but are not limited to, Escherichia coli, Pseudomonas species, and Salmonella species. Specific examples of infectious bacteria include but are not limited to: Helicobacter pyloris, Borrelia burgdorferi, Legionella pneumophilia, Mycobacteria spp. (e.g., M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic spp.), Streptococcus pneumoniae, pathogenic Campylobacter spp., Enterococcus spp., Haemophilus influenzae, Bacillus anthracis, Corynebacterium diphtheriae, Corynebacterium spp., Erysipelothrix rhusiopathiae, Clostridium perfringens, Clostridium tetani, Enterobacter aerogenes, Klebsiella pneumoniae, Pasteurella multocida, Bacteroides spp., Fusobacterium nucleatum, Streptobacillus moniliformis, Treponema pallidum, Treponema pertenue, Leptospira, Mycobacterium leprae, Rickettsia, and Actinomyces israelii. Some examples of pathogenic bacteria causing infections treatable by methods herein include chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lymes disease bacteria.

Some examples of pathogenic bacteria causing infections treatable by methods of the invention include syphilis, chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lymes disease bacteria. The anti-PD-L1 antibody molecules can be used in combination with existing treatment modalities for the aforesaid infections. For example, Treatments for syphilis include penicillin (e.g., penicillin G.), tetracycline, doxycycline, ceftriaxone and azithromycin.

Lyme disease, caused by Borrelia burgdorferi is transmitted into humans through tick bites. The disease manifests initially as a localized rash, followed by flu-like symptoms including malaise, fever, headache, stiff neck and arthralgias. Later manifestations can include migratory and polyarticular arthritis, neurologic and cardiac involvement with cranial nerve palsies and radiculopathy, myocarditis and arrhythmias. Some cases of Lyme disease become persistent, resulting in irreversible damage analogous to tertiary syphilis. Current therapy for Lyme disease includes primarily the administration of antibiotics. Antibiotic-resistant strains may be treated with hydroxychloroquine or methotrexate. Antibiotic refractory patients with neuropathic pain can be treated with gabapentin. Minocycline may be helpful in late/chronic Lyme disease with neurological or other inflammatory manifestations.

Other forms of borreliois, such as those resulting from B. recurentis, B. hermsii, B. turicatae, B. parikeri., B. hispanica, B. duttonii and B. persica, as well leptospirosis (E.g., L. interrogans), typically resolve spontaneously unless blood titers reach concentrations to cause intrahepatic obstruction.

### Fungi and Parasites

In certain embodiments, the anti-PD-L1 antibody molecule, composition, or formulation described herein is used to treat a fungal or parasitic infection or a disease associated with a fungus or a parasite.

Examples of fungi include: Aspergillus spp., Blastomyces dermatitidis, Candida albicans, other Candida spp., Coccidioides immitis, Cryptococcus neoformans, Histoplasma capsulatum, Chlamydia trachomatis, Nocardia spp., Pneumocystis carinii. Some examples of pathogenic fungi causing infections treatable by methods herein include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Parasites include but are not limited to blood-borne and/or tissues parasites such as Babesia microti, Babesia divergens, Entamoeba histolytica, Giardia lamblia, Leishmania tropica, Leishmania spp., Leishmania braziliensis, Leishmania donovani, Plasmodium falciparum, Plasmodium malariae, Plasmodium ovale, Plasmodium vivax, and Toxoplasma gondii, Trypanosoma gambiense and Trypanosoma rhodesiense (African sleeping sickness), Trypanosoma cruzi (Chagas' disease), and Toxoplasma gondii, flat worms, round worms. Some examples of pathogenic parasites causing infections treatable by methods herein include Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

Some examples of pathogenic fungi causing infections treatable by methods of the invention include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum. Some examples of pathogenic parasites causing infections treatable by methods described herein include Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

### Nucleic Acids

The anti-PD-L1 antibody molecules described herein can be encoded by nucleic acids described herein. The nucleic acids can be used to produce the anti-PD-L1 antibody molecules described herein.

In certain embodiments, the nucleic acid comprises nucleotide sequences that encode heavy and light chain variable regions and CDRs of the anti-PD-L1 antibody molecules, as described herein.

For example, the present disclosure features a first and second nucleic acid encoding heavy and light chain variable regions, respectively, of an anti-PD-L1 antibody molecule chosen from one or more of the antibody molecules disclosed herein, e.g., an antibody of Table 1 of US 2016/0108123. The nucleic acid can comprise a nucleotide sequence encoding any one of the amino acid sequences in the tables herein, or a sequence substantially identical thereto (e.g., a sequence at least about 85%, 90%, 95%, 99% or more identical thereto, or which differs by no more than 3, 6, 15, 30, or 45 nucleotides from the sequences provided in Table 1 of US 2016/0108123. For example, disclosed herein is a first and second nucleic acid encoding heavy and light chain variable regions, respectively, of an anti-PD-L1 antibody molecule chosen from one or more of, e.g., BAP058-hum01, BAP058-hum02, BAP058-hum03, BAP058-hum04, BAP058-hum05, BAP058-hum06, BAP058-hum07, BAP058-hum08, BAP058-hum09, BAP058-hum10, BAP058-hum11, BAP058-hum12, BAP058-hum13, BAP058-hum14, BAP058-hum15, BAP058-hum16, BAP058-hum17, BAP058-Clone-K, BAP058-Clone-L, BAP058-Clone-M, BAP058-Clone-N, or BAP058-Clone-0, as summarized in Table 1 of US 2016/0108123, or a sequence substantially identical thereto.

In certain embodiments, the nucleic acid can comprise a nucleotide sequence encoding at least one, two, or three CDRs from a heavy chain variable region having an amino acid sequence as set forth in Table 1 of US 2016/0108123, or a sequence substantially homologous thereto (e.g., a sequence at least about 85%, 90%, 95%, 99% or more identical thereto, and/or having one or more substitutions, e.g., conserved substitutions). In some embodiments, the nucleic acid can comprise a nucleotide sequence encoding at least one, two, or three CDRs from a light chain variable region having an amino acid sequence as set forth in Table 1, or a sequence substantially homologous thereto (e.g., a sequence at least about 85%, 90%, 95%, 99% or more identical thereto, and/or having one or more substitutions, e.g., conserved substitutions). In some embodiments, the nucleic acid can comprise a nucleotide sequence encoding at least one, two, three, four, five, or six CDRs from heavy and light chain variable regions having an amino acid sequence as set forth in Table 1, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs from a heavy chain variable region having the nucleotide sequence as set forth in Table 1 of US 2016/0108123, a sequence substantially homologous thereto (e.g., a sequence at least about 85%, 90%, 95%, 99% or more identical thereto, and/or capable of hybridizing under the stringency conditions described herein). In some embodiments, the nucleic acid can comprise a nucleotide sequence encoding at least one, two, or three CDRs from a light chain variable region having the nucleotide sequence as set forth in Table 1, or a sequence substantially homologous thereto (e.g., a sequence at least about 85%, 90%, 95%, 99% or more identical thereto, and/or capable of hybridizing under the stringency conditions described herein). In certain embodiments, the nucleic acid can comprise a nucleotide sequence encoding at least one, two, three, four, five, or six CDRs from heavy and light chain variable regions having the nucleotide sequence as set forth in Table 1, or a sequence substantially homologous thereto (e.g., a sequence at least about 85%, 90%, 95%, 99% or more identical thereto, and/or capable of hybridizing under the stringency conditions described herein). The nucleic acids disclosed herein include deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a nonnatural arrangement.

In certain embodiments, the nucleotide sequence that encodes the anti-PD-L1 antibody molecule is codon optimized.

In some embodiments, nucleic acids comprising nucleotide sequences that encode heavy and light chain variable regions and CDRs of the anti-PD-L1 antibody molecules, as described herein, are disclosed. For example, the disclosure provides a first and second nucleic acid encoding heavy and light chain variable regions, respectively, of an anti-PD-L1 antibody molecule according to Table 1 of US 2016/0108123 or a sequence substantially identical thereto. For example, the nucleic acid can comprise a nucleotide sequence encoding an anti-PD-L1 antibody molecule according to Table 1, or a sequence substantially identical to that nucleotide sequence (e.g., a sequence at least about 85%, 90%, 95%, 99% or more identical thereto, or which differs by no more than 3, 6, 15, 30, or 45 nucleotides from the aforementioned nucleotide sequence.

In certain embodiments, the nucleic acid can comprise a nucleotide sequence encoding at least one, two, or three CDRs, or hypervariable loops, from a heavy chain variable region having an amino acid sequence as set forth in Table 1, or a sequence substantially homologous thereto (e.g., a sequence at least about 85%, 90%, 95%, 99% or more identical thereto, and/or having one, two, three or more substitutions, insertions or deletions, e.g., conserved substitutions).

In certain embodiments, the nucleic acid can comprise a nucleotide sequence encoding at least one, two, or three CDRs, or hypervariable loops, from a light chain variable region having an amino acid sequence as set forth in Table 1, or a sequence substantially homologous thereto (e.g., a sequence at least about 85%, 90%, 95%, 99% or more identical thereto, and/or having one, two, three or more substitutions, insertions or deletions, e.g., conserved substitutions).

In some embodiments, the nucleic acid can comprise a nucleotide sequence encoding at least one, two, three, four, five, or six CDRs, or hypervariable loops, from heavy and light chain variable regions having an amino acid sequence as set forth in Table 1, or a sequence substantially homologous thereto (e.g., a sequence at least about 85%, 90%, 95%, 99% or more identical thereto, and/or having one, two, three or more substitutions, insertions or deletions, e.g., conserved substitutions).

In some embodiments, the nucleic acid is isolated or recombinant.

The nucleic acids described herein may be present in a single vector or separate vectors present in the same host cell or separate host cell, as described in more detail herein.

### Vectors and Host Cells

The anti-PD-L1 antibody molecules described herein can be produced using host cells and vectors containing the nucleic acids described herein. The nucleic acids may be present in a single vector or separate vectors present in the same host cell or separate host cell.

In one embodiment, the vectors comprise nucleotides encoding an antibody molecule described herein. In one embodiment, the vectors comprise the nucleotide sequences described herein. The vectors include, but are not limited to, a virus, plasmid, cosmid, lambda phage, or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity. Methods and conditions for culturing the resulting transfected cells and for recovering the antibody molecule produced are known to those skilled in the art, and may be varied or optimized depending upon the specific expression vector and mammalian host cell employed, based upon the present description.

In certain embodiments, the host cell comprises a nucleic acid encoding an anti-PD-L1 antibody molecule described herein. In other embodiments, the host cell is genetically engineered to comprise a nucleic acid encoding the anti-PD-L1 antibody molecule.

In one embodiment, the host cell is genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter. In certain embodiments, the host cell comprises a vector described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

In some embodiments, the host cell is a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

Additional antibody molecules, compositions, methods, nucleic acids, and kits that can be used in accordance with the disclsures herein are described in International Application Publication No. WO2016/061142, the content of which is incorporated by refererence in its entirety.

### EXAMPLES

The Examples below are set forth to aid in the understanding of the inventions, but are not intended to, and should not be construed to limit its scope in any way.

### Example 1: Pharmacokinetics and Pharmacodynamics of an Exemplary Anti-PD-L1 Antibody

Pharmacokinetic (Pk) parameters were calculated from serum concentration-time profiles of samples collected from human subjects administered FAZ053 in a dose expansion study. Samples were analyzed from subjects administered FAZ053 via intravenous infusion at doses of 80 mg, 240 mg, 800 mg, 1200 mg, or 1600 mg every 3 weeks (Q3W) and 800 mg, 1200 mg, or 1600 mg every 6 weeks (Q6W). Noncompartmental analysis (NCA) was performed using Phoenix 6.4 (Pharsight, Mountain View, CA). The serum PK samples were analyzed using LC-MS/MS. The limit of quantification (LOQ) of the assay was 0.25 g/mL. A summary of pK parameters is shown in Table 13.

TABLE 13
Summary of pharmacokinetic parameters for FAZ053 by dose, dosing
regimen, and cycle from the dose expansion study. The duration of
1 cycle is 21 days. Geo. Mean represents geometric mean. Geo. CV
% represents geometric coefficient of variation (CV). C504 h
represents the concentration at 504 h (3 weeks) after previous dose.

| | | | | | | AUC | |
|---|---|---|---|---|---|---|---|
| Dose | Dosing | | | Cmax | C504 h^{#} | 0-504 h | AUCtau |
| (mg) | regimen | Cycle | Statistics | (μg/mL) | (μg/mL) | (μg*h/mL) | (μg*h/mL) |
| 80 | Q3W | | N | 6 | 6 | 6 | 6 |
| | | 1 | Geo. Mean | 33.1 | 3.8 | 5110 | 5110 |
| | | | Geo. CV % | 14.7 | 59.9 | 23.8 | 23.8 |
| | | | N | 5 | 4 | 5 | 5 |
| | | 3 | Geo. Mean | 37.2 | 10 | 7250 | 7250 |
| | | | Geo. CV % | 20.8 | 16.8 | 36.7 | 36.7 |
| 240 | Q3W | | N | 5 | 5 | 5 | 5 |
| | | 1 | Geo. Mean | 85.5 | 9.7 | 14500 | 14500 |
| | | | Geo. CV % | 32.7 | 146.6 | 25.7 | 25.7 |
| | | | N | 4 | 4 | 4 | 4 |
| | | 3 | Geo. Mean | 110 | 11.2 | 16700 | 16700 |
| | | | Geo. CV % | 52.8 | 1173.3 | 157.1 | 157.1 |
| 800 | Q3W | | N | 5 | 5 | 5 | 5 |
| | | 1 | Geo. Mean | 244 | 43.1 | 40600 | 40600 |
| | | | Geo. CV % | 59.5 | 69.9 | 52.1 | 52.1 |
| | | | N | 5 | 5 | 5 | 5 |
| | | 3 | Geo. Mean | 302 | 78.6 | 69400 | 69400 |
| | | | Geo. CV % | 49.2 | 58.5 | 33.3 | 33.3 |
| 1200 | Q3W | | N | 3 | 1 | 3 | 3 |
| | | 1 | Geo. Mean | 338 | 60.6 | 59600 | 59600 |
| | | | Geo. CV % | 43.3 | NA | 8.9 | 8.9 |
| | | | N | 0 | 0 | 0 | 0 |
| | | 3 | Geo. Mean | NA | NA | NA | NA |
| | | | Geo. CV % | NA | NA | NA | NA |
| 1600 | Q3W | | N | 6 | 4 | 5 | 5 |
| | | 1 | Geo. Mean | 510 | 95.6 | 91000 | 91000 |
| | | | Geo. CV % | 25.3 | 33.6 | 20.5 | 20.5 |
| | | | N | 5 | 4 | 5 | 5 |
| | | 3 | Geo. Mean | 698 | 211 | 154000 | 154000 |
| | | | Geo. CV % | 28 | 24 | 27.9 | 27.9 |
| 800 | Q6W | | N | 4 | 4 | 5 | 5 |
| | | 1 | Geo. Mean | 275 | 44.1 | 45000 | 60800 |
| | | | Geo. CV % | 44 | 73.7 | 46.3 | 51.9 |
| | | | N | 4 | 2 | 3 | 3 |
| | | 5 | Geo. Mean | 276 | 29 | 39300 | 51700 |
| | | | Geo. CV % | 65.8 | 200.7 | 77.9 | 89.6 |
| 1200 | Q6W | | N | 4 | 3 | 4 | 3 |
| | | 1 | Geo. Mean | 402 | 50.3 | 65400 | 76700 |
| | | | Geo. CV % | 19.9 | 40.2 | 22.1 | 28.8 |
| | | | N | 0 | 0 | 0 | 0 |
| | | 5 | Geo. Mean | NA | NA | NA | NA |
| | | | Geo. CV % | NA | NA | NA | NA |
| 1600 | Q6W | | N | 5 | 4 | 5 | 5 |
| | | 1 | Geo. Mean | 38.1 | 63.9 | 73000 | 98300 |
| | | | Geo. CV % | 43.9 | 101.1 | 42.8 | 42.8 |
| | | | N | 0 | 0 | 0 | 0 |
| | | 5 | Geo. Mean | NA | NA | NA | NA |
| | | | Geo. CV % | NA | NA | NA | NA |

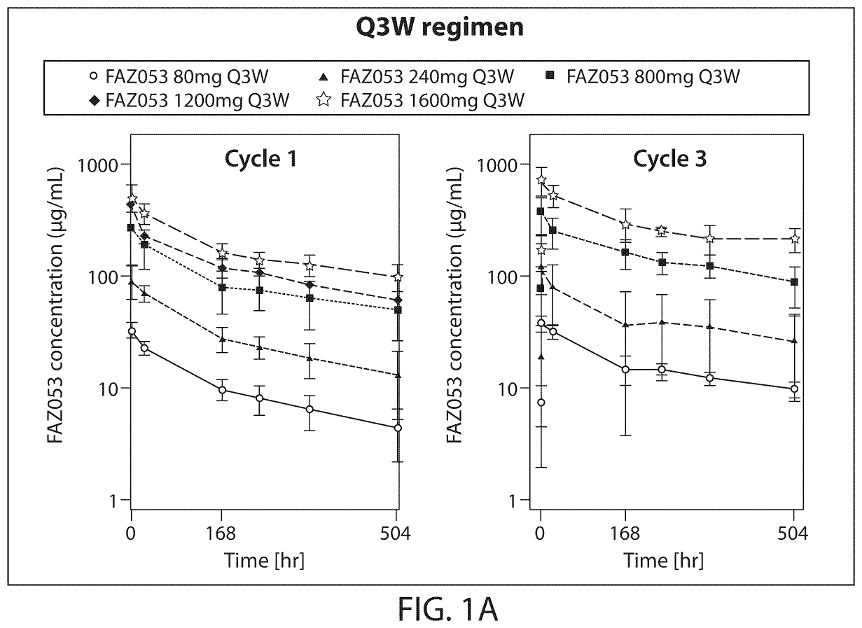

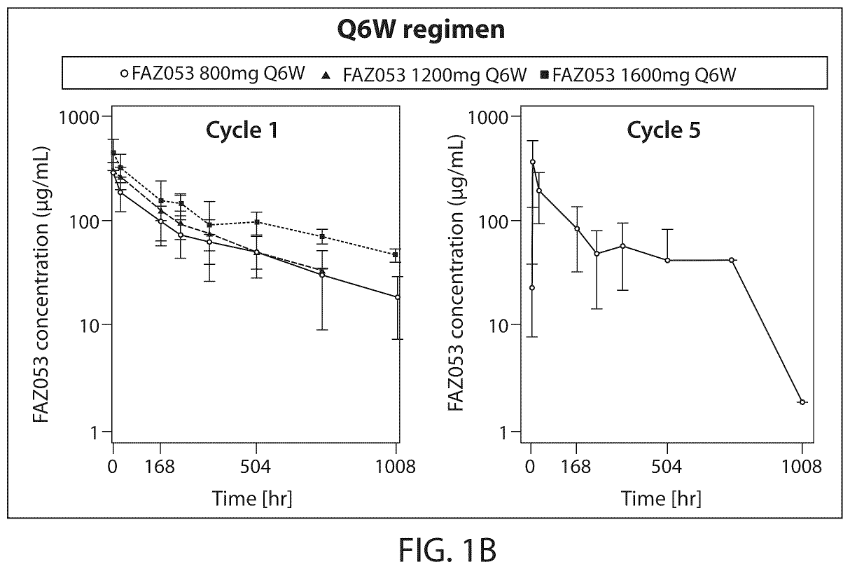

Following administration of FAZ053 via intravenous infusion at dose levels of 80 mg, 240 mg, 800 mg, 1200 mg, and 1600 mg Q3W and 800 mg, 1200 mg, and 1600 mg Q6W, the maximum serum concentrations (Cmax) occurred generally after the end of the infusion (FIGS. 1A-1B). PK variability was low to moderate; between subject variability for the PK parameters (CV % geometric mean) ranged from 14.7 to 59.5% for Cmax (C1D1) and 8.9 to 52.1% for AUC0-504h (C1D1). Approximately dose-proportional increases in exposure (C1D1 AUCₜₐᵤ and Cₘₐₓ) were observed from 80 mg to 1600 mg Q3W and 800 mg to 1600 mg Q6W. Accumulation of approximately 1.5-fold was observed with Q3W dosing; minimal accumulation was observed with the Q6W dosing.

### Example 2: Soluble PD-L1 Binding to an Anti-PD-L1 Antibody Molecule

PD-L1 is a membrane bound protein that is expressed on a wide range of tumors. PD-L1 also exists in the systemic circulation in a soluble form (sPD-L1). To investigate binding of the anti-PD-L1 antibody FAZ053 to sPD-L1, total sPD-L1 (free sPD-L1 plus sPD-L1-FAZ053 complex) samples collected from subjects administered FAZ053 as described in Example 1 were analyzed using an ELISA based method. Samples were analyzed from subjects administered FAZ053 via intravenous infusion at doses of 80 mg, 240 mg, 800 mg, 1200 mg and 1600 mg Q3W and 800 mg, 1200 mg, and 1600 mg every Q6W.

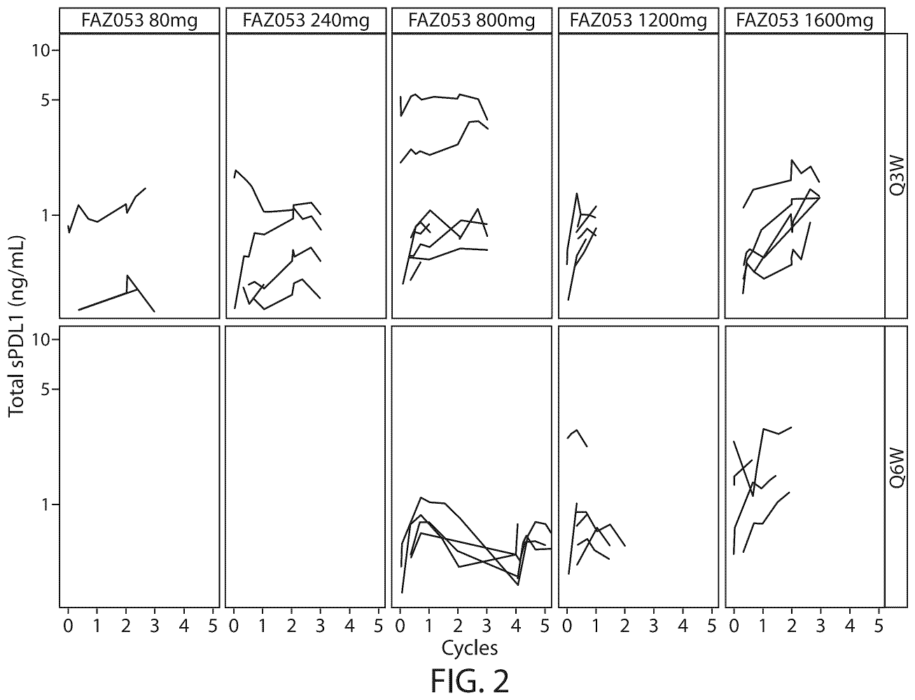

At pre-dose baseline, ˜76% of the samples were below a LOQ of 0.25 ng/mL. Total sPD-L1 concentrations increased upon administration of FAZ053 at doses greater than or equal to 240 mg Q3W, suggesting sPD-L1 binding to FAZ053 (FIG. 2). Doses of greater than or equal to 800 mg Q3W or greater than or equal to 1600 mg Q6W demonstrated sustained total sPD-L1 throughout the dosing interval. Doses less than or equal to 1200 mg Q6W resulted in decreased total sPD-L1 during cycle 2. The duration of 1 cycle was 21 days.

Overall, the total sPD-L1 data support dosing at greater than or equal to 800 mg Q3W or greater than or equal to 1600 mg Q6W for sustained sPD-L1 binding throughout the dosing interval. However, total sPD-L1 demonstrated high inter-individual variability across cohorts.

To estimate receptor occupancy in the tumor at the end of Cycle 1 week 3, the equation below was used.

$$RO = \frac{B \cdot {C}_{504 h}}{B \cdot {C}_{504 h} + Kd}$$

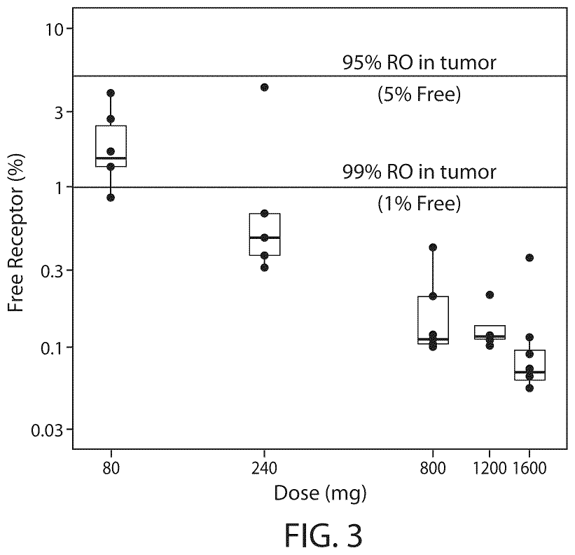

The binding affinity (Kd) for PD-L1 of 0.14 nM was then determined using an in vitro Biacore assay. The drug concentration in the tumor interstitial fluid was assumed to be B=30% of the systemic concentration (see Deng et al. (2016) MAbs; 8(3): 593-603; which is hereby incorporated by reference in its entirety). C504h represents the concentration of FAZ053 in serum at the end of Cycle 1 week 3 (504 h). At 1200 mg, the predicted receptor occupancy at the end of week 3 was predicted to be greater than 99% for all patients in this study (FIG. 3). These results show increased total soluble PD-L1 (free sPD-L1 plus sPD-L1-FAZ053 complexes) due to binding of sPD-L1 by FAZ053.

The recommended dose for expansion (RDE) for FAZ053 of 1200 mg Q3W was selected based on sPD-L1 binding (Total sPD-L1), the predicted greater than 99% receptor occupancy in the tumor for all subjects in the study, and good overall safety of FAZ053 at this dose. Although lower doses of 800 mg Q3W were predicted to achieve similar receptor occupancy, a 1200 mg Q3W dose was chosen as the RDE to increase the likelihood of tumor penetration of FAZ053 and to overcome a possible higher burden of sPD-L1 (antigen sink) in some patients, which was suggested by the observed variability in total sPD-L1.

A dose of 1600 mg Q4W RDE for FAZ053 was then chosen based on the same expected steady state average PK concentration (Cave) as the 1200 mg Q3W regimen. Average steady state concentration (Cave) for FAZ053 was determined from Dose/(clearance (CL)*t), where ti is the dosing frequency and CL is the intrinsic clearance of FAZ053. Based on this formula, Cave=1200 mg/(CL*3 weeks)=1600 mg/(CL*4 weeks). In addition, doses up to 1600 mg Q3W were shown to be safe and generally well tolerated in patients.

### Example 3: Anti-Tumor Activity of an Exemplary Anti-PD-L1 Antibody in Human Subjects

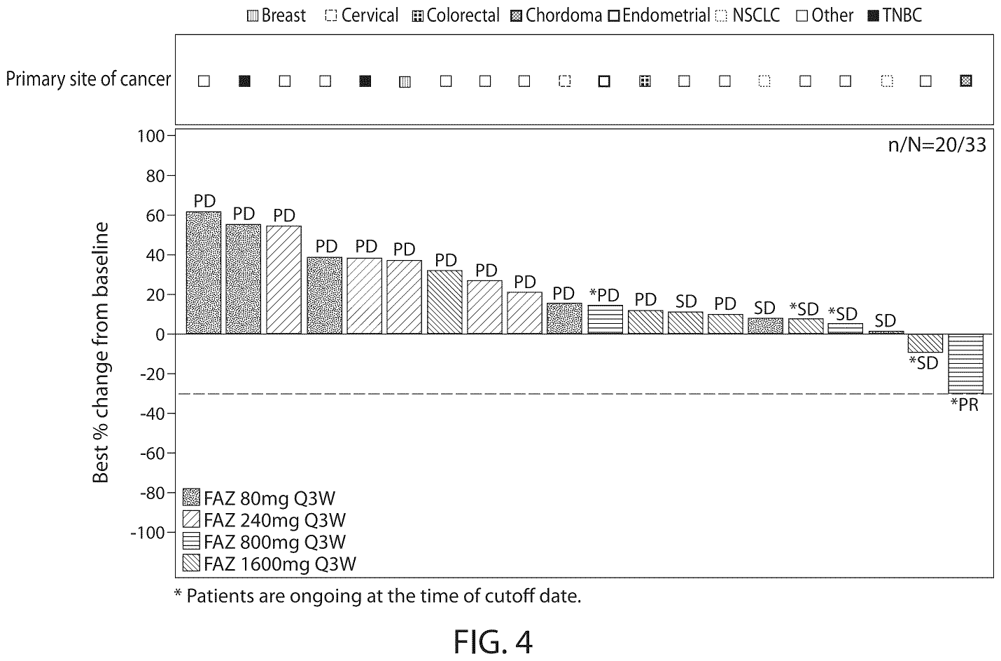

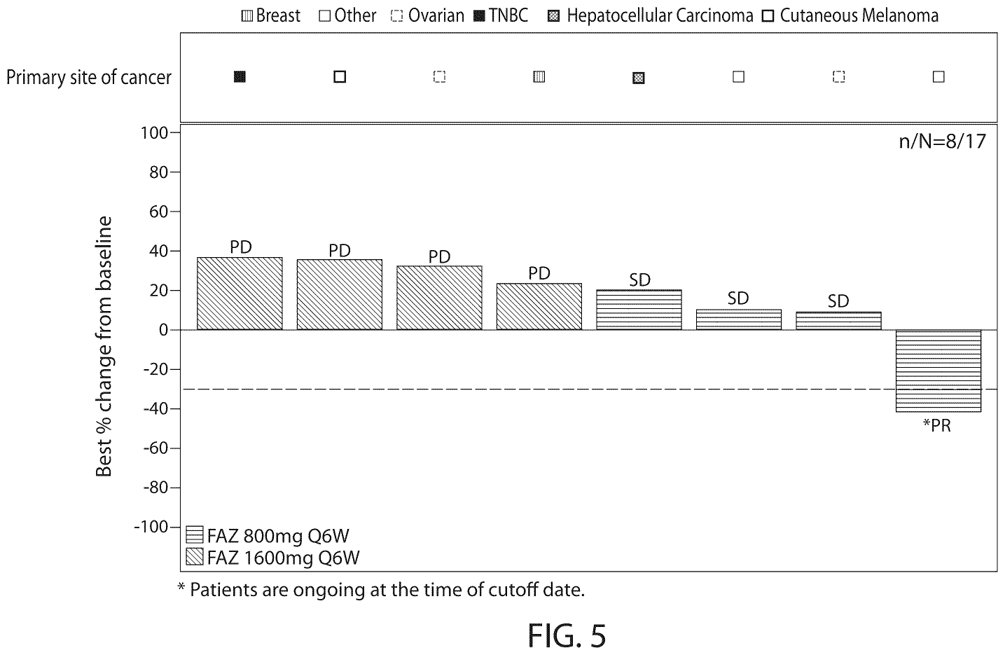

In an ongoing clinical trial, subjects with a variety of tumor types were administered FAZ053 as a monotherapy via intravenous infusion at doses of 80 mg, 240 mg, 800 mg, 1200 mg, and 1600 mg Q3W and 800 mg, 1200 mg, and 1600 mg every Q6W. Subjects had breast cancer, cervical cancer, colorectal cancer, chordoma, endometrial cancer, non-small cell lung cancer (NSCLC), triple negative breast cancer (TNBC), ovarian cancer, hepatocellular carcinoma, and other forms of cancer. Two confirmed partial responses per RECIST v1.1 were observed with FAZ053 treatment as a monotherapy at dose levels of 800 mg Q3W and 800 mg Q6W (FIGS. 4 and 5).

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1-47. (canceled)

48. A method of treating a cancer in a subject, the method comprising administering to the subject an anti-PD-L1 antibody molecule at a dose of about 800 mg or 1200 mg once every three weeks,
wherein the anti-PD-L1 antibody molecule comprises a heavy chain variable region (VH) comprising a VHCDR1 amino acid sequence of SEQ ID NO: 601, a VHCDR2 amino acid sequence of SEQ ID NO: 602, and a VHCDR3 amino acid sequence of SEQ ID NO: 603; and a light chain variable region (VL) comprising a VLCDR1 amino acid sequence of SEQ ID NO: 609, a VLCDR2 amino acid sequence of SEQ ID NO: 610, and a VLCDR3 amino acid sequence of SEQ ID NO: 611.

49. The method of claim 48, wherein the anti-PD-L1 antibody molecule is used at a dose of about 1200 mg once every three weeks.

50. The method of claim 48, wherein the anti-PD-L1 antibody molecule is used at a dose of about 800 mg once every three weeks.

51. The method of claim 48, wherein the antibody molecule comprises a VH comprising the amino acid sequence of SEQ ID NO: 606 and a VL comprising the amino acid sequence of SEQ ID NO: 616.

52. The method of claim 48, wherein the antibody molecule comprises a heavy chain comprising the amino acid sequence of SEQ ID NO: 608 and a light chain comprising the amino acid sequence of SEQ ID NO: 618.

53. The method of claim 48, wherein the antibody molecule comprises a VH comprising the amino acid sequence of SEQ ID NO: 620 and a VL comprising the amino acid sequence of SEQ ID NO: 624.

54. The method of claim 48, wherein the antibody molecule comprises a heavy chain comprising the amino acid sequence of SEQ ID NO: 622 and a light chain comprising the amino acid sequence of SEQ ID NO: 626.

55. The method of claim 48, wherein the cancer is chosen from a bone cancer, a skin cancer, a breast cancer, a cervical cancer, a colorectal cancer, an endometrial cancer, a lung cancer, an ovarian cancer, a liver cancer, a thyroid cancer, or a combination thereof.

56. The method of claim 55, wherein the breast cancer is a breast carcinoma or a triple negative breast cancer (TNBC).

57. The method of claim 48, wherein the subject has, or is identified as having, a cancer that expresses PD-L1.

58. The method of claim 48, wherein administering to the subject the anti-PD-L1 antibody molecule results in 50% or more of the soluble PD-L1 in the serum, or a serum sample, from the subject bound by the anti-PD-L1 antibody molecule.

59. A pharmaceutical composition comprising:
(i) an anti-PD-L1 antibody molecule, and
(ii) one or more selected from histidine, polysorbate 20, and sucrose,
wherein the anti-PD-L1 antibody molecule comprises a heavy chain variable region (VH) comprising a VHCDR1 amino acid sequence of SEQ ID NO: 601, a VHCDR2 amino acid sequence of SEQ ID NO: 602, and a VHCDR3 amino acid sequence of SEQ ID NO: 603; and a light chain variable region (VL) comprising a VLCDR1 amino acid sequence of SEQ ID NO: 609, a VLCDR2 amino acid sequence of SEQ ID NO: 610, and a VLCDR3 amino acid sequence of SEQ ID NO: 611.

60. The pharmaceutical composition of claim 59, wherein the antibody molecule comprises a VH comprising the amino acid sequence of SEQ ID NO: 606 and a VL comprising the amino acid sequence of SEQ ID NO: 616.

61. The pharmaceutical composition of claim 59, wherein the antibody molecule comprises a heavy chain comprising the amino acid sequence of SEQ ID NO: 608 and a light chain comprising the amino acid sequence of SEQ ID NO: 618.

62. The pharmaceutical composition of claim 59, wherein the antibody molecule comprises a VH comprising the amino acid sequence of SEQ ID NO: 620 and a VL comprising the amino acid sequence of SEQ ID NO: 624.

63. The pharmaceutical composition of claim 59, wherein the antibody molecule comprises a heavy chain comprising the amino acid sequence of SEQ ID NO: 622 and a light chain comprising the amino acid sequence of SEQ ID NO: 626.

64. The pharmaceutical composition of claim 59, wherein the pharmaceutical composition comprises histidine/histidine HCl, sucrose, and Polysorbate 20.

65. The pharmaceutical composition of claim 59, wherein the pharmaceutical composition comprises:
about 25 mg/mL to about 250 mg/mL anti-PD-L1 antibody molecule;
about 1 mg/mL to about 10 mg/mL histidine/histidine HCl;
about 20 mg/mL to about 200 mg/mL sucrose; and
about 0.1 mg/mL to about 1.0 mg/mL Polysorbate 20.

66. The pharmaceutical composition of claim 59, wherein the pharmaceutical composition comprises:
about 80 mg/mL to about 120 mg/mL anti-PD-L1 antibody molecule;
about 2 mg/mL to about 6 mg/mL histidine/histidine HCl;
about 50 mg/mL to about 100 mg/mL sucrose; and
about 0.2 mg/mL to about 0.6 mg/mL Polysorbate 20.

67. The pharmaceutical composition of claim 59, wherein the pharmaceutical composition comprises:
about 100 mg/mL anti-PD-L1 antibody molecule;
about 20 mM histidine/histidine HCl;
about 220 mM sucrose; and
about 0.04% Polysorbate 20.
